SEQUENCE LISTING (1 sequence)

SEQ ID NO: 1 - protein, 13 aa, synthetic construct
KRREILSRRPSYR

(19) United States
(12) Patent Application Publication
(10) Pub. No.: US 2026/0184713 A1
(43) Pub. Date: Jul. 2, 2026

(54) KINASE INHIBITORS

(71) Applicant: The United States of America, as represented by the Secretary, Department of Health and Human Servic, Bethesda, MD (US)

(72) Inventors: Barry R. O'Keefe, Frederick, MD (US); Lin Du, Frederick, MD (US); Brice A.P. Wilson, Catonsville, MD (US); Ping Zhang, Frederick, MD (US); Dongdong Wang, Frederick, MD (US); Juliana Martinez Fiesco, Frederick, MD (US); Ning Li, Clarksburg, MD (US); William J. Moore, Bethesda, MD (US)

(73) Assignee: The United States of America, as represented by the Secretary, Department of Health and Human Servic, Bethesda, MD (US)

(21) Appl. No.: 18/995,318

(22) PCT Filed: Jul. 17, 2023
(86) PCT No.: PCT/US2023/070304

§ 371 Date: Jan. 16, 2025

Related U.S. Application Data
(60) Provisional application No. 63/389,937, filed on Jul. 17, 2022.

(51) Int. Cl. C07D 473/34; A61K 31/52; A61K 31/522; A61K 31/541

(52) U.S. Cl. CPC C07D 473/34; A61K 31/52; A61K 31/522; A61K 31/541

(57) ABSTRACT

Disclosed is a class of kinase inhibitors. Related pharmaceutical compositions and methods of making and using the kinase inhibitors are also disclosed.

### CROSS-REFERENCE TO PRIOR APPLICATION

This application claims benefit to U.S. Provisional Patent Application No. 63/389,937, filed Jul. 17, 2022, which is hereby incorporated by reference in its entirety.

### FEDERALLY SPONSORED RESEARCH OR DEVELOPMENT

This invention was made with Government support under project number Z01ZIABC011744 by the National Institutes of Health, National Cancer Institute and project number Z01ZIABC011854 by the Cancer Moonshot NCI Program for Natural Product Discovery. The Government has certain rights in this invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 1,926 Byte Extensible Markup Language (XML) file named “767696.xml,” created on Jul. 12, 2023.

### BACKGROUND OF THE INVENTION

Mammals have enzymes called kinases that are associated with cell functions such as cell signaling, metabolism, and division. Some kinases have been found to be more active in certain types of cancers. Blocking the kinases associated with cancer growth may provide therapeutic advantages to those suffering from cancer. Given that cancer is currently a major health concern and that there is a lack of effective treatments against all cancers, there is an urgent need to identify new kinase inhibitors to treat cancers. There is also an urgent need to identify kinase inhibitors associated with non-cancer pathologies (e.g., infections) in order to treat conditions and disorders associated with non-cancer pathologies.

### BRIEF SUMMARY OF THE INVENTION

An aspect of the invention provides compounds of formula (I)

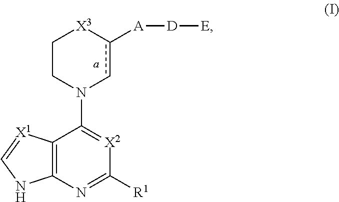

wherein X¹, X², X³, [symbol] , R¹, A, D, and E are defined herein, or a pharmaceutically acceptable salt thereof.

An additional aspect of the invention provides pharmaceutical compositions comprising compounds of aspects of the present invention.

Another aspect of the invention provides methods of inhibiting kinase activity in a subject, the methods comprising administering to the subject compounds or pharmaceutical compositions of aspects of the present invention.

A further aspect of the invention provides methods of suppressing the immune system in a subject, the methods comprising administering to the subject compounds or pharmaceutical compositions of aspects of the present invention.

An additional aspect of the invention provides methods of preventing organ rejection in a subject, the methods comprising administering to the subject compounds or pharmaceutical compositions of aspects of the present invention.

A further aspect of the invention provides methods of treating cancer in a subject, the methods comprising administering to the subject compounds or pharmaceutical compositions of aspects of the present invention.

An additional aspect of the invention provides methods of treating diabetic neuropathic pain in a subject, the method comprising administering to the subject compounds or pharmaceutical compositions of aspects of the present invention.

A further aspect of the invention provides methods of treating malaria in a subject, the method comprising administering to the subject compounds or pharmaceutical compositions of aspects of the present invention.

An additional aspect of the invention provides methods of treating an infection associated with a protozoa in a subject, the method comprising administering to the subject compounds or pharmaceutical compositions of aspects of the present invention.

Another aspect of the invention provides methods of making the compounds of aspects of the present invention.

### BRIEF DESCRIPTION OF THE SEVERAL VIEWS OF THE DRAWING(S)

FIG. 1A is a graph showing the percentage of normalized activities of PKADJ and wild-type PKA (WT) that are treated with Aplithianine A (183A034G PKADJ and 183A034G WT) and Aplithianine B (183A034H PKADJ and 183A034H WT).

FIG. 1B is a graph showing the percentage of normalized activity of PKADJ that is treated with Aplithianine A (Compound 1) and Aplithianine B (Compound 2).

FIG. 2A is a graph showing the percentage of activity of Aplithianine A (183A034G) in the Luciferase assay with or without (Luciferase Only) PKA as the target receptor.

FIG. 2B is a competitive kinetic study to investigate how Aplithianine A interacted with the PKA protein

FIG. 3A shows an example of Ribbon display of a co-crystal structure of Compound 1 and PKADJ.

FIG. 3B shows an example of the ATP binding pocket in the PKADJ crystal structure (PDB #: 4WB7).

FIG. 3C shows an example of the aplithianine A binding pocket in its co-crystal structure with PKADJ.

FIG. 3D shows an example of the aplithianine B binding pocket in its co-crystal structure with PKADJ.

FIG. 4A is a graph showing the normalized activities of PKADJ and wild-type PKA (WT) that are treated with Aplithianine A₁ (183A041B).

FIG. 4B is a graph showing the normalized activities of PKADJ and wild-type PKA (WT) that are treated with Aplithianine A₂ (183A041E).

FIG. 4C is a graph showing the normalized activities of PKADJ and wild-type PKA (WT) that are treated with Aplithianine A₃ (183A041D).

FIG. 4D is a graph showing the normalized activities of PKADJ and wild-type PKA (WT) that are treated with Aplithianine A₄ (183A041C).

FIGS. 5A-5C are an example of kinase mapping of a compound of the present invention. The larger circles indicate more potency. Aplithianine A (1)/183A034G was tested at two concentrations (2 μM and 50 nM) against a panel of 370 human protein kinases. The top 50 hits from the 50 nM test were highlighted on a human kinome tree that was generated using the web application CORAL. The node of PKG1β represents the data for PKG1α. The data for PKG1β is not shown.

FIG. 6A is a graph showing the percentage of normalized activities of ten selected kinases that are treated with Aplithianine A (183A034G).

FIG. 6B is a graph showing the IC₅₀ of Aplithianine A (183A034G) and Aplithianine A₁ (183A041B) against ten selected kinases.

FIG. 6C is another graph showing the IC₅₀ of Aplithianine A (183A034G) and Aplithianine A₁ (183A041B) against 4 selected kinases.

FIG. 7 is an example of synthesis scheme of the total synthesis of aplithianine A.

FIG. 8 is an example of another synthesis scheme of the total synthesis of aplithianine A.

FIG. 9A is the structure of compound 183A041B.

FIG. 9B is the structure of compound 183A041C.

FIG. 9C is the structure of compound 183A041D.

FIG. 9D is the structure of compound 183A041E.

FIG. 9E is a graph showing the percentage of normalized activities of PKADJ and wild-type PKA (WT) that are treated with natural aplithianine A.

FIG. 9F is a graph showing the percentage of normalized activities of PKADJ and wild-type PKA (WT) that are treated with synthetic aplithianine A.

FIG. 9G is a graph showing the percentage of normalized activities of PKADJ and wild-type PKA (WT) that are treated with 183A041B.

FIG. 9H is a graph showing the percentage of normalized activities of PKADJ and wild-type PKA (WT) that are treated with 183A041C.

FIG. 91 is a graph showing the percentage of normalized activities of PKADJ and wild-type PKA (WT) that are treated with 183A041D.

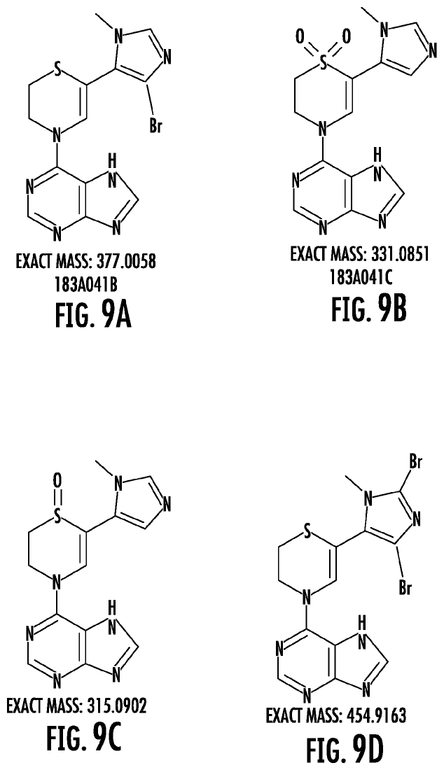

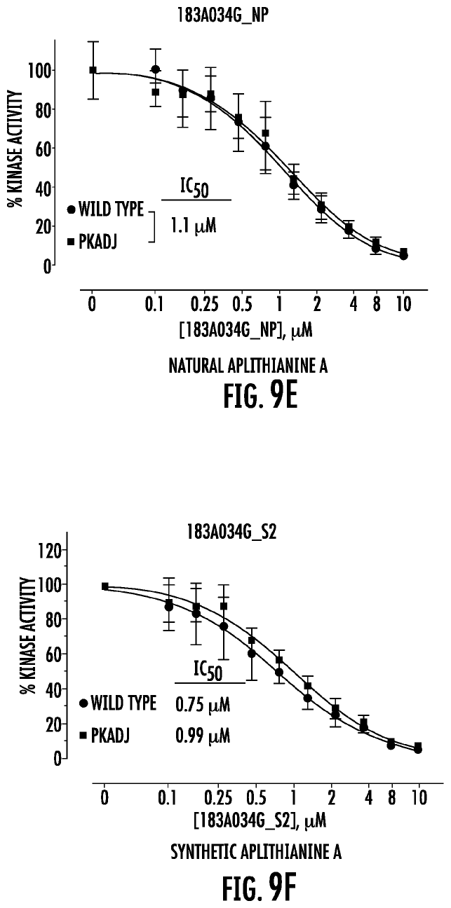

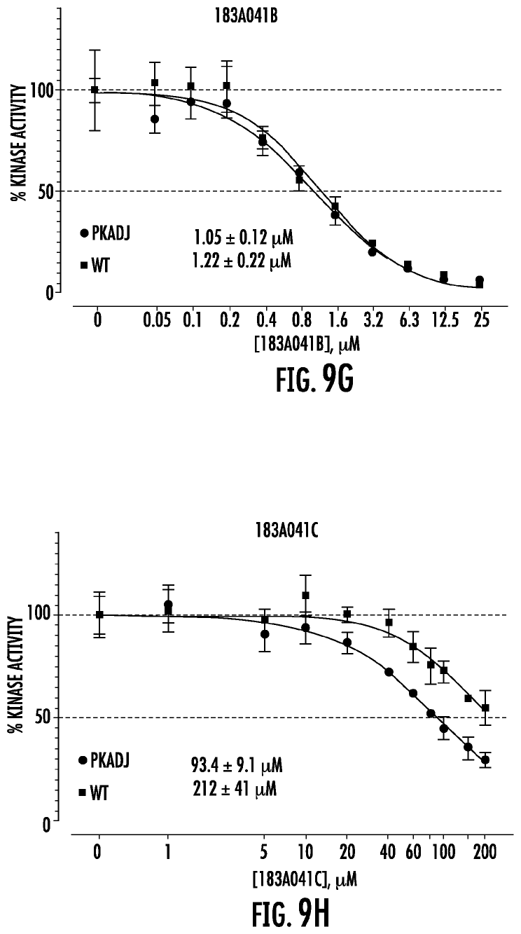

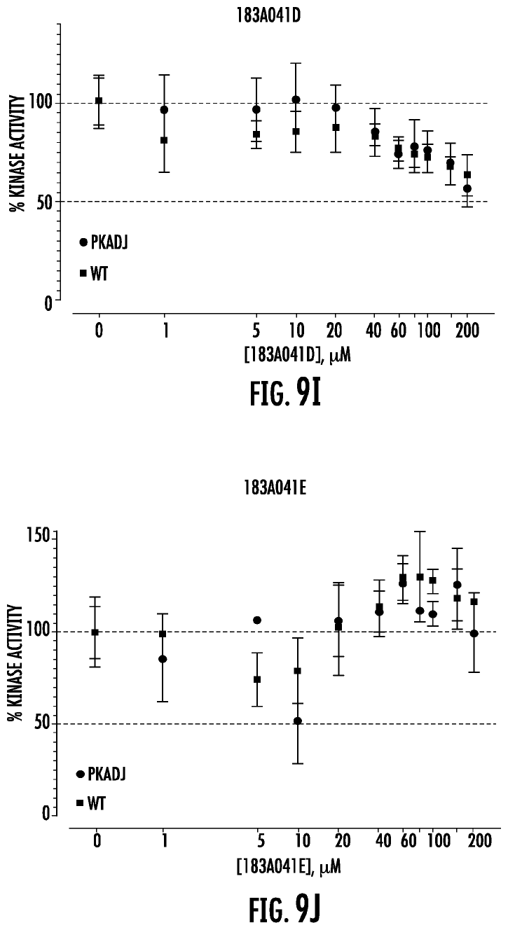

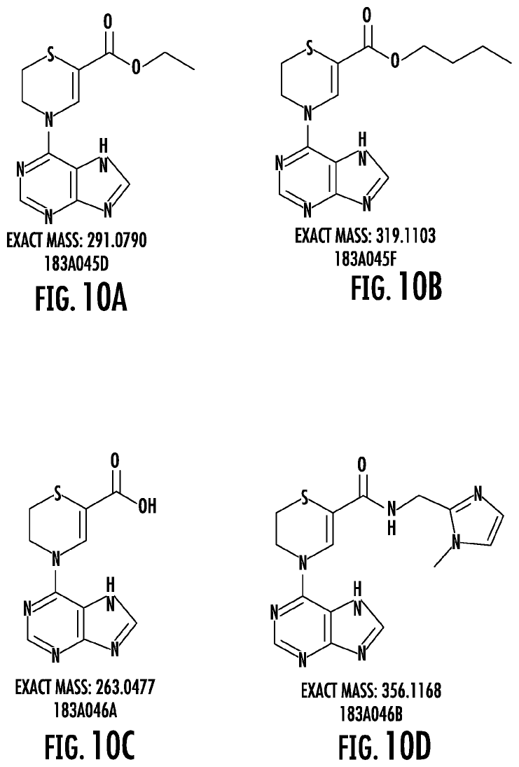

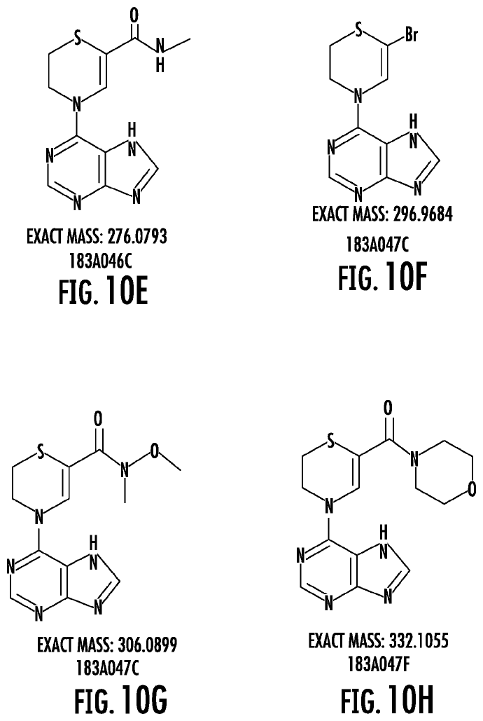

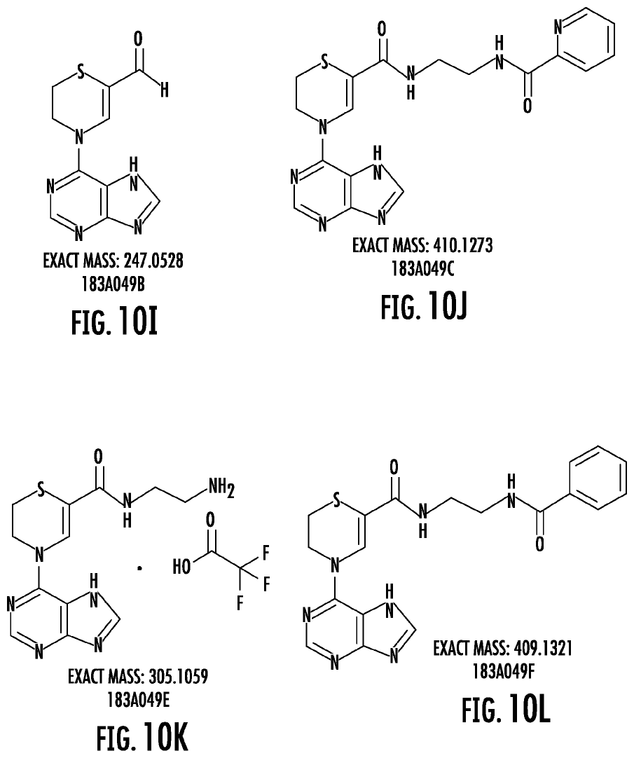

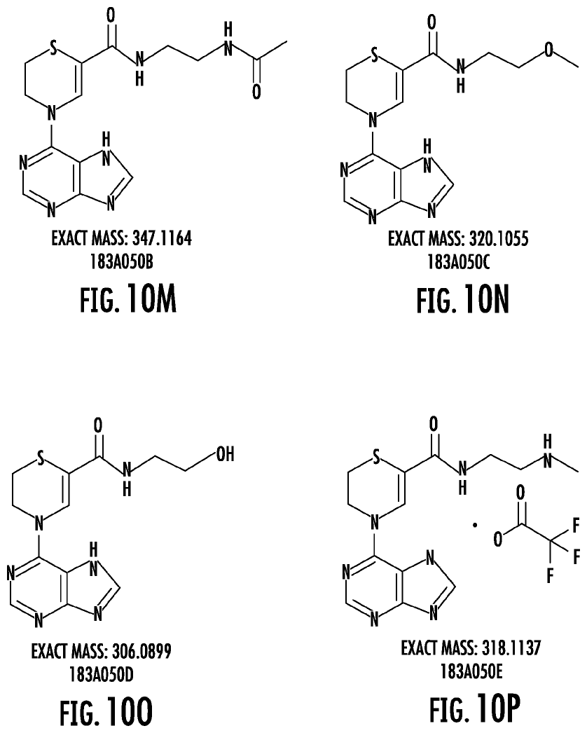

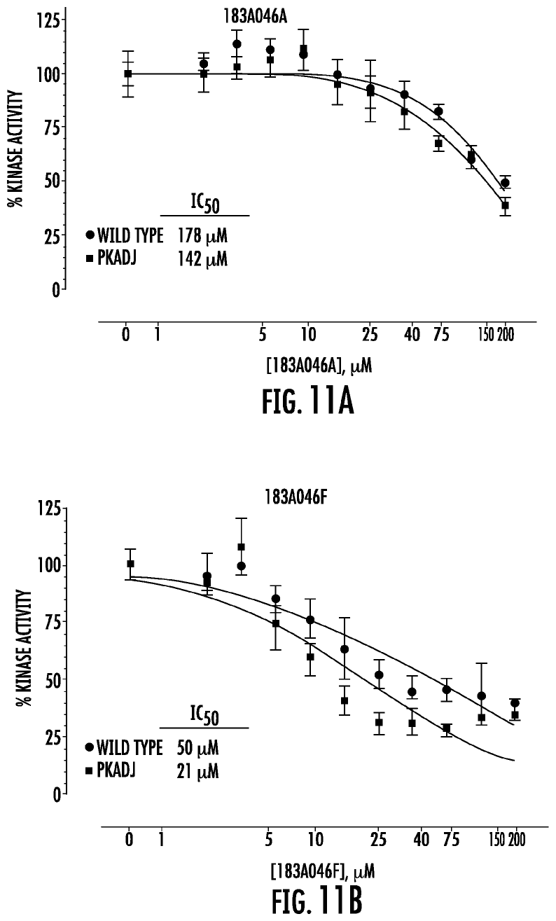

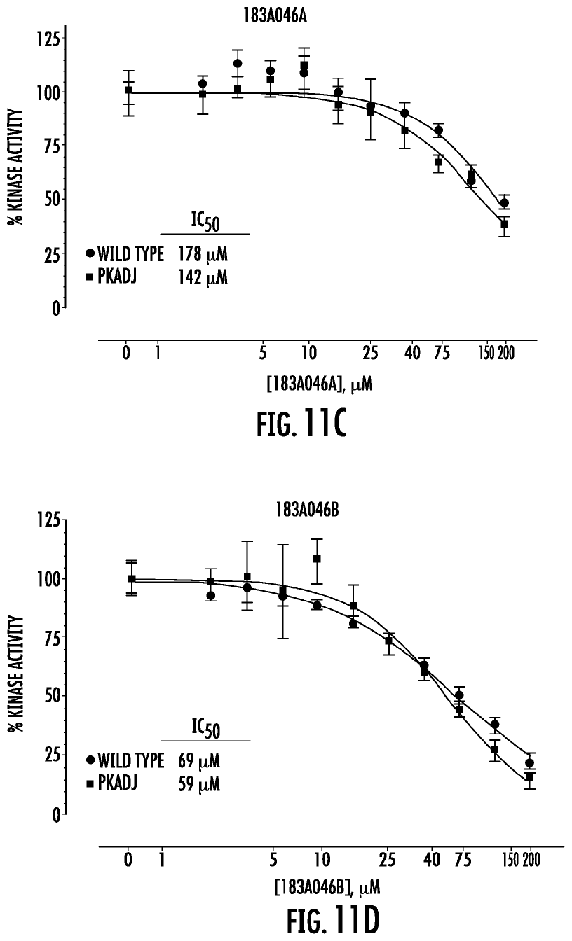

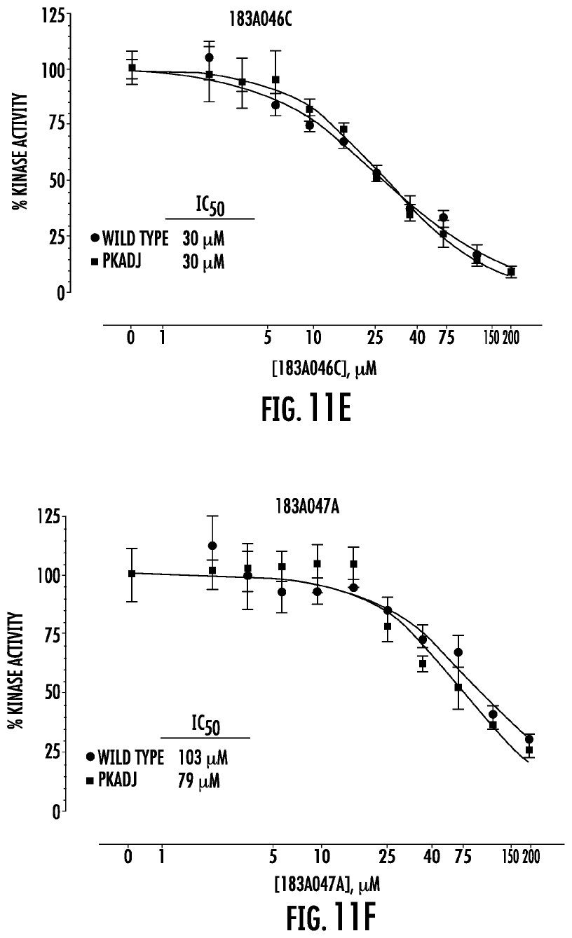

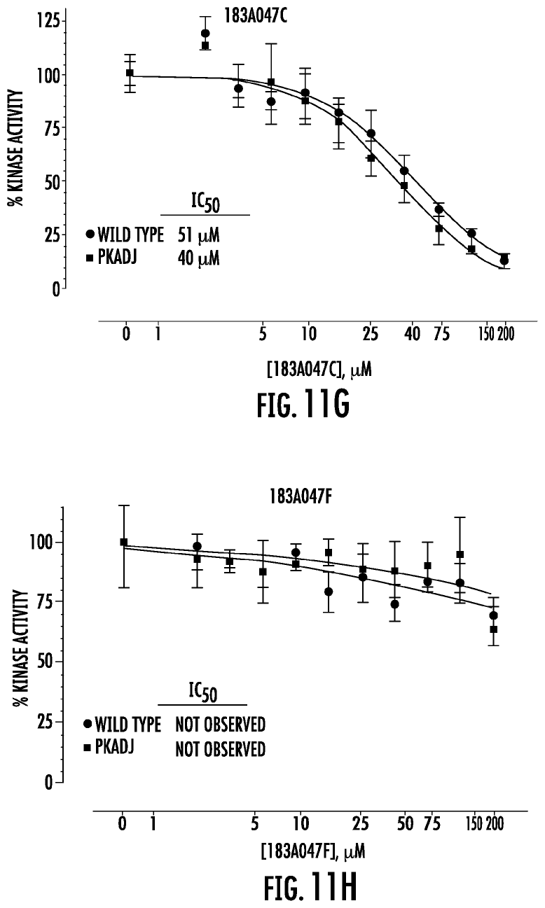

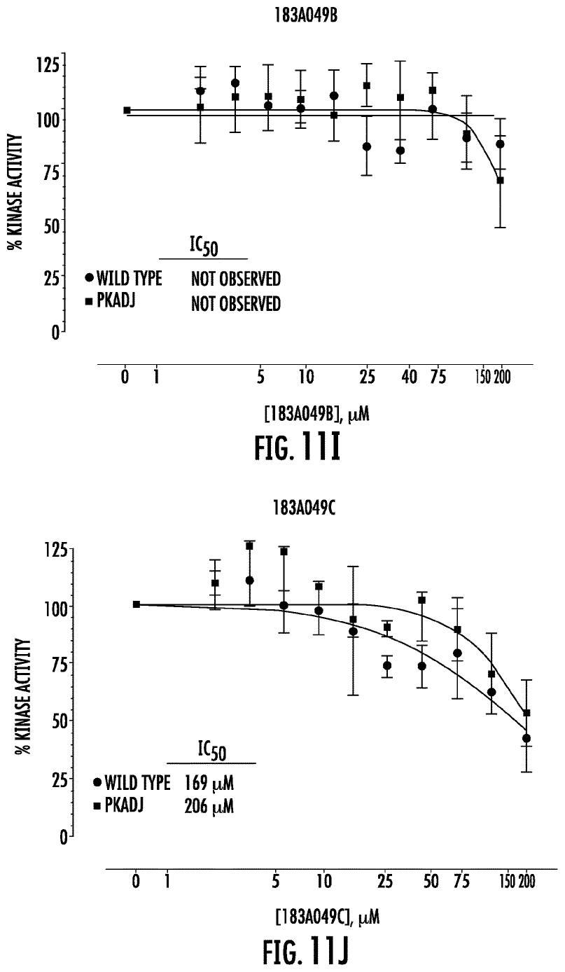

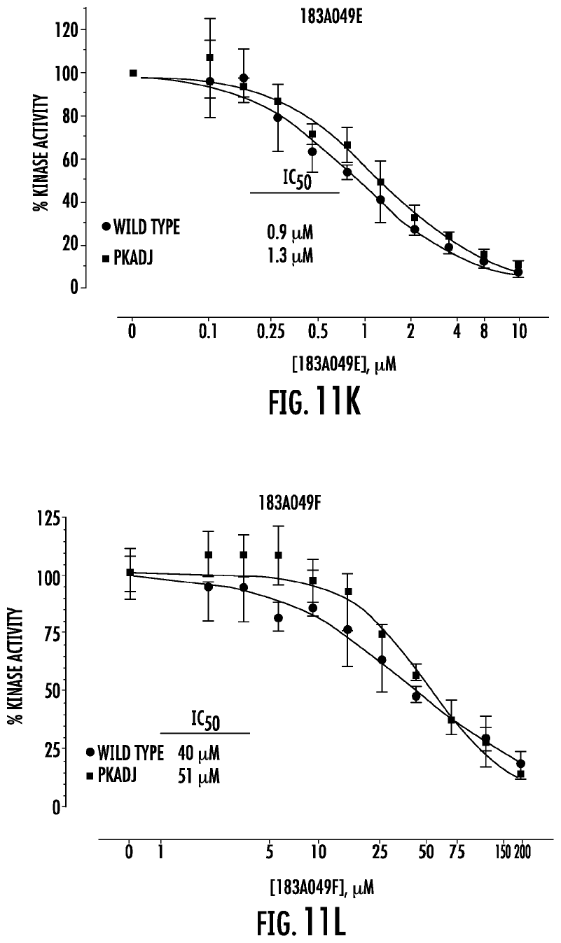

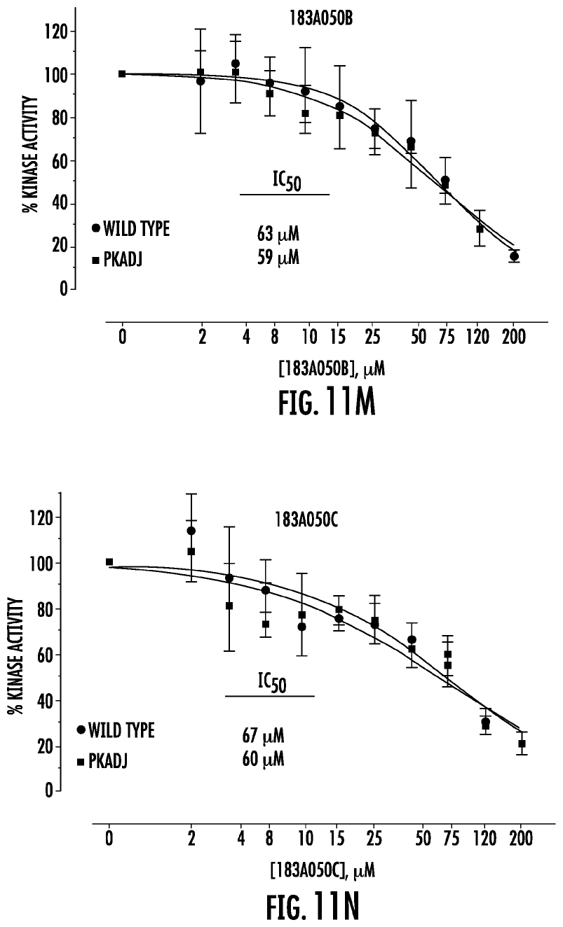

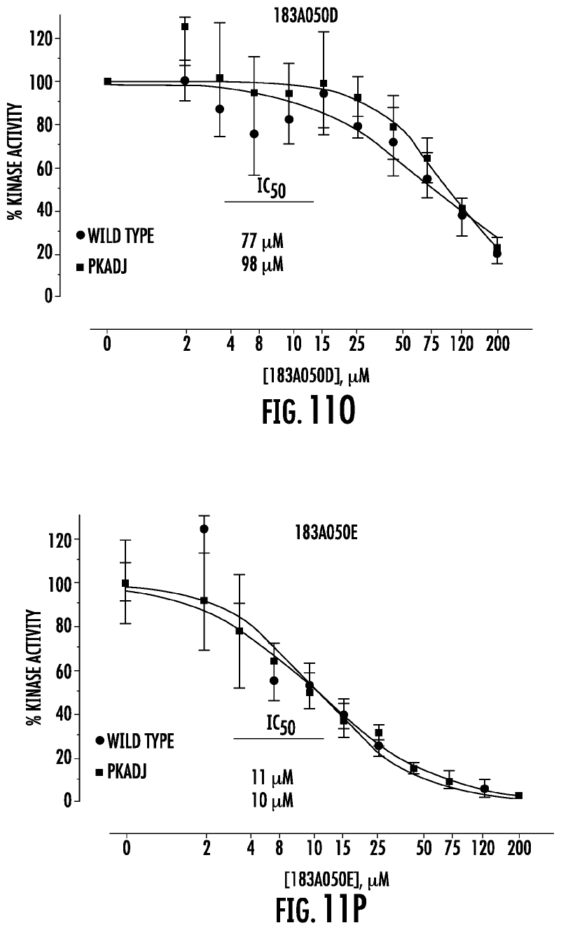

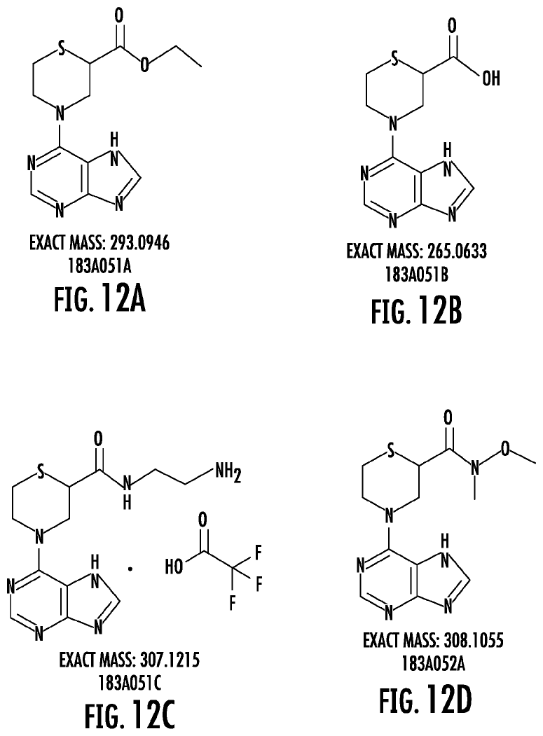

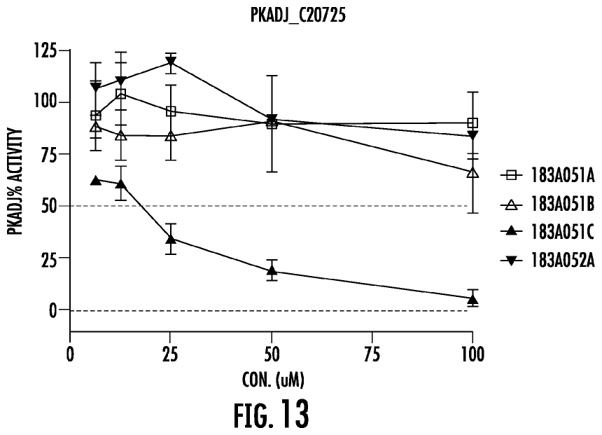

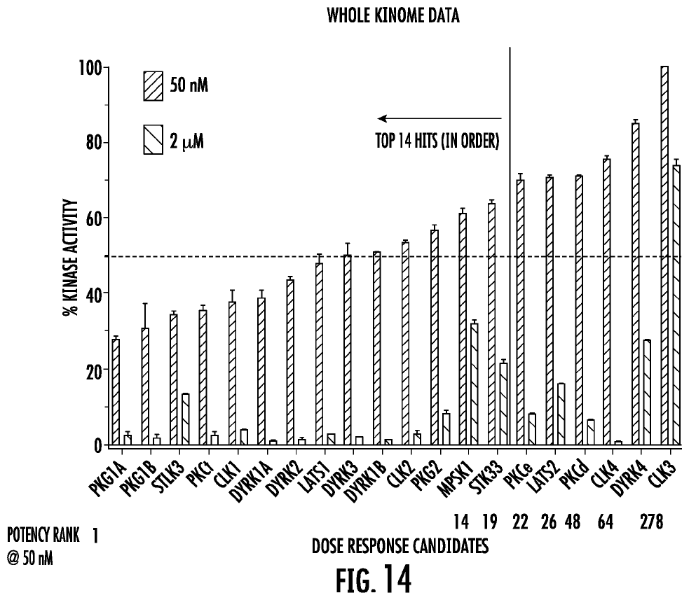

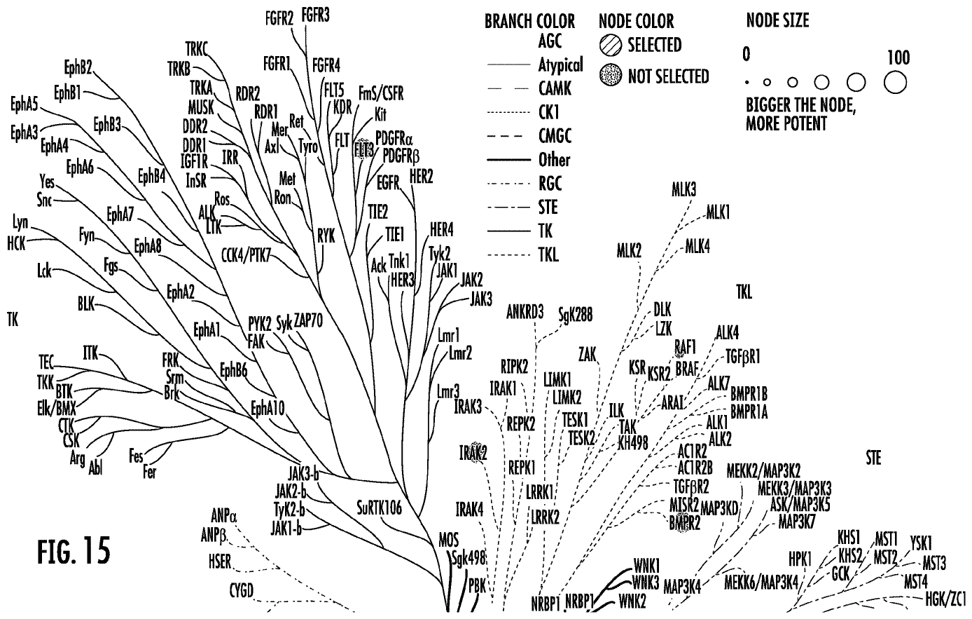

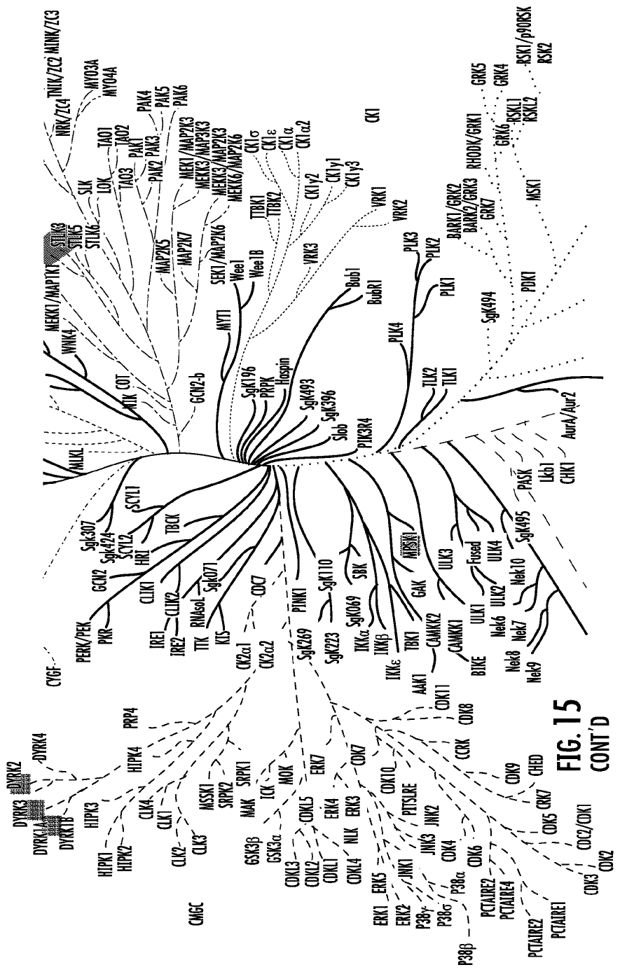

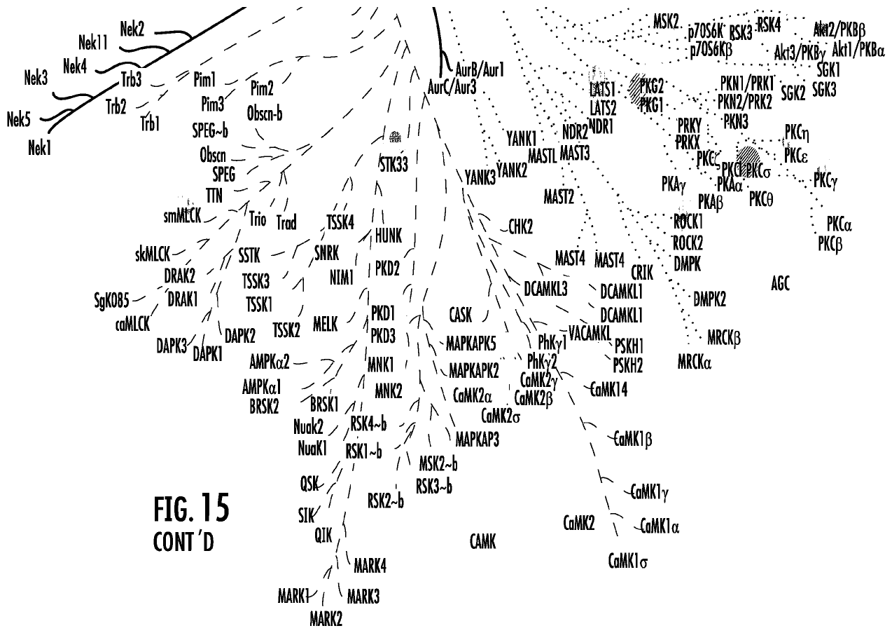

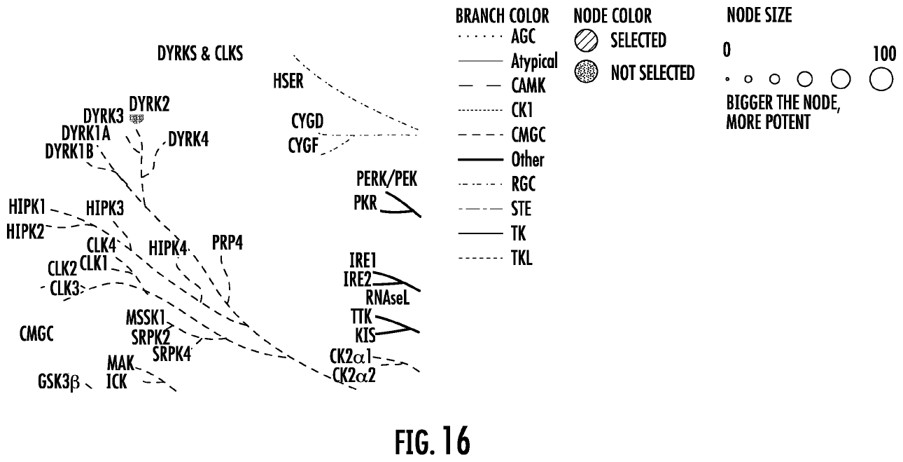

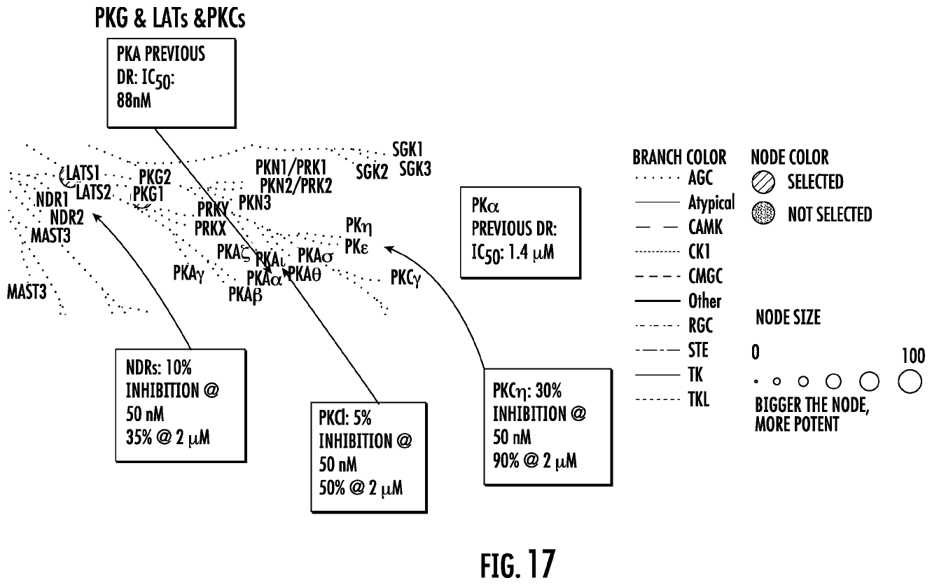

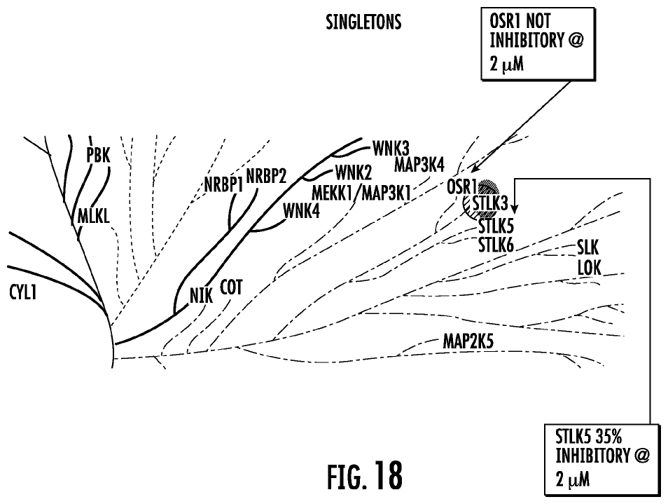

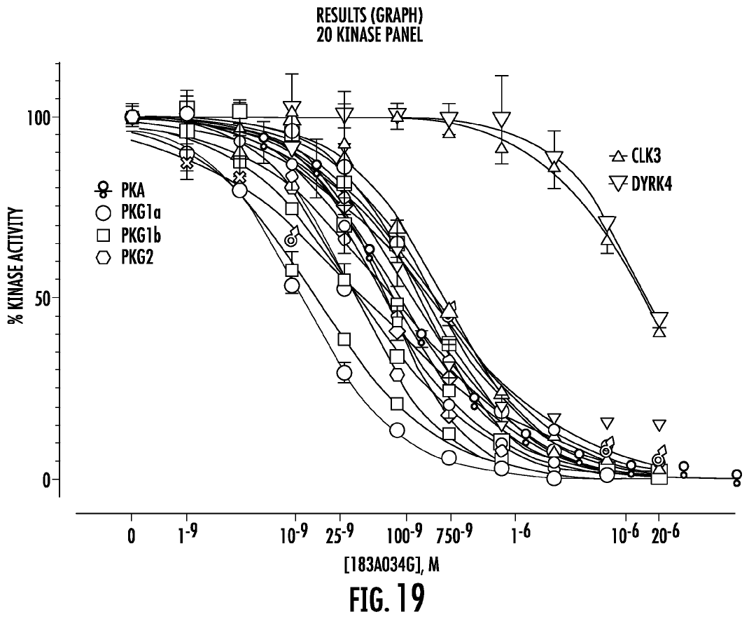

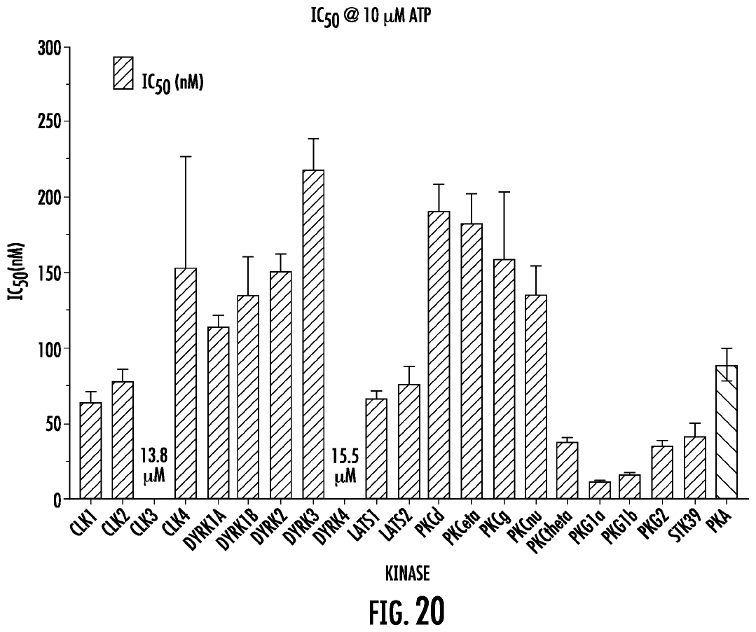

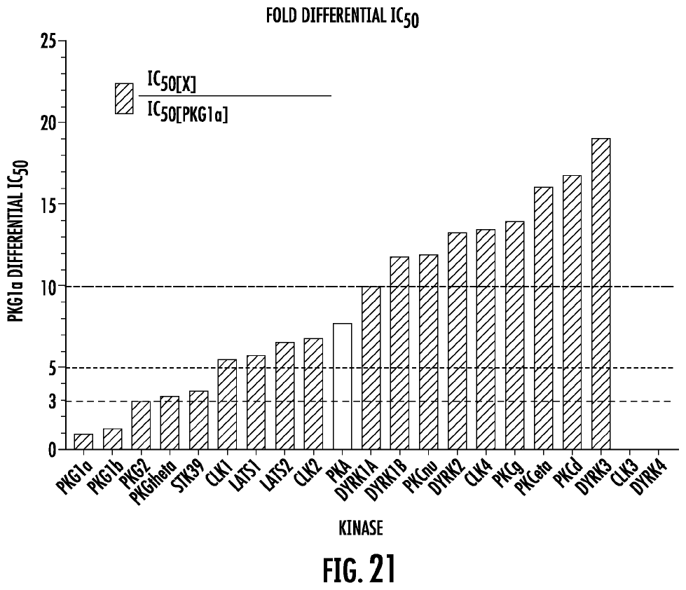

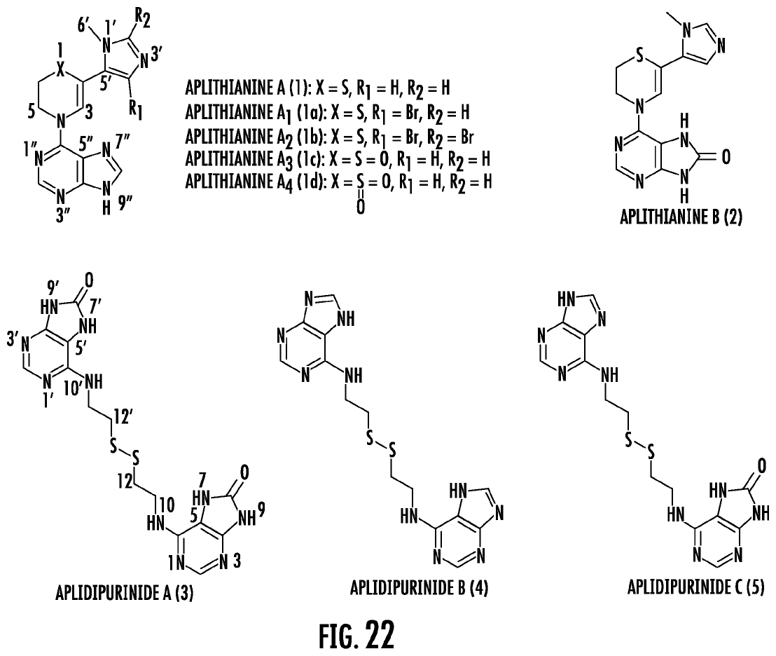

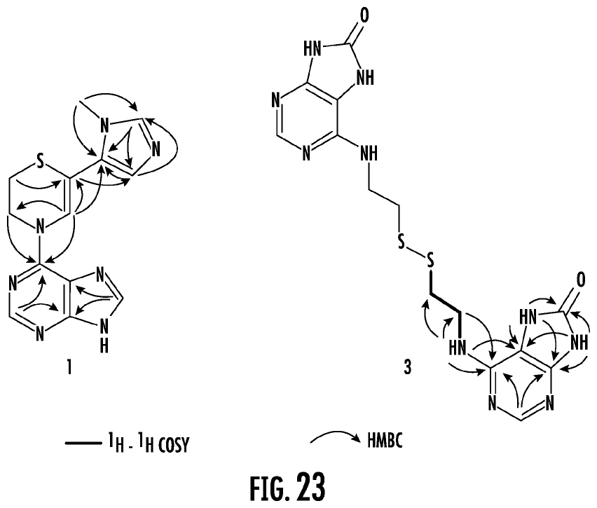

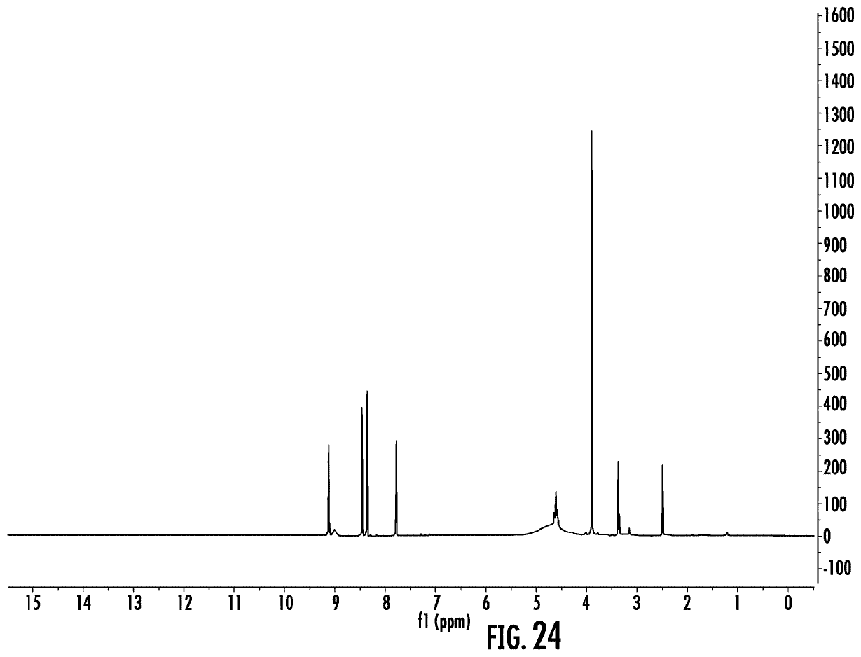

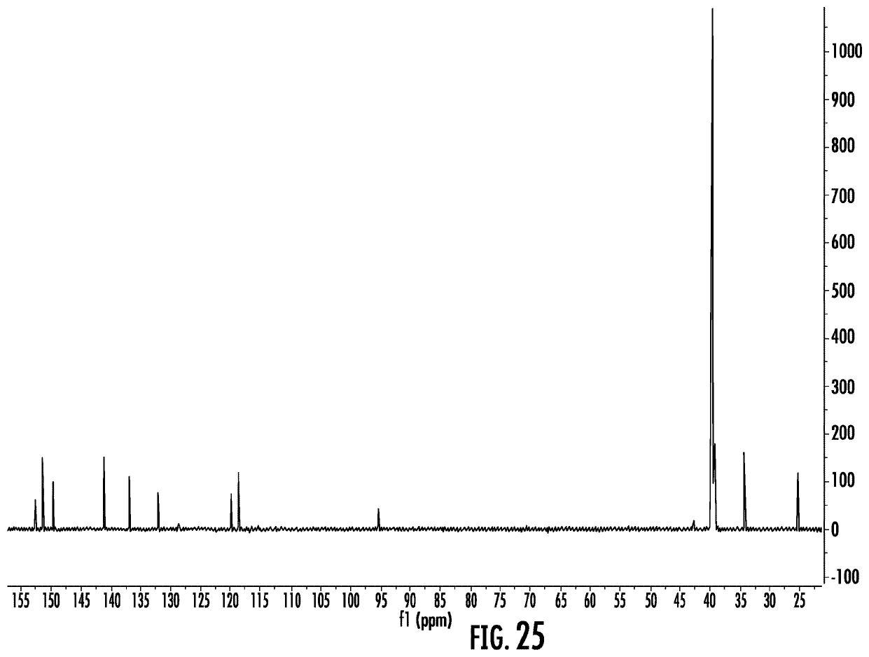

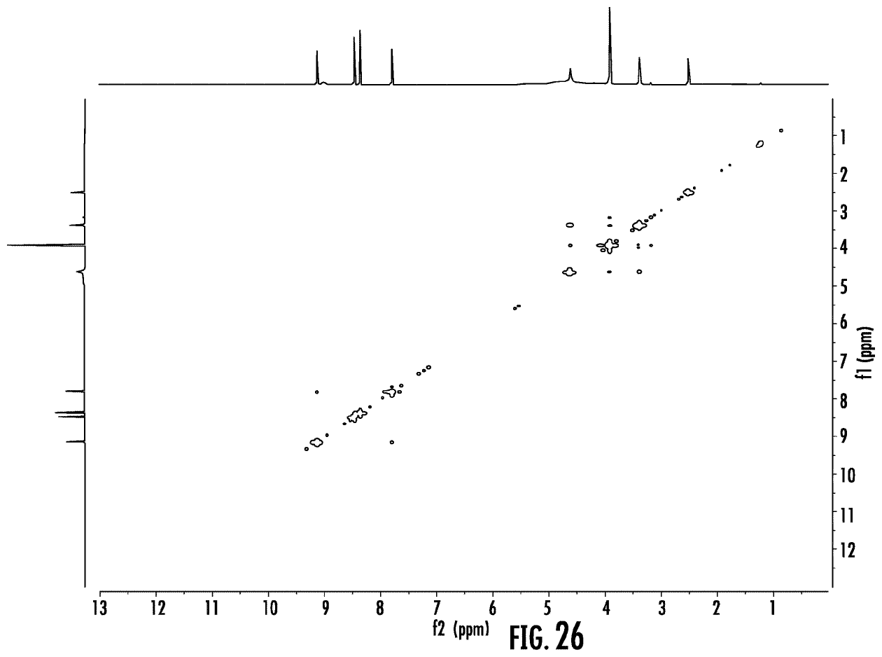

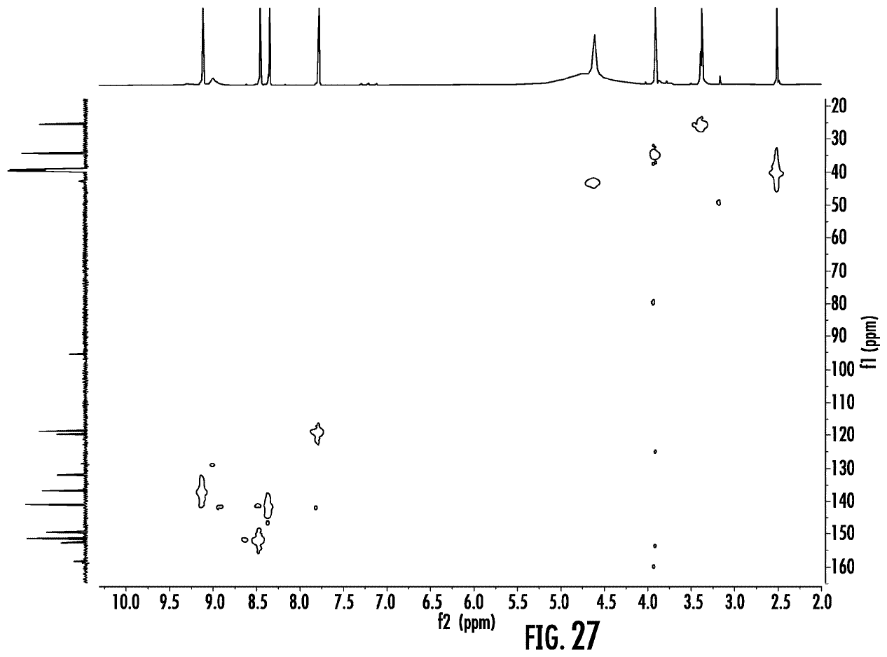

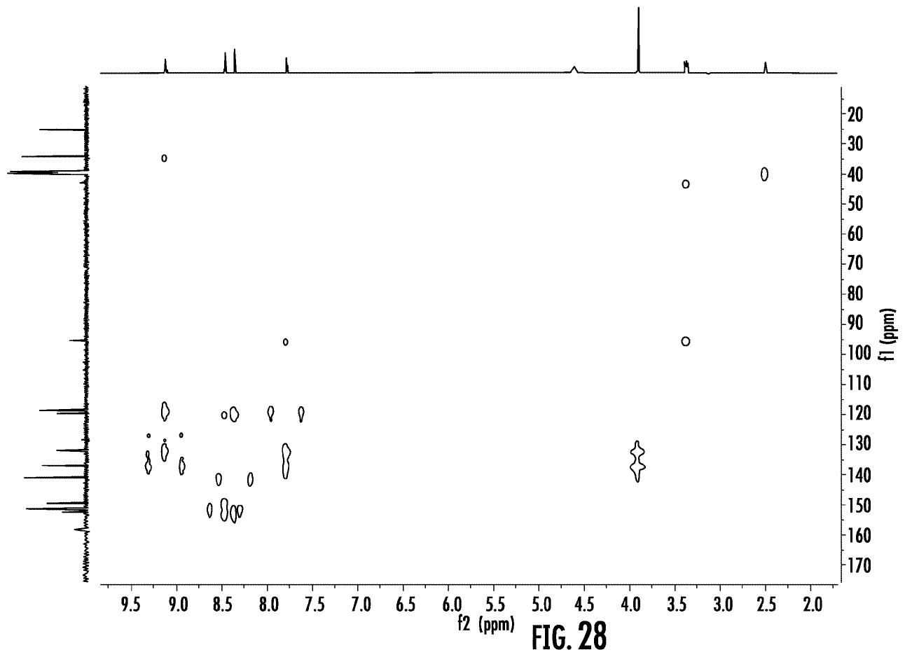

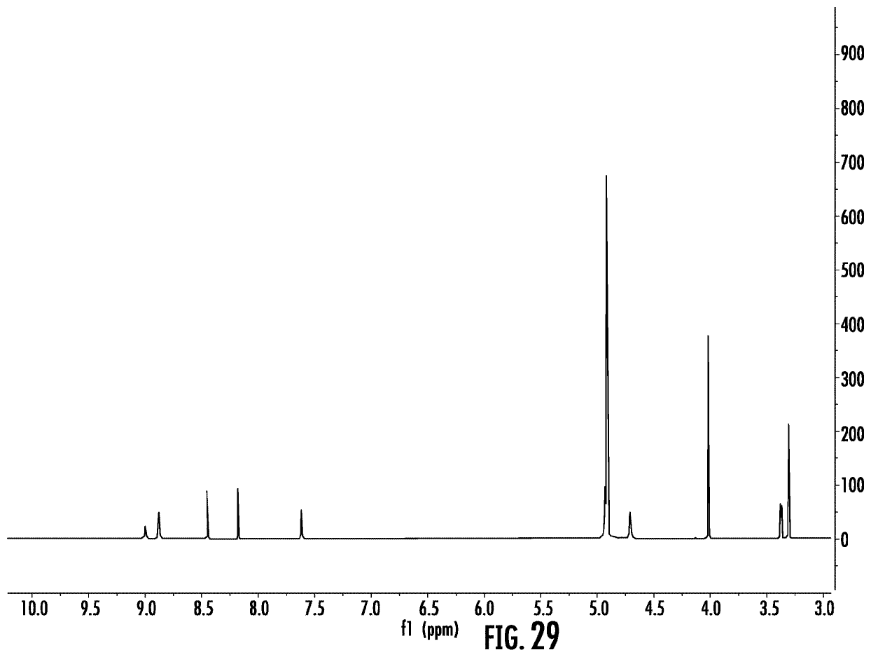

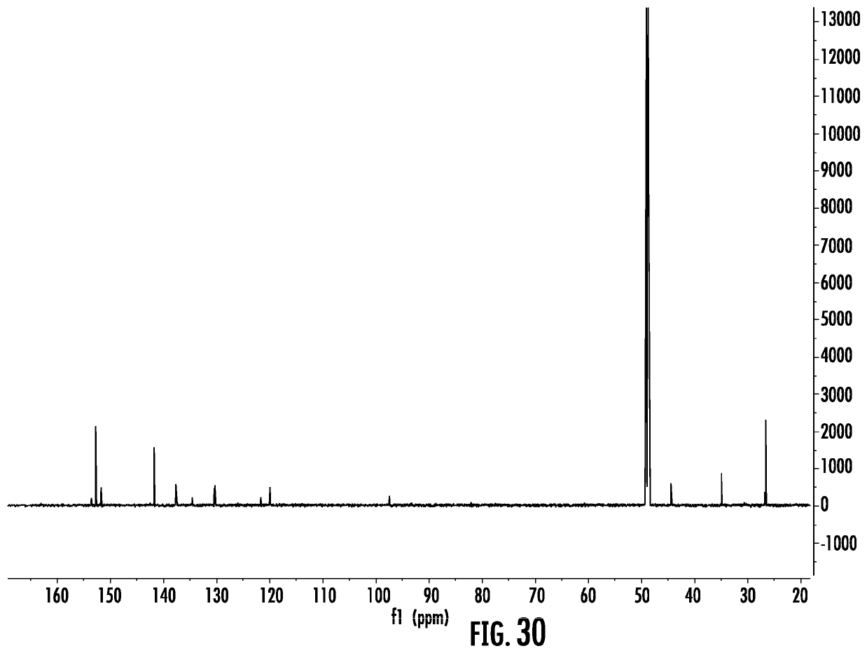

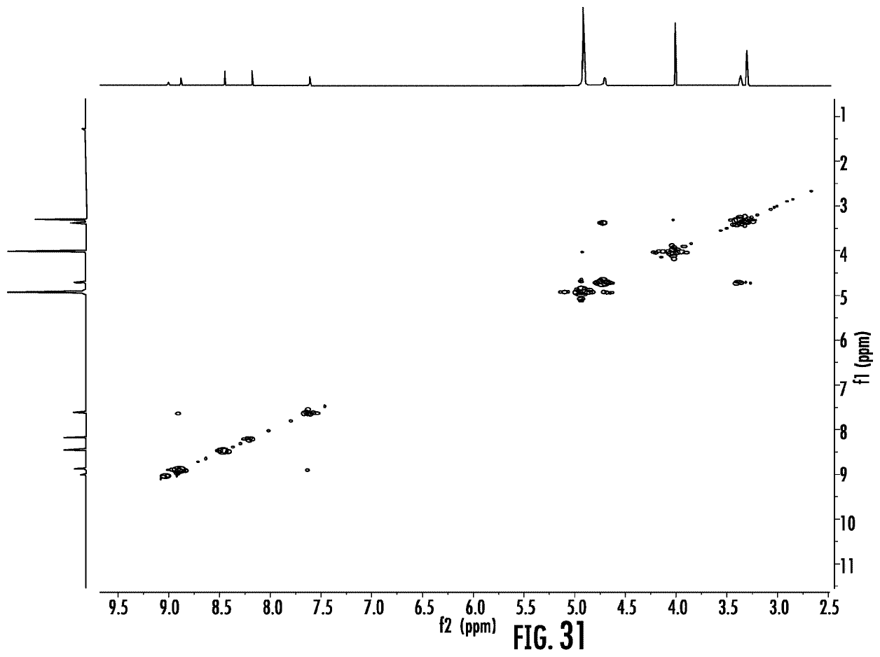

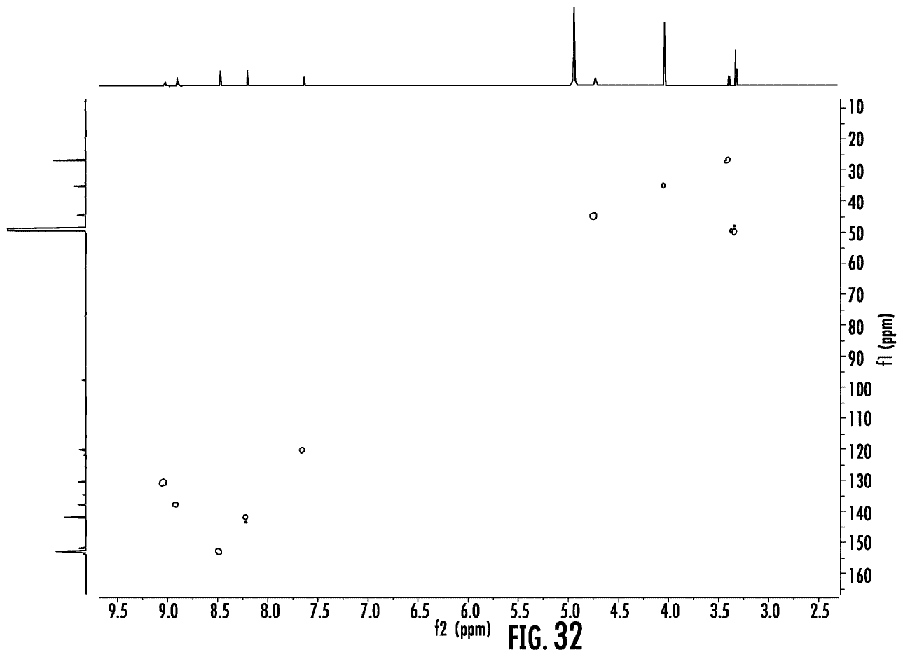

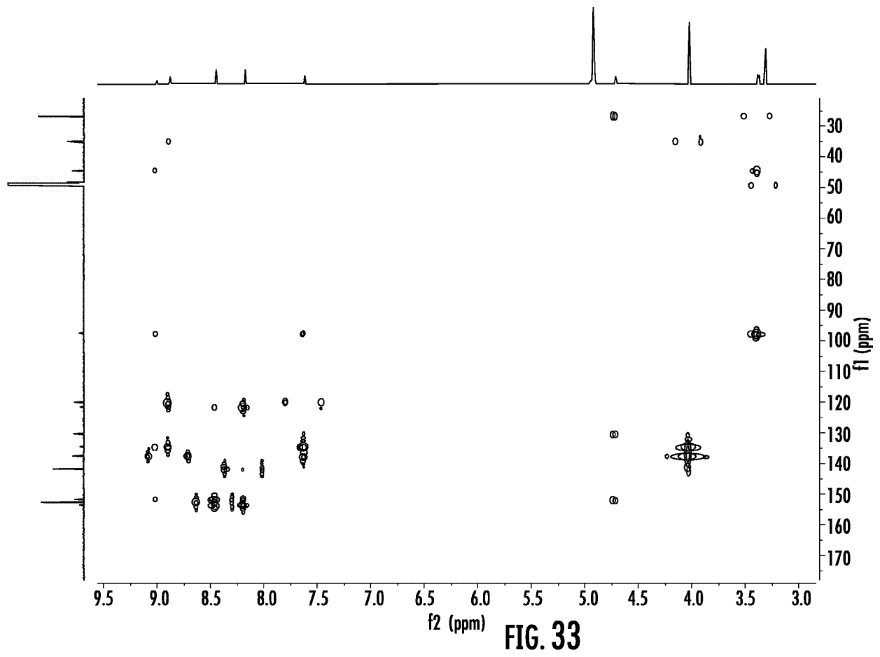

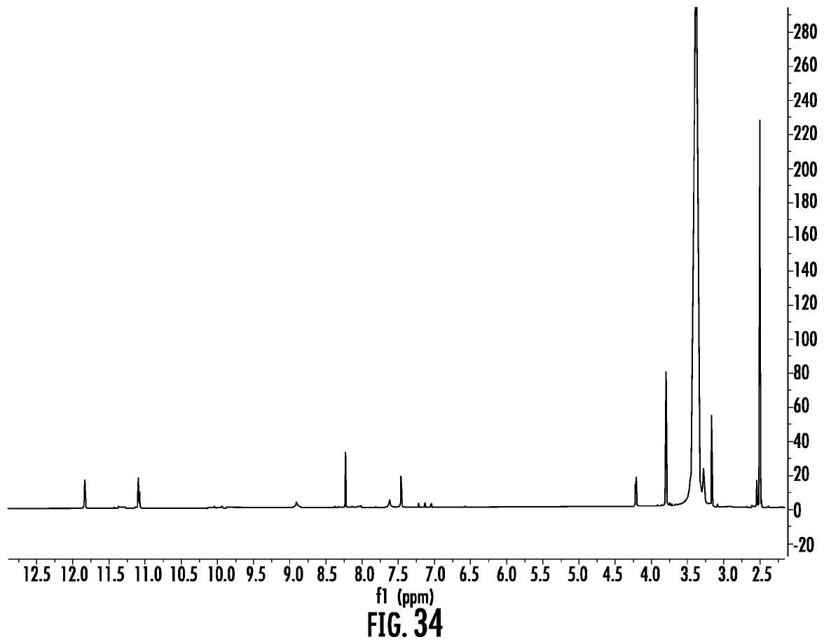

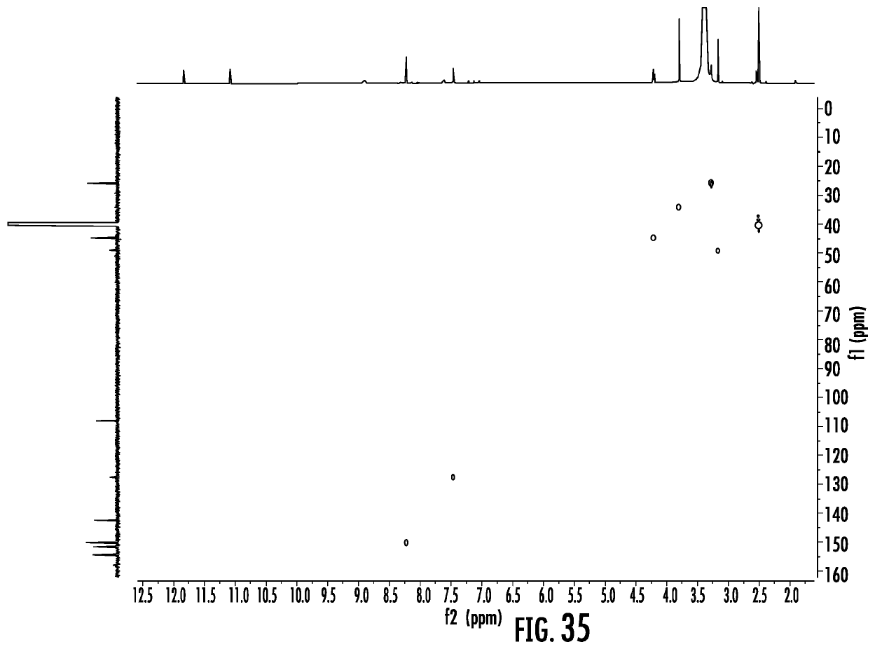

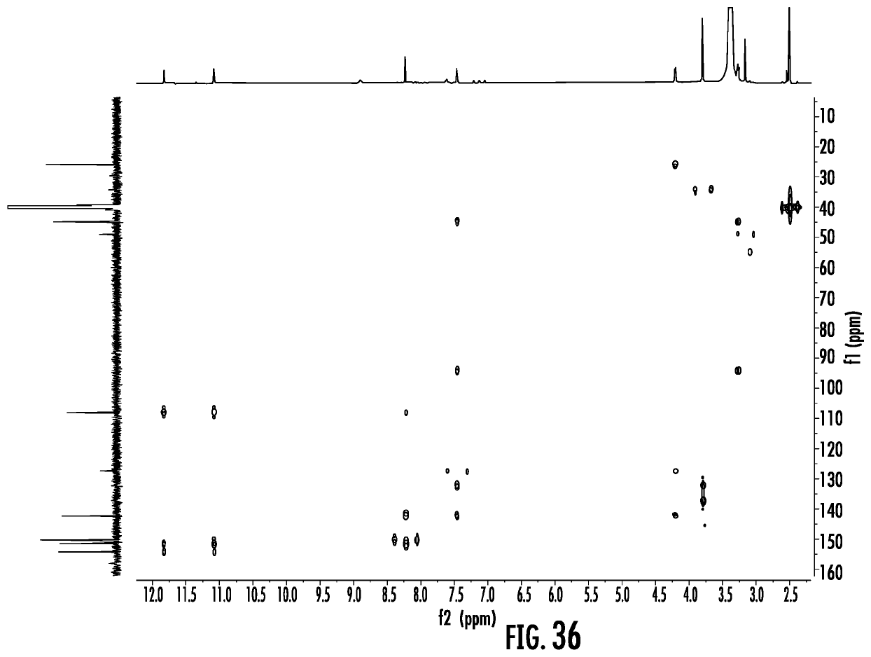

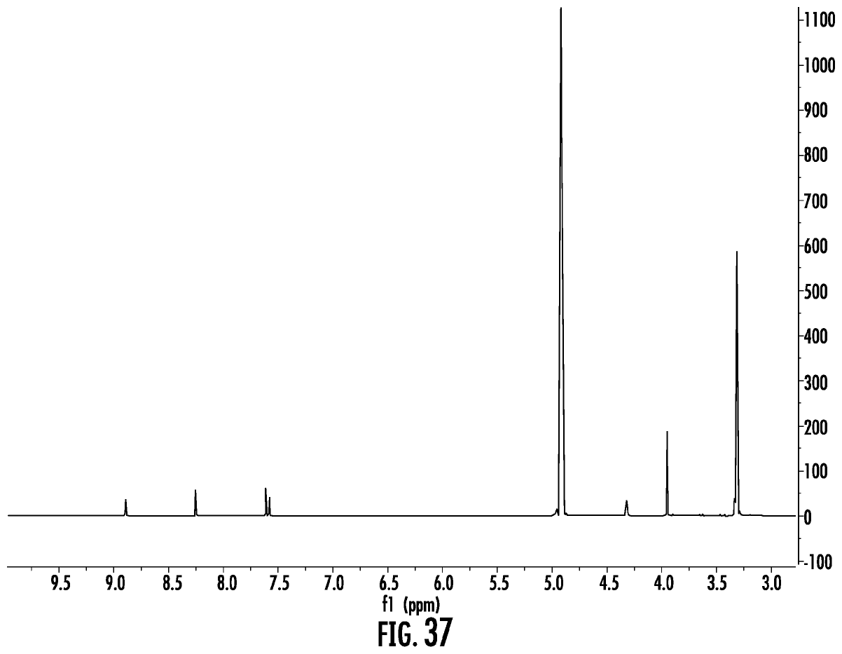

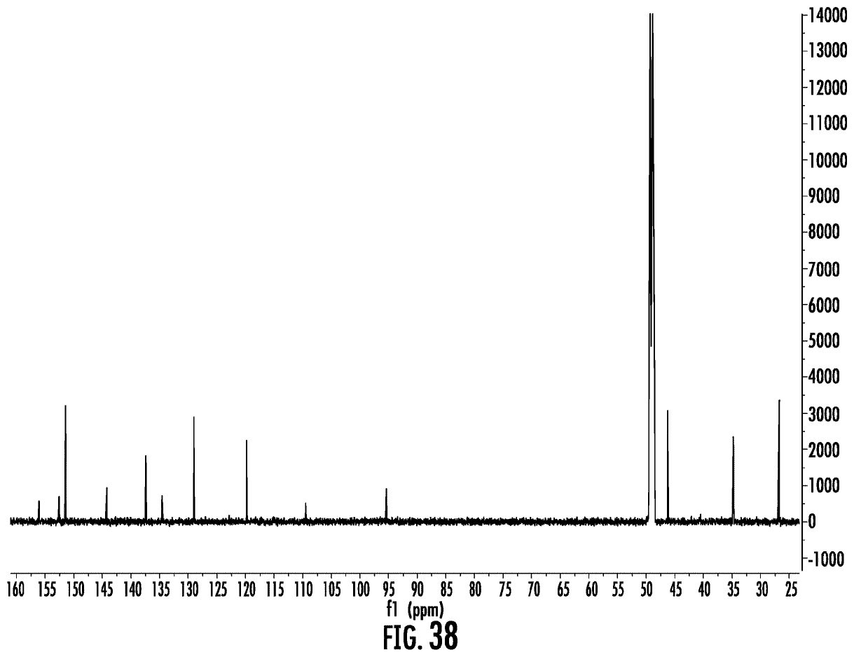

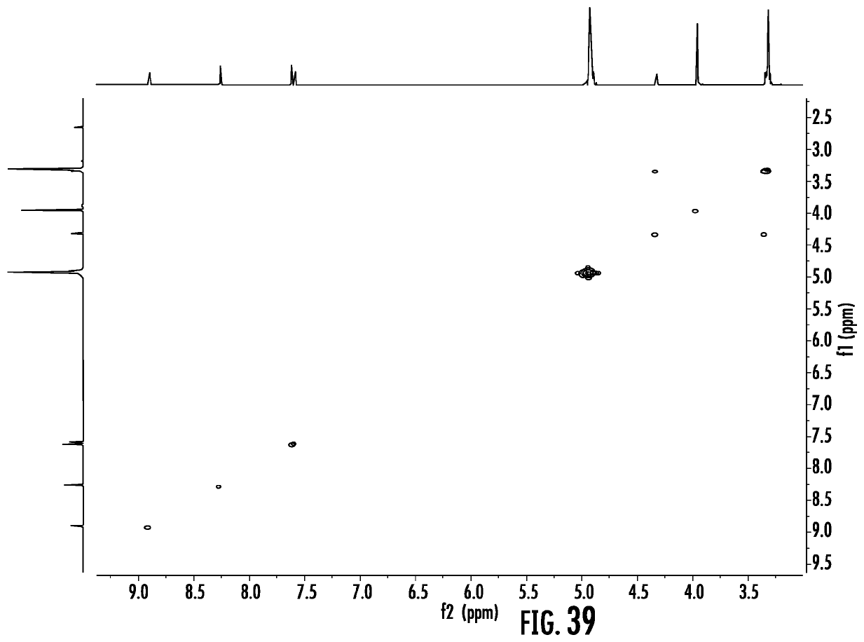

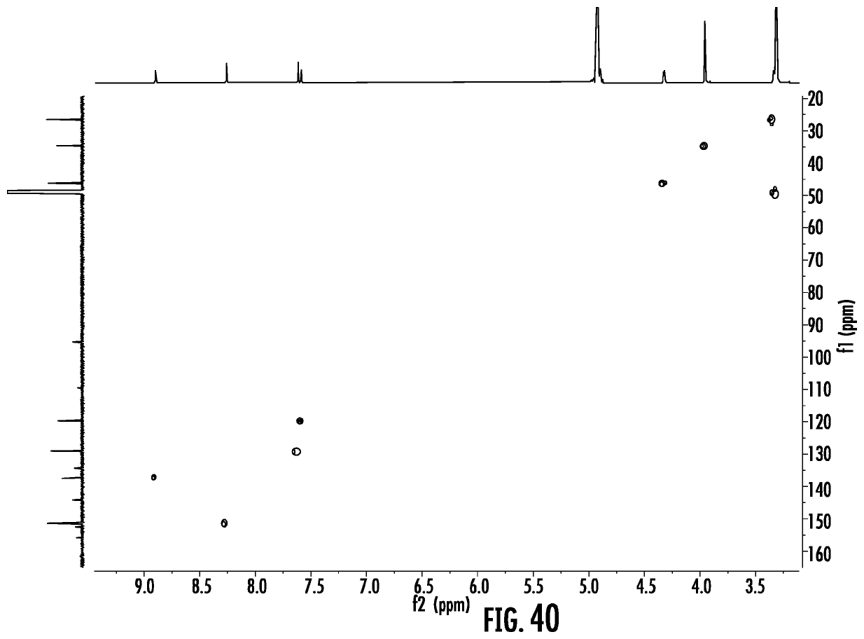

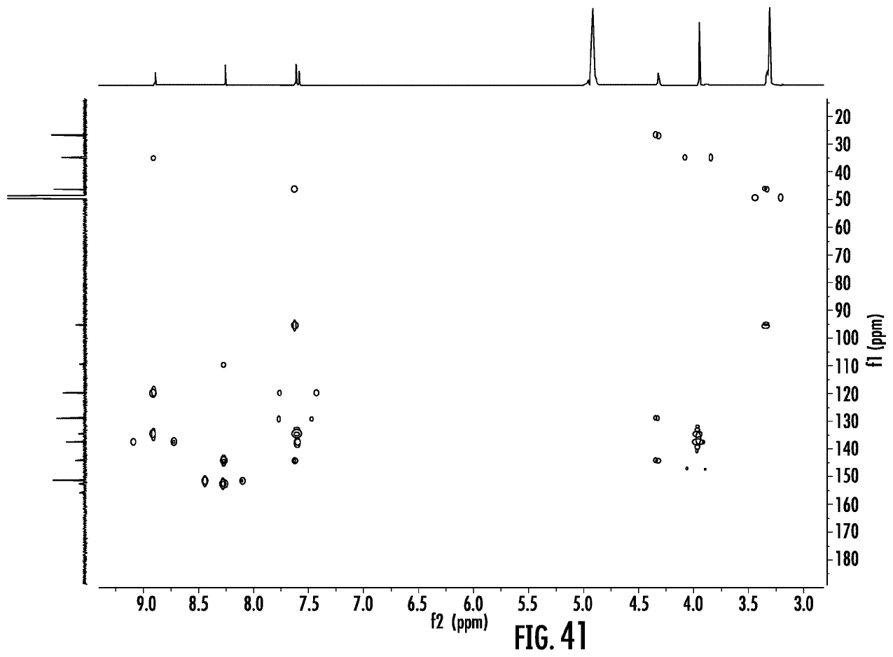

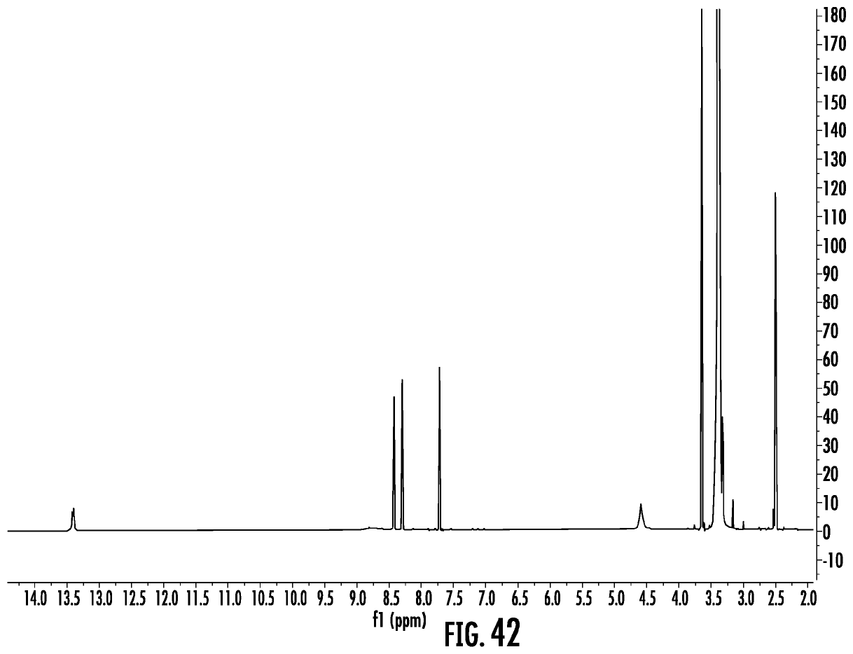

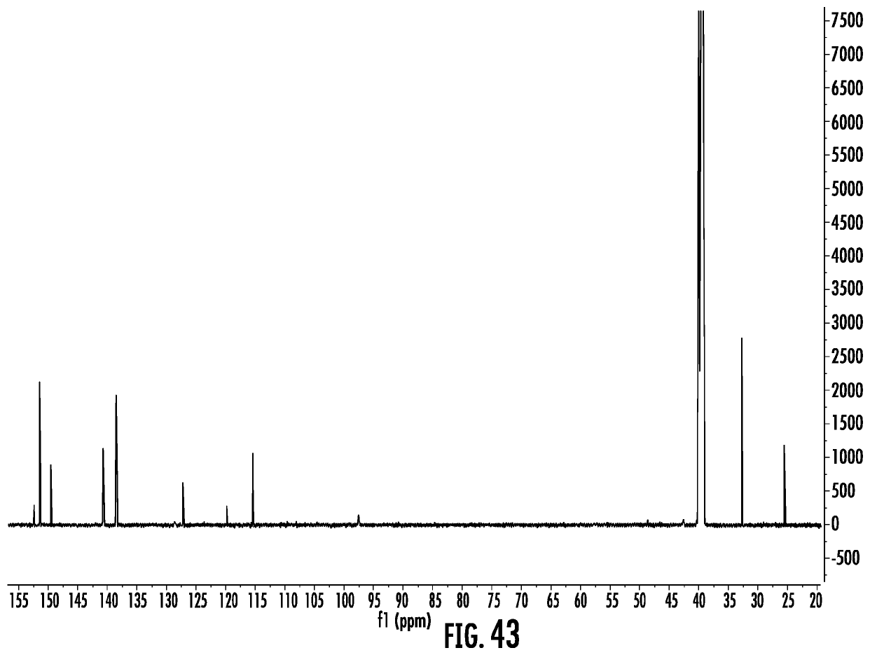

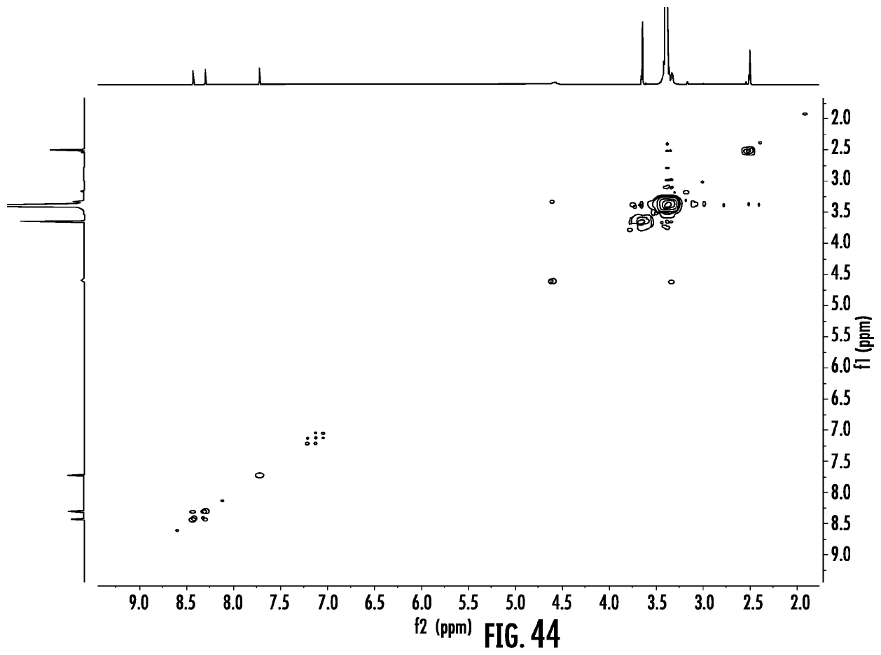

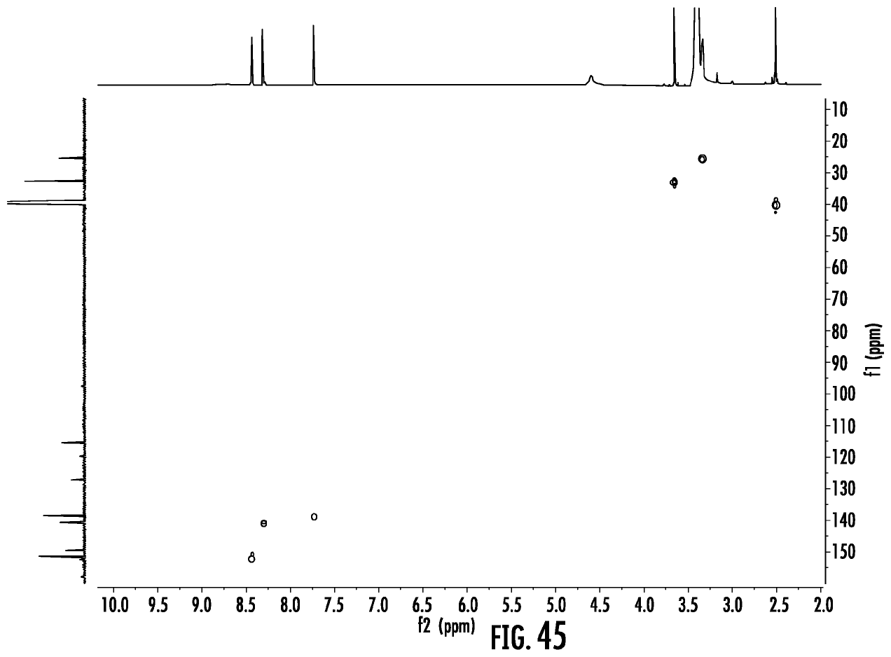

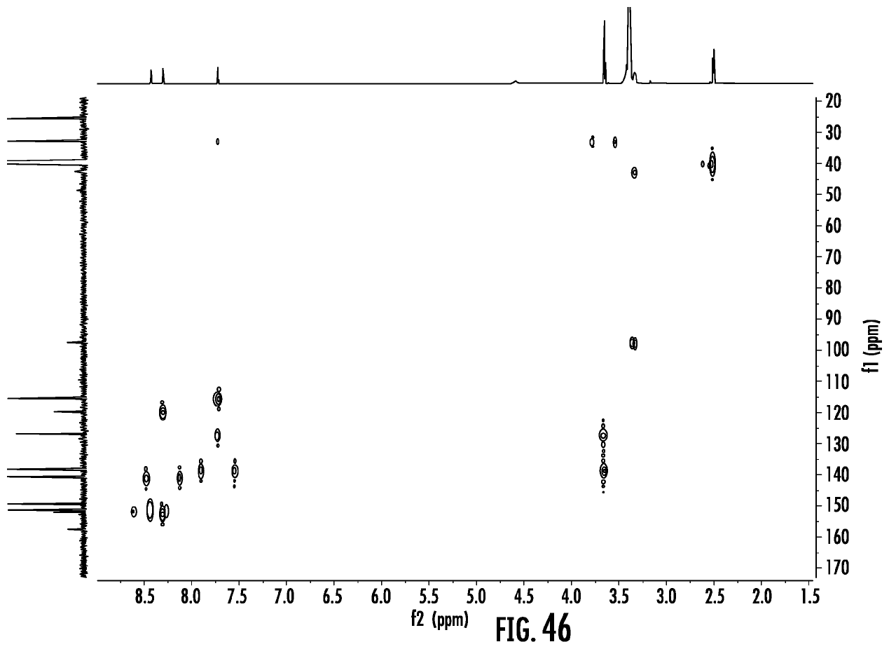

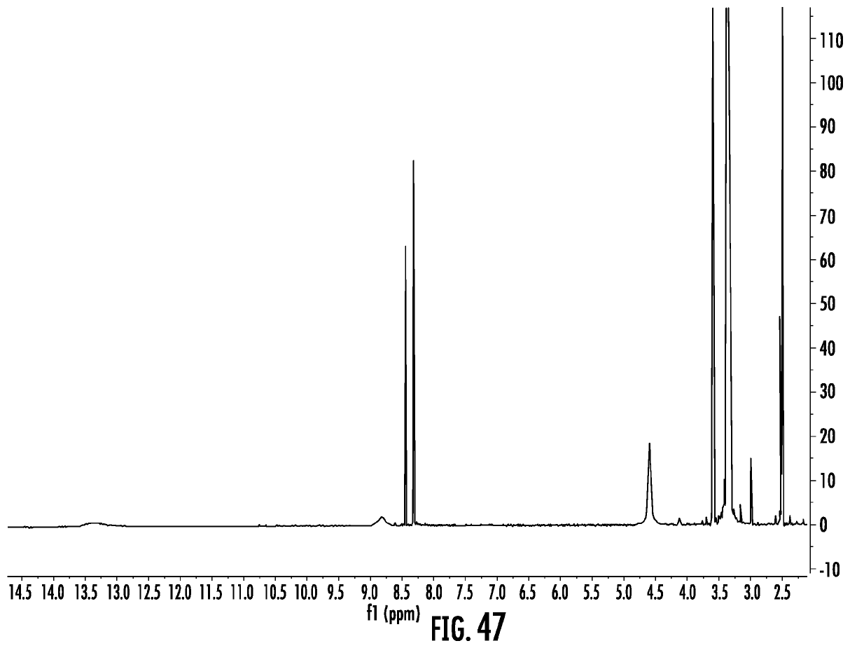

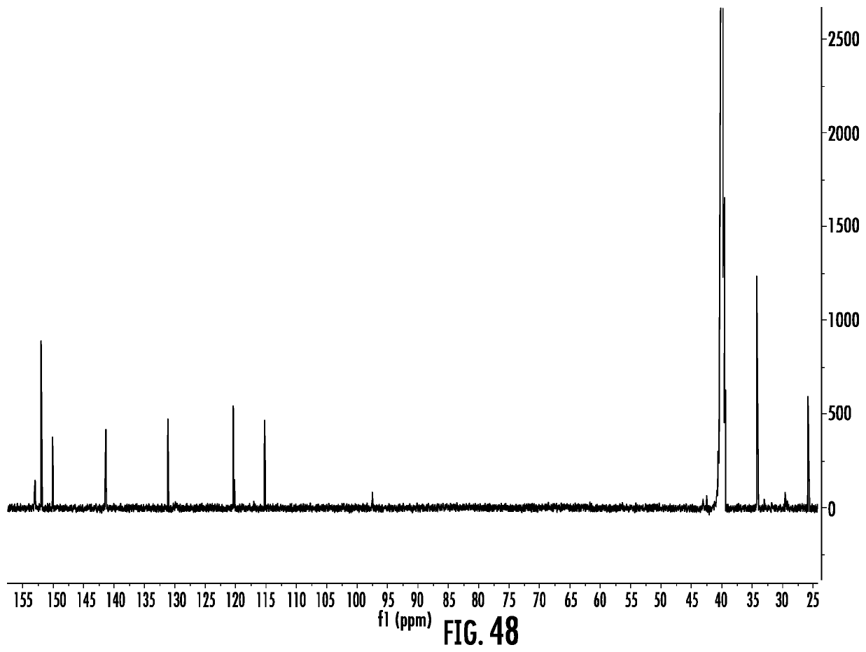

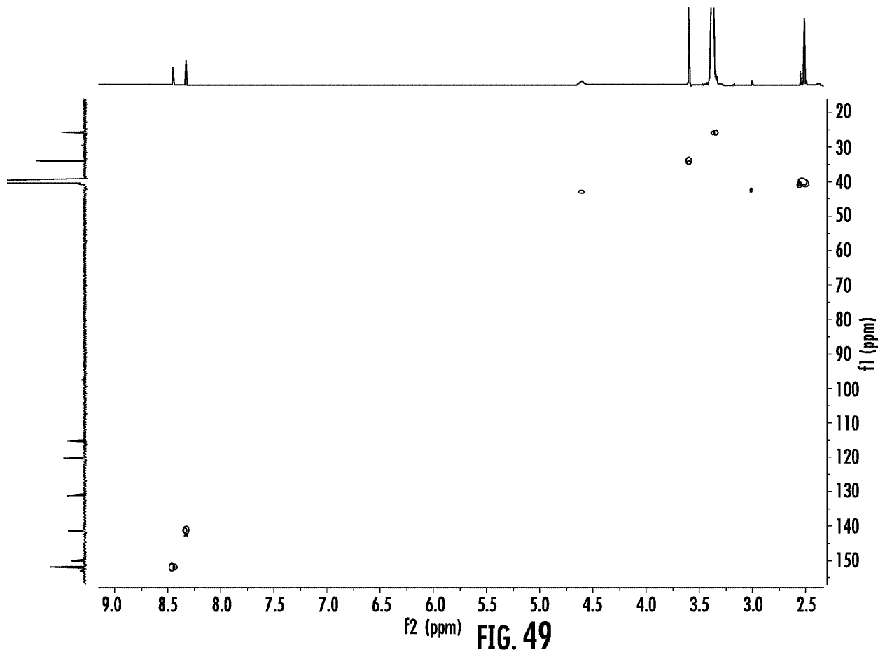

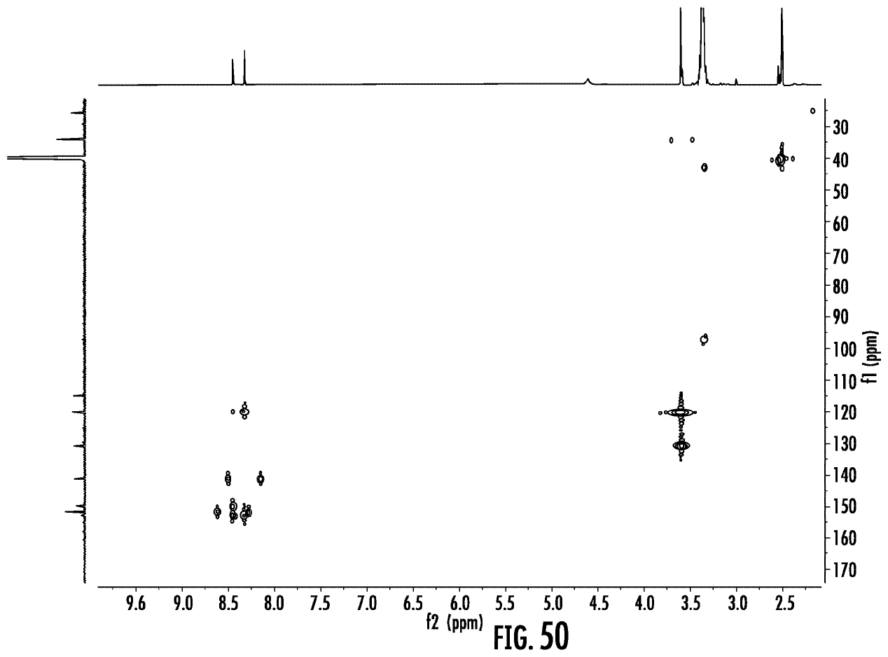

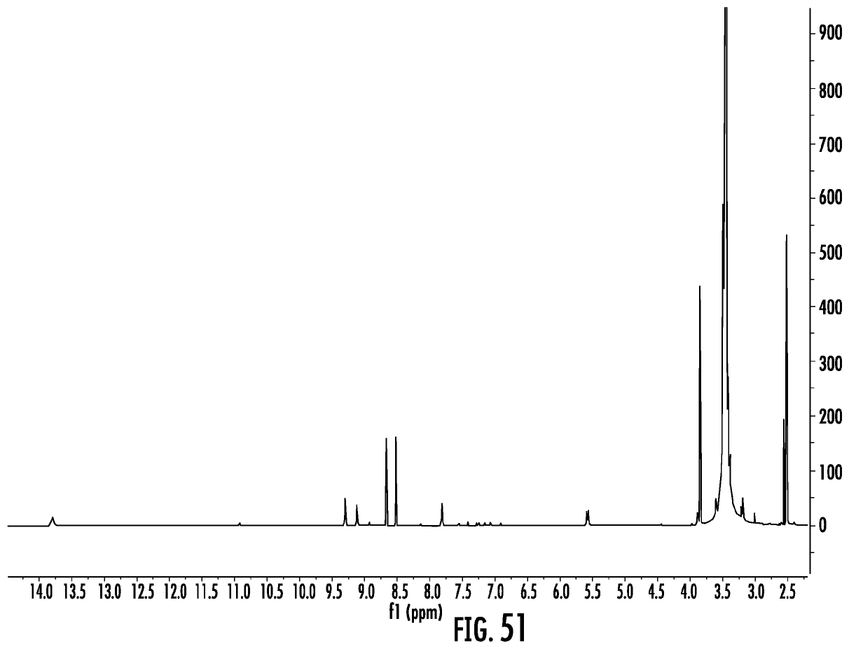

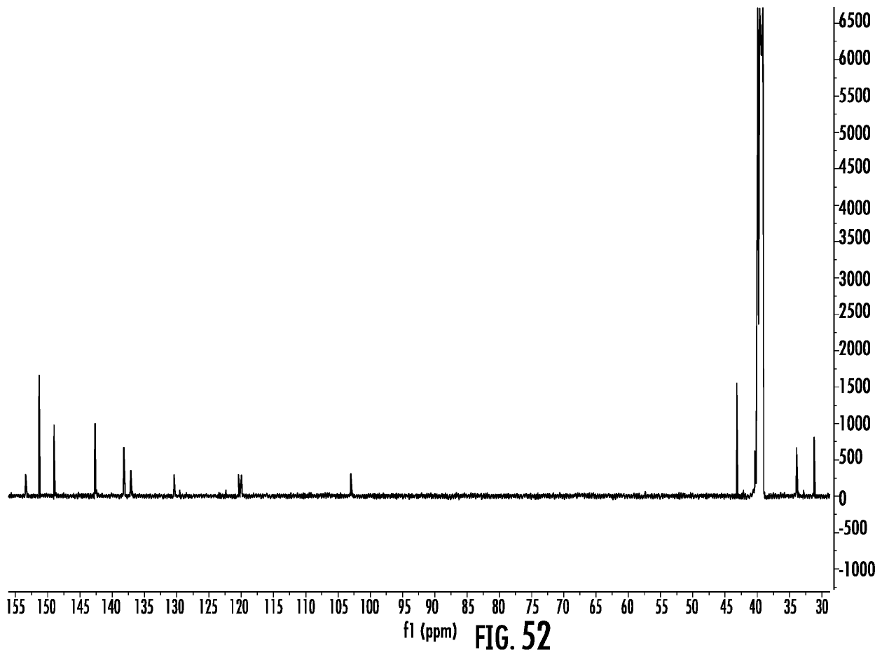

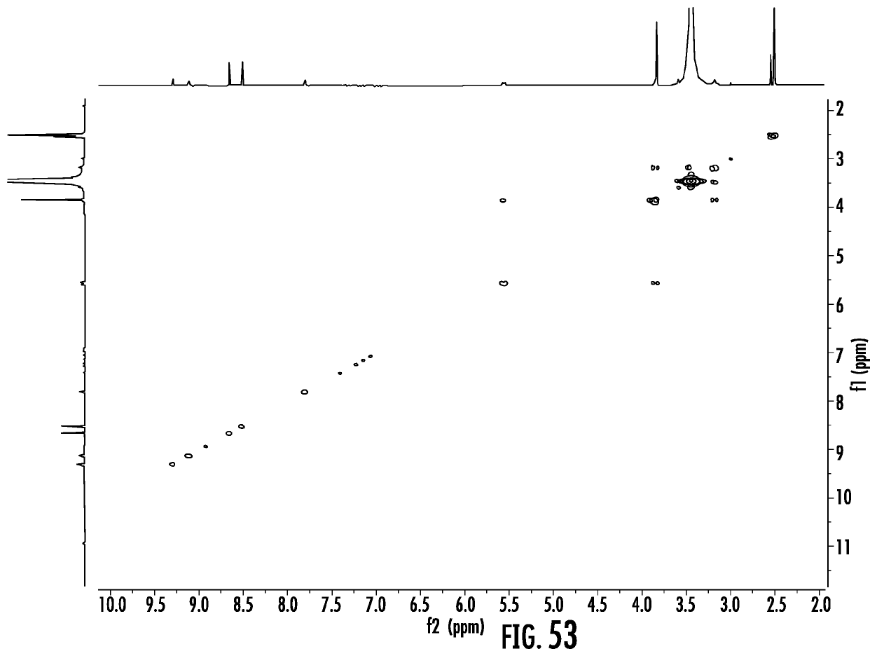

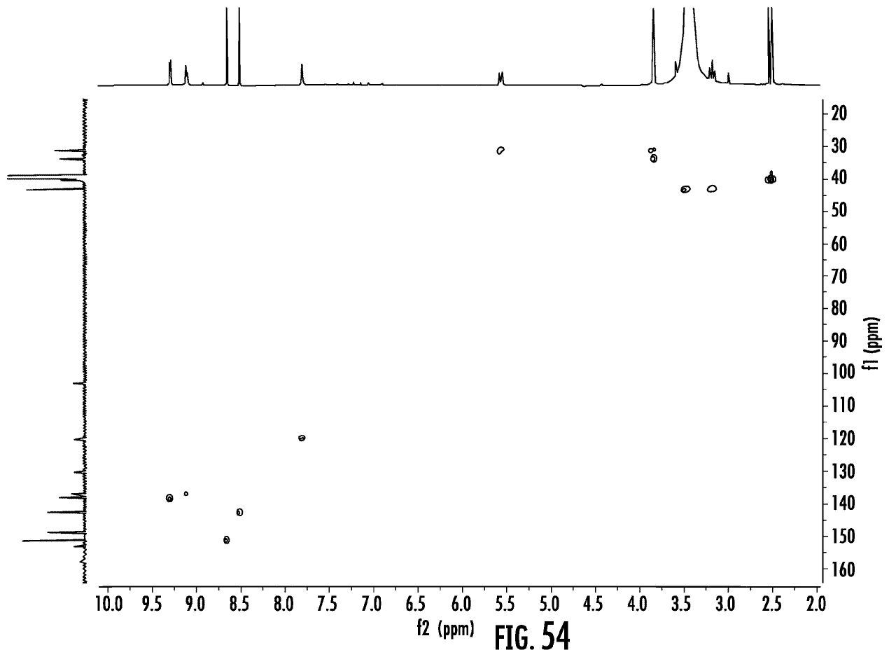

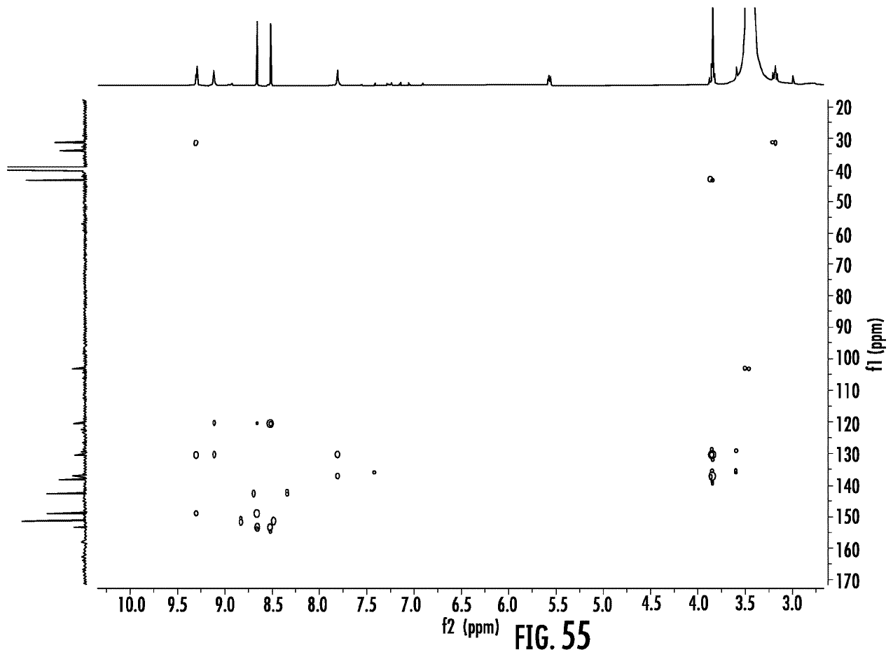

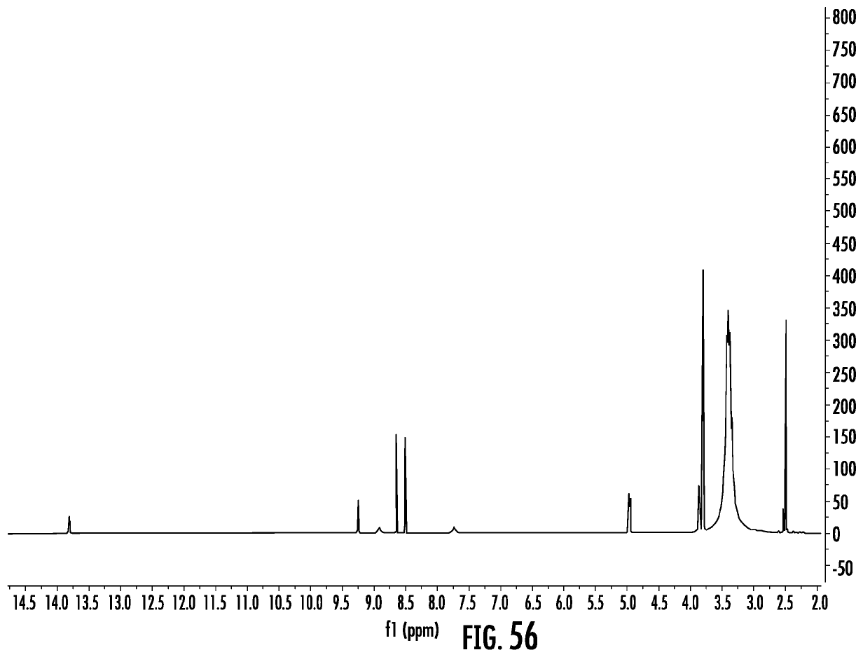

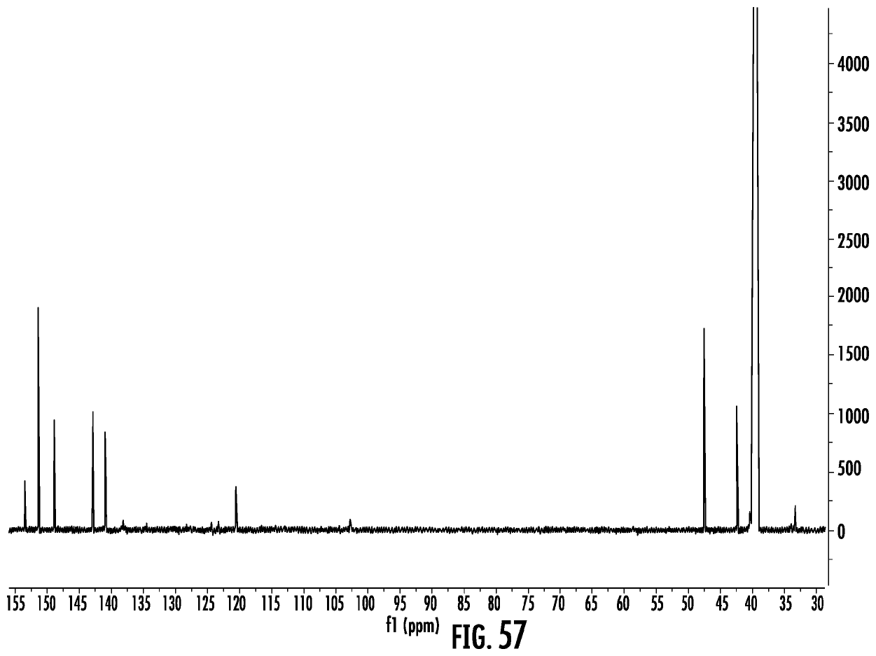

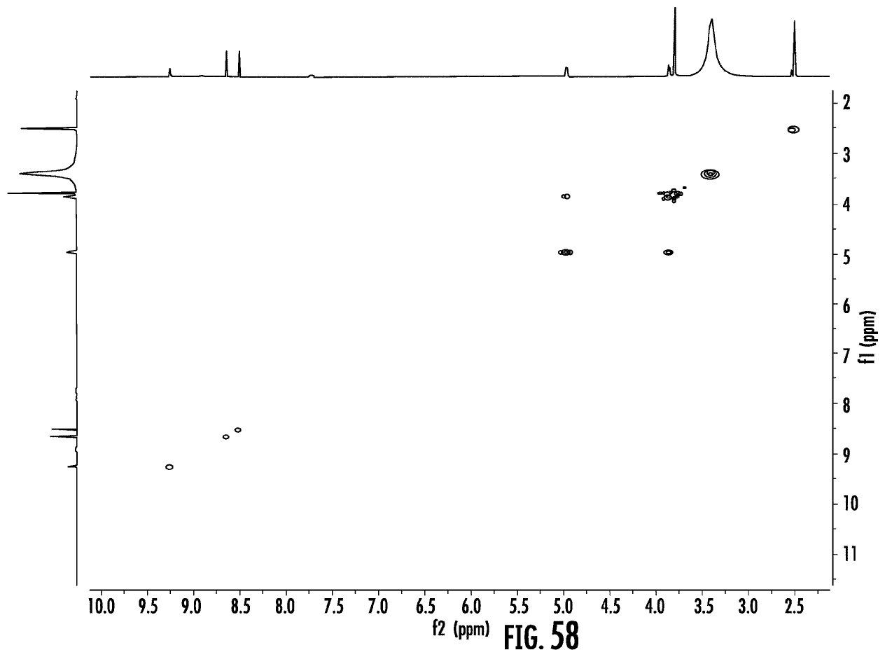

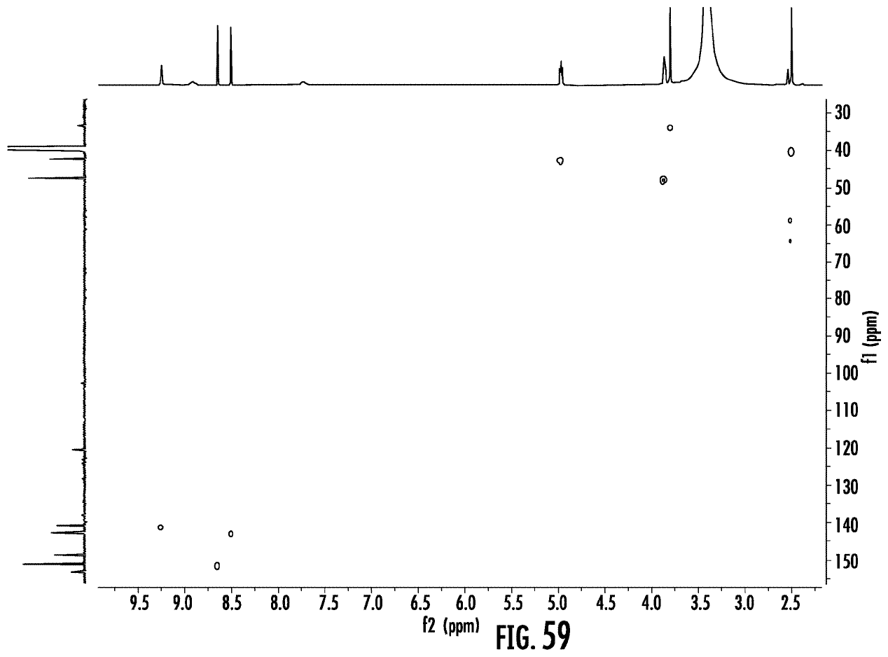

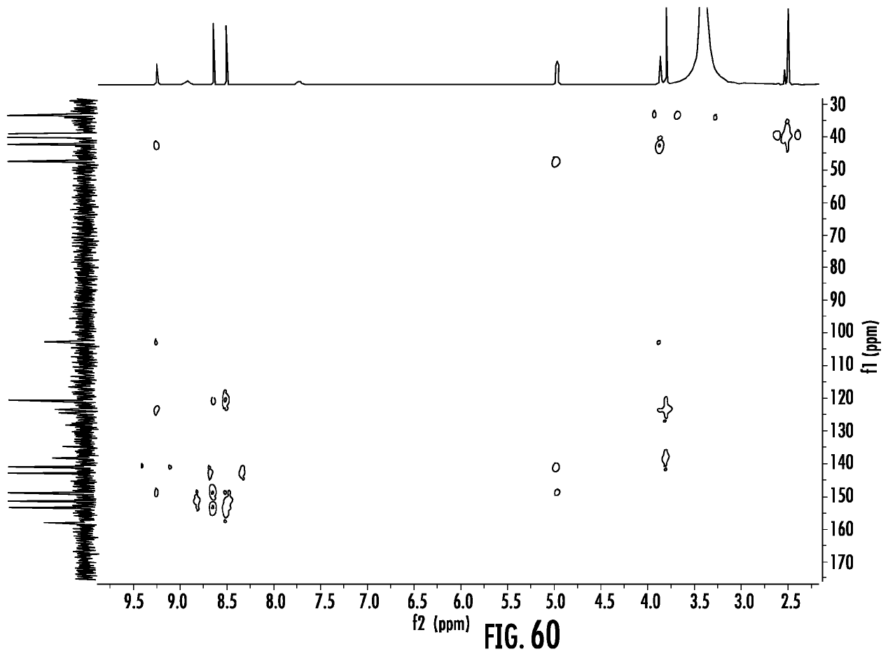

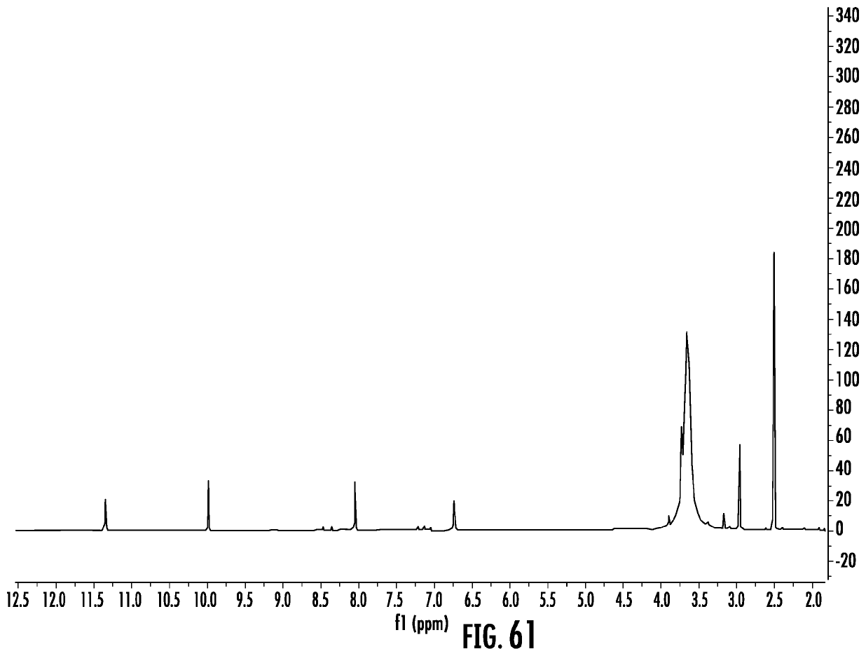

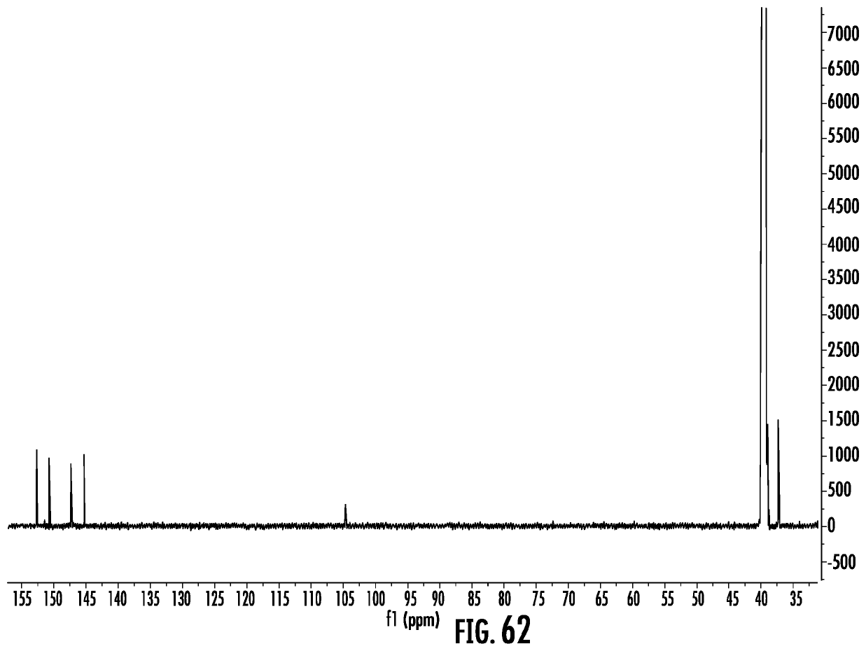

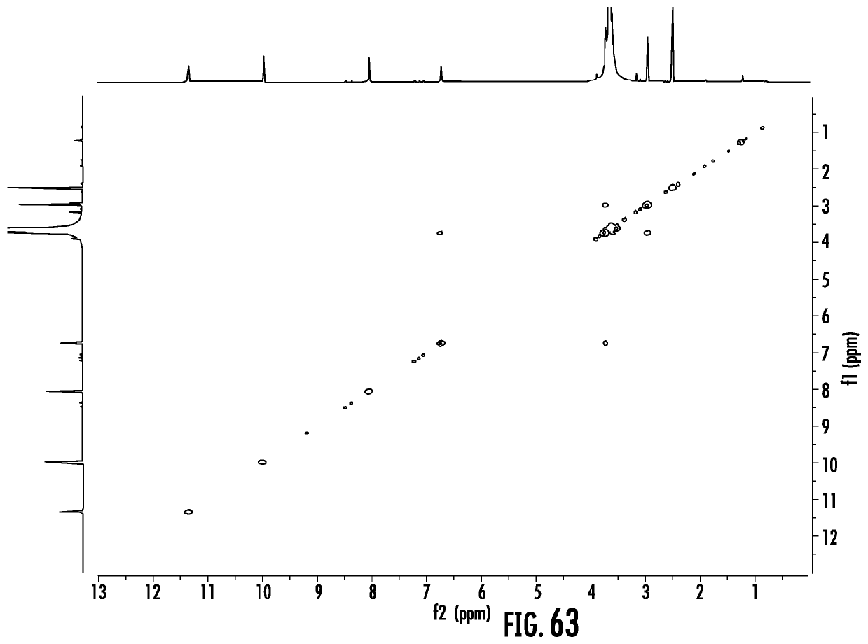

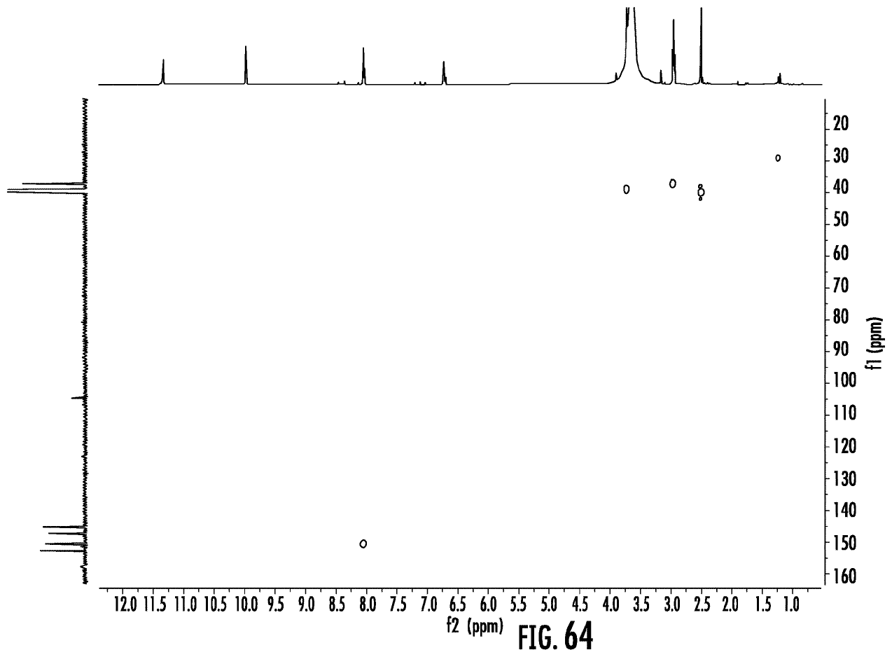

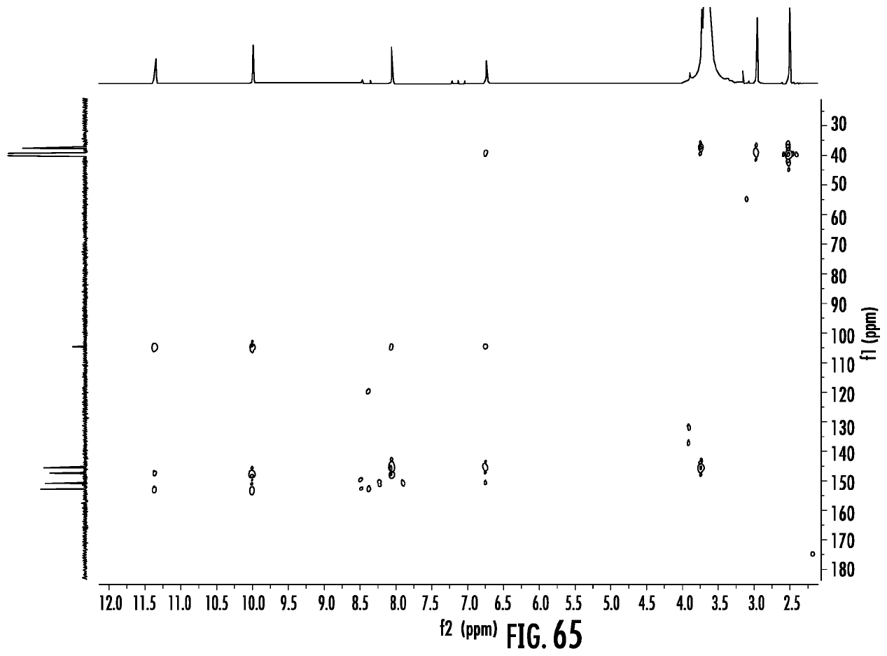

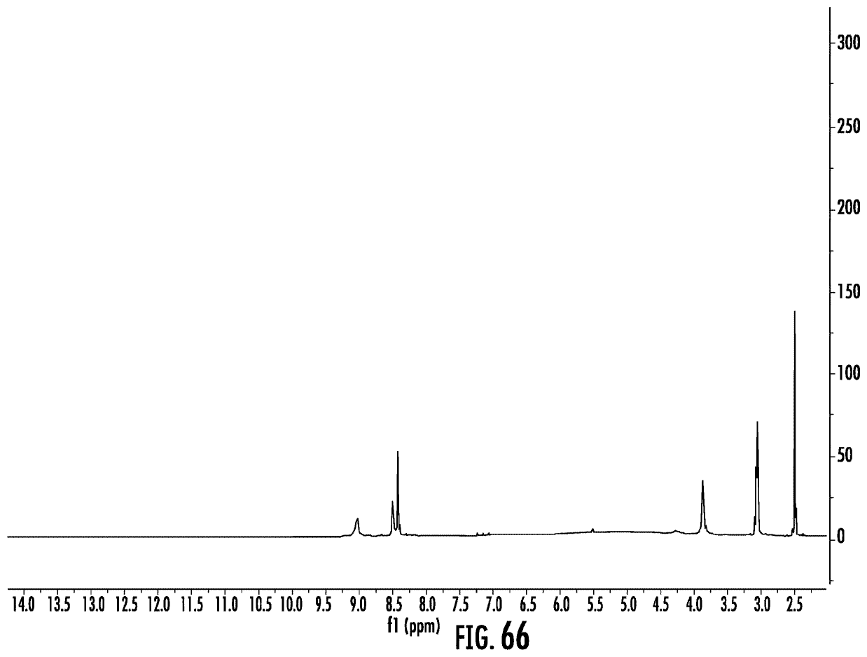

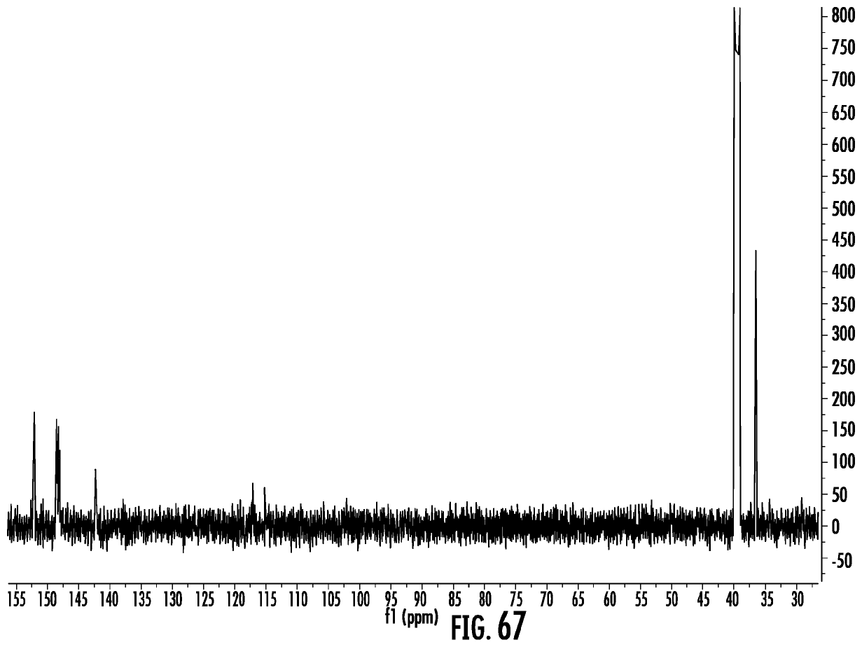

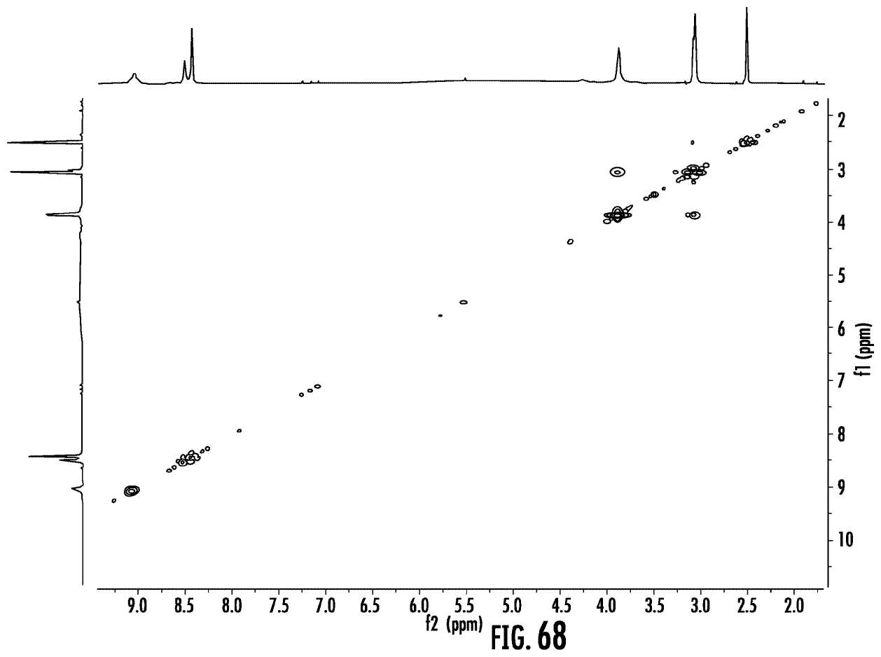

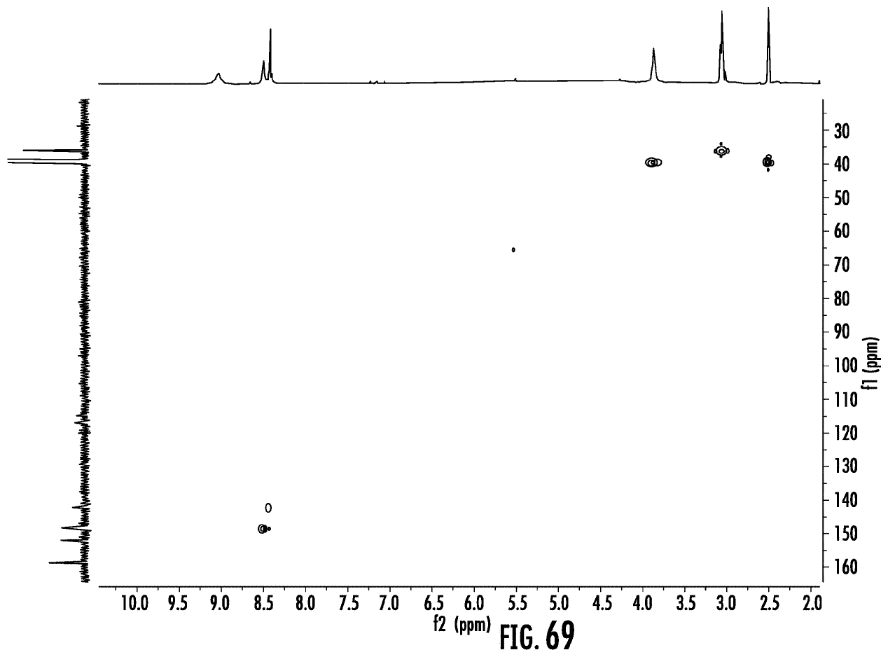

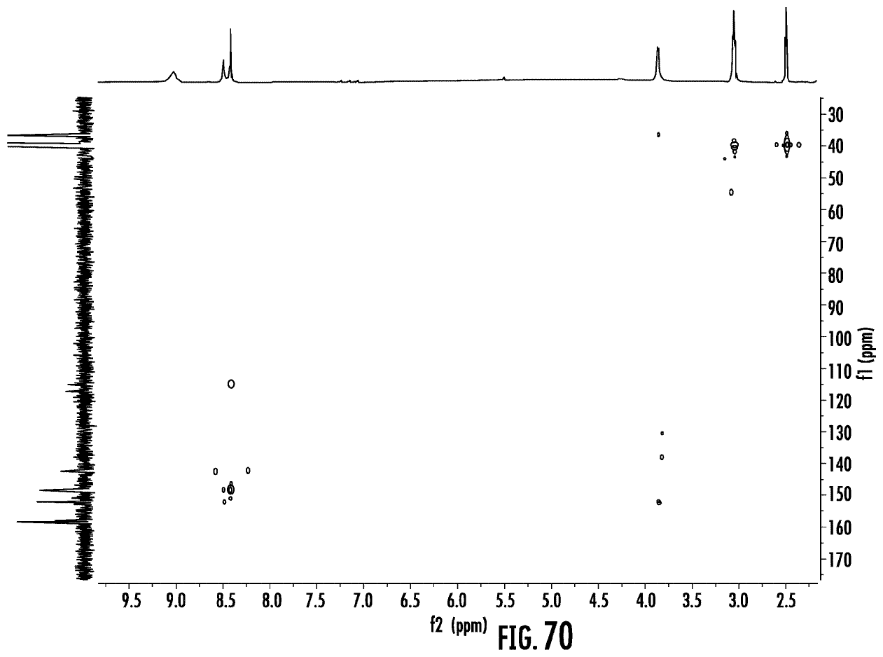

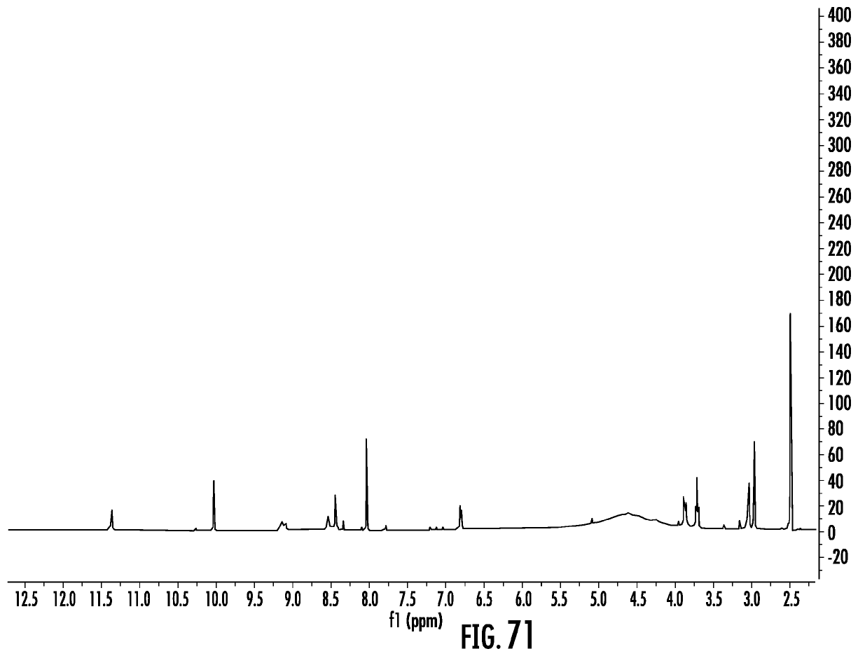

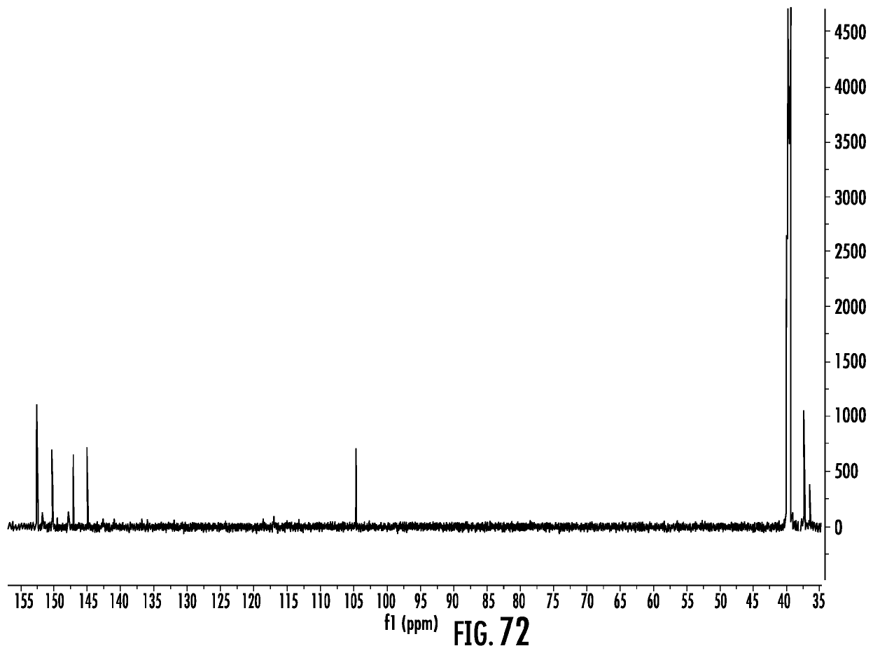

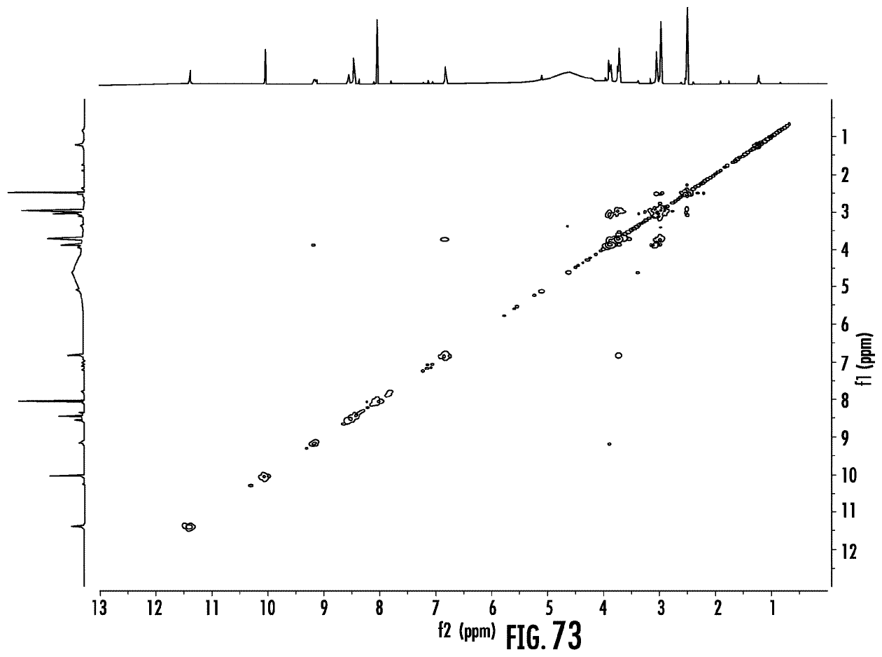

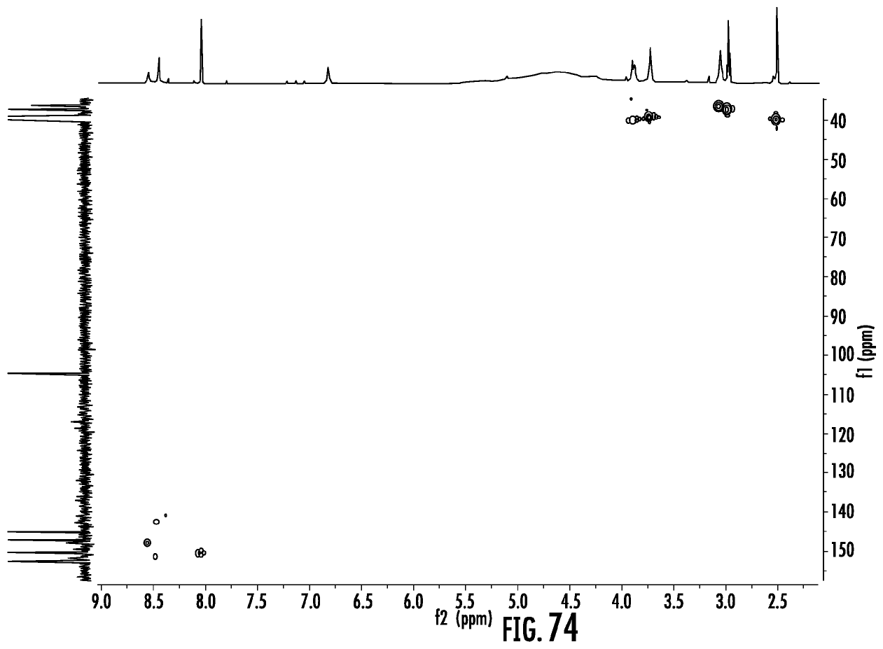

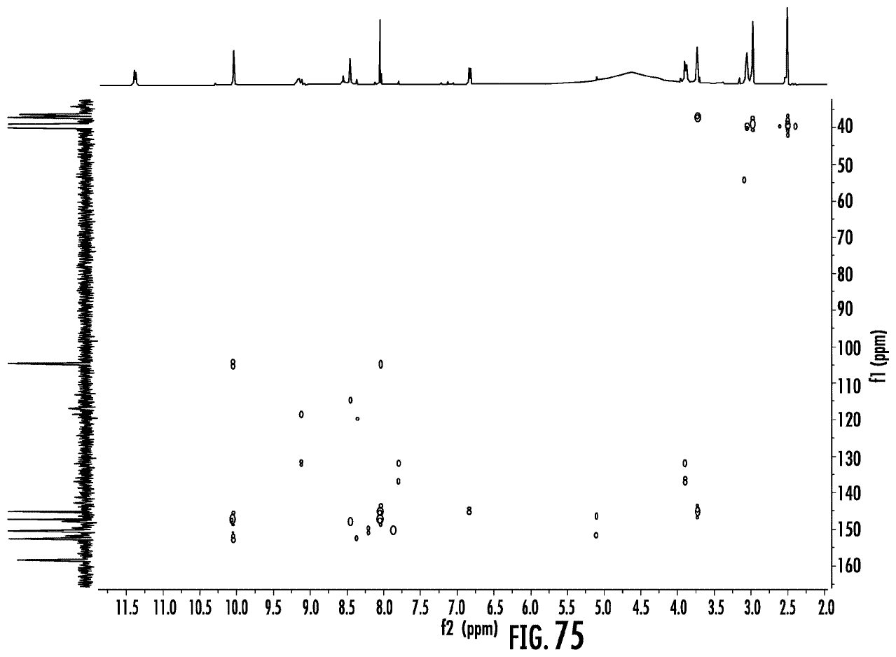

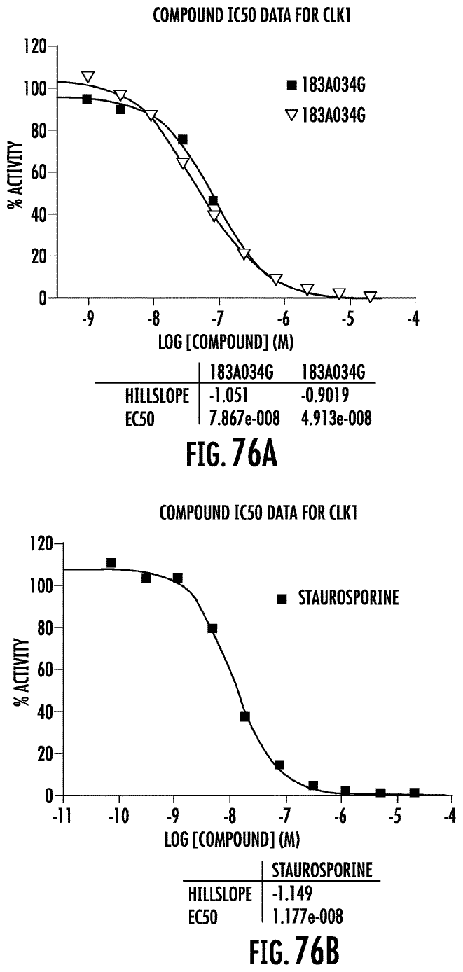

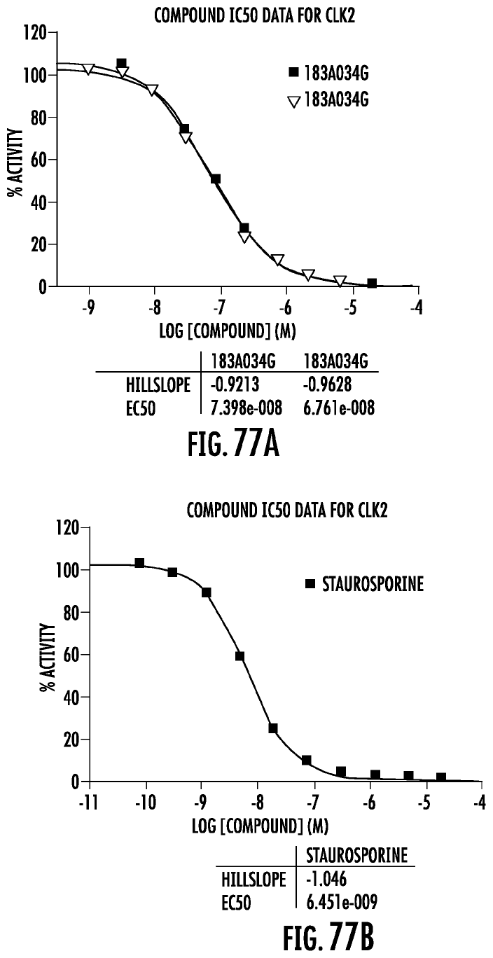

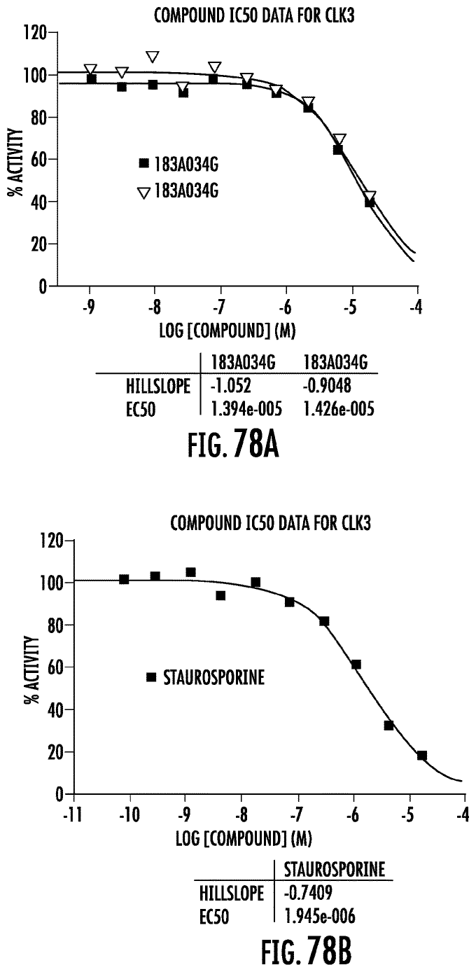

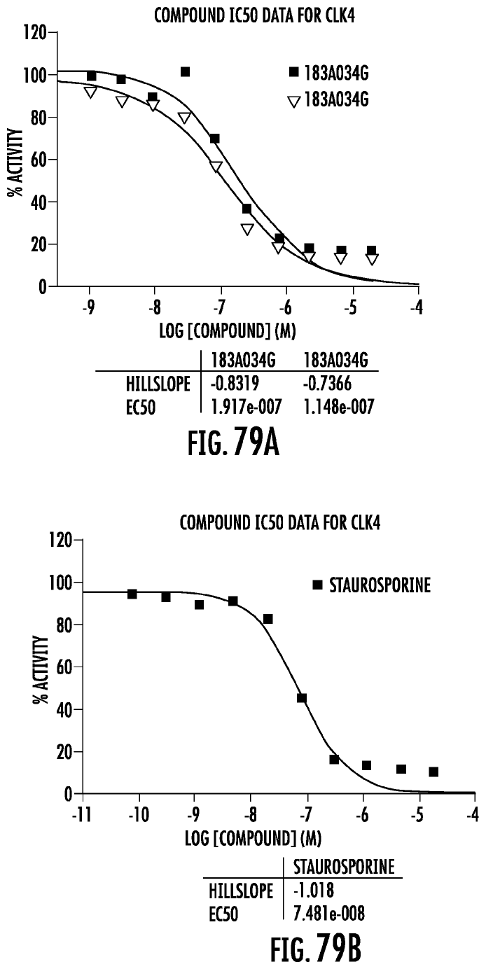

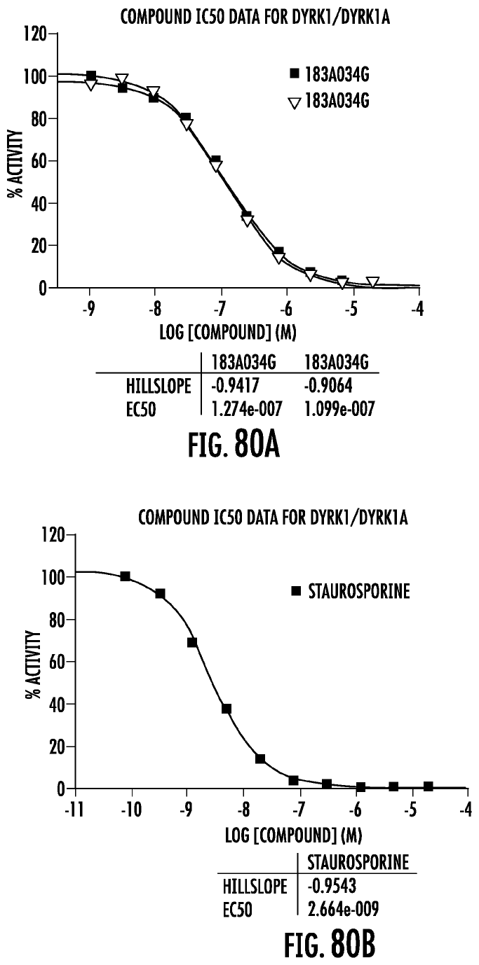

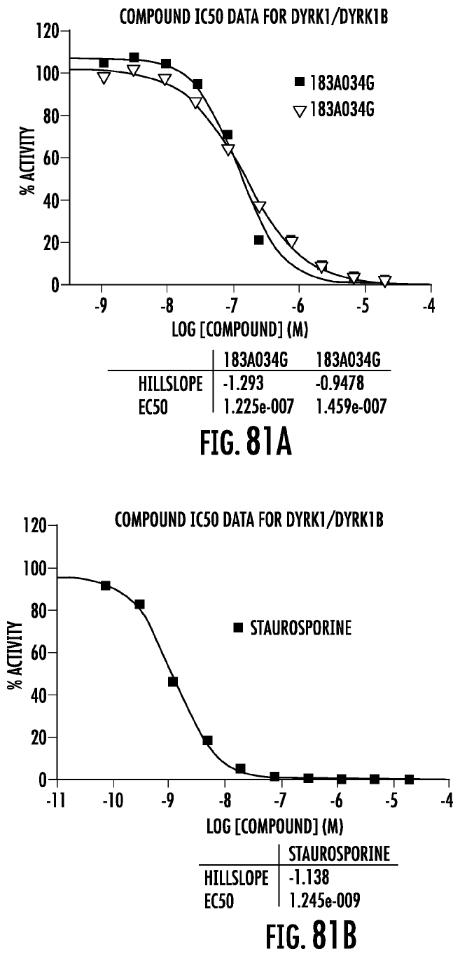

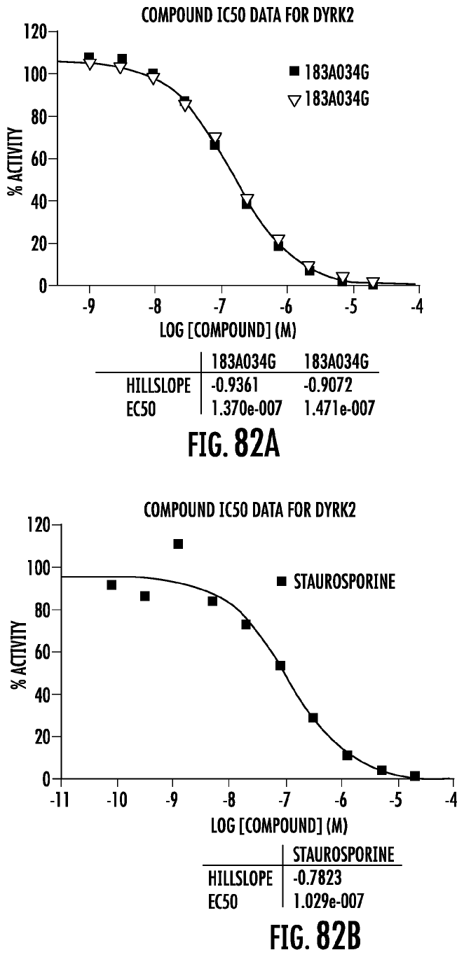

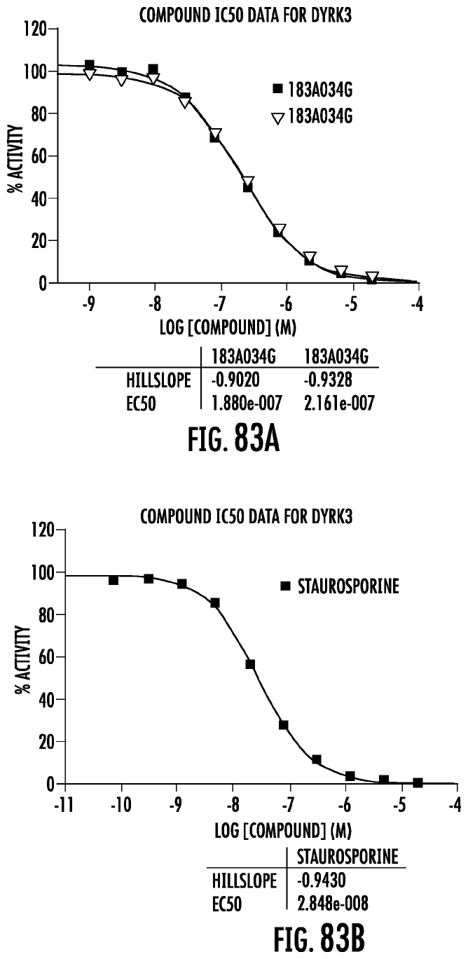

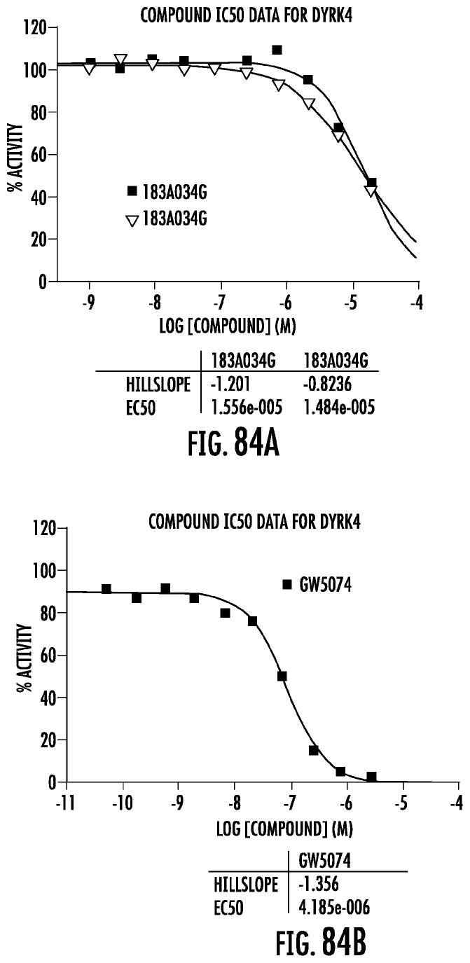

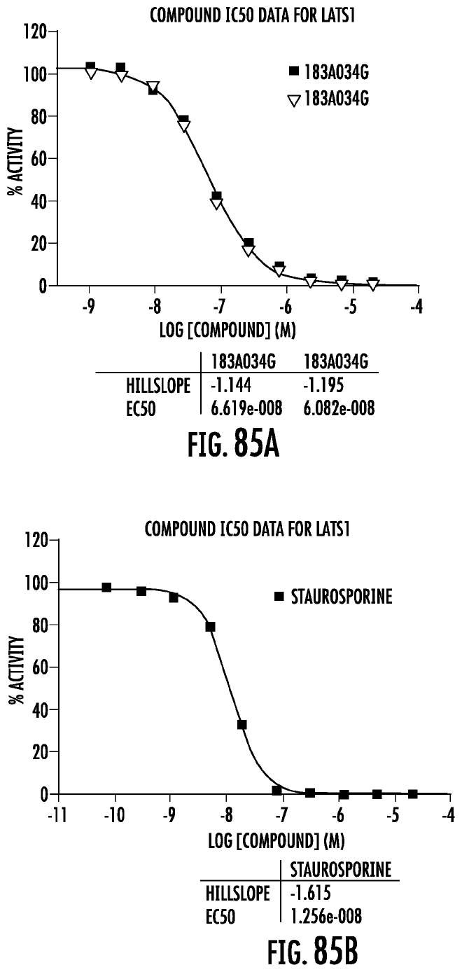

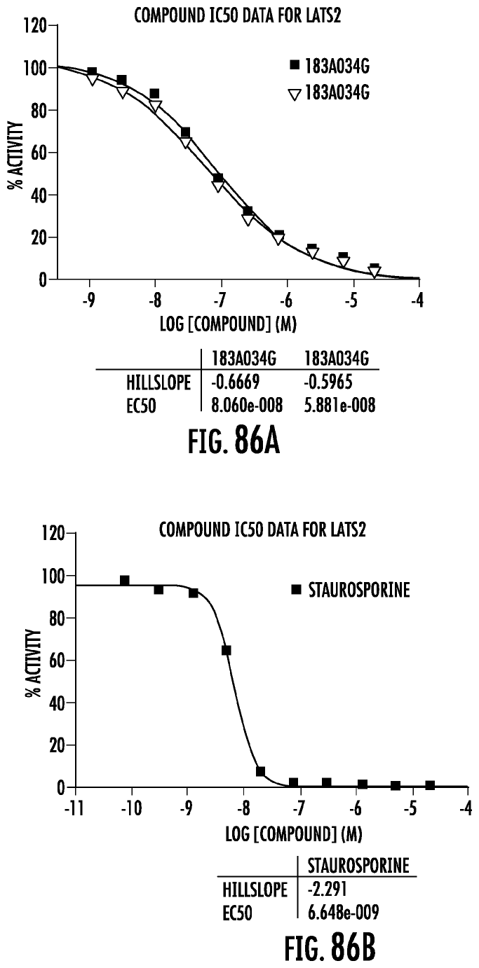

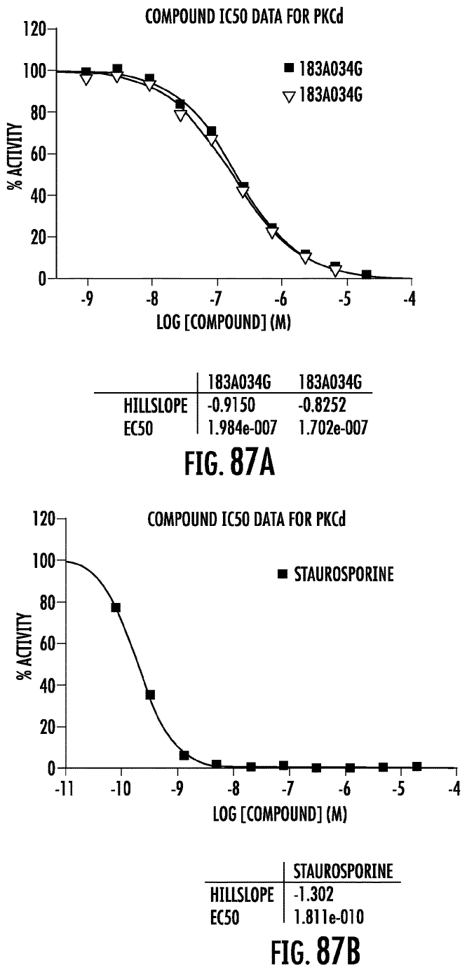

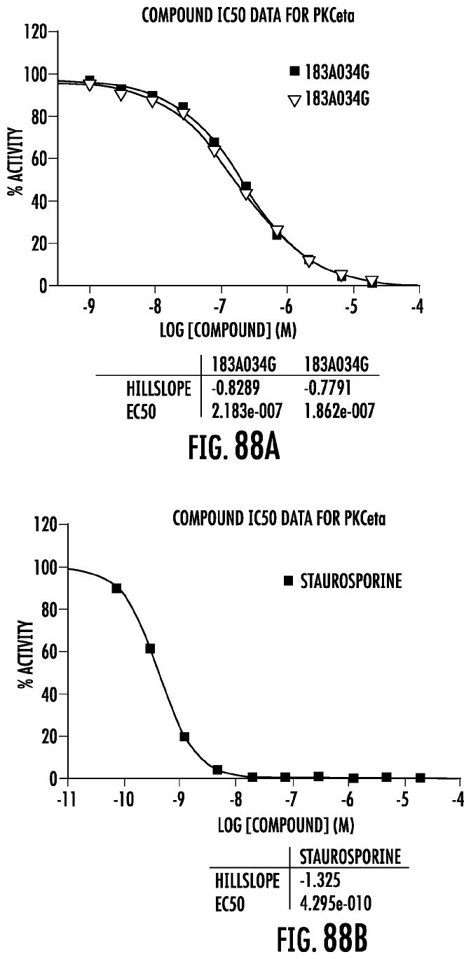

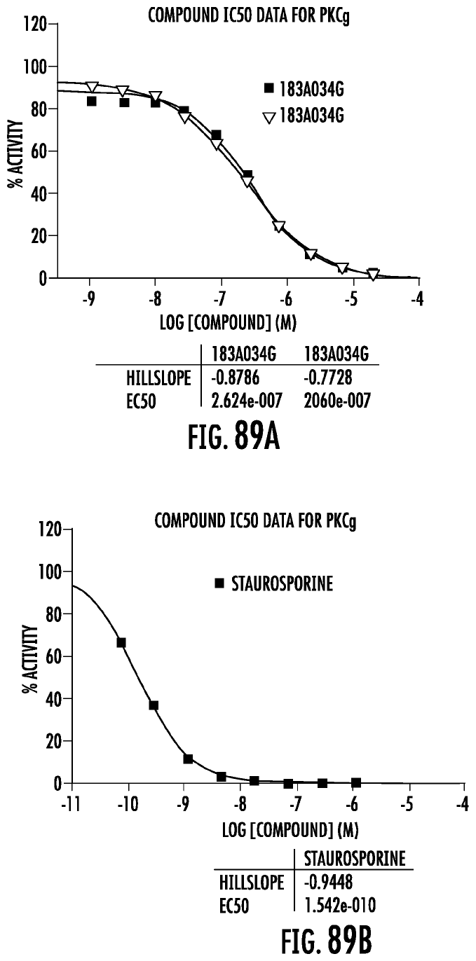

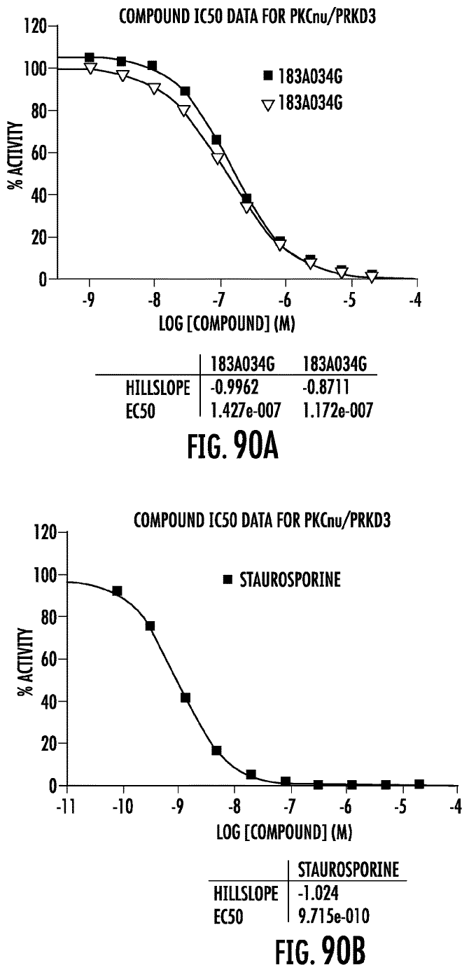

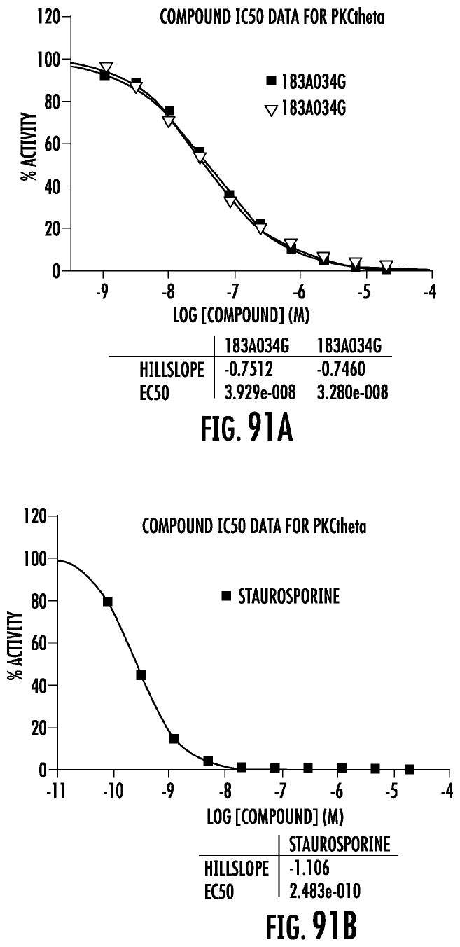

FIG. 9J is a graph showing the percentage of normalized activities of PKADJ and wild-type PKA (WT) that are treated with 183A041E.

FIG. 10A is a structure of a compound of an aspect of the invention.

FIG. 10B is a structure of a compound of an aspect of the invention.

FIG. 10C is a structure of a compound of an aspect of the invention.

FIG. 10D is a structure of a compound of an aspect of the invention.

FIG. 10E is a structure of a compound of an aspect of the invention.

FIG. 10F is a structure of a compound of an aspect of the invention.

FIG. 10G is a structure of a compound of an aspect of the invention.

FIG. 10H is a structure of a compound of an aspect of the invention.

FIG. 10I is a structure of a compound of an aspect of the invention.

FIG. 10J is a structure of a compound of an aspect of the invention.

FIG. 10K is a structure of a compound of an aspect of the invention.

FIG. 10L is a structure of a compound of an aspect of the invention.

FIG. 10M is a structure of a compound of an aspect of the invention.

FIG. 10N is a structure of a compound of an aspect of the invention.

FIG. 10O is a structure of a compound of an aspect of the invention.

FIG. 10P is a structure of a compound of an aspect of the invention.

FIG. 11A is a graph showing the percentage of normalized activities of PKADJ and wild-type PKA (WT) that are treated with 183A046A.

FIG. 11B is a graph showing the percentage of normalized activities of PKADJ and wild-type PKA (WT) that are treated with 183A046F.

FIG. 11C is a graph showing the percentage of normalized activities of PKADJ and wild-type PKA (WT) that are treated with 183A046A.

FIG. 11D is a graph showing the percentage of normalized activities of PKADJ and wild-type PKA (WT) that are treated with 183A046B.

FIG. 11E is a graph showing the percentage of normalized activities of PKADJ and wild-type PKA (WT) that are treated with 183A046C.

FIG. 11F is a graph showing the percentage of normalized activities of PKADJ and wild-type PKA (WT) that are treated with 183A047A.

FIG. 11G is a graph showing the percentage of normalized activities of PKADJ and wild-type PKA (WT) that are treated with 183A047C.

FIG. 11H is a graph showing the percentage of normalized activities of PKADJ and wild-type PKA (WT) that are treated with 183A047F.

FIG. 11I is a graph showing the percentage of normalized activities of PKADJ and wild-type PKA (WT) that are treated with 183A049B.

FIG. 11J is a graph showing the percentage of normalized activities of PKADJ and wild-type PKA (WT) that are treated with 183A049C.

FIG. 11K is a graph showing the percentage of normalized activities of PKADJ and wild-type PKA (WT) that are treated with 183A049E.

FIG. 11L is a graph showing the percentage of normalized activities of PKADJ and wild-type PKA (WT) that are treated with 183A049F.

FIG. 11M is a graph showing the percentage of normalized activities of PKADJ and wild-type PKA (WT) that are treated with 183A050B.

FIG. 11N is a graph showing the percentage of normalized activities of PKADJ and wild-type PKA (WT) that are treated with 183A050C.

FIG. 11O is a graph showing the percentage of normalized activities of PKADJ and wild-type PKA (WT) that are treated with 183A050D.

FIG. 11P is a graph showing the percentage of normalized activities of PKADJ and wild-type PKA (WT) that are treated with 183A050E.

FIG. 12A is a structure of a compound of an aspect of the invention.

FIG. 12B is a structure of a compound of an aspect of the invention.

FIG. 12C is a structure of a compound of an aspect of the invention.

FIG. 12D is a structure of a compound of an aspect of the invention.

FIG. 13 is a graph showing the percentage of normalized activity of PKADJ that is treated with compounds of aspects of the invention.

FIG. 14 is a graph showing the percentage of normalized activities of twenty selected kinases that are treated with aplithianine A₁.

FIG. 15 is an example of kinase mapping.

FIG. 16 is an example of several branches of the kinase mapping of FIG. 15.

FIG. 17 is an example of several branches of the kinase mapping of FIG. 15.

FIG. 18 is an example of several branches of the kinase mapping of FIG. 15.

FIG. 19 is a graph showing the percentage of normalized activities of twenty selected kinases that are treated with aplithianine A, a compound of an aspect of the invention.

FIG. 20 is a graph showing the IC₅₀ values of twenty selected kinases that are treated with aplithianine A.

FIG. 21 is a graph showing the PKG1a differential IC₅₀ values of twenty selected kinases that are treated with aplithianine A.

FIG. 22 shows the structures of Compounds 1-5 and semi-synthetic analogues 1a-1d.

FIG. 23 shows selected ¹H-¹H COSY and HMBC correlations of Compounds 1 and 3.

FIG. 24 shows a ¹H NMR spectrum of Compound 1 in DMSO-d₆.

FIG. 25 shows a ¹³C NMR spectrum of Compound 1 in DMSO-d₆.

FIG. 26 shows a ¹H-¹H COSY spectrum of Compound 1 in DMSO-d₆.

FIG. 27 shows a HSQC spectrum of Compound 1 in DMSO-d₆.

FIG. 28 shows a HMBC spectrum of Compound 1 in DMSO-d₆.

FIG. 29 shows a ¹H NMR spectrum of Compound 1 in methanol-d₄.

FIG. 30 shows a ¹³C NMR spectrum of Compound 1 in methanol-d₄.

FIG. 31 shows a ¹H-¹H COSY spectrum of Compound 1 in methanol-d₄.

FIG. 32 shows a HSQC spectrum of Compound 1 in methanol-d₄.

FIG. 33 shows a HMBC spectrum of Compound 1 in methanol-d₄.

FIG. 34 shows a ¹H NMR spectrum of Compound 2 in DMSO-d₆.

FIG. 35 shows a HSQC spectrum of Compound 2 in DMSO-d₆.

FIG. 36 shows a HMBC spectrum of Compound 2 in DMSO-d₆.

FIG. 37 shows a ¹H NMR spectrum of Compound 2 in methanol-d₄.

FIG. 38 shows a ¹³C NMR spectrum of Compound 2 in methanol-d₄.

FIG. 39 shows a ¹H-¹H COSY spectrum of Compound 2 in methanol-d₄.

FIG. 40 shows a HSQC spectrum of Compound 2 in methanol-d₄.

FIG. 41 shows a HMBC spectrum of Compound 2 in methanol-d₄.

FIG. 42 shows a ¹H NMR spectrum of Compound 1a in DMSO-d₆.

FIG. 43 shows a ¹³C NMR spectrum of Compound 1a in DMSO-d₆.

FIG. 44 shows a ¹H-¹H COSY spectrum of Compound 1a in DMSO-d₆.

FIG. 45 shows a HSQC spectrum of Compound 1a in DMSO-d₆.

FIG. 46 shows a HMBC spectrum of Compound 1a in DMSO-d₆.

FIG. 47 shows a ¹H NMR spectrum of Compound 1b in DMSO-d₆.

FIG. 48 shows a ¹³C NMR spectrum of Compound 1b in DMSO-d₆.

FIG. 49 shows a HSQC spectrum of Compound 1b in DMSO-d₆.

FIG. 50 shows a HMBC spectrum of Compound 1b in DMSO-d₆.

FIG. 51 shows a ¹H NMR spectrum of Compound 1c in DMSO-d₆.

FIG. 52 shows a ¹³C NMR spectrum of Compound 1c in DMSO-d₆.

FIG. 53 shows a ¹H-¹H COSY spectrum of Compound 1c in DMSO-d₆

FIG. 54 shows a HSQC spectrum of Compound 1c in DMSO-d₆.

FIG. 55 shows a HMBC spectrum of Compound 1c in DMSO-d₆.

FIG. 56 shows a ¹H NMR spectrum of Compound 1d in DMSO-d₆.

FIG. 57 shows a ¹³C NMR spectrum of Compound 1d in DMSO-d₆.

FIG. 58 shows a ¹H-¹H COSY spectrum of Compound 1d in DMSO-d₆.

FIG. 59 shows a HSQC spectrum of Compound 1d in DMSO-d₆.

FIG. 60 shows a HMBC spectrum of Compound 1d in DMSO-d₆.

FIG. 61 shows a ¹H NMR spectrum of Compound 3 in DMSO-d₆.

FIG. 62 shows a ¹³C NMR spectrum of Compound 3 in DMSO-d₆.

FIG. 63 shows a ¹H-¹H COSY spectrum of Compound 3 in DMSO-d₆.

FIG. 64 shows a HSQC spectrum of Compound 3 in DMSO-d₆.

FIG. 65 shows a HMBC spectrum of Compound 3 in DMSO-d₆.

FIG. 66 shows a ¹H NMR spectrum of Compound 4 in DMSO-d₆.

FIG. 67 shows a ¹³C NMR spectrum of Compound 4 in DMSO-d₆.

FIG. 68 shows a ¹H-¹H COSY spectrum of Compound 4 in DMSO-d₆.

FIG. 69 shows a HSQC spectrum of Compound 4 in DMSO-d₆.

FIG. 70 shows a HMBC spectrum of Compound 4 in DMSO-d₆.

FIG. 71 shows a ¹H NMR spectrum of Compound 5 in DMSO-d₆.

FIG. 72 shows a ¹³C NMR spectrum of Compound 5 in DMSO-d₆.

FIG. 73 shows a ¹H-¹H COSY spectrum of Compound 5 in DMSO-d₆.

FIG. 74 shows a HSQC spectrum of Compound 5 in DMSO-d₆.

FIG. 75 shows a HMBC spectrum of Compound 5 in DMSO-d₆.

FIG. 76A is a graph showing the percent enzyme activity (relative to controls) and log curve fit.

FIG. 76B is a graph showing the percent enzyme activity (relative to controls) and log curve fit.

FIG. 77A is a graph showing the percent enzyme activity (relative to controls) and log curve fit.

FIG. 77B is a graph showing the percent enzyme activity (relative to controls) and log curve fit.

FIG. 78A is a graph showing the percent enzyme activity (relative to controls) and log curve fit.

FIG. 78B is a graph showing the percent enzyme activity (relative to controls) and log curve fit.

FIG. 79A is a graph showing the percent enzyme activity (relative to controls) and log curve fit.

FIG. 79B is a graph showing the percent enzyme activity (relative to controls) and log curve fit.

FIG. 80A is a graph showing the percent enzyme activity (relative to controls) and log curve fit.

FIG. 80B is a graph showing the percent enzyme activity (relative to controls) and log curve fit.

FIG. 81A is a graph showing the percent enzyme activity (relative to controls) and log curve fit.

FIG. 81B is a graph showing the percent enzyme activity (relative to controls) and log curve fit.

FIG. 82A is a graph showing the percent enzyme activity (relative to controls) and log curve fit.

FIG. 82B is a graph showing the percent enzyme activity (relative to controls) and log curve fit.

FIG. 83A is a graph showing the percent enzyme activity (relative to controls) and log curve fit.

FIG. 83B is a graph showing the percent enzyme activity (relative to controls) and log curve fit.

FIG. 84A is a graph showing the percent enzyme activity (relative to controls) and log curve fit.

FIG. 84B is a graph showing the percent enzyme activity (relative to controls) and log curve fit.

FIG. 85A is a graph showing the percent enzyme activity (relative to controls) and log curve fit.

FIG. 85B is a graph showing the percent enzyme activity (relative to controls) and log curve fit.

FIG. 86A is a graph showing the percent enzyme activity (relative to controls) and log curve fit.

FIG. 86B is a graph showing the percent enzyme activity (relative to controls) and log curve fit.

FIG. 87A is a graph showing the percent enzyme activity (relative to controls) and log curve fit.

FIG. 87B is a graph showing the percent enzyme activity (relative to controls) and log curve fit.

FIG. 88A is a graph showing the percent enzyme activity (relative to controls) and log curve fit.

FIG. 88B is a graph showing the percent enzyme activity (relative to controls) and log curve fit.

FIG. 89A is a graph showing the percent enzyme activity (relative to controls) and log curve fit.

FIG. 89B is a graph showing the percent enzyme activity (relative to controls) and log curve fit.

FIG. 90A is a graph showing the percent enzyme activity (relative to controls) and log curve fit.

FIG. 90B is a graph showing the percent enzyme activity (relative to controls) and log curve fit.

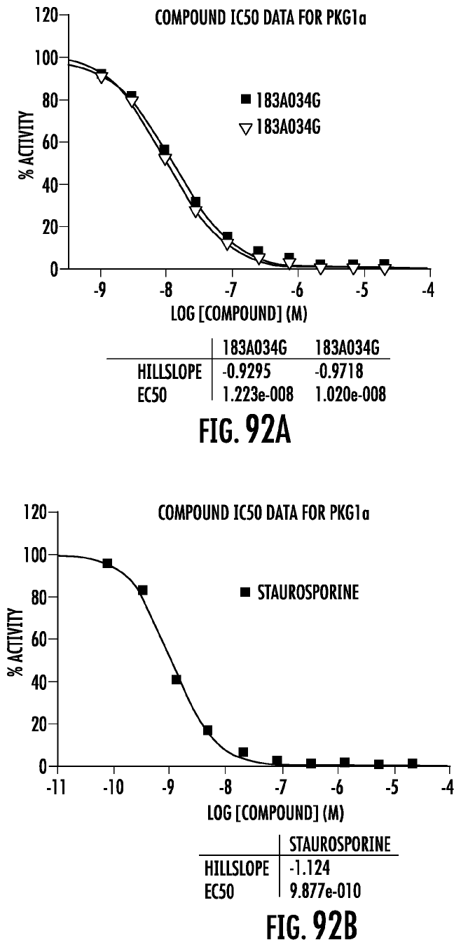

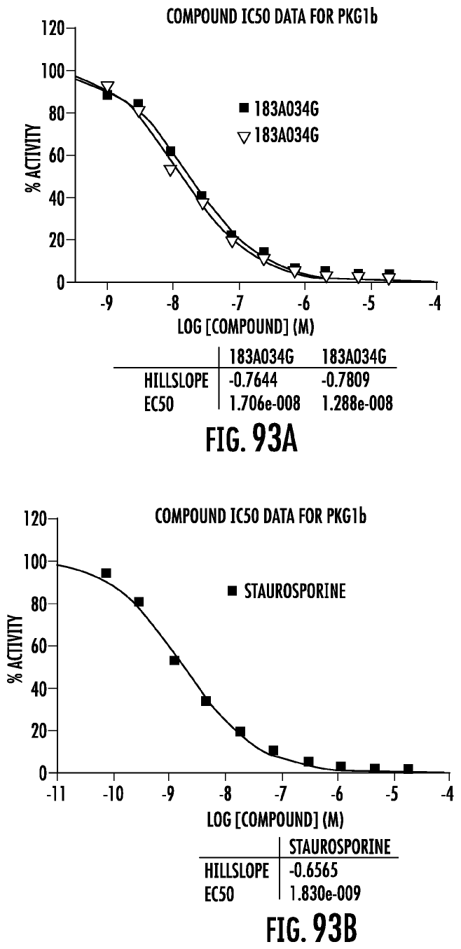

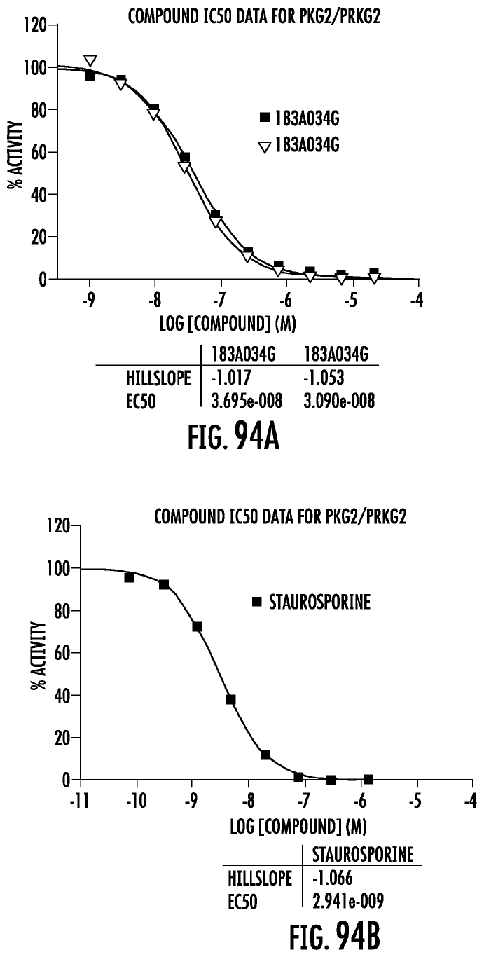

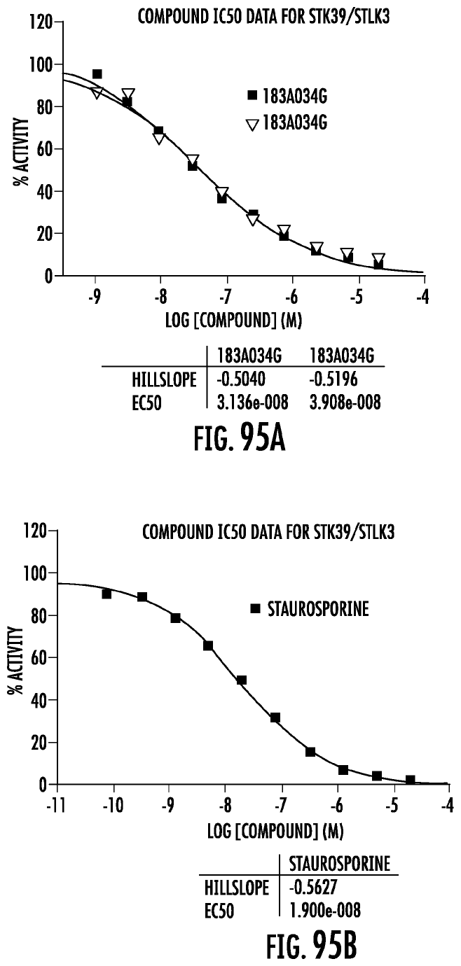

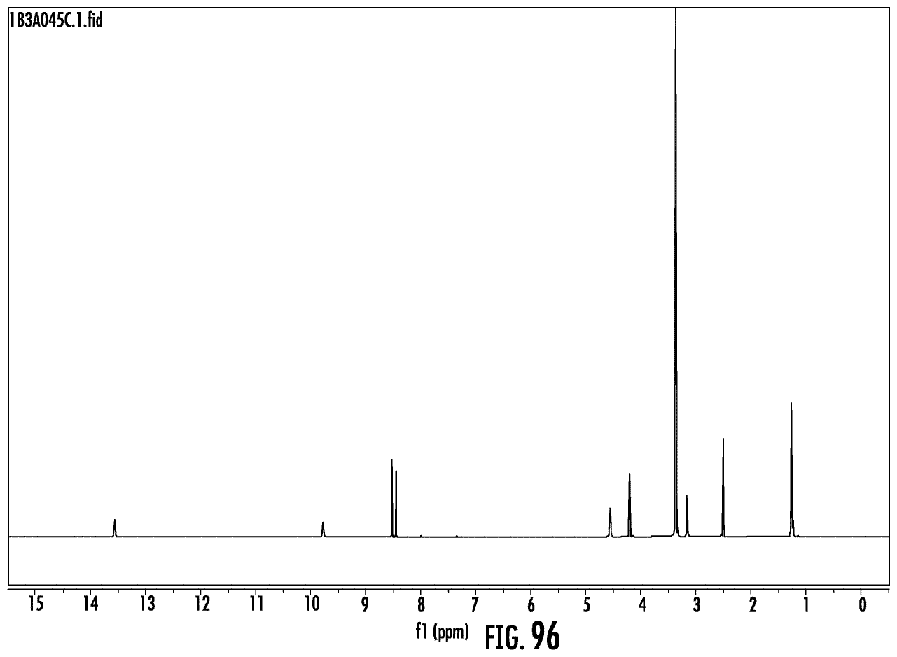

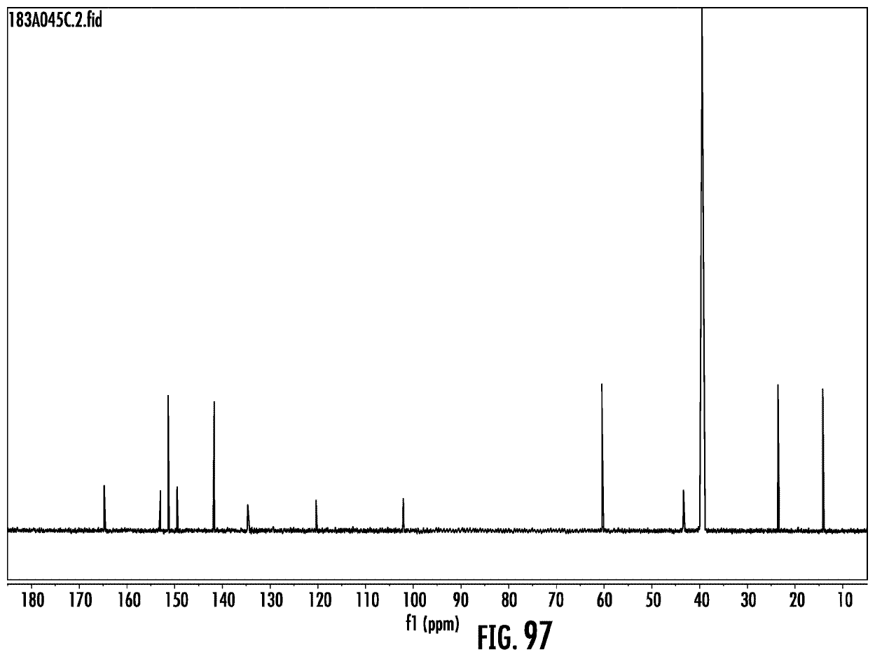

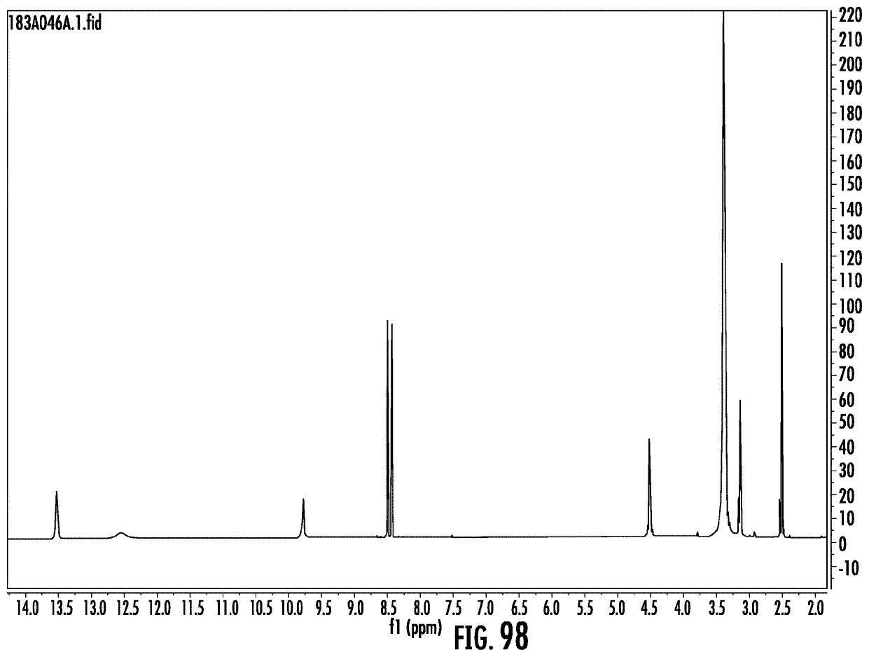

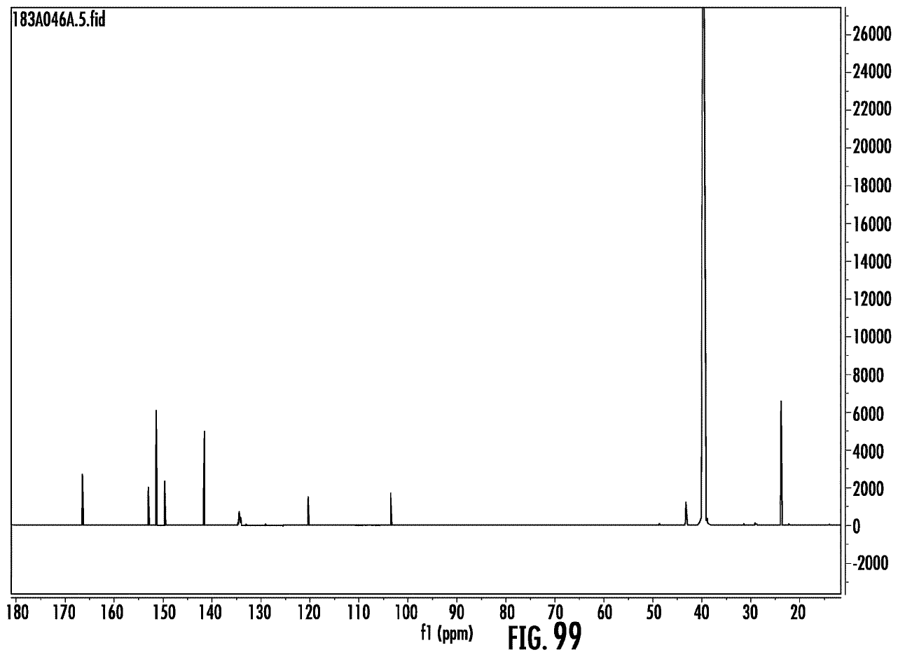

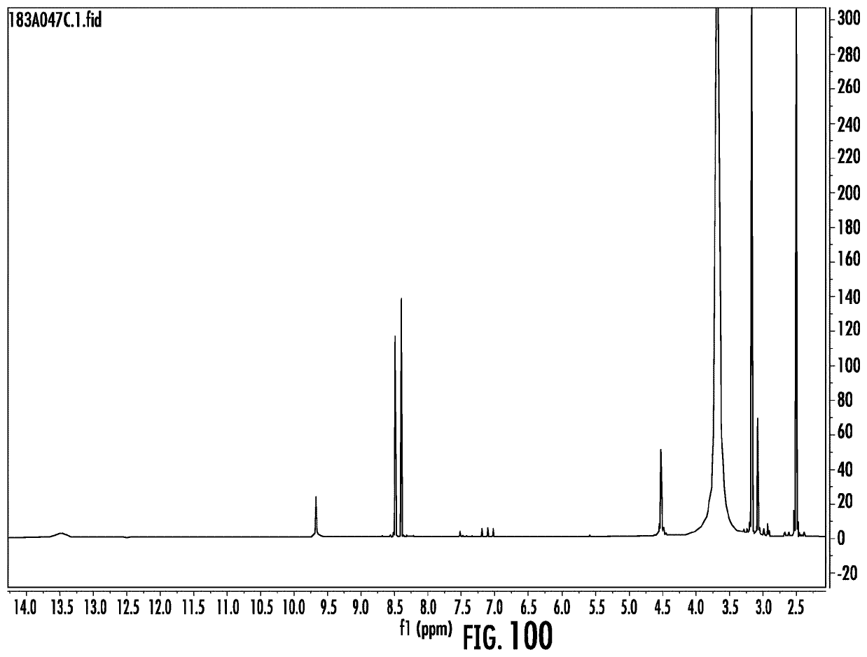

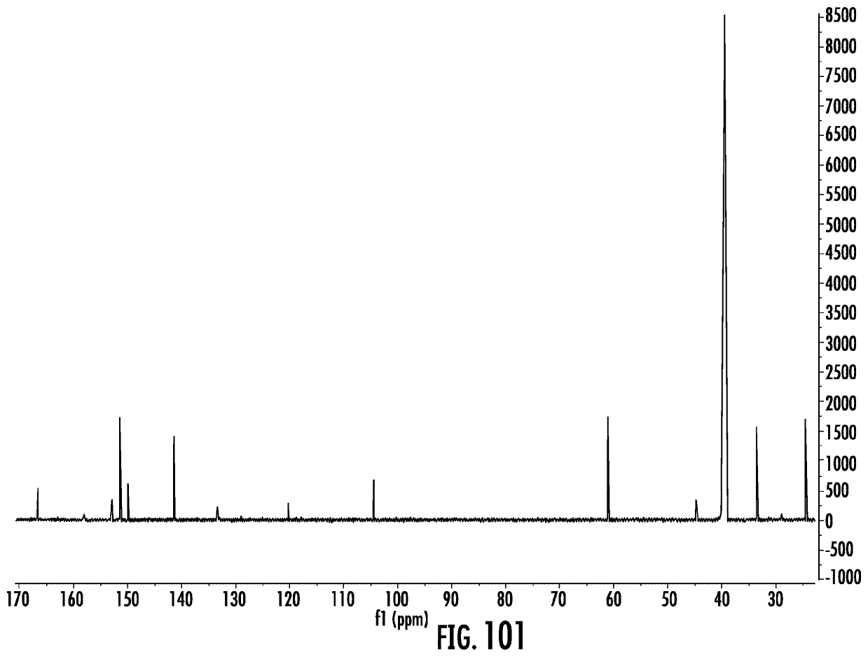

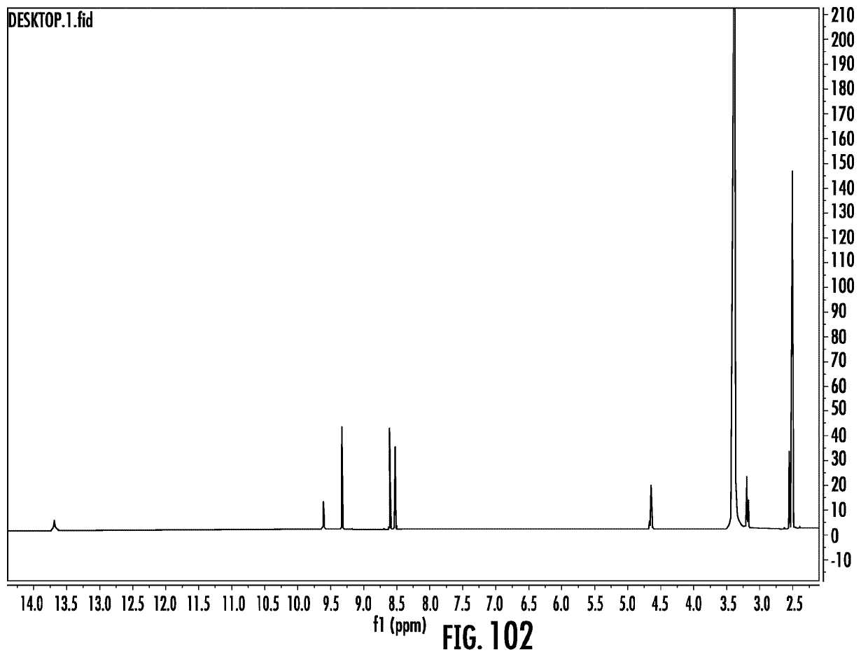

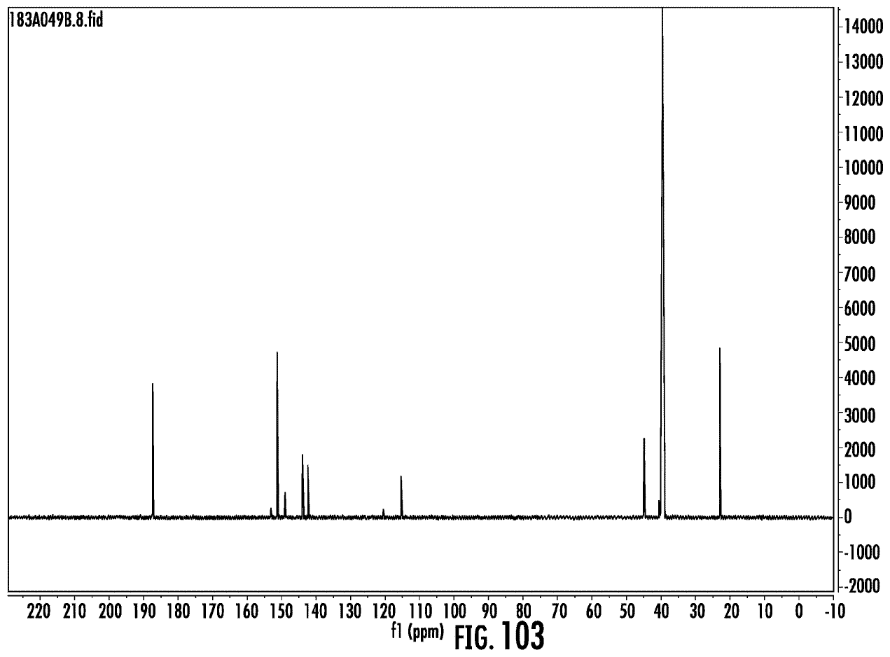

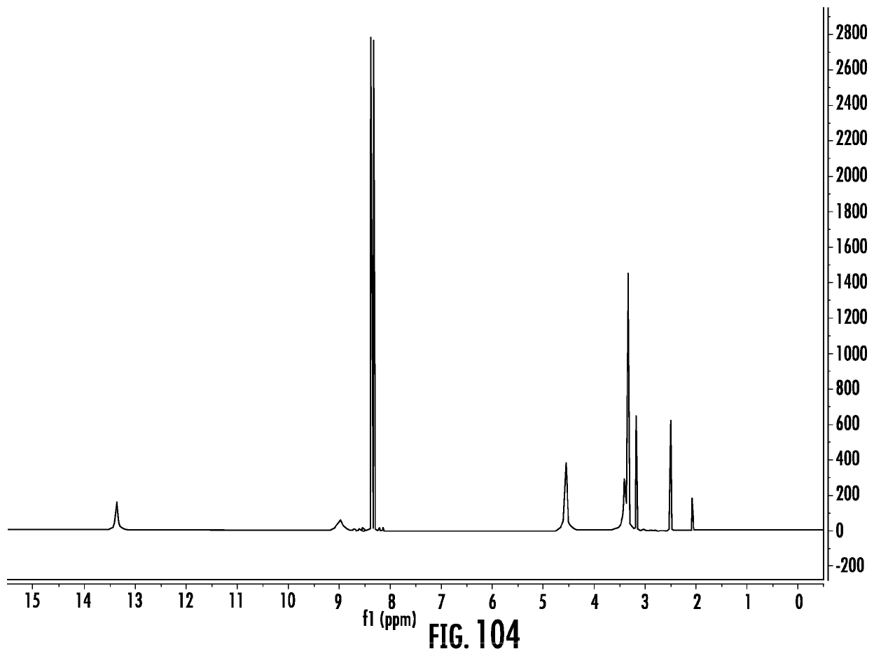

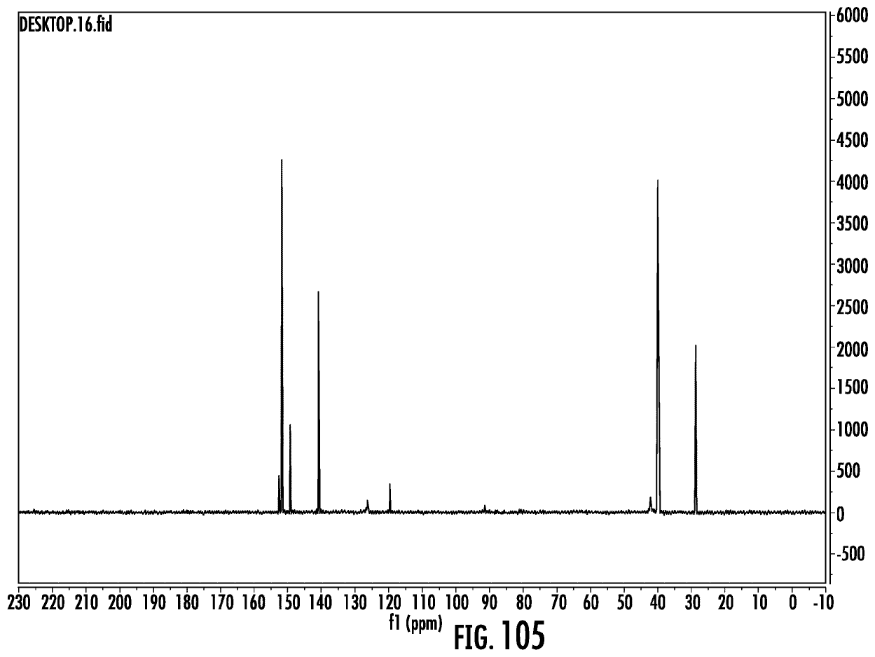

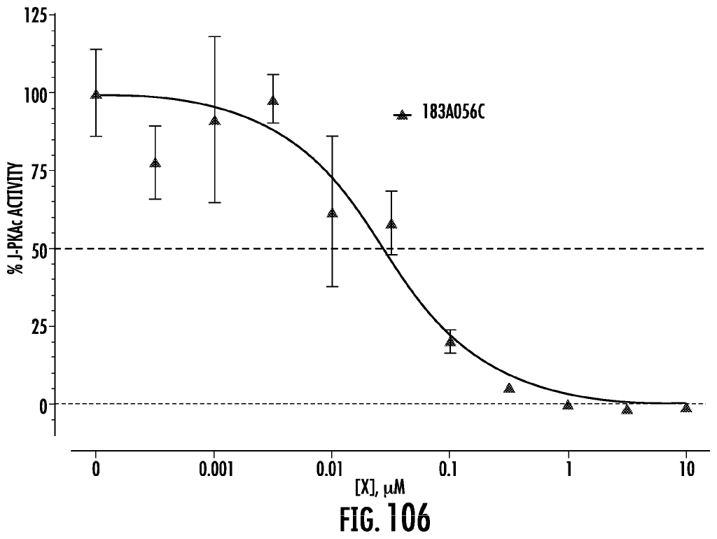

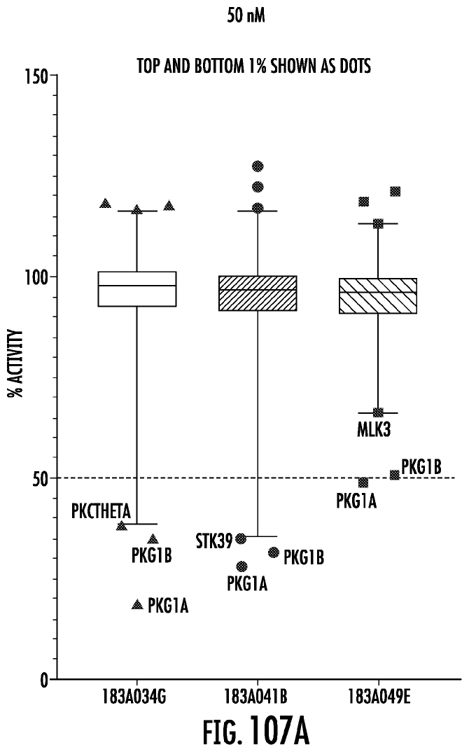

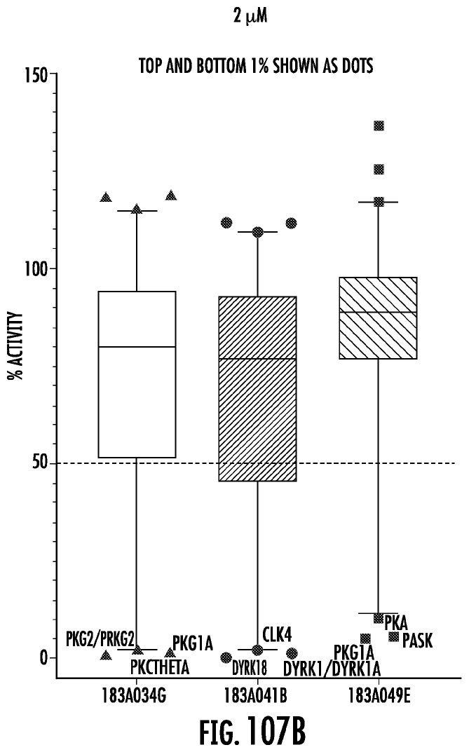

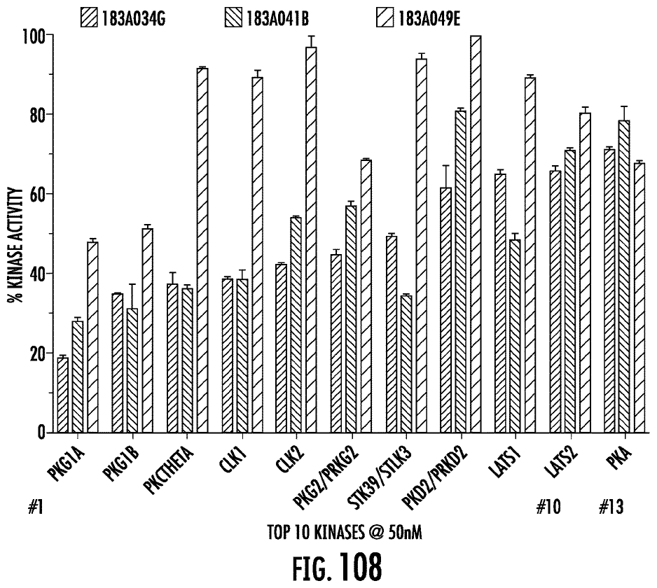

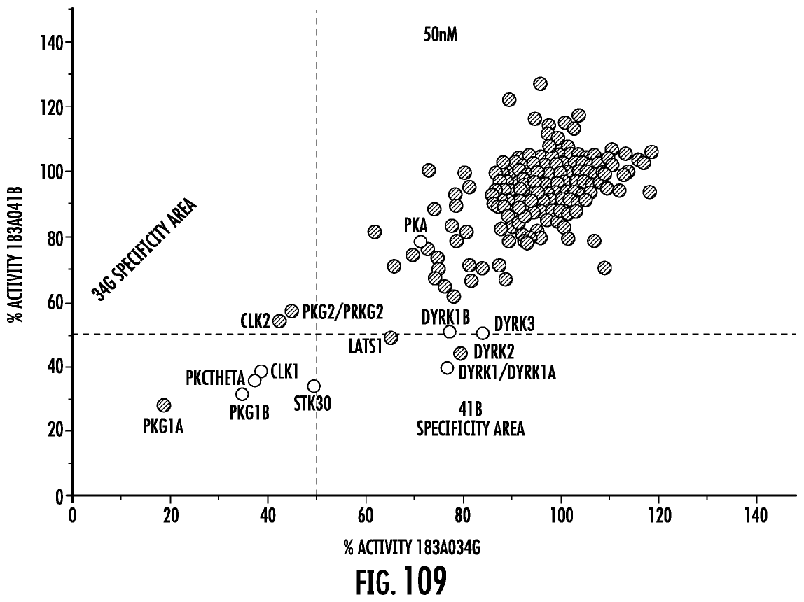

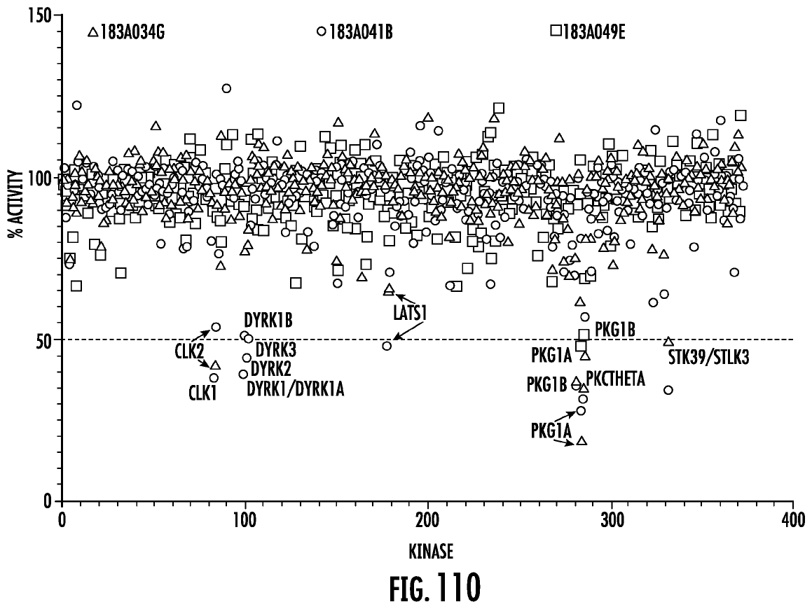

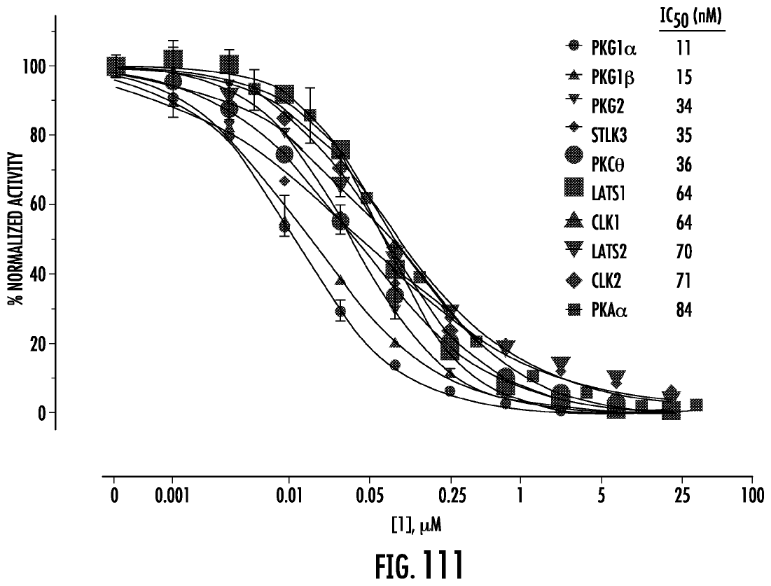

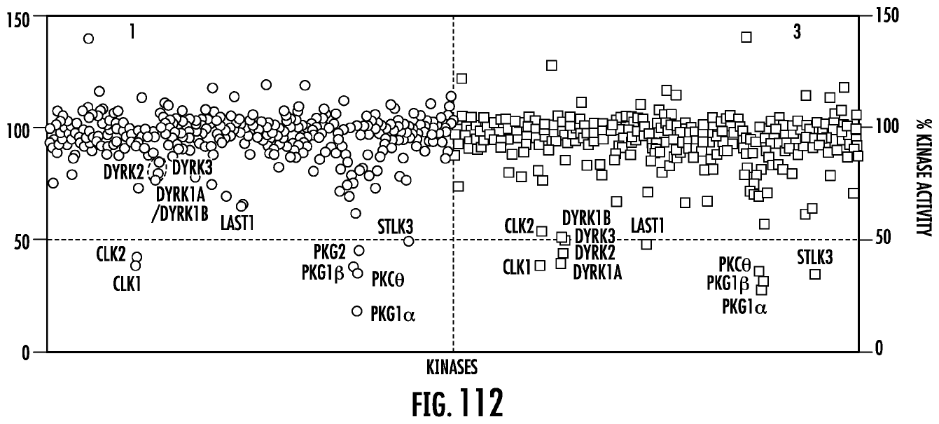

FIG. 91A is a graph showing the percent enzyme activity (relative to controls) and log curve fit.

FIG. 92B is a graph showing the percent enzyme activity (relative to controls) and log curve fit.

FIG. 93A is a graph showing the percent enzyme activity (relative to controls) and log curve fit.

FIG. 93B is a graph showing the percent enzyme activity (relative to controls) and log curve fit.

FIG. 94A is a graph showing the percent enzyme activity (relative to controls) and log curve fit.

FIG. 94B is a graph showing the percent enzyme activity (relative to controls) and log curve fit.

FIG. 95A is a graph showing the percent enzyme activity (relative to controls) and log curve fit.

FIG. 95B is a graph showing the percent enzyme activity (relative to controls) and log curve fit.

FIG. 96 shows a ¹H NMR spectrum of 8 in DMSO-d₆.

FIG. 97 shows a ¹³C NMR spectrum of 8 in DMSO-d₆.

FIG. 98 shows a ¹H NMR spectrum of 9 in DMSO-d₆.

FIG. 99 shows a ¹³C NMR spectrum of 9 in DMSO-d₆.

FIG. 100 shows a ¹H NMR spectrum of 10 in DMSO-d₆.

FIG. 101 shows a ¹³C NMR spectrum of 10 in DMSO-d₆.

FIG. 102 shows a ¹H NMR spectrum of 11 in DMSO-d₆.

FIG. 103 shows a ¹³C NMR spectrum of 11 in DMSO-d₆.

FIG. 104 shows a ¹H NMR spectrum of 12 in DMSO-d₆.

FIG. 105 shows a ¹³C NMR spectrum of 12 in DMSO-d₆.

FIG. 106 is a graph showing the normalized % JPKAcα Activity curve for compound 183A056C.

FIG. 107A shows a graph with results of a kinase testing using 50 nM concentrations of compounds of an aspect of the invention.

FIG. 107B grows a graph with the results of kinase testing using 2 μM concentrations of compounds of an aspect of the invention.

FIG. 108 shows a graph with the results of kinase testing using 50 nM concentrations of compounds of an aspect of the invention.

FIG. 109 shows a graph with the results of kinase testing using 50 nM concentrations of compounds of an aspect of the invention.

FIG. 110 shows a graph with the results of a kinase testing using 50 nM concentrations of compounds of an aspect of the invention.

FIG. 111 shows a graph with the results of kinase testing of Compound 1.

FIG. 112 shows a graph with the results of kinase testing of Compounds 1 and 3.

### DETAILED DESCRIPTION OF THE INVENTION

The Molecular Targets Program of the United States' National Cancer Institute (NCI) completed a screen of ˜150,000 pre-fractionated natural products from the NCI Program for Natural Product Discovery (NPNPD) (Thornburg et al., ACS Chem. Biol., 13: 2484-2497 (2018)). A class of active compounds identified were isolated form the marine organism Aplidium sp. These compounds, named Aplithianines A & B, were shown to potently inhibit both (1) oncogenic gene fusion DNAJB1-PRKACA (PKADJ) and (2) wild type protein kinase A (PKA) at nanomolar concentrations. Aplithianine A was shown to potently and selectively inhibit a broad range of kinases, not just PKADJ or PKA, broadening its potential utility. Further kinetic analysis showed that Aplithianine A was a competitive inhibitor of kinases, competing with ATP for binding to PKA. Additional structural studies showed that aplithianine A bound to the catalytic pocket in PKADJ where ATP normally binds, further proving the competitive mechanism of inhibition and providing structural insights for further synthetic modification of this compound class. Additional semisynthetic efforts in the MTP have created non-natural aplithianine derivatives, one of which (a brominated derivative) was found to be equally active in comparison to the natural product. Additional derivatives were also created providing further SAR data on structure-activity-relationships for this compound class. Two synthesis plans have been designed and completed for the total synthesis of aplithianine A.

The aplithianine structural class is a group of potent kinase inhibitors with broad potential applicability to numerous kinases of importance, e.g., for cancer chemotherapy. For example, gene fusions (a genetic lesion ligating two normally non-adjacent portions of the genome next to one another) were one of the earliest recognized biomarkers of cancer. Approximately 20% of all solid malignancies have at least one identifiable gene fusion. The experience with the BCR-ABL1 kinase inhibitor imatinib (Savage, et al., N. Engl. J. Med., 346(9): 683-93 (2002)), and a continuing emphasis on precision medicine suggests that focusing on gene fusion associated pharmaceutical development could produce disease specific medicines. One such fusion is the recently identified PKADJ oncogenic gene fusion associated with fibrolamellar hepatocellular carcinoma (FL-HCC) (Honeyman, et al., Science, 343: 1010-14 (2014) and Kastenhuber, et al., PNAS USA, 114: 13076-84 (2017)). Among liver cancers, FL-HCC is unusually tragic in that its patient population is young (<35 years of age) and lacks any successful disease specific chemotherapeutic regime, with a 5 year survival rate of only approximately 34% (Riggle, et al., Pediatr. Blood Cancer, 63: 1163-7 (2016)). The biological understanding of FL-HCC improved in 2014 when for the first time it was shown that all FL-HCC patients carried an in-frame intrachromosomal gene fusion between the first exon of the gene encoding the Heat Shock Protein 40 (HSP40) family member DNAJB1 and the second exon of the gene for the adenosine 3′,5′-monophosphate (cAMP)-dependent PKA catalytic subunit alpha, PRKACA (Honeyman, et al., Science, 343: 1010-14 (2014)). The DNAJB1-PRKACA gene fusin produces an enzymatically active chimeric protein DNAJ. Studies have demonstrated that PKA activity was required for tumor formation. Equivalent expression of PKAca or expression of a kinase-dead version of the oncogenic fusion protein is not sufficient for transformation and the tumorigenicity of PKAJ is dependent on its kinase activity (Kastenhuber, et al., PNAS USA, 114: 13076-84 (2017)).

DNAJ fusion complexes may present novel small molecule binding sites which can be exploited for the treatment of FL-HCC (Tomasini, et al., Scientific Reports, 8: 720 (2018); Cheung, et al., PNAS USA, 112: 1374-79 (2015); and Averill, et al., J Cell Biochem., 120: 13783-91 (2019)). Accordingly, a modified sandwich ELISA assay was developed using a biotinylated peptide derived from a PKA substrate (see e.g., Example 4). The reaction and readout components of this assay were than optimized including optimal reactant concentrations for the kinase reaction suitable to identify both inhibitors and activators of PKAJ. The same system was also used to test active samples for inhibition of WT-PKA to identify any compounds with selectivity for the fusion protein (see e.g., Example 6).

### Compounds

In an aspect, the invention provides a compound of (formula (I)

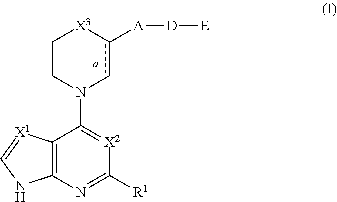

wherein
[symbol] is a single or double bond,
X¹ and X² are each independently CH, CR⁶, or N;
X³ is S, S═O, or S(═O)₂;
R¹ is H or —NR²R³;
R² is H or C₁-C₃ alkyl;
R³ is an aryl;
R⁶ is C₁-C₃ alkyl;
A is optional and, when present, is —C(O)—, —C(O)O—, —C(O)NH—, —C(O)N(C₁-C₃-akyl)-; —C(O)NH—(C₁-C₆ alkyl)-NHC(O)—, —NH;
D is optional and, when present, is a C₁-C₆ alkyl, C₃-C₈ cycloalkyl, C₃-C₈ heterocycloalkyl, —(C₁-C₆ alkyl)-O—, —(C₁-C₆ alkyl)-NH—, —(C₁-C₃ alkyl)-O—(C₁-C₃ alkyl)-, or —(C₁-C₃ alkyl)-NH—(C₁-C₃ alkyl)-, wherein the alkyl group or cycloalkyl group of any of the foregoing is optionally substituted with one or more substituents selected from hydroxy, C₁-C₆ alkyl, amino, C₁-C₆ alkylamino, or di-C₁-C₆ alkyl-amino; —NH-aryl, and a combination thereof; and E is:
   aryl or heteroaryl, optionally substituted with one or more substituents selected from C₁-C₆ alkyl or alkoxy, —(C₁-C₆ alkyl)-OH, —(C₁-C₆ alkyl)-COOH, —(C₁-C₆ alkyl)-NH₂, halo, nitro, hydroxy, amino, C₁-C₆ alkylamino, di-C₁-C₆ alkyl-amino; —NH-aryl, C₁-C₆ haloalkyl, C₃-C₈ cycloalkyl or heterocycloalkyl, fused C₃-C₈ cycloalkyl or heterocycloalkyl, aryl or heteroaryl, fused aryl or heteroaryl, —CN, —(C₁-C₃ alkyl)-CN, carbonyl, and a combination thereof;
   amino, C₁-C₆ alkylamino, or di-C₁-C₆ alkyl-amino; or —NH-aryl;
   C₁-C₆ alkyl or alkoxy, optionally substituted with one or more substituents selected from hydroxy, C₁-C₆ alkyl, amino, C₁-C₆ alkylamino, or di-C₁-C₆ alkyl-amino; —NH-aryl, and a combination thereof;
   C₃-C₈ cycloalkyl or heterocycloalkyl, optionally substituted with one or more substituents selected from hydroxy, C₁-C₆ alkyl, amino, C₁-C₆ alkylamino, di-C₁-C₆ alkyl-amino; —NH-aryl, C₃-C₈ cycloalkyl or heterocycloalkyl, fused C₃-C₈ cycloalkyl or heterocycloalkyl, aryl or heteroaryl, fused aryl or heteroaryl, and a combination thereof;
   —C(O)NH₂;
   —C(O)OH;
   —C(O)H;
   —N—(C₁-C₆ alkyl)-acrylamide;
   halogen; or
   hydrogen;
or a pharmaceutically acceptable salt thereof.

In some embodiments, the invention provides a compound of formula (I)

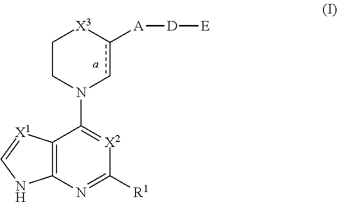

wherein
[symbol] is a single or double bond,
X¹ and X² are each independently CH or N;
X³ is S, S═O, or S(═O)₂;
R¹ is H or —NR²R³;
R² is H or C₁-C₃ alkyl;
R³ is an aryl;
A is optional and, when present, is-C(O)—, —C(O)O—, —C(O)NH—, —C(O)N(C₁-C₃-akyl)-; —C(O)NH—(C₁-C₆ alkyl)-NHC(O)—, —NH;
D is optional and, when present, is a C₁-C₆ alkyl, —(C₁-C₆ alkyl)-O—, —(C₁-C₆ alkyl)-NH—, —(C₁-C₃ alkyl)-O—(C₁-C₃ alkyl)-, or —(C₁-C₃ alkyl)-NH—(C₁-C₃ alkyl)-, wherein the alkyl group of any of the foregoing is optionally substituted with hydroxy; and
E is:
aryl or heteroaryl, optionally substituted with C₁-C₆ alkyl or alkoxy, halo, nitro, hydroxy, C₁-C₆ haloalkyl, —CN, —(C₁-C₃ alkyl)-CN, or carbonyl;
amino, C₁-C₆ alkylamino, or di-C₁-C₆ alkyl-amino; or —NH-aryl;
C₁-C₆ alkyl or alkoxy, optionally substituted with hydroxy;
C₃-C₈ cycloalkyl or heterocycloalkyl, optionally substituted with hydroxy;
—C(O)NH₂;
—C(O)OH;
—C(O)H;
—N—(C₁-C₆ alkyl)-acrylamide;
halogen; or
hydrogen;
or a pharmaceutically acceptable salt thereof.

In an aspect of the invention, [symbol] a is a single bond.

In an aspect of the invention, [symbol] is a double bond.

In an aspect of the invention, the compound of formula (I) is of formula (Ia):

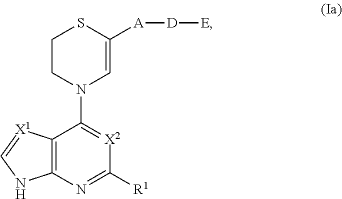

or a pharmaceutically acceptable salt thereof.

In an aspect of the invention, the compound of formula (I) is of formula (Ib):

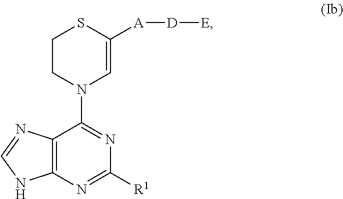

or a pharmaceutically acceptable salt thereof.

In an aspect of the invention, R¹ is H.

In some aspects,
(i) A and D are absent, and E is halogen; —C(O)OH; —C(O)H; aryl or heteroaryl, optionally substituted with C₁-C₃ alkyl, or halo; or C₃-C₈ cycloalkyl or heterocycloalkyl, optionally substituted with hydroxy;
(ii) A is absent; D is a C₁-C₆ alkyl, optionally a C₁-C₃ alkyl; and E is —C(O)NH₂ or a C₃-C₈ cycloalkyl or heterocycloalkyl, optionally substituted with hydroxy;
(iii) A is —NH—, —C(O)NH— or —C(O)N(C₁-C₃-akyl)-; D is absent or is a C₁-C₆ alkyl, optionally a C₁-C₃ alkyl; and E is amino, C₁-C₆ alkylamino, or di-C₁-C₆ alkyl-amino; or —NH-aryl; C₁-C₆ alkyl or alkoxy or C₁-C₃ alkyl or alkoxy, optionally substituted with hydroxy; or aryl or heteroaryl, optionally substituted with C₁-C₃ alkyl or alkoxy, halo, nitro, hydroxy, C₁-C₃ haloalkyl, —CN, —(C₁-C₃ alkyl)-CN, or carbonyl;
(iv) A is absent, D is —(C₁-C₆ alkyl)-O—, —(C₁-C₆ alkyl)-NH—, —(C₁-C₃ alkyl)-O—(C₁-C₃ alkyl)-, or —(C₁-C₃ alkyl)-NH—(C₁-C₃ alkyl)-, wherein the alkyl groups of any of the foregoing is optionally substituted with hydroxy and wherein the alkyl groups of any of the foregoing are optionally branched; and E is C₁-C₆ alkyl or C₃-C₈ cycloalkyl or heterocycloalkyl, optionally substituted with hydroxy;
(v) A is —C(O)NH—(C₁-C₆ alkyl)-NHC(O)—, optionally C(O)NH—(C₁-C₃ alkyl)-NHC(O)—; D is absent; and E is C₁-C₆ alkyl or C₁-C₃ alkyl; or aryl or heteroaryl, optionally substituted with C₁-C₃ alkyl or alkoxy, halo, nitro, hydroxy, C₁-C₆ haloalkyl, —CN, —(C₁-C₃ alkyl)-CN, or carbonyl;
(vi) A is —C(O)O—; D is absent; and E is C₁-C₆ alkyl; or
(vii) A is —C(O)—; D is absent; and E is heterocyloalkyl.

In an aspect of the invention, the compound of formula (I) is of formula (Ic):

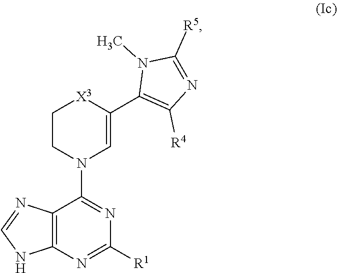

wherein R⁴ and R⁵ are the same or different and each is H or halo (e.g., bromine, fluorine, chlorine, or iodine), or a pharmaceutically acceptable salt thereof.

In an aspect of the invention, one of R⁴ and R⁵ is halo. In an aspect of the invention, both R⁴ and R⁵ are halo.

In an aspect of the invention, R⁴ and R⁵ are both hydrogen. In some embodiments of the foregoing aspects, the halogen is bromine.

In an aspect of the invention, the compound is not aplithianine A (Compound 1)

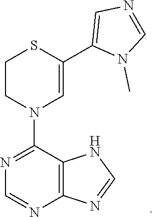 [SMILES: Cn1cncc1C1=CN(c2ncnc3nc[nH]c23)CCS1]

Aspects of the invention provide the following compounds:

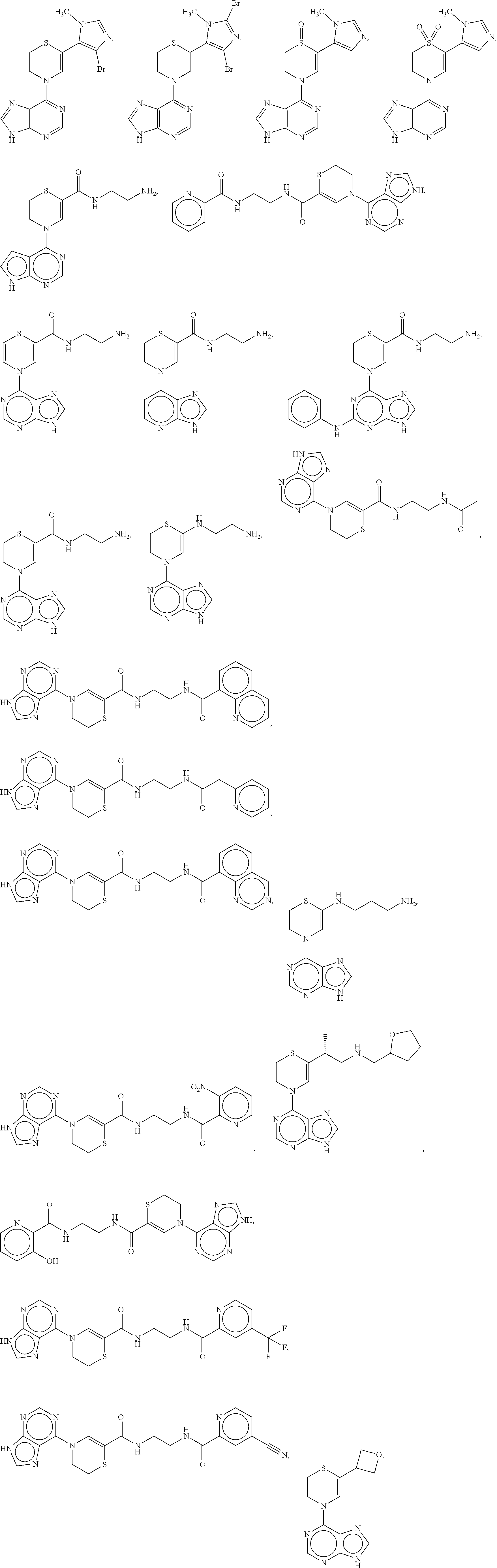

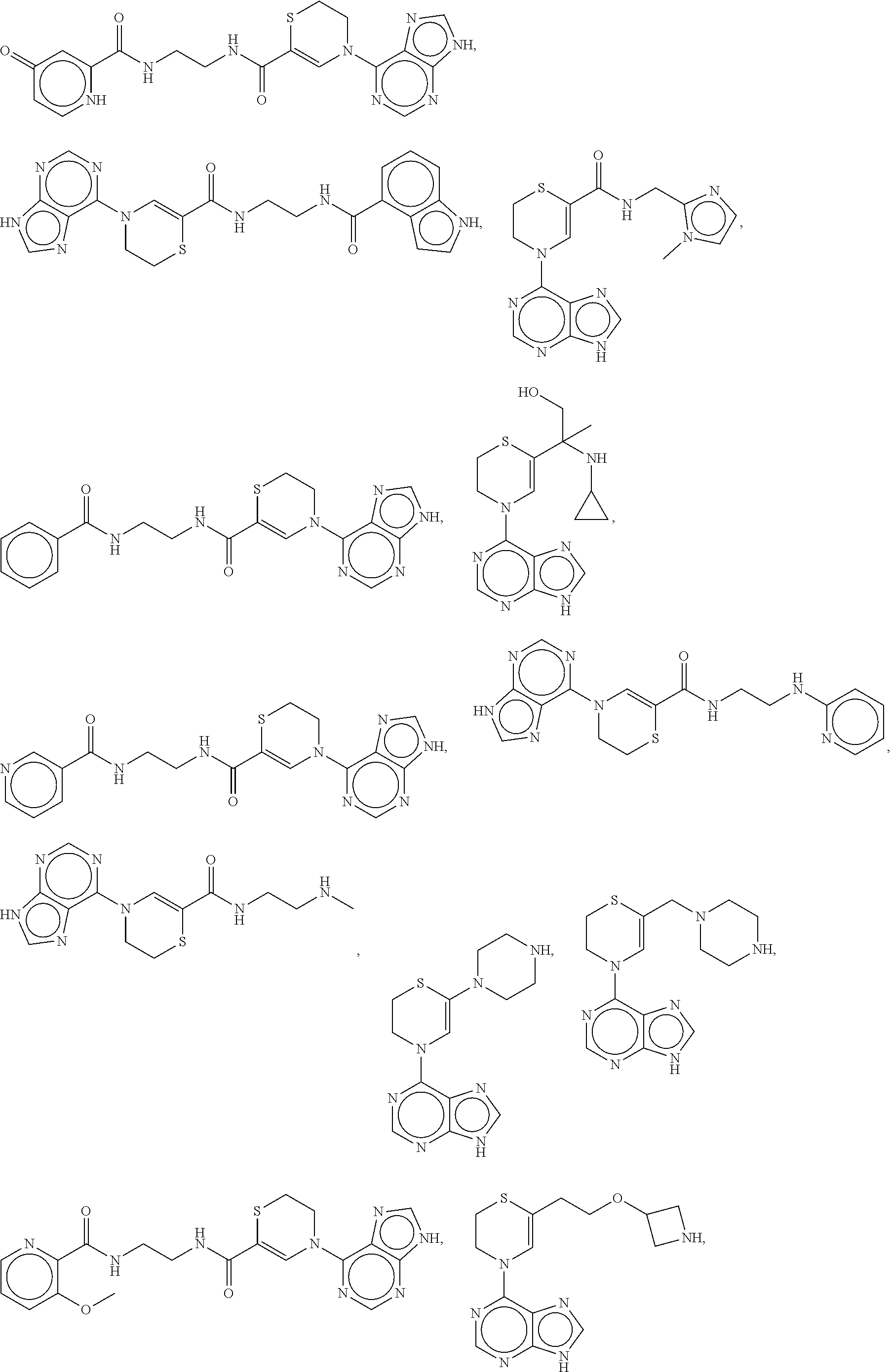

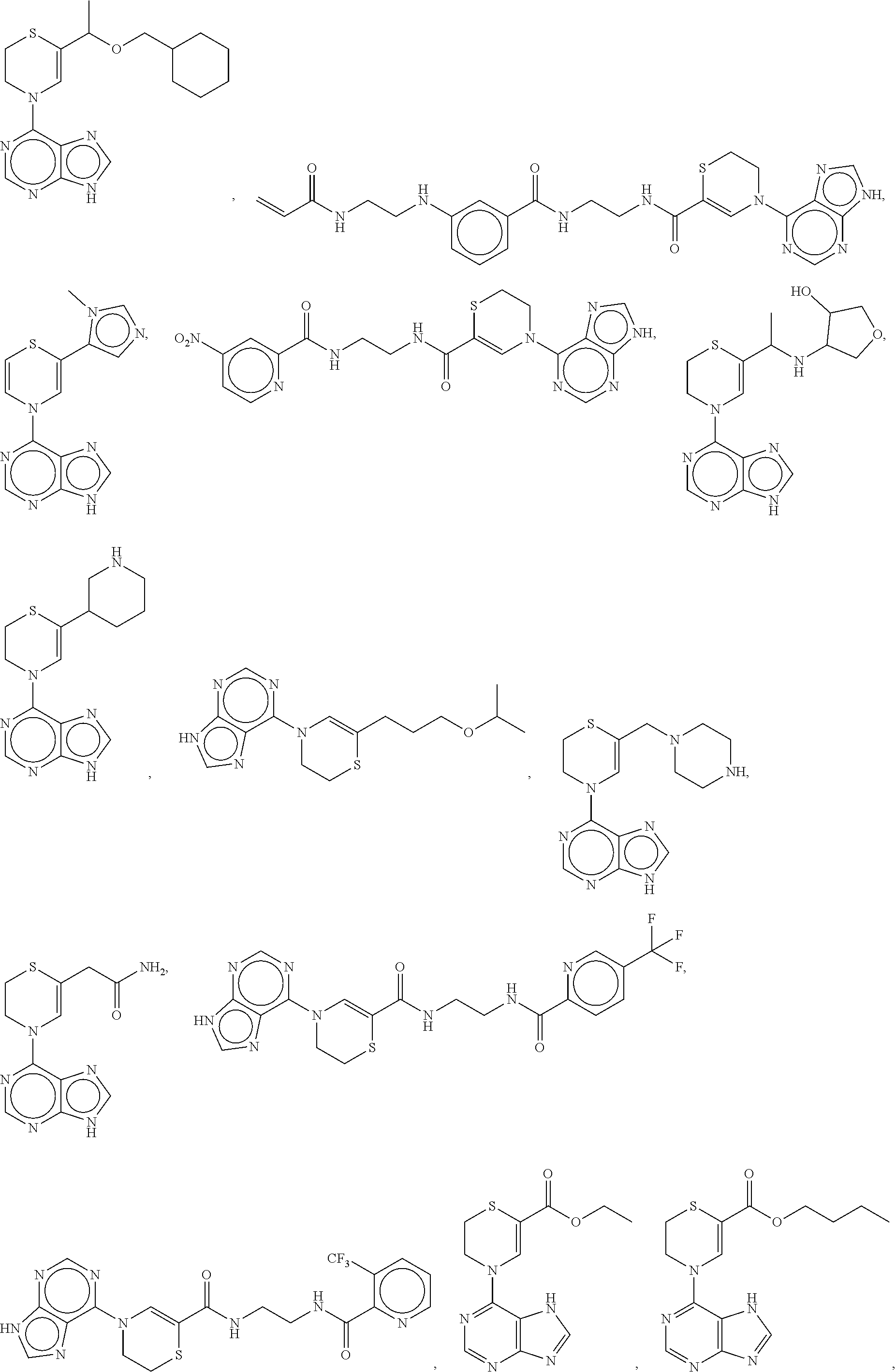

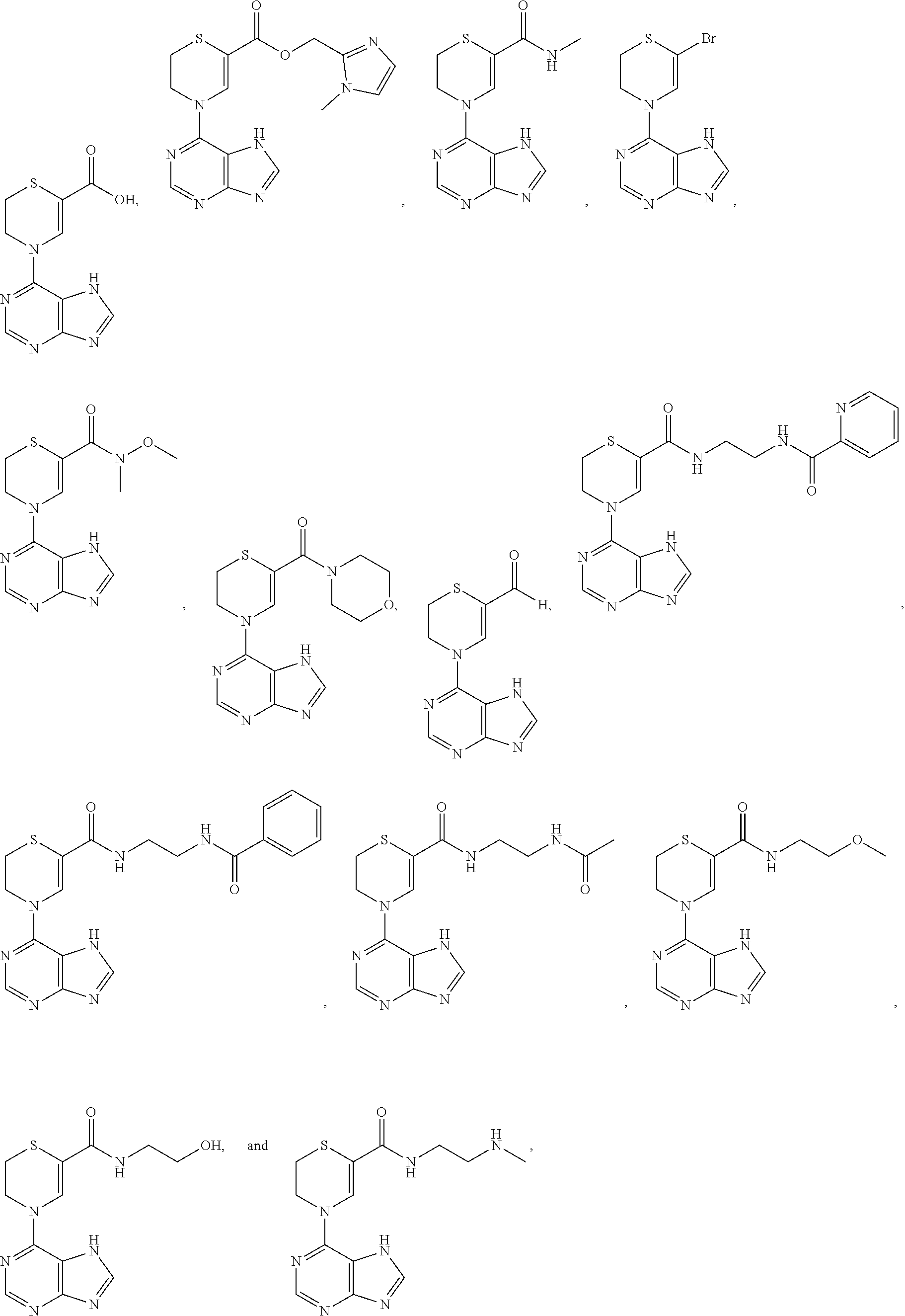

or a pharmaceutically acceptable salt thereof.

Aspects of the invention provide the following compounds:

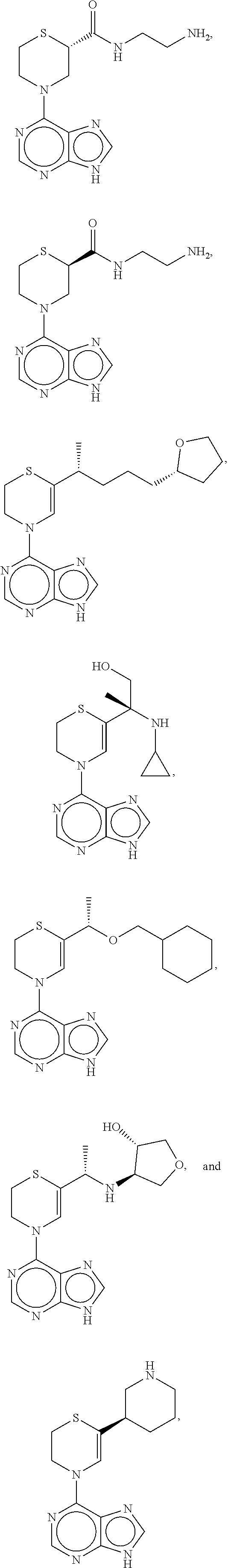

or a pharmaceutically acceptable salt thereof.

Aspects of the invention provide the following compounds:

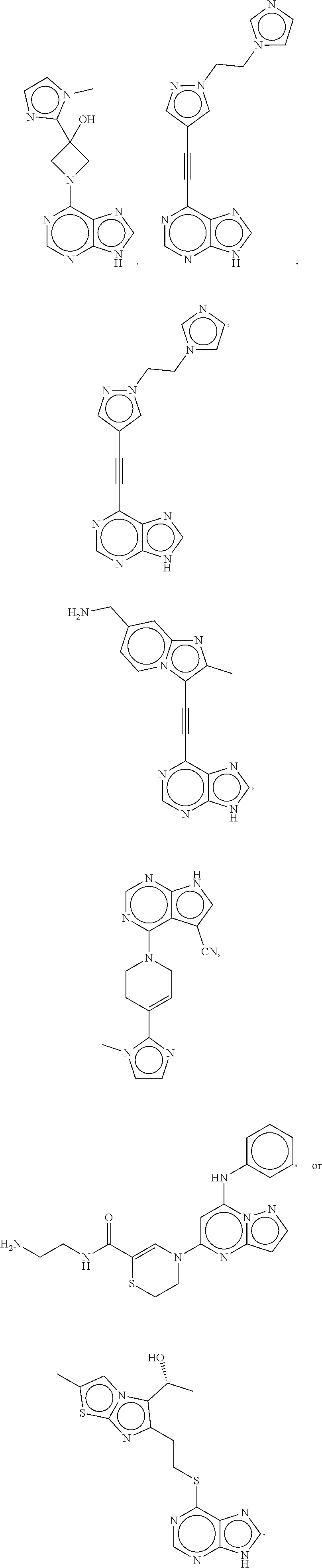

or a pharmaceutically acceptable salt thereof.

Aspects of the invention provide the compounds of FIGS. 9A-9J.

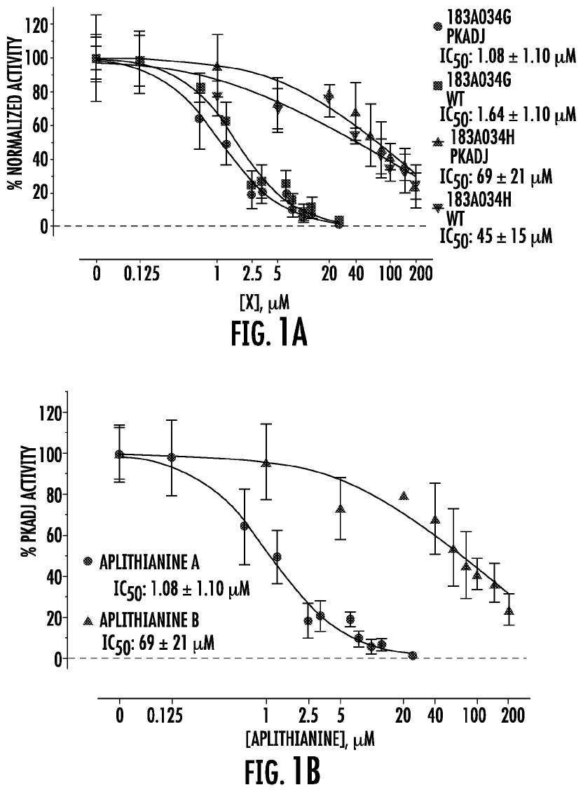

Aspects of the invention provide the compounds of FIGS. 10A-1OP.

Aspects of the invention provide the compounds of FIGS. 12A-12D.

Further aspects of the invention provide enantiomers of the compounds disclosed herein.

In any of the aspects above, the term “alkyl” implies a straight-chain or branched alkyl substituent containing from, for example, from about 1 to about 6 carbon atoms, e.g., from about 1 to about 4 carbon atoms. Examples of alkyl group include methyl, ethyl, n-propyl, isopropyl, n-butyl, sec-butyl, isobutyl, tert-butyl, n-pentyl, isopentyl, n-hexyl, and the like. This definition also applies wherever “alkyl” occurs as part of a group, such as, e.g., in C₃-C₆ cycloalkylalkyl, hydroxyalkyl, haloalkyl (e.g., monohaloalkyl, dihaloalkyl, and trihaloalkyl), cyanoalkyl, aminoalkyl, alkylamino, dialkylamino, alkylaminoalkyl, dialkylaminoalkyl, arylcarbonylalkyl (-(alkyl)C(O)aryl), arylalkyl, etc. The alkyl can be substituted or unsubstituted, as described herein. Even in instances in which the alkyl is an alkylene chain (e.g., —(CH₂)ₙ—), the alkyl group can be substituted or unsubstituted.

In any of the aspects above, the term “alkenyl,” as used herein, means a linear alkenyl substituent containing from, for example, about 2 to about 6 carbon atoms (branched alkenyls are about 3 to about 6 carbons atoms), e.g., from about 3 to about 5 carbon atoms (branched alkenyls are about 3 to about 6 carbons atoms). In accordance with an aspect of the invention, the alkenyl group is a C₂-C₄ alkenyl. Examples of alkenyl group include ethenyl, allyl, 2-propenyl, 1-butenyl, 2-butenyl, 1-pentenyl, 2-pentenyl, 3-pentenyl, 1-hexenyl, and the like. The alkenyl can be substituted or unsubstituted, as described herein.

In any of the aspects above, the term “cycloalkyl,” as used herein, means a cyclic alkyl moiety containing from, for example, 3 to 6 carbon atoms or from 5 to 6 carbon atoms. Examples of such moieties include cyclopropyl, cyclobutyl, cyclopentyl, cyclohexyl, and the like. The cycloalkyl may contain a carbonyl such that there is an exocylclic (═O) group. The cycloalkyl can be substituted or unsubstituted, as described herein. The cycloalkyl may also be fused to a neighboring substituent (e.g., a cycloalkyl, a heterocycloalkyl, an aryl, or a heteroaryl), i.e, sharing two atoms and a bond with a neighboring substituent.

In any of the aspects above, the term “aryl” refers to a mono, bi, or tricyclic carbocyclic ring system having one, two, or three aromatic rings, for example, phenyl, naphthyl, anthracenyl, or biphenyl. The term “aryl” refers to an unsubstituted or substituted aromatic carbocyclic moiety, as commonly understood in the art, and includes monocyclic and polycyclic aromatics such as, for example, phenyl, biphenyl, naphthyl, anthracenyl, pyrenyl, and the like. An aryl moiety generally contains from, for example, 6 to 30 carbon atoms, from 6 to 18 carbon atoms, from 6 to 14 carbon atoms, or from 6 to 10 carbon atoms. It is understood that the term aryl includes carbocyclic moieties that are planar and comprise 4n+2 π electrons, according to Huckel's Rule, wherein n=1, 2, or 3. This definition also applies wherever “aryl” occurs as part of a group, such as, e.g., in haloaryl (e.g., monohaloaryl, dihaloaryl, and trihaloaryl), arylalkyl, etc. The aryl can be substituted or unsubstituted, as described herein. The aryl may also be fused to a neighboring substituent (e.g., a cycloalkyl, a heterocycloalkyl, an aryl, or a heteroaryl), i.e,. sharing two atoms and a bond with a neighboring substituent.

In any of the aspects above, the term “heteroaryl” refers to aromatic 5 or 6 membered monocyclic groups, 9 or 10 membered bicyclic groups, and 11 to 14 membered tricyclic groups which have at least one heteroatom (O, S, or N) in at least one of the rings. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. The fused rings completing the bicyclic and tricyclic groups may contain only carbon atoms and may be saturated, partially saturated, or unsaturated. The nitrogen and sulfur atoms may optionally be oxidized, and the nitrogen atoms may optionally be quaternized. Heteroaryl groups which are bicyclic or tricyclic must include at least one fully aromatic ring but the other fused ring or rings may be aromatic or non-aromatic. The heteroaryl group may be attached at any available nitrogen or carbon atom of any ring. Illustrative examples of heteroaryl groups are pyridinyl, pyridazinyl, pyrimidyl, pyrazinyl, benzimidazolyl, triazinyl, imidazolyl, (1,2,3)-and (1,2,4)-triazolyl, pyrazinyl, tetrazolyl, furyl, pyrrolyl, thienyl, isothiazolyl, thiazolyl, isoxazolyl, and oxadiazolyl. The heteroaryl can be substituted or unsubstituted, as described herein. The heteroaryl may also be fused to a neighboring substituent (e.g., a cycloalkyl, a heterocycloalkyl, an aryl, or a heteroaryl), i.e,. sharing two atoms and a bond with a neighboring substituent.

The term “heterocycloalkyl” means a stable, saturated, or partially unsaturated monocyclic, bicyclic, and spiro ring system containing 3 to 7 ring members of carbon atoms and other atoms selected from nitrogen, sulfur, and/or oxygen. In an aspect, a heterocycloalkyl is a 5, 6, or 7-membered monocyclic ring and contains one, two, or three heteroatoms selected from nitrogen, oxygen, and sulfur. The heterocycloalkyl may be attached to the parent structure through a carbon atom or through any heteroatom of the heterocycloalkyl that results in a stable structure. Alternatively, or additionally, the heterocycloalkyl may contain a carbonyl such that there is an exocylclic (═O) group. Examples of such heterocycloalkyl rings are isoxazolyl, thiazolinyl, imidazolidinyl, piperazinyl, homopiperazinyl, pyrrolyl, pyrrolinyl, pyrazolyl, pyranyl, piperidyl, oxazolyl, and morpholinyl. The heterocycloalkyl can be substituted or unsubstituted, as described herein.

In any of the aspects above, the term “hydroxy” refers to the group —OH.

In any of the aspects above, the term “cyano” refers to the group —CN, whereas the term “thiocyano” refers to —SCN.

In any of the aspects above, the terms “alkoxy” and “cycloalkyloxy” embrace linear or branched alkyl and cycloalkyl groups, respectively, that are attached to a divalent oxygen. The alkyl and cycloalkyl groups are the same as described herein.

In any of the aspects s above, the term “halo” refers to a halogen selected from fluorine, chlorine, bromine, and iodine.

In any of the aspects above, the term “carboxylato” refers to the group —C(O)OH.

In any of the aspects above, the term “amino” refers to the group —NH₂. The term “alkylamino” refers to —NHR, whereas the term “dialkylamino” refers to —NRR′. R and R′ are the same or different and each is a substituted or unsubstituted alkyl group, as described herein.

In any of the aspects above, the term “amido” refers to the group —C(O)NRR′, which R and R′ are the same or different and each is hydrogen or a substituted or unsubstituted alkyl group, as described herein.

In any of the aspects s above, the term “phosphonato” refers to the group —P(O)(OR)₂, which R is hydrogen or a substituted or unsubstituted alkyl group, as described herein.

In other aspects, any substituent that is not hydrogen (e.g., C₁-C₆ alkyl, C₂-C₆ alkenyl, C₃-C₆ cycloalkyl, C₃-C₆ cycloalkylalkyl, aryl, arylalkyl, heteroaryl, heteroarylalkyl, heterocycloalkyl, or heterocycloalkylalkyl) can be an optionally substituted moiety. The substituted moiety typically comprises at least one substituent (e.g., 1, 2, 3, 4, 5, 6, etc.) in any suitable position (e.g., 1-, 2-, 3-, 4-, 5-, or 6-position, etc.). When an aryl group is substituted with a substituent, e.g., halo, amino, alkyl, OH, alkoxy, and others, the aromatic ring hydrogen is replaced with the substituent and this can take place in any of the available hydrogens, e.g., 2, 3, 4, 5, and/or 6-position wherein the 1-position is the point of attachment of the aryl group in the compound of the present invention. Suitable substituents include, e.g., halo, alkyl, alkenyl, hydroxy, nitro, cyano, amino, alkylamino, alkoxy, aryloxy, aralkoxy, carboxyl, carboxyalkyl, carboxyalkyloxy, amido, alkylamido, haloalkylamido, aryl, heteroaryl, heterocycloalkyl, cycloalkyl, fused aryl, fused heteroaryl, fused heterocycloalkyl, and fused cycloalkyl, each of which is described herein. In some instances, the substituent is at least one alkyl, halo, and/or haloalkyl (e.g., 1 or 2).

In any of the aspects above, whenever a range of the number of atoms in a structure is indicated (e.g., a C₁₋₁₂, C₁₋₈, C₁₋₆, C₁₋₄, etc.), it is specifically contemplated that any sub-range or individual number of carbon atoms falling within the indicated range also can be used. Thus, for instance, the recitation of a range of 1-8 carbon atoms (e.g., C₁-C₈), 1-6 carbon atoms (e.g., C₁-C₆), 1-4 carbon atoms (e.g., C₁-C₄), 1-3 carbon atoms (e.g., C₁-C₃), or 2-8 carbon atoms (e.g., C₂-C₈) as used with respect to any chemical group (e.g., alkyl, cycloalkyl, etc.) referenced herein encompasses and specifically describes 1, 2, 3, 4, 5, 6, 7, and/or 8 carbon atoms, as appropriate, as well as any sub-range thereof (e.g., 1-2 carbon atoms, 1-3 carbon atoms, 1-4 carbon atoms, 1-5 carbon atoms, 1-6 carbon atoms, 1-7 carbon atoms, 1-8 carbon atoms, 2-3 carbon atoms, 2-4 carbon atoms, 2-5 carbon atoms, 2-6 carbon atoms, 2-7 carbon atoms, 2-8 carbon atoms, 3-4 carbon atoms, 3-5 carbon atoms, 3-6 carbon atoms, 3-7 carbon atoms, 3-8 carbon atoms, 4-5 carbon atoms, 4-6 carbon atoms, 4-7 carbon atoms, 4-8 carbon atoms, etc., as appropriate).

In any of the aspects above, the phrase “salt” or “pharmaceutically acceptable salt” is intended to include nontoxic salts synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. For example, an inorganic acid (e.g., hydrochloric acid, sulfuric acid, phosphoric acid, or hydrobromic acid), an organic acid (e.g., oxalic acid, malonic acid, citric acid, fumaric acid, lactic acid, malic acid, succinic acid, tartaric acid, acetic acid, trifluoroacetic acid, gluconic acid, ascorbic acid, methylsulfonic acid, or benzylsulfonic acid), an inorganic base (e.g., sodium hydroxide, potassium hydroxide, calcium hydroxide, magnesium hydroxide, or ammonium hydroxide), an organic base (e.g., methylamine, diethylamine, triethylamine, triethanolamine, ethylenediamine, tris(hydroxymethyl)methylamine, guanidine, choline, or cinchonine), or an amino acid (e.g., lysine, arginine, or alanine) can be used. Generally, nonaqueous media such as ether, ethyl acetate, ethanol, isopropanol, or acetonitrile are typical. Lists of suitable salts are found in Remington's Pharmaceutical Sciences, 18th ed., Mack Publishing Company, Easton, PA, 1990, p. 1445, and Journal of Pharmaceutical Science, 66, 2-19 (1977). For example, they can be a salt of an alkali metal (e.g., sodium or potassium), alkaline earth metal (e.g., calcium), or ammonium of salt. In an aspect, the salt is a trifluoroacetate salt.

### Pharmaceutical Compositions

An aspect of the invention provides pharmaceutical compositions comprising a compound of the present invention. The pharmaceutical compositions contain a pharmaceutically acceptable carrier.

An aspect of the invention provides pharmaceutical compositions comprising a compound of formula I.

An aspect of the invention provides pharmaceutical compositions comprising

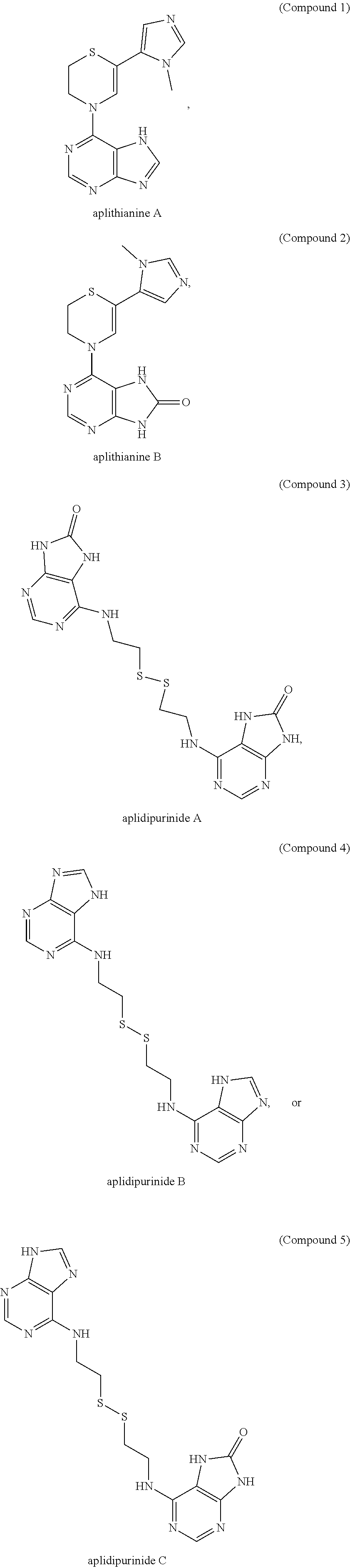 [SMILES: Cn1cncc1C1=CN(c2ncnc3nc[nH]c23)CCS1 ; Cn1cncc1C1=CN(c2ncnc3[nH]c(=O)[nH]c23)CCS1 ; O=c1[nH]c2ncnc(NCCSSCCNc3ncnc4[nH]c(=O)[nH]c34)c2[nH]1 ; c1nc(NCCSSCCNc2ncnc3nc[nH]c23)c2[nH]cnc2n1 ; O=c1[nH]c2ncnc(NCCSSCCNc3ncnc4[nH]cnc34)c2[nH]1]

with at least about 80% purity, and a pharmaceutical carrier.

In an aspect, the compound has a purity of at least about 85% (e.g., at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, at least about 99.6%, at least about 99.7%, at least about 99.8%, or at least about 99.9%).

The pharmaceutical composition can comprise a compound of the present invention in combination with one or more other pharmaceutically active agents or drugs, such as a chemotherapeutic agents, e.g., a topoisomerase I inhibitor, asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

Preferably, the carrier is a pharmaceutically acceptable carrier. With respect to pharmaceutical compositions, the carrier can be any of those conventionally used and is limited only by chemico-physical considerations, such as solubility and lack of reactivity with the active compound(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use.

The choice of carrier will be determined in part by the particular compounds, as well as by the particular method used to administer the compounds. Accordingly, there are a variety of suitable formulations of the pharmaceutical composition of the invention. The compounds, a pharmaceutically acceptable salts thereof, can be administered in any suitable manner (e.g., orally, intravenously, intramuscularly, intrathecally, subcutaneously, sublingually, buccally, rectally, vaginally, by ocular route, by otic route, nasally, by inhalation, by nebulization, topically, systemically, transdermally, or a combination thereof). In an embodiment, the pharmaceutical composition of the invention is administered orally.

The following formulations for administration are exemplary and are in no way limiting. More than one route can be used to administer the compounds, and in certain instances, a particular route can provide a more immediate and more effective response than another route.

Formulations suitable for administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compounds can be administered in a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-1,3-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in formulations include petroleum, animal, vegetable, or synthetic oils. Specific examples of oils include peanut, soybean, sesame, cottonseed, corn, olive, petrolatum, and mineral. Suitable fatty acids for use in formulations include oleic acid, stearic acid, and isostearic acid. Ethyl oleate and isopropyl myristate are examples of suitable fatty acid esters.

Suitable soaps for use in formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quatemary ammonium salts, and (e) mixtures thereof.

The formulations will typically contain from about 0.5% to about 25% by weight of the compounds in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described. The requirements for effective pharmaceutical carriers for compositions are well-known to those of ordinary skill in the art (see, e.g., Lloyd et al. (eds.), Remington: The Science and Practice of Pharmacy, 22nd Ed., Pharmaceutical Press (2012)).

It will be appreciated by one of skill in the art that, in addition to the above-described pharmaceutical compositions, the compounds of the invention can be formulated as inclusion complexes, such as cyclodextrin inclusion complexes, or liposomes.

For purposes of the invention, the amount or dose of the compounds administered should be sufficient to effect a desired response, e.g., a therapeutic or prophylactic response, in the mammal over a reasonable time frame. For example, the dose of the compounds should be sufficient to inhibit growth of a target cell or treat or prevent cancer in a period of from about 2 hours or longer, e.g., 12 to 24 or more hours, from the time of administration. In certain aspects, the time period could be even longer. The dose will be determined by the efficacy of the particular compounds and the condition of the mammal (e.g., human), as well as the body weight of the mammal (e.g., human) to be treated.

Many assays for determining an administered dose are known in the art. An administered dose may be determined in vitro (e.g., cell cultures) or in vivo (e.g., animal studies). For example, an administered dose may be determined by determining the IC₅₀ (the dose that achieves a half-maximal inhibition of symptoms), LD₅₀ (the dose lethal to 50% of the population), the ED₅₀ (the dose therapeutically effective in 50% of the population), and the therapeutic index in cell culture and/or animal studies. The therapeutic index is the ratio of LD₅₀ to ED₅₀ (i.e., LD₅₀/ED₅₀).

The dose of the compounds also will be determined by the existence, nature, and extent of any adverse side effects that might accompany the administration of a particular compounds. Typically, the attending physician will decide the dosage of the compounds with which to treat each individual patient, taking into consideration a variety of factors, such as age, body weight, general health, diet, sex, compounds to be administered, route of administration, and the severity of the condition being treated. By way of example and not intending to limit the invention, the dose of the compounds can be about 0.001 to about 1000 mg/kg body weight of the subject being treated/day, from about 0.01 to about 10 mg/kg body weight/day, about 0.01 mg to about 1 mg/kg body weight/day, from about 1 to about to about 1000 mg/kg body weight/day, from about 5 to about 500 mg/kg body weight/day, from about 10 to about 250 mg/kg body weight/day, about 25 to about 150 mg/kg body weight/day, or about 10 mg/kg body weight/day.

In an aspect, the concentration of the compounds in the pharmaceutical composition is at least 0.05 mg/ml (e.g., at least about 0.1 mg/ml, at least about 0.2 mg/ml. at least about 0.5 mg/ml, or at least about 1 mg/ml). This concentration is greater than the naturally occurring concentration of the compounds in their natural environment (e.g., in a sea sponge).

### Methods of Use

In an aspect, the invention provides methods of inhibiting kinase activity in a subject, the method comprising administering to the subject a compound or pharmaceutical composition of the invention. As used herein, “inhibiting” does not necessarily mean 100% reduction in activity, but can mean about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% reduction in activity.

In an aspect of the invention, the kinase is a PKA, a PKG, a PKC, a STK, a CLK, a DYRK, or LATS.

In an aspect of the invention, the kinase is PKA, PKA/DNAJ, PKG1a, PKG1b, PKG2, PKC-θ, PKC-nu, PKC-d, PKC-eta, PKC-g, STK39, CLK1, CLK2, CLK3, CLK4, DYRK1A, DYRK1B, DYRK2, DYRK3, DYRK4, LATS1, or LATS2.

In an aspect of the invention, the kinase is PKA, PKA/DNAJ, or cAMP-PKA.

In an aspect of the invention, the kinase is protein kinase A (PKA). In an aspect, PKA is inhibited resulting in therapeutic benefits to the subject. In an aspect, PKA is inhibited resulting in treatment of a cancer. In an aspect, PKA is inhibited resulting in treatment of liver cancer, for example, hepatocellular carcinoma (HCC) and fibrolamellar hepatocellular carcinoma. In an aspect, PKA is inhibited resulting in treatment of diabetic neuropathic pain (Ma, et al., Neuroscience Letters, 750: 135763 (2021)).

In an aspect of the invention, the kinase is PKA/DNAJ. In an aspect, PKA/DNAJ is inhibited resulting in therapeutic benefits to the subject. In an aspect, PKA/DNAJ is inhibited resulting in treatment of a cancer. In an aspect, PKA/DNAJ is inhibited resulting in treatment of liver cancer, for example, hepatocellular carcinoma (HCC) and fibrolamellar hepatocellular carcinoma. In an aspect, PKA/DNAJ is inhibited resulting in treatment of diabetic neuropathic pain.

In an aspect of the invention, the kinase is cyclic adenosine monophosphate-protein kinase A (cAMP-PKA). In an aspect, cAMP-PKA is inhibited resulting in therapeutic benefits to the subject. In an aspect, cAMP-PKA is inhibited resulting in treatment of a cancer. In an aspect, cAMP-PKA is inhibited resulting in treatment of liver cancer, for example, hepatocellular carcinoma (HCC) and fibrolamellar hepatocellular carcinoma. In an aspect, cAMP-PKA is inhibited resulting in treatment of diabetic neuropathic pain.

In an aspect, the kinase inhibited by compounds of aspects of the invention is a protein kinase G (PKG). In an aspect, a PKG is inhibited resulting in therapeutic benefits to the subject. In an aspect, a PKG is inhibited resulting in treatment of a cancer, for example, gastric cancer or colon cancer (Wu, et al., Molecular Medicine Reports, 14: 1849-1856 (2016); Islam, et al., Carcinogenesis, 43(6): 584-593 (2022)). In an aspect, a PKG is inhibited resulting in treatment and/or prevention of an infection, for example, a parasite infection, for example, malaria (i.e., infection caused by a Plasmodium) (Eck, et al., ChemBioChem, 23(7): 1-8 (2022)).

In an aspect of the invention, the kinase is PKG1a, PKG1b, PKG2, or PfPKG.

In an aspect of the invention, the kinase is PKG1a. In an aspect, PKG1a is inhibited resulting in therapeutic benefits to the subject. In an aspect, PKG1a is inhibited resulting in treatment of a cancer, for example, gastric cancer or colon cancer. In an aspect, PKG1a is inhibited resulting in treatment and/or prevention of an infection, for example, malaria.

In an aspect of the invention, the kinase is PKG1b. In an aspect, PKG1b is inhibited resulting in therapeutic benefits to the subject. In an aspect, PKG1b is inhibited resulting in treatment of a cancer, for example, gastric cancer or colon cancer. In an aspect, PKG1b is inhibited resulting in treatment and/or prevention of an infection, for example, malaria.

In an aspect of the invention, the kinase is PKG2. In an aspect, PKG2 is inhibited resulting in therapeutic benefits to the subject. In an aspect, PKG2 is inhibited resulting in treatment of a cancer, for example, gastric cancer or colon cancer. In an aspect, PKG2 is inhibited resulting in treatment and/or prevention of an infection, for example, malaria.

In an aspect of the invention, the kinase is PfPKG. In an aspect, PfPKG is inhibited resulting in therapeutic benefits to the subject. In an aspect, PfPKG is inhibited resulting in treatment and/or prevention of an infection, for example, malaria.

In an aspect of the invention, the kinase is a protein kinase C (PKC).

In an aspect, a PKC is inhibited resulting in therapeutic benefits to the subject. In an aspect, a PKC is inhibited resulting in treatment of a cancer.

In an aspect of the invention, the kinase is PKC-θ, PKC-nu, PKC-d, PKC-eta, or PKC-g. In an aspect of the invention, the kinase is PKC-θ. In an aspect of the invention, the kinase is PKC-nu. In an aspect of the invention, the kinase is PKC-d. In an aspect of the invention, the kinase is PKC-eta. In an aspect of the invention, the kinase is PKC-g.

In an aspect of the invention, the kinase is a serine/threonine kinase (STK). In an aspect, a STK is inhibited resulting in therapeutic benefits to the subject. In an aspect, a STK is inhibited resulting in treatment of a cancer, for example, breast cancer.

In an aspect, the kinase is STK39. In an aspect, STK39 is inhibited resulting in therapeutic benefits to the subject. In an aspect, STK39 is inhibited resulting in treatment of a cancer, for example, breast cancer.

In an aspect, the kinase inhibited by compounds of aspects of the invention is a dual-specificity tyrosine-regulated kinase (DYRK). In an aspect, a DYRK is inhibited resulting in therapeutic benefits to the subject. In an aspect, a DYRK is inhibited resulting in treatment of a cancer, for example, gastric cancer or colon cancer (Boni, et al., Cancers, 12: 1-26 (2020); Henderson, et al., J Med Chem., 64: 11709-11728 (2021)). In an aspect, a DYRK is inhibited resulting in treatment and/or prevention of an infection, for example, an infection caused by a protozoa (Loaec, et al., Mar. Drugs, 15(316): 1-15 (2017)) or parasite (e.g., Trypanosoma brucei; Cayla, et al., eLife, 1-34 (2020)).

In an aspect of the invention, the kinase is DYRK1A, DYRK1B, DYRK2, DYRK3, or DYRK4.

In an aspect of the invention, the kinase is DYRK1A. In an aspect, DYRK1A is inhibited resulting in therapeutic benefits to the subject. In an aspect, DYRK1A is inhibited resulting in treatment of a cancer, for example, gastric cancer or colon cancer. In an aspect, DYRK1A is inhibited resulting in treatment and/or prevention of an infection, for example, an infection caused by a protozoa or parasite.

In an aspect of the invention, the kinase is DYRK1B. In an aspect, DYRK1B is inhibited resulting in therapeutic benefits to the subject. In an aspect, DYRK1B is inhibited resulting in treatment of a cancer, for example, gastric cancer or colon cancer. In an aspect, DYRK1B is inhibited resulting in treatment and/or prevention of an infection, for example, an infection caused by a protozoa or parasite.

In an aspect of the invention, the kinase is DYRK2. In an aspect, DYRK2 is inhibited resulting in therapeutic benefits to the subject. In an aspect, DYRK2 is inhibited resulting in treatment of a cancer, for example, gastric cancer or colon cancer. In an aspect, DYRK2 is inhibited resulting in treatment and/or prevention of an infection, for example, an infection caused by a protozoa or parasite.

In an aspect of the invention, the kinase is DYRK3. In an aspect, DYRK3 is inhibited resulting in therapeutic benefits to the subject. In an aspect, DYRK3 is inhibited resulting in treatment of a cancer, for example, gastric cancer or colon cancer. In an aspect, DYRK3 is inhibited resulting in treatment and/or prevention of an infection, for example, an infection caused by a protozoa or parasite.

In an aspect of the invention, the kinase is DYRK4. In an aspect, DYRK4 is inhibited resulting in therapeutic benefits to the subject. In an aspect, DYRK4 is inhibited resulting in treatment of a cancer, for example, gastric cancer or colon cancer. In an aspect, DYRK4 is inhibited resulting in treatment and/or prevention of an infection, for example, an infection caused by a protozoa or parasite.

In an aspect, the kinase inhibited by compounds of aspects of the invention is a Cdc2-like kinase (CLK). In an aspect, a CLK is inhibited resulting in therapeutic benefits to the subject. In an aspect, a CLK is inhibited resulting in treatment of gastric cancer. In an aspect, a CLK is inhibited resulting in prevention of memory impairments and neurotoxicityinduced by oligomeric A025-35 peptide administration (Naert, et al., European Neuropsychopharmacology, 2170-2182 (2015); Tam, et al., Cancer Letters, 473: 186-197 (2020); Moyano, et al., Int. J. Mol. Sci., 21: 7549 (2020); and Qin, et al., J. Med. Chem., 64: 13191-13211 (2021)).

In an aspect of the invention, the kinase is CLK1, CLK2, CLK3, or CLK4.

In an aspect, CLK1 is inhibited resulting in therapeutic benefits to the subject. In an aspect, CLK1 is inhibited resulting in treatment of a cancer, for example, gastric cancer. In an aspect, CLK1 is inhibited resulting in prevention of memory impairments and neurotoxicity induced by oligomeric Aβ25-35 peptide administration.

In an aspect, CLK2 is inhibited resulting in therapeutic benefits to the subject. In an aspect, CLK2 is inhibited resulting in treatment of a cancer, for example, gastric cancer. In an aspect, CLK2 is inhibited resulting in prevention of memory impairments and neurotoxicity induced by oligomeric Aβ025-35 peptide administration.

In an aspect, CLK3 is inhibited resulting in therapeutic benefits to the subject. In an aspect, CLK3 is inhibited resulting in treatment of a cancer, for example, gastric cancer. In an aspect, CLK3 is inhibited resulting in prevention of memory impairments and neurotoxicity induced by oligomeric Aβ25-35 peptide administration.

In an aspect, CLK4 is inhibited resulting in therapeutic benefits to the subject. In an aspect, CLK4 is inhibited resulting in treatment of a cancer, for example, gastric cancer. In an aspect, CLK4 is inhibited resulting in prevention of memory impairments and neurotoxicity induced by oligomeric Aβ25-35 peptide administration.

In an aspect, the kinase inhibited by compounds of aspects of the invention is a LATS (Large Tumor Suppressor Kinase). In an aspect, a LATS is inhibited resulting in therapeutic benefits to the subject. In an aspect, a LATS is inhibited resulting in treatment of a cancer.

In an aspect, the kinase inhibited by compounds of aspects of the invention is LATS1 (Large Tumor Suppressor Kinase 1). In an aspect, LATS1 is inhibited resulting in therapeutic benefits to the subject. In an aspect, a LATS1 is inhibited resulting in treatment of a cancer.

In an aspect, the kinase inhibited by compounds of aspects of the invention is LATS2 (Large Tumor Suppressor Kinase 2). In an aspect, LATS2 is inhibited resulting in therapeutic benefits to the subject. In an aspect, a LATS2 is inhibited resulting in treatment of a cancer.

In an aspect, the invention provides methods of suppressing the immune system in a subject, the method comprising administering to the subject a compound or pharmaceutical composition of an aspect of the invention.

In an aspect, the invention provides methods preventing organ rejection in a subject, the method comprising administering to the subject a compound or pharmaceutical composition of an aspect of the invention.

In an aspect, the invention provides methods of treating diabetic neuropathic pain in a subject, the method comprising administering to the subject a compound or pharmaceutical composition of an aspect of the invention.

In an aspect, the invention provides methods of treating malaria in a subject, the method comprising administering to the subject a compound or pharmaceutical composition of an aspect of the invention.

In an aspect, the invention provides methods of treating an infection associated with a protozoa in a subject, the method comprising administering to the subject a compound or pharmaceutical composition of an aspect of the invention.

In an aspect, the invention provides methods of treating a neurodegenerative disease, the method comprising administering to the subject a compound or pharmaceutical composition of an aspect of the invention. In an aspect, the invention provides methods of treating Down syndrome, the method comprising administering to the subject a compound or pharmaceutical composition of an aspect of the invention. In an aspect, the invention provides methods of treating Alzheimer's disease, the method comprising administering to the subject a compound or pharmaceutical composition of an aspect of the invention.

In an aspect, the invention provides methods of treating cardiac disease, the method comprising administering to the subject a compound or pharmaceutical composition of an aspect of the invention. In an aspect, the invention provides methods of treating heart failure, the method comprising administering to the subject a compound or pharmaceutical composition of an aspect of the invention.

In an aspect, the invention provides methods of treating Cushing's syndrome, the method comprising administering to the subject a compound or pharmaceutical composition of an aspect of the invention.

In an aspect, the invention provides methods of treating McCune-Albright Syndrome, the method comprising administering to the subject a compound or pharmaceutical composition of an aspect of the invention.

In an aspect, the invention provides methods of treating Carney complex, the method comprising administering to the subject a compound or pharmaceutical composition of an aspect of the invention.

An aspect of the invention provides compounds and pharmaceutically compositions for use in treating or preventing cancer. Without being bound by a particular theory or mechanism, it is believed that the compounds inhibit kinases.

The terms “treat” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the methods of an aspect of the invention can provide any amount of any level of treatment or prevention of cancer in a mammal. Furthermore, the treatment or prevention provided by the method of an aspect of the invention can include treatment or prevention of one or more conditions or symptoms of the disease, e.g., cancer, being treated or prevented. Also, for purposes herein, “prevention” can encompass delaying the onset of the disease, or a symptom or condition thereof.

With respect to the methods of an aspect of the invention, the cancer can be any cancer, including any of adrenal gland cancer, sarcomas (e.g., synovial sarcoma, osteogenic sarcoma, leiomyosarcoma uteri, angiosarcoma, fibrosarcoma, rhabdomyosarcoma, liposarcoma, myxoma, rhabdomyoma, fibroma, lipoma, and teratoma), lymphomas (e.g., small lymphocytic lymphoma, Hodgkin lymphoma, and non-Hodgkin lymphoma), hepatocellular carcinoma, glioma, head cancers (e.g., squamous cell carcinoma), neck cancers (e.g., squamous cell carcinoma), acute lymphocytic cancer, leukemias (e.g., hairy cell leukemia, myeloid leukemia (acute and chronic), lymphatic leukemia (acute and chronic), prolymphocytic leukemia (PLL), myelomonocytic leukemia (acute and chronic), and lymphocytic leukemia (acute and chronic)), bone cancer (osteogenic sarcoma, fibrosarcoma, malignant fibrous histiocytoma, chondrosarcoma, Ewing's sarcoma, malignant lymphoma (reticulum cell sarcoma), multiple myeloma, malignant giant cell tumor, chordoma, osteochondroma (osteocartilaginous exostoses), benign chondroma, chondroblastoma, chondromyxoid fibroma, osteoid osteoma, and giant cell tumors), brain cancer (astrocytoma, medulloblastoma, glioma, ependymoma, germinoma (pinealoma), glioblastoma multiforme, oligodendroglioma, schwannoma, and retinoblastoma), fallopian tube cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva (e.g., squamous cell carcinoma, intraepithelial carcinoma, adenocarcinoma, and fibrosarcoma), myeloproliferative disorders (e.g., chronic myeloid cancer), colon cancers (e.g., colon carcinoma), esophageal cancer (e.g., squamous cell carcinoma, adenocarcinoma, leiomyosarcoma, and lymphoma), cervical cancer (cervical carcinoma and pre-invasive cervical dysplasia), gastric cancer, gastrointestinal carcinoid tumor, hypopharynx cancer, larynx cancer, liver cancers (e.g., hepatocellular carcinoma, fibrolamellar hepatocellular carcinoma, cholangiocarcinoma, hepatoblastoma, angiosarcoma, hepatocellular adenoma, and hemangioma), lung cancers (e.g., bronchogenic carcinoma (squamous cell, undifferentiated small cell, undifferentiated large cell, and adenocarcinoma), alveolar (bronchiolar) carcinoma, bronchial adenoma, chondromatous hamartoma, small cell lung cancer, non-small cell lung cancer, and lung adenocarcinoma), malignant mesothelioma, skin cancer (e.g., melanoma, basal cell carcinoma, squamous cell carcinoma, Kaposi's sarcoma, nevi, dysplastic nevi, lipoma, angioma, dermatofibroma, and keloids), multiple myeloma, nasopharynx cancer, ovarian cancer (e.g., ovarian carcinoma (serous cystadenocarcinoma, mucinous cystadenocarcinoma, endometrioid carcinoma, and clear cell adenocarcinoma), granulosa-theca cell tumors, Sertoli-Leydig cell tumors, dysgerminoma, and malignant teratoma), pancreatic cancer (e.g., ductal adenocarcinoma, insulinoma, glucagonoma, gastrinoma, carcinoid tumors, and VIPoma), peritoneum, omentum, mesentery cancer, pharynx cancer, prostate cancer (e.g., adenocarcinoma and sarcoma), rectal cancer, kidney cancer (e.g., adenocarcinoma, Wilms tumor (nephroblastoma), and renal cell carcinoma), small intestine cancer (adenocarcinoma, lymphoma, carcinoid tumors, Kaposi's sarcoma, leiomyoma, hemangioma, lipoma, neurofibroma, and fibroma), soft tissue cancer, stomach cancer (e.g., carcinoma, lymphoma, and leiomyosarcoma), testicular cancer (e.g., seminoma, teratoma, embryonal carcinoma, teratocarcinoma, choriocarcinoma, sarcoma, Leydig cell tumor, fibroma, fibroadenoma, adenomatoid tumors, and lipoma), cancer of the uterus (e.g., endometrial carcinoma), thyroid cancer, and urothelial cancers (e.g., squamous cell carcinoma, transitional cell carcinoma, adenocarcinoma, ureter cancer, and urinary bladder cancer). In an aspect of the invention, the cancer is hepatocellular carcinoma. In an aspect of the invention, the cancer is fibrolamellar hepatocellular carcinoma. In an aspect of the invention, the cancer is liver cancer. In an aspect of the invention, the cancer is breast cancer. In an aspect of the invention, the cancer is gastric cancer. In an aspect of the invention, the cancer is colon cancer.

In certain aspects of the invention, the compounds of aspects of the invention, or pharmaceutically acceptable salts thereof, can be co-administered with an anti-cancer agent (e.g., a chemotherapeutic agent) and/or radiation therapy. In an aspect, the compounds of aspects of the invention, or pharmaceutically acceptable salts thereof, are administered in an amount that is effective to sensitize the cancer cells to one or more therapeutic regimens (e.g., chemotherapy or radiation therapy). The terms “co-administered” or “co-administration” refer to simultaneous or sequential administration. The compounds of aspects of the invention, or pharmaceutically acceptable salts thereof, can be administered before, concurrently with, or after administration of another anti-cancer agent (e.g., a chemotherapeutic agent).

One or more than one, e.g., two, three, or more anti-cancer agents can be administered. In this regard, the present invention is directed a pharmaceutical composition comprising a pharmaceutically acceptable carrier and a combination of the compounds of aspects of the invention, or pharmaceutically acceptable salts thereof, and at least one anti-cancer agent (e.g., chemotherapeutic agent).

Examples of anti-cancer agents include platinum compounds (e.g., cisplatin, carboplatin, oxaliplatin), alkylating agents (e.g., cyclophosphamide, ifosfamide, chlorambucil, nitrogen mustard, thiotepa, melphalan, busulfan, procarbazine, streptozocin, temozolomide, dacarbazine, bendamustine), antitumor antibiotics (e.g., daunorubicin, doxorubicin, idarubicin, epirubicin, mitoxantrone, bleomycin, mitomycin C, plicamycin, dactinomycin), taxanes (e.g., paclitaxel and docetaxel), antimetabolites (e.g., 5-fluorouracil, cytarabine, pemetrexed, thioguanine, floxuridine, capecitabine, and methotrexate), nucleoside analogues (e.g., fludarabine, clofarabine, cladribine, pentostatin, nelarabine), topoisomerase inhibitors (e.g., topotecan and irinotecan), hypomethylating agents (e.g., azacitidine and decitabine), proteosome inhibitors (e.g., bortezomib), epipodophyllotoxins (e.g., etoposide and teniposide), DNA synthesis inhibitors (e.g., hydroxyurea), vinca alkaloids (e.g., vincristine, vindesine, vinorelbine, and vinblastine), tyrosine kinase inhibitors (e.g., imatinib, dasatinib, nilotinib, sorafenib, sunitinib), monoclonal antibodies (e.g., rituximab, cetuximab, panitumumab, tositumomab, trastuzumab, alemtuzumab, gemtuzumab ozogamicin, bevacizumab), nitrosoureas (e.g., carmustine, fotemustine, and lomustine), enzymes (e.g., L-Asparaginase), biological agents (e.g., interferons and interleukins), hexamethylmelamine, mitotane, angiogenesis inhibitors (e.g., thalidomide, lenalidomide), steroids (e.g., prednisone, dexamethasone, and prednisolone), hormonal agents (e.g., tamoxifen, raloxifene, leuprolide, bicalutamide, granisetron, flutamide), aromatase inhibitors (e.g., letrozole and anastrozole), arsenic trioxide, tretinoin, nonselective cyclooxygenase inhibitors (e.g., nonsteroidal anti-inflammatory agents, salicylates, aspirin, piroxicam, ibuprofen, indomethacin, naprosyn, diclofenac, tolmetin, ketoprofen, nabumetone, oxaprozin), selective cyclooxygenase-2 (COX-2) inhibitors, cellular immunotherapy (e.g., chimeric antigen receptor T cell therapy, tumor-infiltrating lymphocyte therapy), or any combination thereof. In some aspects, the anti-cancer agent is cisplatin, cytarabine, methotrexate, doxorubicin, or a combination thereof.

In certain aspects of the invention, the compounds of aspects of the invention, or pharmaceutically acceptable salts thereof, can be attached to targeting molecules. Such targeting molecules include antibodies (for ADCs) and small molecules that target other regions of kinases to afford more selectivity (i.e., a second molecule that binds the DNAJ domain of the PKADJ fusion protein).

In certain aspects of the invention, the compounds of aspects of the invention, or pharmaceutically acceptable salts thereof, can be attached to an E3 ligase binding molecule to make a proteolysis-targeting chimeras (PROTAC).

As used herein, the term “mammal” refers to any mammal, including, but not limited to, mammals of the order Rodentia, including mice and hamsters, mammals of the order Logomorpha, including rabbits, mammals from the order Camivora, including Felines (cats) and Canines (dogs), mammals from the order Artiodactyla, including Bovines (cows) and Swines (pigs), mammals from the order Perssodactyla, including Equines (horses), mammals of the order Primates, Ceboids, or Simoids (monkeys), and mammals of the order Anthropoids (humans and apes). An especially preferred mammal is the human.

### Methods of Preparation

The compounds of aspects of the invention can be prepared by any of a number of conventional techniques.

In certain aspects, the invention provides methods of making aplithianine A

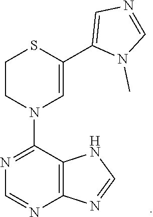 [SMILES: Cn1cncc1C1=CN(c2ncnc3nc[nH]c23)CCS1]

comprising coupling a purine-thiazine conjugate having the following structure:

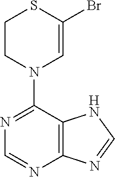 [SMILES: BrC1=CN(c2ncnc3nc[nH]c23)CCS1]

with an imidazole having the following structure:

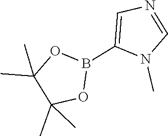

to provide apithianine A.

In an aspect of the invention, the coupling is performed in the presence of a catalyst.

In certain aspects, the invention provides methods of preparing a compound:

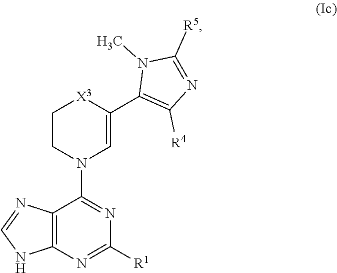

wherein at least one of R³ or R⁴ is halogen;
which method comprises halogenating a compound of formula:

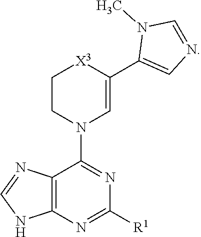

In an aspect, the compound Ic is aplithianine A.

In an aspect, aplithianine A is brominated using N-bromosuccinimide to provide:

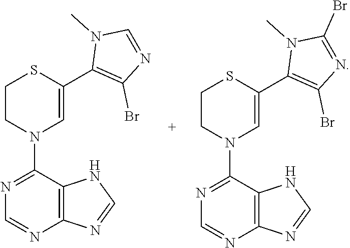 [SMILES: Cn1cnc(Br)c1C1=CN(c2ncnc3nc[nH]c23)CCS1 ; Cn1c(Br)nc(Br)c1C1=CN(c2ncnc3nc[nH]c23)CCS1]

### Examples of Non-Limiting Aspects of the Disclosure

Aspects, including embodiments, of the present subject matter described herein may be beneficial alone or in combination, with one or more other aspects or embodiments. Without limiting the foregoing description, certain non-limiting aspects of the disclosure numbered 1-38 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

(1) A compound of formula (I)

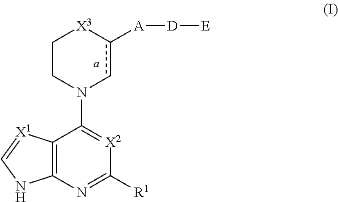

wherein
[symbol] is a single or double bond,
X¹ and X² are each independently CH, CR⁶, or N;
X³ is S, S═O, or S(═O)₂;
R¹ is H or —NR²R³,
R² is H or C₁-C₃ alkyl;
R³ is an aryl;
R⁶ is C₁-C₃ alkyl;
A is optional and, when present, is —C(O)—, —C(O)O—, —C(O)NH—, —C(O)N(C₁-C₃-akyl)-; —C(O)NH—(C₁-C₆ alkyl)-NHC(O)—, —NH;
D is optional and, when present, is a C₁-C₆ alkyl, C₃-C₈ cycloalkyl, C₃-C₈ heterocycloalkyl, —(C₁-C₆ alkyl)-O—, —(C₁-C₆ alkyl)-NH—, —(C₁-C₃ alkyl)-O—(C₁-C₃ alkyl)-, or —(C₁-C₃ alkyl)-NH—(C₁-C₃ alkyl)-, wherein the alkyl group or cycloalkyl group of any of the foregoing is optionally substituted with one or more substituents selected from hydroxy, C₁-C₆ alkyl, amino, C₁-C₆ alkylamino, or di-C₁-C₆ alkyl-amino; —NH-aryl, and a combination thereof; and
E is:
   aryl or heteroaryl, optionally substituted with one or more substituents selected from C₁-C₆ alkyl or alkoxy, —(C₁-C₆ alkyl)-OH, —(C₁-C₆ alkyl)-COOH, —(C₁-C₆ alkyl)-NH₂, halo, nitro, hydroxy, amino, C₁-C₆ alkylamino, di-C₁-C₆ alkyl-amino; —NH-aryl, C₁-C₆ haloalkyl, C₃-C₈ cycloalkyl or heterocycloalkyl, fused C₃-C₈ cycloalkyl or heterocycloalkyl, aryl or heteroaryl, —CN, —(C₁-C₃ alkyl)-CN, carbonyl, and a combination thereof;
   amino, C₁-C₆ alkylamino, or di-C₁-C₆ alkyl-amino; or —NH-aryl;
   C₁-C₆ alkyl or alkoxy, optionally substituted with one or more substituents selected from hydroxy, C₁-C₆ alkyl, amino, C₁-C₆ alkylamino, or di-C₁-C₆ alkyl-amino; —NH-aryl, and a combination thereof;
   C₃-C₈ cycloalkyl or heterocycloalkyl, optionally substituted with one or more substituents selected from hydroxy, C₁-C₆ alkyl, amino, C₁-C₆ alkylamino, di-C₁-C₆ alkyl-amino; —NH-aryl, aryl or heteroaryl, fused aryl or heteroaryl, and a combination thereof;
   —C(O)NH₂;
   —C(O)OH;
   —C(O)H;
   —N—(C₁-C₆ alkyl)-acrylamide;
   halogen; or
   hydrogen;
or a pharmaceutically acceptable salt thereof.

(2) The compound of aspect 1,
wherein
[symbol] is a single or double bond,
X¹ and X² are each independently CH or N;
X³ is S, S═O, or S(═O)₂;
R¹ is H or —NR²R³;
R² is H or C₁-C₃ alkyl;
R³ is an aryl;
A is optional and, when present, is-C(O)—, —C(O)O—, —C(O)NH—, —C(O)N(C₁-C₃-akyl)-; —C(O)NH—(C₁-C₆ alkyl)-NHC(O)—, —NH—;
D is optional and, when present, is a C₁-C₆ alkyl, —(C₁-C₆ alkyl)-O—, —(C₁-C₆ alkyl)-NH—, —(C₁-C₃ alkyl)-O—(C₁-C₃ alkyl)-, or —(C₁-C₃ alkyl)-NH—(C₁-C₃ alkyl)-, wherein the alkyl group of any of the foregoing is optionally substituted with hydroxy; and
E is:
   aryl or heteroaryl, optionally substituted with C₁-C₆ alkyl or alkoxy, halo, nitro, hydroxy, C₁-C₆ haloalkyl, —CN, —(C₁-C₃ alkyl)-CN, or carbonyl;
   amino, C₁-C₆ alkylamino, or di-C₁-C₆ alkyl-amino; or —NH-aryl;
   C₁-C₆ alkyl or alkoxy, optionally substituted with hydroxy;
   C₃-C₈ cycloalkyl or heterocycloalkyl, optionally substituted with hydroxy;
   —C(O)NH₂;
   —C(O)OH;
   —C(O)H;
   —N—(C₁-C₆ alkyl)-acrylamide;
   halogen; or
   hydrogen;
or a pharmaceutically acceptable salt thereof.

(3) The compound of aspect 1 or 2, wherein [symbol] is a double bond.

(4) The compound of any one of aspects 1-3, wherein the compound of formula (I) is of formula (Ia):

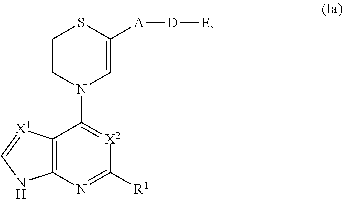

or a pharmaceutically acceptable salt thereof

(5) The compound of any one of aspects 1-4, wherein the compound of formula (I) is of formula (Ib):

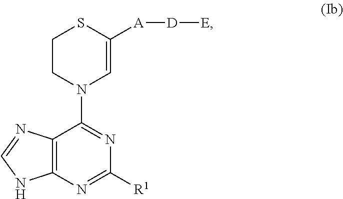

or a pharmaceutically acceptable salt thereof.

(6) The compound of any one of aspects 1-5, wherein R¹ is H.

(7) The compound of any one of aspects 1-6, wherein:
(i) A and D are absent, and E is halogen; —C(O)OH; —C(O)H; aryl or heteroaryl, optionally substituted with C₁-C₃ alkyl, or halo; or C₃-C₈ cycloalkyl or heterocycloalkyl, optionally substituted with hydroxy;
(ii) A is absent; D is a C₁-C₆ alkyl, optionally a C₁-C₃ alkyl; and E is —C(O)NH₂ or a C₃-C₈ cycloalkyl or heterocycloalkyl, optionally substituted with hydroxy;
(iii) A is —NH—, —C(O)NH— or —C(O)N(C₁-C₃-akyl)-; D is absent or is a C₁-C₆ alkyl, optionally a C₁-C₃ alkyl; and E is amino, C₁-C₆ alkylamino, or di-C₁-C₆ alkyl-amino; or —NH-aryl; C₁-C₆ alkyl or alkoxy or C₁-C₃ alkyl or alkoxy, optionally substituted with hydroxy; or aryl or heteroaryl, optionally substituted with C₁-C₃ alkyl or alkoxy, halo, nitro, hydroxy, C₁-C₃ haloalkyl, —CN, —(C₁-C₃ alkyl)-CN, or carbonyl;
(iv) A is absent, D is —(C₁-C₆ alkyl)-O—, —(C₁-C₆ alkyl)-NH—, —(C₁-C₃ alkyl)-O—(C₁-C₃ alkyl)-, or —(C₁-C₃ alkyl)-NH—(C₁-C₃ alkyl)-, wherein the alkyl groups of any of the foregoing is optionally substituted with hydroxy and wherein the alkyl groups of any of the foregoing are optionally branched; and E is C₁-C₆ alkyl or C₃-C₈ cycloalkyl or heterocycloalkyl, optionally substituted with hydroxy;
(v) A is —C(O)NH—(C₁-C₆ alkyl)-NHC(O)—, optionally C(O)NH—(C₁-C₃ alkyl)-NHC(O)—; D is absent; and E is C₁-C₆ alkyl or C₁-C₃ alkyl; or aryl or heteroaryl, optionally substituted with C₁-C₃ alkyl or alkoxy, halo, nitro, hydroxy, C₁-C₆ haloalkyl, —CN, —(C₁-C₃ alkyl)-CN, or carbonyl;
(vi) A is —C(O)O—; D is absent; and E is C₁-C₆ alkyl; or
(vii) A is —C(O)—; D is absent; and E is heterocyloalkyl.

(8) The compound of aspect 1, wherein the compound of formula (I) is of formula (Ic):

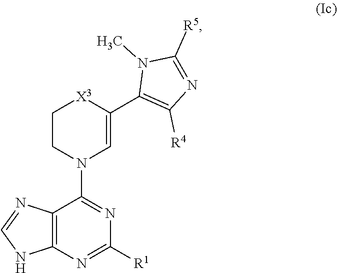

wherein R⁴ and R⁵ are the same or different and each is H or halo, or a pharmaceutically acceptable salt thereof.

(9) The compound of aspect 8, wherein one of R⁴ and R⁵ is halo, or wherein both R⁴ and R⁵ are halo.

(10) The compound of aspect 8, wherein R⁴ and R⁵ are both hydrogen.

(11) The compound of any of aspects 1-10, wherein the compound is not aplithianine A

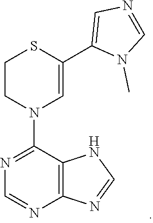 [SMILES: Cn1cncc1C1=CN(c2ncnc3nc[nH]c23)CCS1]

(12) The compound of aspect 1, wherein the compound of formula (I) is

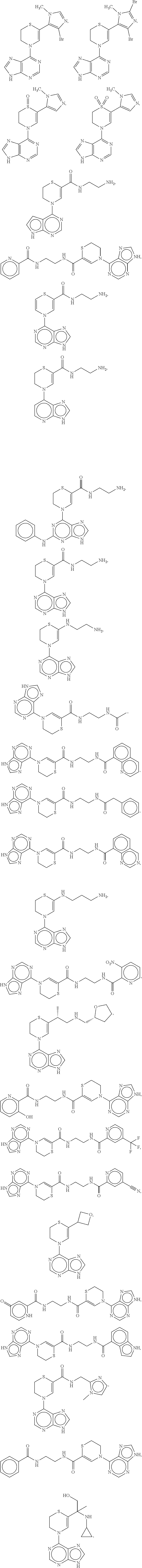

[chemical structure]

or a pharmaceutically acceptable salt thereof.

(13) The compound of aspect 12, wherein the compound of formula (I) is selected from

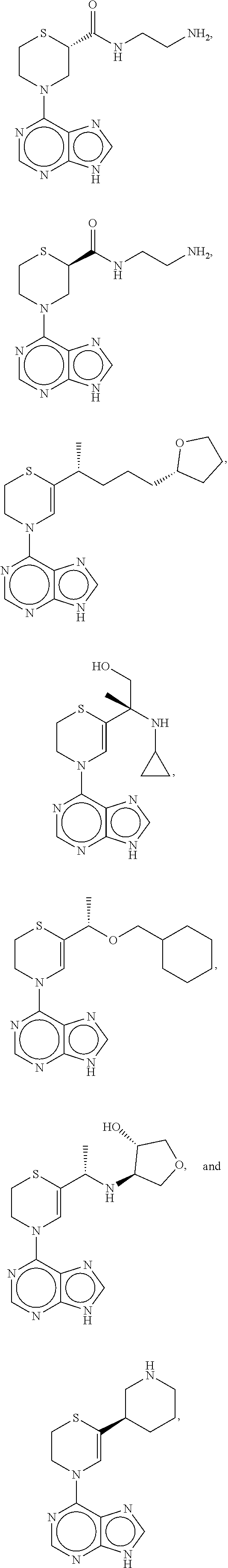

or a pharmaceutically acceptable salt thereof.

(14) A compound

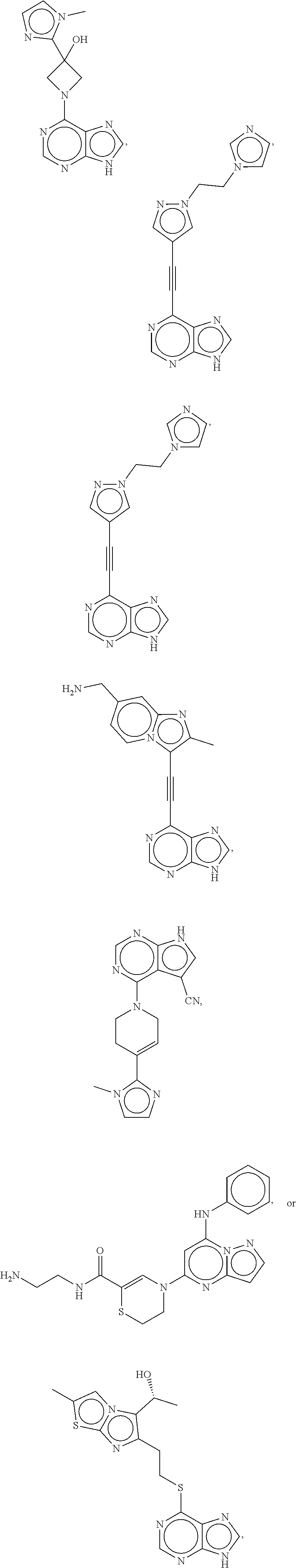

or a pharmaceutically acceptable salt thereof.

(15) A pharmaceutical composition comprising a compound of any one of aspects 1-14 and a pharmaceutical carrier.

(16) A pharmaceutical composition comprising

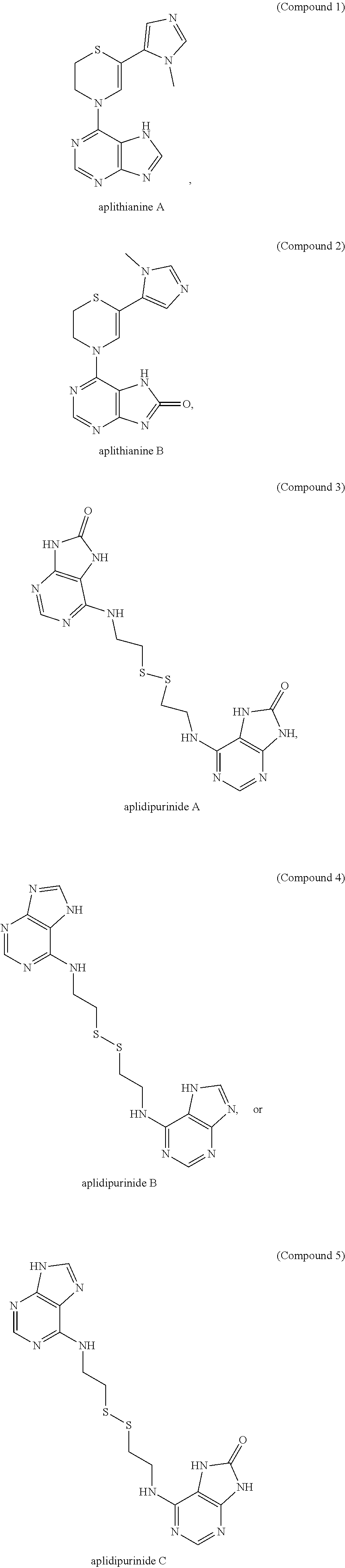

with at least 80% purity, and a pharmaceutical carrier.

(17) A method of inhibiting kinase activity in a subject, the method comprising administering to the subject a compound of any one of aspects 1-14 or the pharmaceutical composition of aspects 15 or 16.

(18) The method of aspect 17, wherein the kinase is PKA, PKA/DNAJ, cAMP-PKA, PKG1a, PKG1b, PKG2, PfPKG, PKC-θ, PKC-nu, PKC-d, PKC-eta, PKC-g, STK39, CLK1, CLK2, CLK3, CLK4, DYRK1A, DYRK1B, DYRK2, DYRK3, DYRK4, LATS1, or LATS2.

(19) The method of aspect 17, wherein the kinase is PKA, PKA/DNAJ, or cAMP-PKA.

(20) The method of aspect 17, wherein the kinase is PKG1a, PKG1b, PKG2, or PfPKG.

(21) The method of aspect 17, wherein the kinase is DYRK1A, DYRK1B, DYRK2, DYRK3, or DYRK4.

(22) The method of aspect 17, wherein the kinase is CLK1, CLK2, CLK3, or CLK4.

(23) A method of suppressing the immune system in a subject, the method comprising administering to the subject a compound of any one of aspects 1-14 or the pharmaceutical composition of aspects 15 or 16.

(24) A method of preventing organ rejection in a subject, the method comprising administering to the subject a compound of any one of aspects 1-14 or the pharmaceutical composition of aspects 15 or 16.

(25) A method of treating cancer in a subject, the method comprising administering to the subject a compound of any one of aspects 1-14 or the pharmaceutical composition of aspects 15 or 16.

(26) The method of aspect 25, wherein the cancer is fibrolamellar carcinoma (FLC).

(27) The method of aspect 25, wherein the cancer is fibrolamellar hepatocellular carcinoma (FL-HCC).

(28) The method of aspect 25, wherein the cancer is gastric cancer.

(29) The method of aspect 25, wherein the cancer is colon cancer.

(30) A method of treating diabetic neuropathic pain in a subject, the method comprising administering to the subject a compound of any one of aspects 1-14 or the pharmaceutical composition of aspects 15 or 16.

(31) A method of treating malaria in a subject, the method comprising administering to the subject a compound of any one of aspects 1-14 or the pharmaceutical composition of aspects 15 or 16.

(32) A method of treating an infection associated with a protozoa in a subject, the method comprising administering to the subject a compound of any one of aspects 1-14 or the pharmaceutical composition of aspects 15 or 16.

(33) The method of any one of aspects 17-32, wherein the subject is human.

(34) A method of making aplithianine A

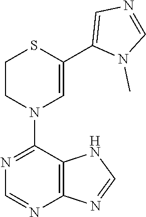 [SMILES: Cn1cncc1C1=CN(c2ncnc3nc[nH]c23)CCS1]

comprising coupling a purine-thiazine conjugate having the following structure:

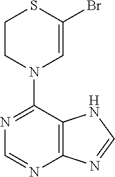 [SMILES: BrC1=CN(c2ncnc3nc[nH]c23)CCS1]

with an imidazole having the following structure:

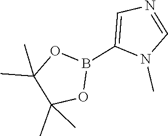

to provide apithianine A.

(35) The method of aspect 34, wherein the coupling is performed in the presence of a catalyst.

(36) A method of preparing a compound of aspect 8:

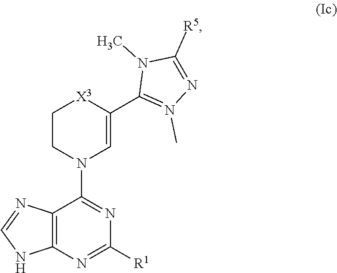

wherein at least one of R³ or R⁴ is halogen;
which method comprises halogenating a compound of formula:

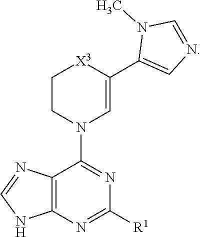

(37) The method of aspect 36, wherein compound Ic is aplithianine A.

(38) The method of aspect 37, wherein aplithianine A is brominated using N-bromosuccinimide to provide:

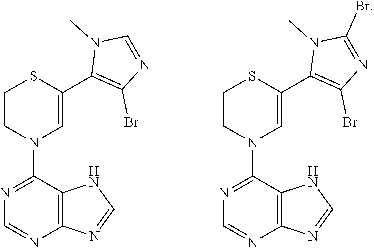 [SMILES: Cn1cnc(Br)c1C1=CN(c2ncnc3nc[nH]c23)CCS1 ; Cn1c(Br)nc(Br)c1C1=CN(c2ncnc3nc[nH]c23)CCS1]

The following examples further illustrate the invention but, of course, should not be construed as in any way limiting its scope.

### EXAMPLES

UV data were measured with a VARIAN CARY™ 50 UV-Vis Spectrophotometer. IR spectrum was recorded with a Bruker ALPHA II FT-IR spectrometer. NMR data were obtained on a Bruker Avance III NMR spectrometer equipped with a 3 mm cryogenic probe (600 MHz for ¹H, 150 MHz for ¹³C). HRESIMS data were collected on an Agilent Technology 6530 Accurate-mass Q-TOF LC/MS. HPLC separations were performed on a Shimadzu system equipped with a CBM-40 controller, an SPD-M40 PDA detector, and two LC-20AR pumps.

All solvents were of LC-MS grade or better.

Specimens of the tunicate Aplidium sp. were collected from a reef in South Africa in September 2000, and kept frozen until extraction. The collection was carried out by the Coral Reef Research Foundation under contract with the Natural Products Branch, U.S. National Cancer Institute. A voucher specimen (voucher ID #0CDN7423) was deposited at the Smithsonian Institution, Washington, D.C. The animal material (234 g, wet weight) was ground and processed using the standard NCI method (McCloud, Molecules, 15: 4526-4563 (2010)) for marine samples to provide 3.27 g of organic extract (NSC #C020725) and 16.2 g of aqueous extract (NSC #C020724).

### Example 1

This example demonstrates that compounds of the present invention can be extracted and purified.

The organic crude Aplidium sp. extract (NSC #C020725, 1 g, described above) was subjected to a C8 solid-phase extraction (SPE) process (250 mg extract loading on each 2g C8 SPE cartridge eluted with gradients of 5%, 20%, 40%, 60%, 80%, and 100% MeOH in H₂O and 50% MeOH in MeCN) to generate seven fractions. The active fraction Fr. 5 was further separated by prep-HPLC using a Kinetex 5 μm EVO C18 column (110 Å, 250×21.2 mm) with flow rate of 10 mL/min (eluted with 5%-100% MeCN with 0.1% TFA) to give 20 fractions. Fraction Fr. 5-14 was further purified by semi-prep HPLC using a Kinetex 5 μm F5 column (110 Å, 250×10 mm) with flow rate of 4 mL/min (eluted with 8% MeCN with 0.1% TFA) to produce compound 2 (0.7 mg). Fraction Fr. 5-15 was purified by semi-prep HPLC using a Kinetex 5 μm F5 column (110 Å, 250×10 mm) with flow rate of 4 mL/min (eluted with 12% MeCN with 0.1% TFA) to produce compounds 1 (6.9 mg), 3 (0.8 mg), 4 (2.6 mg), and 5 (1.9 mg). The structures of compounds 1-5 are provided in FIG. 22.

To accumulate the active compound 1 for chemical modification, 1.38 g of organic extract (NSC #C020725) and 15.4 g of aqueous extract (NSC #C020724) were requested from the NCI Natural Products Repository. Similar isolation procures were conducted on the organic extract leading to the yield of 46 mg of 1. The aqueous extract was first desalted by HP20ss VLC (H₂O wash followed by MeOH elution). The fraction (660 mg) eluted by MeOH was further purified by prep-HPLC using a Kinetex 5 μm F5 column (110 Å, 250×21.2 mm) with flow rate of 10 mL/min (eluted with 15% MeCN with 0.1% TFA) to give 66 mg of 1.

Aplithianine A (1): white solid; UV (MeOH) λₘₐₓ (log ε) 242 (3.98), 333 (4.23); IR (neat) νₘₐₓ 3093, 2974, 2919, 2849, 2828, 1685, 1573, 1452, 1415, 1359, 1330, 1288, 1253, 1209, 1183, 1129, 1027, 975, 937, 912, 844, 803, 724, 643 cm⁻¹; ¹H and ¹³C NMR data, see Tables 1 and 2; HRESIMS m/z 300.1028, [M+H]⁺ (calcd for C₁₃H₁₄N₇S, 300.1031).

Aplithianine B (2): white solid; UV (MeOH) λₘₐₓ (log ε) 250 (3.82), 326 (4.08); IR (neat) νₘₐₓ 2919, 2850, 1718, 1682, 1595, 1413, 1295, 190, 1132, 1074, 1060, 1033, 940, 832, 797, 762, 720, 570, 534 cm⁻¹; ¹H and ¹³C NMR data, see Tables 1 and 2; HRESIMS m/z 316.0974, [M+H]⁺ (calcd for C₁₃H₁₄N₇OS, 316.0981).

Aplidipurinide A (3): white solid; UV (MeOH) λₘₐₓ (log ε) 212 (4.40), 277 (4.23); IR (neat) νₘₐₓ 3217, 2922, 1694, 1638, 1489, 1441, 1350, 1209, 1185, 1137, 842, 805, 725, 650, 606 cm⁻¹; ¹H and ¹³C NMR data, see Table 3; HRESIMS m/z 421.0981, [M+H]⁺ (calcd for C₁₄H₁₇N₁₀O₂S₂, 421.0977).

Aplidipurinide B (4): white solid; UV (MeOH) λₘₐₓ (log ε) 212 (4.41), 271 (4.38); JR (neat) νₘₐₓ 2976, 2849, 1678, 1488, 1435, 1402, 1352, 1286, 1204, 1136, 1054, 1032, 842, 801, 724, 644 cm⁻¹; ¹H and ¹³C NMR data, see Table 3; HRESIMS m/z 389.1077, [M+H]⁺ (calcd for C₁₄H₁₇N₁₀S₂, 389.1079).

Aplidipurinide C (5): white solid; UV (MeOH) λₘₐₓ (log ε) 211 (4.56), 273 (4.11); IR (neat) νₘₐₓ 2922, 2852, 1683, 1615, 1585, 1443, 1368, 1296, 1209, 1183, 1138, 1032, 844, 804, 771, 725, 647 cm⁻¹; ¹H and ¹³C NMR data, see Table 3; HRESIMS m/z 405.1025, [M+H]⁺ (calcd for C₁₄H₁₇N₁₀OS₂, 405.1028).

TABLE 1
¹³C NMR (150 MHz, δ ppm) data for compounds 1, 2, and 1a-1d.

| No. | 1^{a} | 1^{b} | 2^{b} | 1a^{a} | 1b^{a} | 1c^{a} | 1d^{a} |
|---|---|---|---|---|---|---|---|
| 2 | 95.4 | 97.6 | 95.3 | 97.5 | 97.0 | 103.1 | 102.8 |
| 3 | 128.7 | 130.4 | 129.0 | 129.0 | 129.4 | 138.3 | 140.9 |
| 5 | 42.8 | 44.6 | 46.3 | 43.0 | 42.7 | 31.3 | 42.4 |
| 6 | 25.3 | 26.8 | 26.7 | 25.5 | 25.3 | 43.2 | 47.5 |
| 2′ | 137.0 | 137.7 | 137.4 | 138.5 | 119.9 | 137.1 | 138.1 |
| 4′ | 118.6 | 120.0 | 119.8 | 115.4 | 114.7 | 120.1 | 124.1 |
| 5′ | 132.0 | 134.5 | 134.1 | 127.2 | 130.6 | 130.4 | 123.3 |
| 6′ | 34.3 | 35.1 | 34.8 | 32.7 | 33.7 | 34.0 | 33.4 |
| 2″ | 151.4 | 152.7 | 151.4 | 151.5 | 151.4 | 151.4 | 151.3 |
| 4″ | 152.6 | 153.6 | 152.6 | 152.4 | 152.5 | 153.4 | 153.4 |
| 5″ | 119.8 | 121.6 | 110.0 | 119.7 | 119.7 | 120.5 | 120.5 |
| 6″ | 149.6 | 151.8 | 144.2 | 149.7 | 149.6 | 149.0 | 148.8 |
| 8″ | 141.1 | 141.7 | 155.6 | 140.7 | 140.9 | 142.7 | 142.7 |

^{a}Measured in DMSO-d₆;
^{b}Measured in methanol-d₄.

TABLE 2
¹H NMR (600 MHz, δ ppm, J in Hz) data for compounds 1, 2, and 1a-1d.

| No. | 1^{a} | 1^{b} | 2^{a} | 2^{b} | 1a^{a} | 1b^{a} | 1c^{a} | 1d^{a} |
|---|---|---|---|---|---|---|---|---|
| 3 | 9.00, br s | 9.00, s | 7.46, s | 7.61, s | 8.78, br s | 8.83, br s | 9.29, s | 9.25, s |
| 5 | 4.61, br s | 4.71, m | 4.20, m | 4.32, m | 4.59, br s | 4.60, br s | 5.56, m | 4.97, m |
| | | | | | | | 3.84, m | |
| 6 | 3.37, m | 3.38, m | 3.28, m | 3.33, m | 3.33, m | 3.36, m | 3.42, m | 3.85, m |
| | | | | | | | 3.18, m | |
| 2′ | 9.12, s | 8.88, s | 8.91, s | 8.90, s | 7.72, s | | 9.11, s | 8.92, br s |
| 4′ | 7.78, s | 7.62, s | 7.62, s | 7.58, s | | | 7.80, s | 7.73, br s |
| 6′ | 3.90, s | 4.02, s | 3.80, s | 3.95, s | 3.65, s | 3.59, s | 3.83, s | 3.80, s |
| 2″ | 8.46, s | 8.45, s | 8.23, s | 8.26, s | 8.43, s | 8.44, s | 8.65, s | 8.65, s |
| 7″ | 13.6, br s | | 11.1, s | | 13.41, br s | 13.3, br s | 13.8, br s | 13.8, br s |
| 8″ | 8.35, s | 8.18, s | | | 8.30, s | 8.32, s | 8.51, s | 8.51, s |
| 9″ | | | 11.9, s | | | | | |

^{a}Measured in DMSO-d₆;
^{b}Measured in methanol-d₄.

TABLE 3
¹H and ¹³C NMR data for compounds 3-5 in DMSO-d₆ (600
MHz for ¹H and 150 MHz for ¹³C, respectively, δ ppm).

| | 3 | | 4 | | 5 | |
|---|---|---|---|---|---|---|
| | | δ_{H} | | δ_{H} | | δ_{H} |
| No. | δ_{C} | (J in Hz) | δ_{C} | (J in Hz) | δ_{C} | (J in Hz) |
| 2 | 150.7 | 8.05, s | 148.5 | 8.50, s | 150.4 | 8.05, s |
| 4 | 147.3 | | 148.1 | | 147.2 | |
| 5 | 104.7 | | 115.3 | | 104.6 | |
| 6 | 145.3 | | 152.1 | | 145.1 | |
| 7 | | 9.98, s | | | | 10.04, s |
| 8 | 152.7 | | 142.3 | 8.42, s | 152.7 | |
| 9 | | 11.35, s | | | | 11.38, s |
| 10 | | 6.73, t | | 9.03, br s | | 6.82, t |
| | | (5.8) | | | | (5.8) |
| 11 | 39.0 | 3.72, m | 40.0 | 3.87, br s | 39.1 | 3.72, m |
| 12 | 37.3 | 2.96, t | 36.5 | 3.06, t | 37.3 | 2.97, t |
| | | (6.5) | | (6.8) | | (6.5) |
| 2′ | 150.7 | 8.05, s | 148.5 | 8.50, s | 147.9 | 8.55, s |
| 4′ | 147.3 | | 148.1 | | 147.8 | |
| 5′ | 104.7 | | 115.3 | | 114.8 | |
| 6′ | 145.3 | | 152.1 | | 151.81 | |
| 7′ | | 9.98, s | | | | |
| 8′ | 152.7 | | 142.3 | 8.42, s | 142.7 | 8.45, s |
| 9′ | | 11.35, s | | | | |
| 10′ | | 6.73, t | | 9.03, br s | | 9.16, br s |
| | | (5.8) | | | | |
| 11′ | 39.0 | 3.72, m | 40.0 | 3.87, br s | 40.0 | 3.87, br s |
| 12′ | 37.3 | 2.96, t | 36.5 | 3.06, t | 36.4 | 3.05, t |
| | | (6.5) | | (6.8) | | (6.7) |

### Structure Elucidation of Compounds 1-5 (FIG. 22)

Compound 1 was isolated as a white solid. The molecular formula was determined as C₁₃H₁₃N₇S based on the analysis of the HRESIMS and ¹H and ¹³C NMR data (Tables 1 and 2). When the NMR data was collected in DMSO-d₆, broad and weak ¹H and ¹³C signals were observed for a methine (δ_{H} 9.00, δ_{C} 128.7) and a methylene (δ_{H} 4.61, δ_{C} 42.8) which afforded inadequate 2D NMR (HSQC and HMBC) correlations (FIGS. 24-28). The problem was overcome by changing the solvent to methanol-d₄ enabling the unambiguous elucidation of the structure of 1 (FIG. 22). Analysis of the ¹H and ¹³C NMR and the HSQC data revealed the presence of five aromatic methines, two connected methylenes, and a N-methyl group (δ_{H} 4.02, δ_{C} 35.1). By comprehensive analysis of the HMBC correlations (FIG. 23) and compassion of the ¹H and ¹³C chemical shifts with reported data, three heterocyclic systems were established including two common natural product building blocks, a 6-substituted purine (δ_{H} 8.45, 8.18; δ_{C} 153.6, 152.7, 151.8, 141.7, 121.6) (Schram, et al., Nature Reviews Clinical Oncology, 14: 735-48 (2017)) and a 5-substituted N-Me imidazole (δ_{H} 8.88, 7.62, 4.02; δ_{C} 137.7, 134.5, 120.0, 35.1) (Savage, et al., N. Engl. J. Med., 346: 683-93 (2002)). The remaining portion of the structure was constructed as a disubstituted dihydro-1,4-thiazine moiety evidenced by the key ¹H-¹H COSY correlations between 2H-5 (δ_{H} 4.71) and 2H-6 (δ_{H} 3.38) and HMBC correlations from H-3 (δ_{II} 9.00) to C-2 (δ_{C} 97.6) and C-5 (δ_{C} 44.6) and from 2H-6 to C-2. The dihydro-1,4-thiazine moiety was connected with the N-Me imidazole moiety between C-2 and C-5′ (δ_{C} 134.5) via a single bond supported by the HMBC correlations from H-3 to C-5′ and from H-4′ (δ_{H} 7.62) to C-2. Finally, the structure elucidation of 1 was completed by link the dihydro-1,4-thiazine moiety with the purine moiety between N-4 and C-6″ (δ_{C} 151.8) based on the key HMBC correlations from H-3 and 2H-5 to C-6″ (FIGS. 29-33). Compound 1, named as aplithianine A, represents the first example of a new class of purine-thiazine-imidazole interlinked alkaloids.

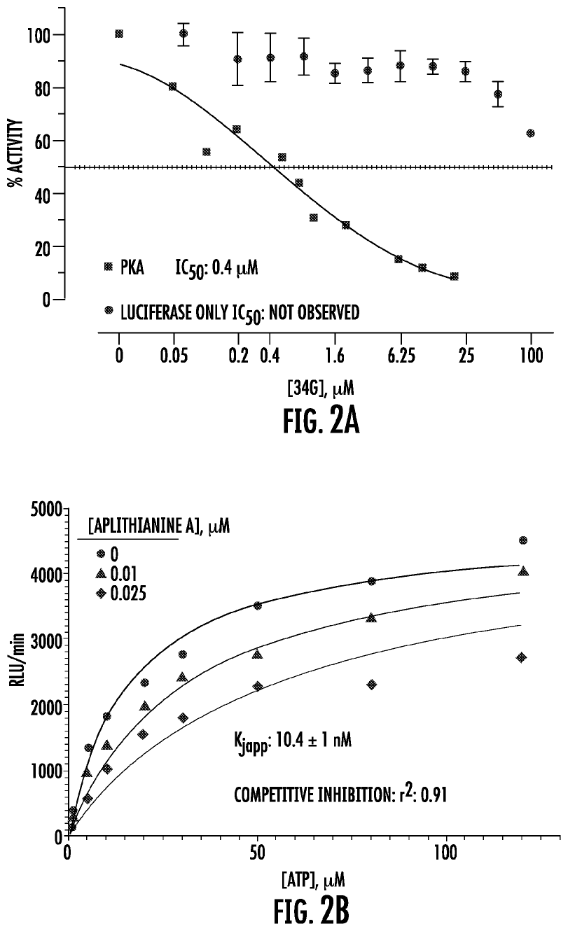

Compound 2 was isolated as a white solid. The molecular formular, C₁₃H₁₃N₇OS, was determined based on the HRESIMS data. Comparison of the 1D (¹H and ¹³C, Tables 1 and 2) and 2D (¹H-1H COSY, HSQC, and HMBC, FIG. 2) NMR data of 2 and 1 revealed the same structure skeleton except for a minor alteration in the structure of the purine moieties. The C-8″ methine (δ_{H} 8.18, ϵ_{C} 141.7) of 1 was changed to a carbonyl at δ_{C} 151.8 for 2. The assignment was supported by the presence of the exchangeable protons for both NH-7″ (δ_{H} 11.1) and NH-9″ (δ_{H} 11.9) in the ¹H NMR data of 2 (Table 2) measured in DMSO-d₆ and the key HMBC correlations from the two protons to C-8′ (FIGS. 34-41). Thus, the structure of 2, which was given a trivial name as aplithianine B, was determined as the 8″-oxopurine analogue of 1.

The molecular formular of 3 was determined as C₁₄H₁₆N₁₀O₂S₂ by interpretation of the HRESIMS data. The ¹H and ¹³C NMR spectra showed only 8 protons and 7 carbons implying the homodimeric nature of the structure. Analysis of the ¹H and ¹³C NMR data (Table 3) and the ¹H, ¹H-COSY and HMBC correlations quickly revealed the presence of a cysteamine moiety (—S—CH₂—, δ_{H} 2.96 and δ_{C} 37.3; —NH—CH₂—, δ_{H} 3.72, 6.73, and δ_{C} 39.0) (Honeyman, et al., Science, 343: 1010-14 (2014)) and an 8-oxopurine moiety (δ_{H} 8.05, 9.98, 11.35, and δ_{C} 104.7, 145.3, 147.3, 150.7, 152.7), the same as that in the structure of 2 (Tables 1 and 2). The two moieties were connected between N-10 and C-6 based on the key HMBC correlations from NH-10 (δ_{H} 6.73) and 2H-11 (δ_{H} 3.72) to C-6 (δ_{C} 145.3). As the NMR analysis established the half structure of the molecule accounting for C₇H₈N₅OS, the homodimer can only be formed by linking the two half structures via a disulfide bond (FIGS. 61-65). Thus, the structure of 3 was determined as a new disulfide-linked nucleobase homodimer, named as aplidipurinide A.

Both compounds 4 and 5 had very similar UV spectra (UV maxima at ˜211 and 273 nm) and molecular formulars (C₁₄H₁₆N₁₀S₂ for 4 and C₁₄H₁₆N₁₀OS₂ for 5) compared to those of 3 indicating they were structural analogues. The ¹H and ¹³C NMR spectra (Table 3) of 4 also displayed signals for only half of the molecule and the nucleobase portion was changed to a 6-N-purine moiety evidenced by the lack of oxygen atoms in the molecule and the presence of the 8-methine signals (δ_{H} 8.42, δ_{C} 142.3). In contrast to 3 and 4, the ¹H and ¹³C NMR data (Table 3) of 5 showed two sets of signals, which resembled the NMR features of both 3 and 4, respectively. Thus, compound 4 was determined to be a homodimeric analogue of 3 with the nucleobase portions changed to 6-N-purines while the structure of 5 was deduced as a heterodimer constructed by linking the half structures of 3 and 4 via a disulfide bond (FIG. 22). The structural determination was supported by the analysis of the 2D (¹H, ¹H-COSY, HSQC, and HMBC) NMR correlations (FIGS. 66-75). Compound 4 was first reported as a synthetic compound (Kastenhuber, et al., PNAS USA, 114: 13076-84 (2017)) but it had never been discovered from a natural source. Thus, compounds 4 and 5 were determined as new natural analogues of 3, named as aplidipurinides B and C, respectively.

### Example 2

This example demonstrates that compounds of the present invention can be synthesized.

Chemical investigation of the bulkAplidium sp. extracts led to the accumulation of 116 mg of 1 enabling the generation of semi-synthetic aplithianine analogues to acquire some preliminary insights into the structure-activity relationships. Considering the limited supply of natural-derived 1, the initial design of semi-synthesis only focused on the optimization of certain robust reactions that were expected to yield simple product profiles.

### Total Synthesis of Aplithianine A

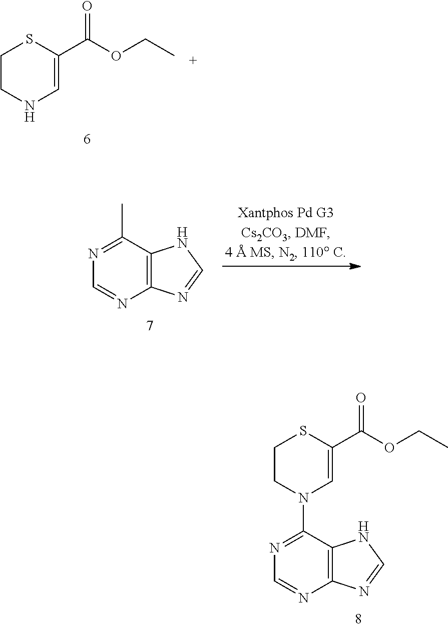

To the mixture of ethyl 3,4-dihydro-2H-1,4-thiazine-6-carboxylate (6, 1 equiv., 100 mg) and 6-bromopurine (7, 1.5 equiv., 170 mg) was added Xantphos Pd G3 (10 mol %, 55 mg), Cs₂CO₃ (3 equiv., 560 mg), DMF (5 mL), and 4 Å molecular sieves. The reaction vial was filled with N₂ and capped tightly. The reaction mixture was then stirred vigorously at 110° C. overnight before it was dried down under vacuum. The residue was re-dissolved in DMSO followed by preparative HPLC purification using a Gemini 5 μm NX-C18 column (110 Å, 250×21.2 mm) with flow rate of 10 mE/min (eluted with 10% to 100% MeCN in 0.1% TFA) to yield 8 (61 mg, 36% yield).

Ethyl 4-(7H-purin-6-yl)-3,4-dihydro-2H-1,4-thiazine-6-carboxylate (8): pale orange solid; ¹H NMR (600 MHz, DMSO-d₆): δ 13.56 (s, 1H), 9.77 (s, 1H), 8.52 (s, 1H), 8.44 (s, 1H), 4.55 (br s, 2H), 4.20 (q, J=7.1 Hz, 2H), 3.16 (m, 2H), 1.24 (t, J=7.1 Hz, 3H); ¹³C NMR (150 MHz, DMSO-d₆): δ 164.7, 153.0, 151.3, 149.5, 141.7, 134.6, 120.4, 102.1, 60.5, 43.5, 23.8, 14.4.

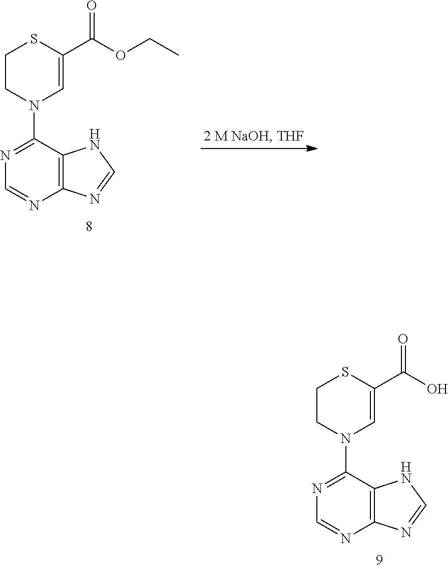 [SMILES: CCOC(=O)C1=CN(c2ncnc3nc[nH]c23)CCS1 ; O=C(O)C1=CN(c2ncnc3nc[nH]c23)CCS1]

Compound 8 (61 mg) was dissolved in 2M NaOH (3.5 mL) and THF (3.5) and stirred at room temperature overnight. The reaction solution was acidified with 2M HCl (5 mL) and then dried down under vacuum. The crude product was then washed and desalted with H₂O (1 mL×3 times) to yield 9 (49 mg, 90% yield). FIG. 96 shows a ¹H NMR spectrum of 8 in DMSO-d₆. FIG. 97 shows a ¹³C NMR spectrum of 8 in DMSO-d₆.

4-(7H-purin-6-yl)-3,4-dihydro-2H-1,4-thiazine-6-carboxylic acid (9): pale orange solid; ¹H NMR (600 MHz, DMSO-d₆): δ 13.53 (s, 1H), 12.56 (brs, 1H), 9.78 (s, 1H), 8.50 (s, 1H), 8.43 (s, 1H), 4.51 (br s, 2H), 3.13 (m, 2H); ¹³C NMR (150 MHz, DMSO-d₆): δ 166.3, 152.9, 151.3, 149.5, 141.6, 134.3, 120.2, 103.2, 43.1, 23.7. FIG. 98 shows a ¹H NMR spectrum of 9 in DMSO-d₆. FIG. 99 shows a ¹³C NMR spectrum of 9 in DMSO-d₆.

### Route 1

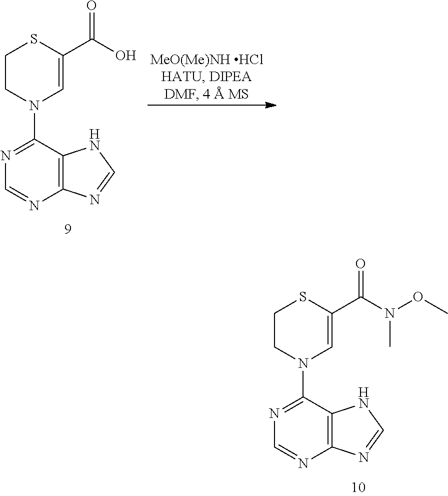 [SMILES: O=C(O)C1=CN(c2ncnc3nc[nH]c23)CCS1 ; CON(C)C(=O)C1=CN(c2ncnc3nc[nH]c23)CCS1]

To the mixture of 9 (1 equiv., 37 mg) and N,O-dimethylhydroxylamine hydrochloride (3 equiv., 40 mg) was added HATU (1.5 equiv., 71 mg), DIPEA (10 equiv., 228 μL), DMF (5 mL), and 4 Å MS. The reaction mixture was stirred vigorously at room temperature overnight before it was dried down under vacuum. The residue was re-dissolved in DMSO followed by preparative HPLC purification using a Gemini 5 μm NX-C18 column (110 Å, 250×21.2 mm) with flow rate of 10 mL/min (eluted with 30% MeCN in 0.1% TFA) to yield 10 (40 mg, 93% yield).

N-methoxy-N-methyl-4-(7H-purin-6-yl)-3,4-dihydro-2H-1,4-thiazine-6-carboxamide (10): pale orange solid; ¹H NMR (600 MHz, DMSO-d₆): δ 13.50 (s, 1H), 9.68 (s, 1H), 8.49 (s, 1H), 8.40 (s, 1H), 4.52 (br s, 2H), 3.70 (s, 3H), 3.17 (s, 3H), 3.08 (m, 2H); ¹³C NMR (150 MHz, DMSO-d₆): δ 166.5, 152.8, 151.3, 149.8, 141.3, 133.2, 120.2, 104.4, 61.1, 44.8, 33.6, 24.5. FIG. 100 shows a ¹H NMR spectrum of 10 in DMSO-d₆. FIG. 101 shows a ¹³C NMR spectrum of 10 in DMSO-d₆.

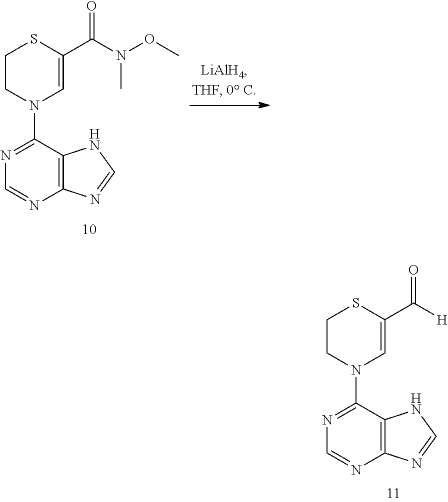 [SMILES: CON(C)C(=O)C1=CN(c2ncnc3nc[nH]c23)CCS1 ; O=CC1=CN(c2ncnc3nc[nH]c23)CCS1]

Compound 10 (1 equiv., 40 mg) was stirred in THF (5 mL) at 0° C. followed by addition of 2M LiAlH₄ in THF (4 equiv., 260 μL). The reaction was halted after 1 hour by the addition of H₂O (1 mL). The reaction mixture was dried down under vacuum. The residue was re-dissolved in DMSO followed by preparative HPLC purification using a Gemini 5 μm NX-C18 column (110 Å, 250×21.2 mm) with flow rate of 10 mL/min (eluted with 30% MeCN in 0.1% TFA) to yield 11 (22 mg, 68% yield).

4-(7H-purin-6-yl)-3,4-dihydro-2H-1,4-thiazine-6-carbaldehyde (11): pale orange solid; ¹H NMR (600 MHz, DMSO-d₆): δ 13.69 (s, 1H), 9.61 (s, 1H), 9.32 (s, 1H), 8.60 (s, 1H), 8.52 (s, 1H), 4.64 (m, 2H), 3.18 (m, 2H); ¹³C NMR (150 MHz, DMSO-d₆): δ 187.4, 153.2, 151.3, 149.1, 144.0, 142.4, 120.7, 115.4, 44.8, 22.9. FIG. 102 shows a ¹H NMR spectrum of 11 in DMSO-d₆. FIG. 103 shows a ¹³C NMR spectrum of 11 in DMSO-d₆.

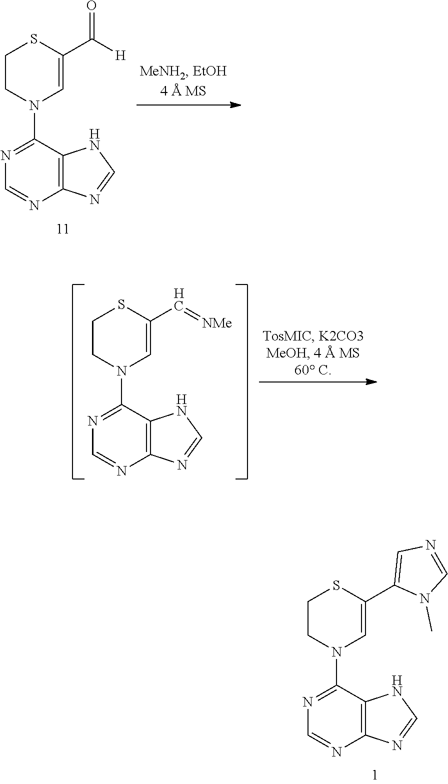

Compound 11 (1 equiv., 22 mg) was stirred in 33% methylamine in EtOH (5 mL) in the presence of 4 Å molecular sieves at room temperature for 4 hours. The reaction mixture was dried down under vacuum followed by the addition of toluenesulfonylmethyl isocyanide (2 equiv., 55 mg), K2CO₃ (2 equiv., 23 mg), MeOH (5 mL), and 4 Å molecular sieves. The reaction mixture was stirred at 60° C. overnight before it was dried down under vacuum. The residue was re-dissolved in DMSO followed by preparative HPLC purification using a Synergi 5 μm Hydro-RP column (110 Å, 250×21.2 mm) with flow rate of 10 mL/min (eluted with 19% MeCN in 0.1% TFA) to yield 1 (4 mg, 15% yield).

### Route 2

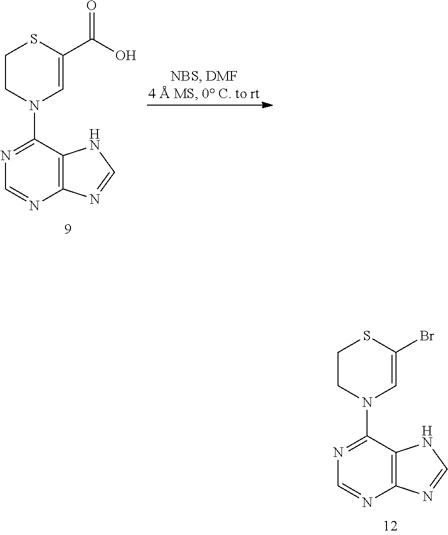 [SMILES: O=C(O)C1=CN(c2ncnc3nc[nH]c23)CCS1 ; BrC1=CN(c2ncnc3nc[nH]c23)CCS1]

The reaction vial containing a DMF (5 mL) solution of 9 (1 equiv., 49 mg) with 4 Å molecular sieves was filled with N₂ and cooled down in an ice bath. A 10 mg/mL MeCN solution of N-bromosuccinimide (1.2 equiv., 3.8 mL) was added dropwise into the reaction vial. The reaction mixture was stirred at 0° C. for 2 h and then warmed up to room temperature for continued stir overnight. The reaction mixture was dried down under vacuum. The residue was re-dissolved in DMSO followed by preparative HPLC purification using a Gemini 5 μm NX-C18 column (110 Å, 250×21.2 mm) with flow rate of 10 m/min (eluted with 10-100% MeCN in 0.10% TFA) to yield 12 (25 mg, 45% yield).

6-bromo-4-(7H-purin-6-yl)-3,4-dihydro-2H-1,4-thiazine (12): pale orange solid; ¹H NMR (600 MHz, DMSO-d₆): δ 13.36 (s, 1H), 8.98 (s, 1H), 8.37 (s, 1H), 8.31 (s, 1H), 4.56 (br s, 2H), 3.32 (m, 2H); ¹³C NMR (150 MHz, DMSO-d₆): δ 152.3, 151.3, 149.0, 140.4, 125.7, 119.3, 91.1, 41.7, 28.2. FIG. 104 shows a ¹H NMR spectrum of 12 in DMSO-d₆. FIG. 105 shows a ¹³C NMR spectrum of 12 in DMSO-d₆.

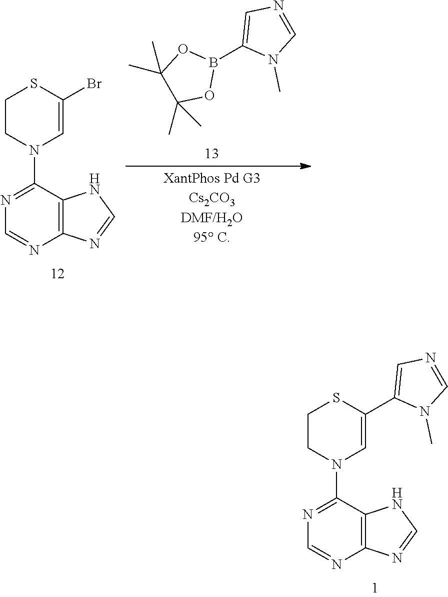

To the mixture of 12 (1 equiv., 14.5 mg) and 1-methyl-5-(4,4,5,5-tetramethyl-1,3,2-dioxaborolan-2-yl)-1H-imidazole (4 equiv., 58 mg) was added Xantphos Pd G3 (20 mol %, 8.7 mg), Cs₂CO₃ (3 equiv., 560 mg), DMF (3 mL), H₂O (0.3 mL), and 4 Å molecular sieves. The reaction vial was filled with N₂ and capped tightly. The reaction mixture was then stirred vigorously at 95° C. overnight before it was dried down under vacuum. The residue was re-dissolved in DMSO followed by successive preparative HPLC purifications using a Gemini 5 μm NX-C18 column (110 Å, 250×21.2 mm) with flow rate of 10 mL/min (eluted with 10% to 100% MeCN in 0.1% TFA) and a Synergi 5 μm Hydro-RP column (110 Å, 250×21.2 mm) with flow rate of 10 mL/min (eluted with 19% MeCN in 0.1% TFA) to yield 1 (9.8 mg, 67% yield).

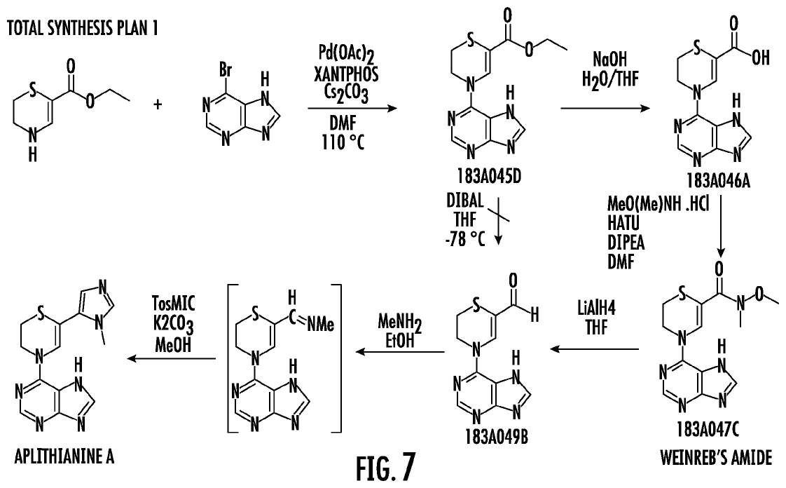

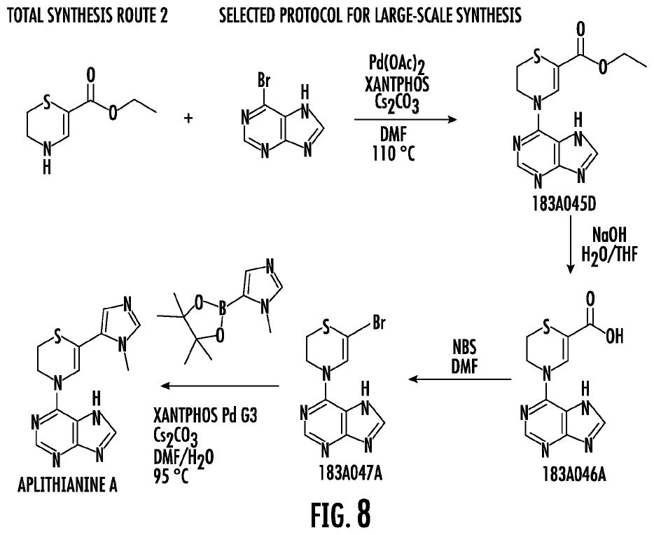

To continue developing aplithianine A (1) as a drug candidate, a stable supply of the molecule needed to be addressed. Thus, two interlapped synthesis routes were designed for the total synthesis of aplithianine A (1) (Scheme 1, FIG. 7). Both routes started with the Buchwald-Hartwig coupling of the commercial precursors ethyl 3,4-dihydro-2H-1,4-thiazine-6-carboxylate (6) and 6-bromopurine (7) followed by the basic hydrolysis of the resulted ester 8 to yield the carboxylic acid 9 quantitatively. The yield of the Buchwald-Hartwig coupling reaction reached about 36% by extensive optimization of the reaction conditions, including the catalysts/precatalysts [e.g. Pd₂(dba)₃, Pd(OAc)₂, XantPhos Pd G3, XantPhos Pd G4, XPhos Pd G4, and P(t-Bu)₃ Pd G4), ligands (e.g. Xantphos, BINAP, JohnPhos, DavePhos, XPhos, RuPhos), bases (e.g. Cs₂CO₃, sodium tert-butoxide, and LiHMDS), solvents (e.g. DMF, THF, toluene, and MeCN), and temperatures (e.g. 25 to 130° C.). The combination of the precatalyst Xantphos Pd G₃ with the base Cs₂CO₃ in DMF as the solvent provided the optimal yield at 110° C. comparing to most other tested conditions (distinct combinations of aforementioned reaction factors) which generally resulted in less than 5% yield of 1. For route 1, the carboxylic acid 9 was converted to its Weinreb amide 10 through the typical HATU/DIPEA/DMF amidation system. Reduction of 10 by LiAlH₄ provided the aldehyde 11 which was further subjected to the Van Leusen imidazole synthesis in two steps to yield the final product 1. Unfortunately, the total yield of 1 from the six-step route 1 was only about 3% majorly due to the low yield of the last step Van Leusen imidazole synthesis (15% yield). Thus, a second, shorter synthesis route was designed by directly coupling of a purine-thiazine conjugate with an imidazole unit. The carboxylic acid 9 was converted to the brominated 12 by decarboxylative bromination with NBS/DMF. A similar catalytic system (i.e., XantPhos Pd G3/Cs₂CO3/DMF) to that of step 1 (Buchwald-Hartwig coupling of 6 and 7) was applied to directly conjugate the brominated 12 with the boronic ester 1-methyl-5-(4,4,5,5-tetramethyl-1,3,2-dioxaborolan-2-yl)-1H-imidazole (13) to provide 1 in an improved 10% yield for the total of 4 reaction steps (see FIG. 8).

### Example 3

This example demonstrates that compounds of the present invention can be brominated.

### Bromination of Aplithianine A (1)

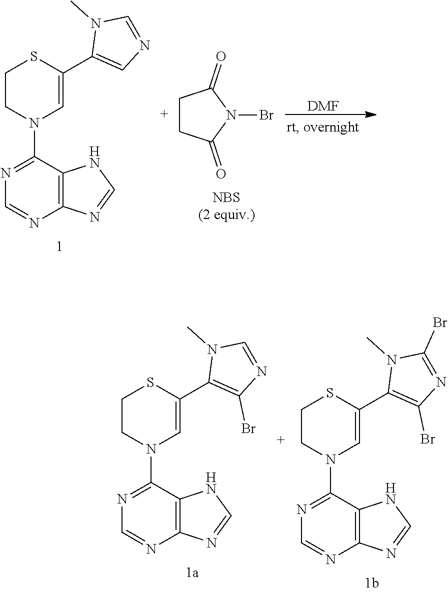

To the dry mixture of N-bromosuccinimide (5 mg, 2 equiv.) and compound 1 (4 mg, 1 equiv.), 2 mL anhydrous DMF was added. The resulting solution was stirred overnight at room temperature. The reaction solution was dried down under vacuum and the residue was re-dissolved with DMSO. The products were purified by semi-prep HPLC using a Synergy 5 μm Polar-RP column (110 Å, 250×10 mm) with flow rate of 4 mL/min (eluted with 20-100% MeCN in 0.1% TFA) to yield 1a (1.3 mg) and 1b (0.7 mg).

Aplithianine A₁ (1a): white solid; UV (MeOH) λₘₐₓ (log ε) 240 (3.84), 329 (4.15); IR (neat) νₘₐₓ 3066, 3007, 2921, 2847, 1607, 1571, 1498, 1451, 1373, 1291, 1191, 1139, 1076, 1034, 934, 855, 793, 776, 571, 553, 534 cm⁻¹; ¹H and ¹³C NMR data, see Tables 1 and 2 and FIGS. 42-46; HRESIMS m/z 378.0128, [M+H]⁺ (calcd for C₁₃H₁₃BrN₇S, 378.0137).

Aplithianine A₂ (1b): white solid; UV (MeOH) λₘₐₓ (log ε) 242 (4.14), 329 (4.42); IR (neat) νₘₐₓ 3074, 3009, 2923, 2850, 1608, 1572, 1505, 1453, 1404, 1364, 1330, 1294, 1192, 1141, 1077, 1062, 1033, 934, 867, 793, 778, 642, 571, 554, 535 cm⁻¹; ¹H and ¹³C NMR data, see Tables 1 and 2 and FIGS. 47-50; HRESIMS m/z 455.9238, [M+H]⁺ (calcd for C₁₃H₁₂Br₂N₇S, 455.9242).

Reaction of 1 with 2 equiv. N-bromosuccinimide (NBS) yielded the mono-brominated product 1a and the di-brominated product 1b with the bromination exclusively happening on the imidazole moiety.

### Example 4

This example demonstrates that compounds of the present invention can be oxidated using different methods.

Oxidation of Aplithianine A (1) with SELECTFLUOR.

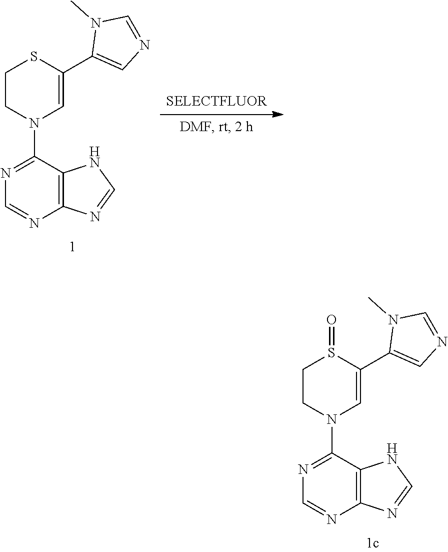 [SMILES: Cn1cncc1C1=CN(c2ncnc3nc[nH]c23)CCS1 ; Cn1cncc1C1=CN(c2ncnc3nc[nH]c23)CCS1=O]

SELECTFLUOR (4.4 mg, 1 equiv.) and compound 1 (3.7 mg, 1 equiv.) were stirred in 2 mL anhydrous DMF at room temperature for 2 h. The reaction solution was dried down under vacuum and the residue was re-dissolved with MeOH. The products were purified by semi-prep HPLC using a Synergy 5 μm Polar-RP column (110 Å, 250×10 mm) with flow rate of 4 m/min (eluted with 10% MeCN in 0.1% TFA) to yield 1c (1.4 mg).

Aplithianine A₃ (1c): white solid; UV (MeOH) λₘₐₓ (log ε) 219 (3.87), 322 (4.06); IR (neat) νₘₐₓ 3116, 3057, 2963, 2920, 2849, 1681, 1632, 1596, 1569, 1452, 1414, 1367, 1288, 1258, 1203, 1182, 1128, 1050, 1031, 935, 837, 799, 721, 644 cm⁻¹; ¹H and ¹³C NMR data, see Tables 1 and 2 and FIGS. 51-55; HRESIMS m/z 316.0975, [M+H]⁺ (calcd for C₁₃H₁₄N₇OS, 316.0981).

### Oxidation of Aplithianine A (1) with H₂O₂

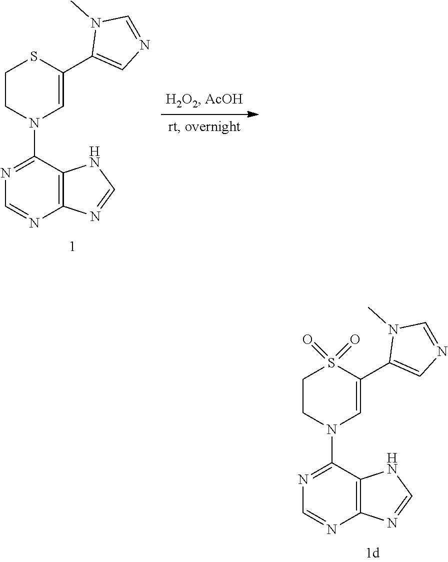 [SMILES: Cn1cncc1C1=CN(c2ncnc3nc[nH]c23)CCS1 ; Cn1cncc1C1=CN(c2ncnc3nc[nH]c23)CCS1(=O)=O]

The solution of compound 1 (4 mg) in acetic acid (4 mL) was added with 1 mL 30% H₂O₂ solution. The reaction mixture was dried down overnight at room temperature. The reaction solution was dried down under vacuum and the residue was re-dissolved with MeOH. The products were purified by semi-prep HPLC using a Kinetex 5 μm EVO C18 column (110 Å, 250×10 mm) with flow rate of 4 mL/min (eluted with 7% MeCN in 0.1% TFA) to yield 1d (1.5 mg).

Aplithianine A₄ (1d): white solid; UV (MeOH) λₘₐₓ (log ε) 220 (4.00), 312 (4.22); IR (neat) νₘₐₓ 3117, 3010, 2921, 2826, 1674, 1640, 1598, 1571, 1454, 1413, 1366, 1328, 1290, 1194, 1176, 1120, 1077, 1040, 934, 840, 795, 719, 643, 571, 534 cm⁻¹; ¹H and ¹³C NMR data, see Tables 1 and 2 and FIGS. 56-60; HRESIMS m/z 332.0923, [M+H]⁺ (calcd for C₁₃H₁₄N₇O₂S, 332.0930).

Efforts to generate fluorinated analogue using 1 equiv. SELECTFLUOR led to the complete conversion of 1 into the partially oxidized sulfoxide analogue 1c while the complete oxidation of 1 in H₂O₂/AcOH provided the sulfone analogue 1d.

### Example 5

This example demonstrates that compounds of the present invention are active against PKADJ/PKA.

To evaluate the activities of compounds 1-5 against PKADJ, they were tested in a modified sandwich ELISA assay. The PKADJ holoenzyme was treated with the test compounds in the reaction step where the holoenzyme was dissociated to release the activated catalytic unit (PKADJc). The activity of the dissociated PKADJc was quantified by measuring the phosphorylation of a biotinylated peptide substrate (KRREILSRRPSYR (SEQ ID NO: 1)) through immunofluorescence. Compound 1 showed potent inhibition against the activity of PKADJ with the IC₅₀ value at 1.1 μM (see FIGS. 1A-1B). In contrast, the analogue 2 only weakly inhibited PKADJ (IC₅₀=69 μM), more than 60-fold less potent than 1. The nucleobase dimers 3-5 were inactive in this assay with IC_{50S}>90 μM.

Both 1 and 2 were further evaluated for their activities against the wide-type PKA (wt-PKA) using the same assay. Both compounds showed almost equivalent potency (IC₅₀ values 1.64 μM for 1 and 45 μM for 2) against PKA when compared to their inhibition against PKADJ. Despite of the lack of selectivity against PKADJ, the aplithianines represented a new class of naturally derived kinase inhibitors with an unprecedented structural skeleton. It warranted the further investigation of the mechanism of actions and structure-activity relationships of this new class of kinase inhibitors (see FIGS. 11A-I 1P).

### Example 6

This example demonstrates that compounds of the present invention inhibit PKA activity.

To investigate the mechanism of actions of aplithianine A (1), a luciferase assay was first carried out to confirm if 1 truly inhibited PKA activity without interfering with the assay. Compound 1 potently inhibited the PKA activity with a IC₅₀ value of 0.4 μM while no activity was observed when PKA was absent in the assay. Therefore, a competitive kinetic study was further conducted to investigate how 1 interacted with the PKA protein. At the concentrations from 0 to 25 nM, compound 1 competitively inhibited the PKA activity induced by 0-100 μM ATP in a dose-dependent manner with the Kᵢₐₚₚ and r² values at 10.4 nM and 0.91, respectively. Thus, all the results supported that 1 was likely to function as an ATP-competitive inhibitor by direct interacting with the PKA catalytic unit (see FIGS. 2A-2B).

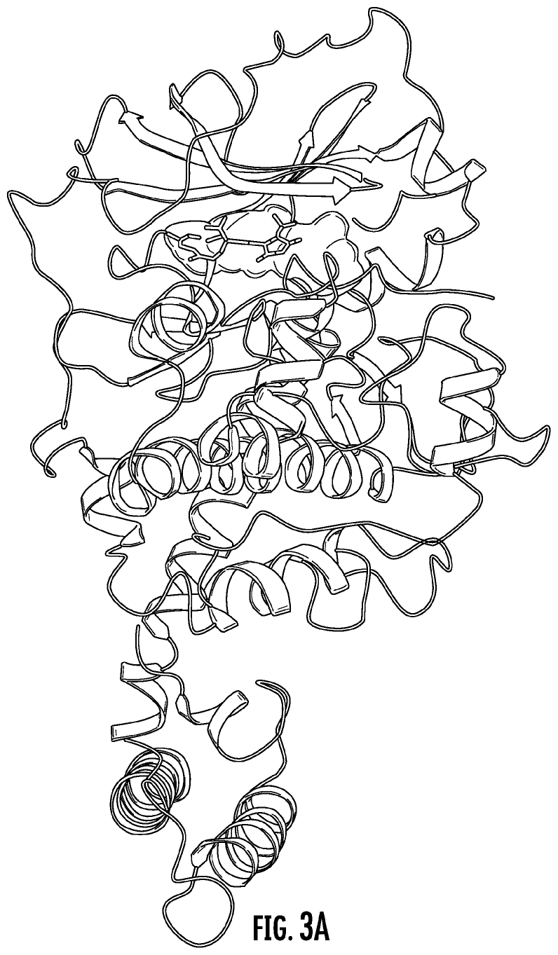

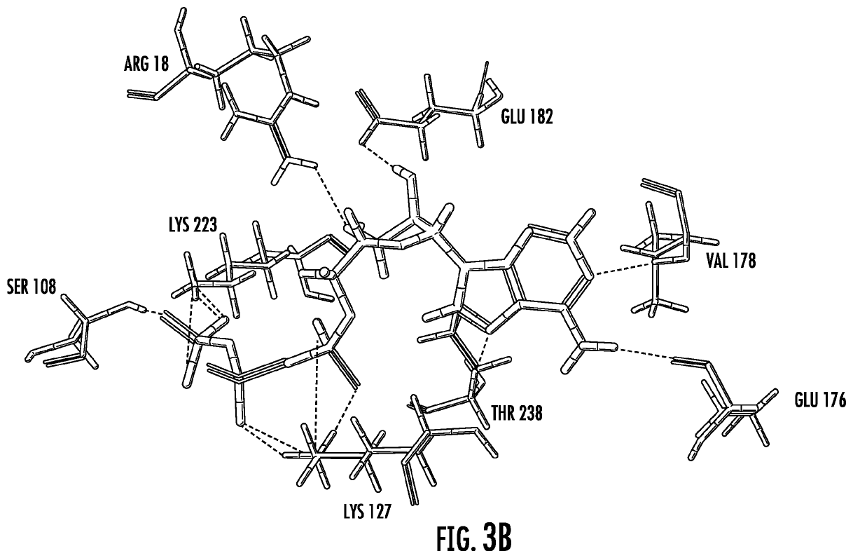

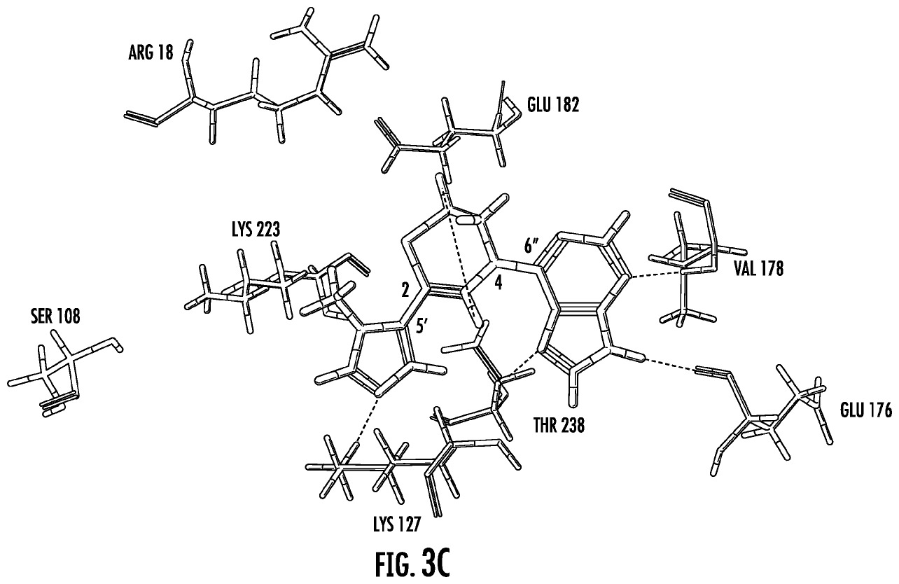

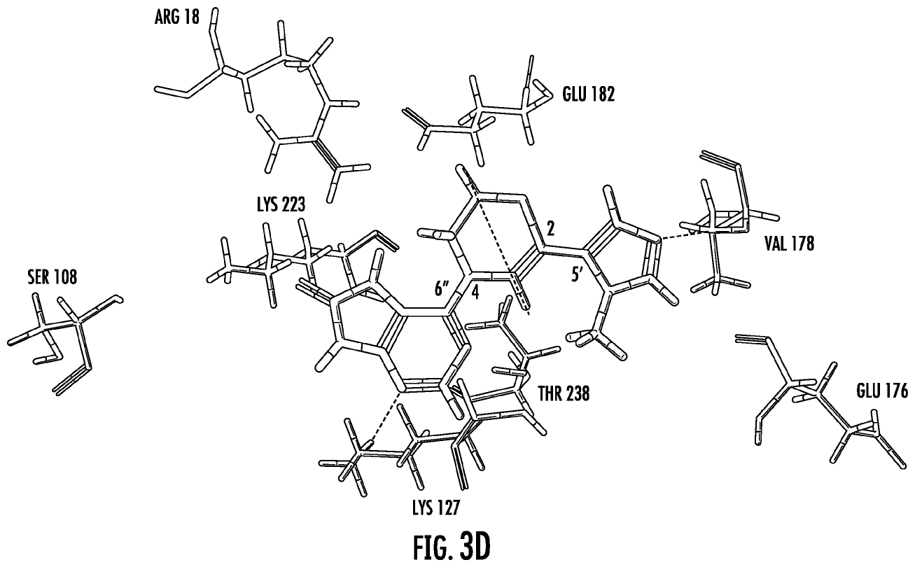

To further investigate the mechanism of aplithianines binding to PKA, both 1 and 2 were subjected to co-crystallization experiments with the DNAJ-PKAc fusion protein. The X-ray diffraction experiments on the co-crystals of 1 and 2 with DNAJ-PKAc, respectively, revealed distinct binding modes. Compound 1 partially occupied the ATP-binding pocket with three H-bonds predicted from N-3″, NH-9″, and N-7″ of the purine moiety to the PKAc residues Val178, Glu176, and Thr238, respectively, and with another one between the imidazole N-3′ and Lys127. In contrast, the adenine moiety of ATP was repositioned in the bind pocket with N-1, NH₂-6, and N-7 coordinating with Val178, Glu176, and Thr238, respectively. Surprisingly, the oxidation of C-8″ in the structure of 2 leading to a completely reversed binding mode as compared to that of 1 in the ATP binding pocket. In contrast to 1, the structure of 2 flipped over around the C-3/C-6 axis and both the purine and imidazole rings rotated around the N-4/C-6″ and C-2/C-5′ bonds, respectively, to afford two H-bonds between purine N-3″ and Lys127 and between imidazole N-3′ and Val178, respectively. Thus, both 1 and 2 may inhibit PKAc activity by competitively binding to the ATP pocket, nevertheless, the slight modification of the purine C-8″ completely reversed their binding poses probably resulting in varied binding affinities to PKAc and the differential potencies for PKAc inhibition (see FIGS. 3A-3D).

### Example 7

This example demonstrates the activity of analogues 1a-1d.

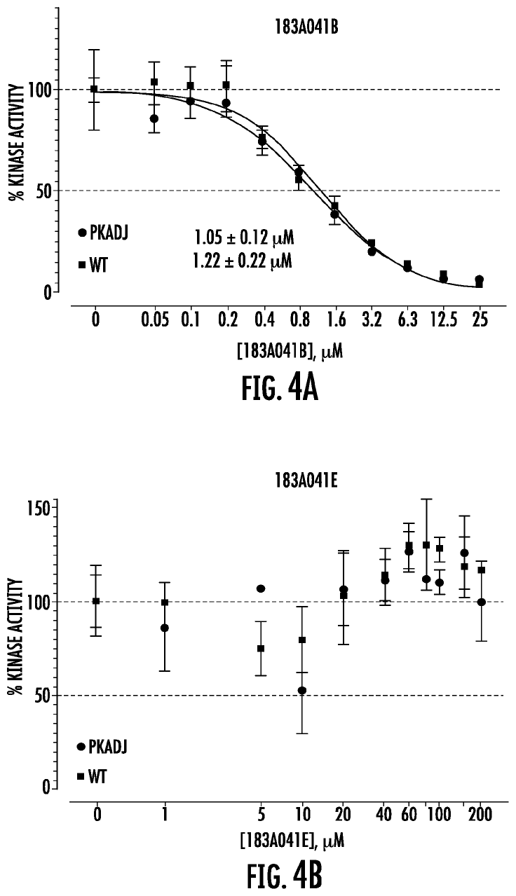

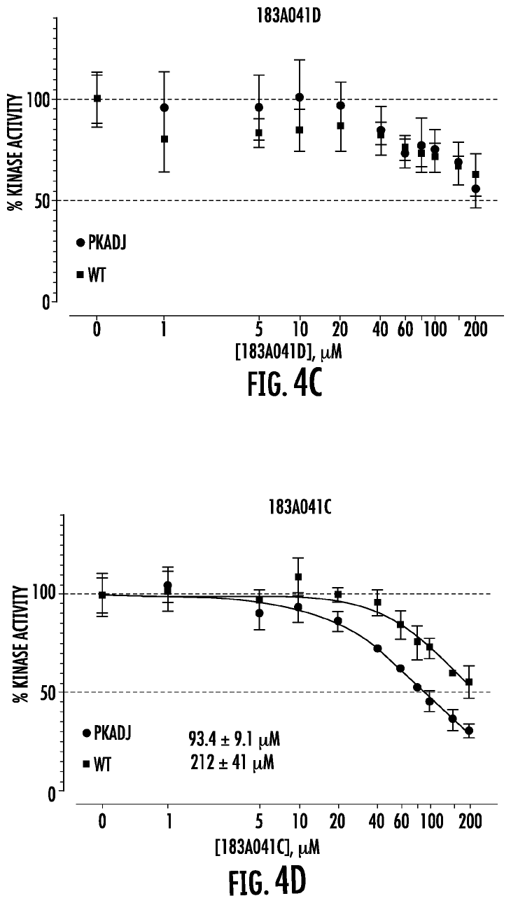

Analogues 1a-1d (Examples 3 and 4) were tested in the same manner as compound 1 as described in Example 5. Among the four semi-synthetic analogues 1a, 1b, 1c, and 1d (Examples 3 and 4 above), only 1a showed almost equivalent potency as compared to 1 against both PKADJ (IC₅₀=1.05 μM) and WT-PKA (IC₅₀=1.22 μM). Analogues 1b-1d were inactive against either PKADJ or WT-PKA with IC₅₀ values >90 μM. Further co-crystallization and X-ray diffraction experiments also revealed almost identical binding mode for 1a and 1 in line with their equivalent potency against PKADJ and PKA (see FIGS. 4A-4D).

### Example 8

This example demonstrates that compounds of the present invention inhibit kinases.

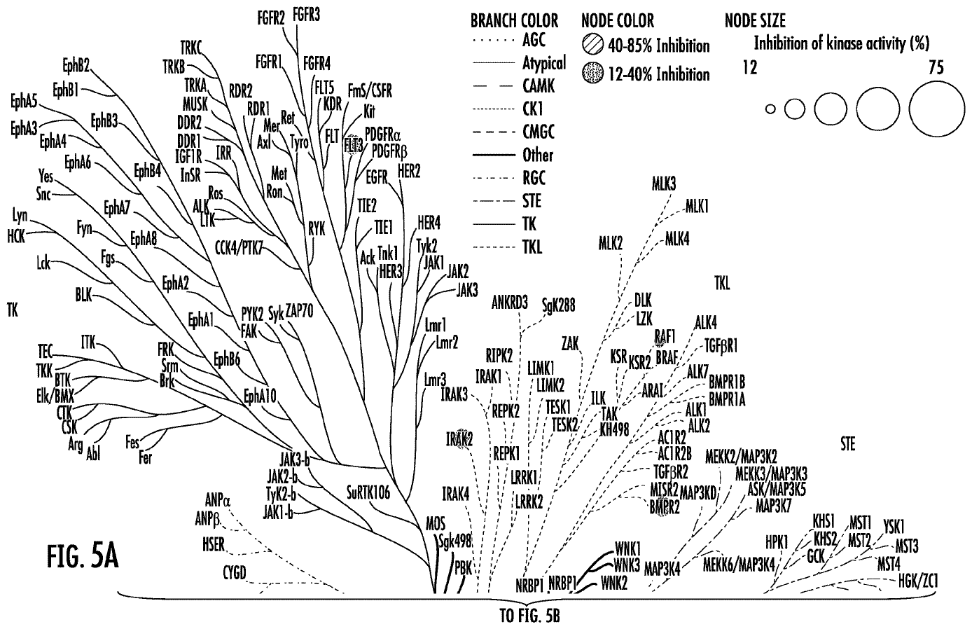

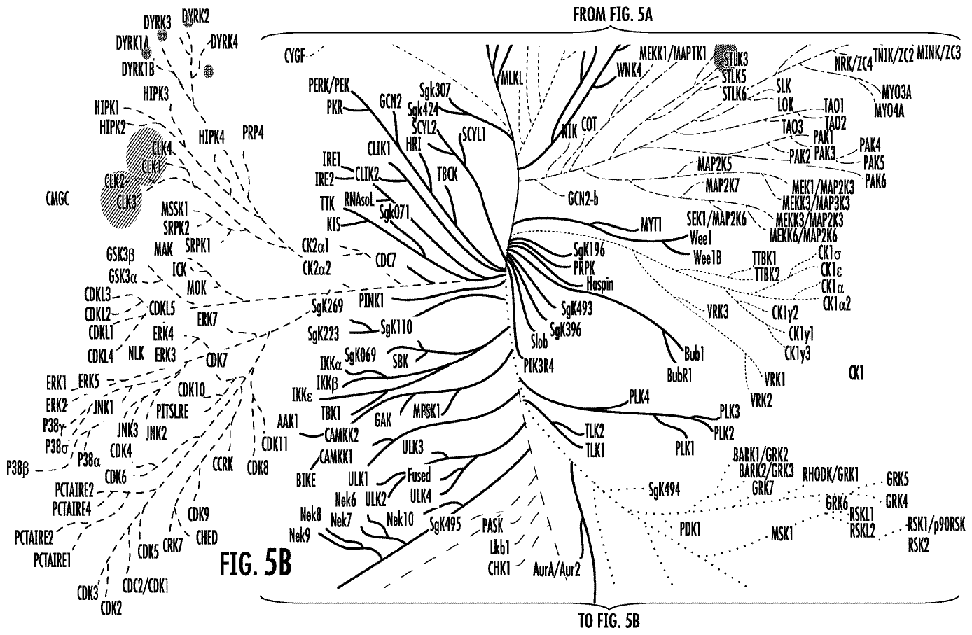

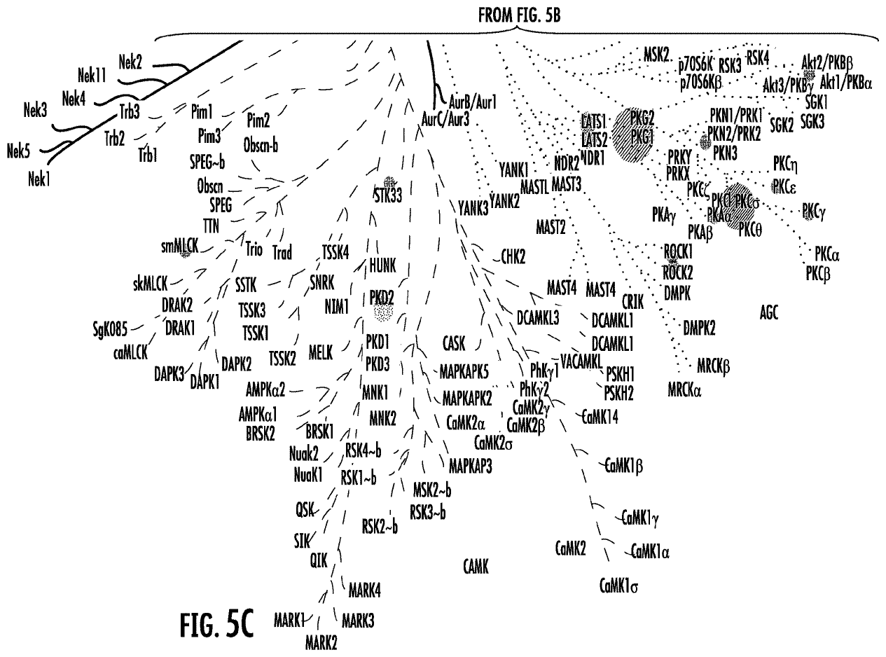

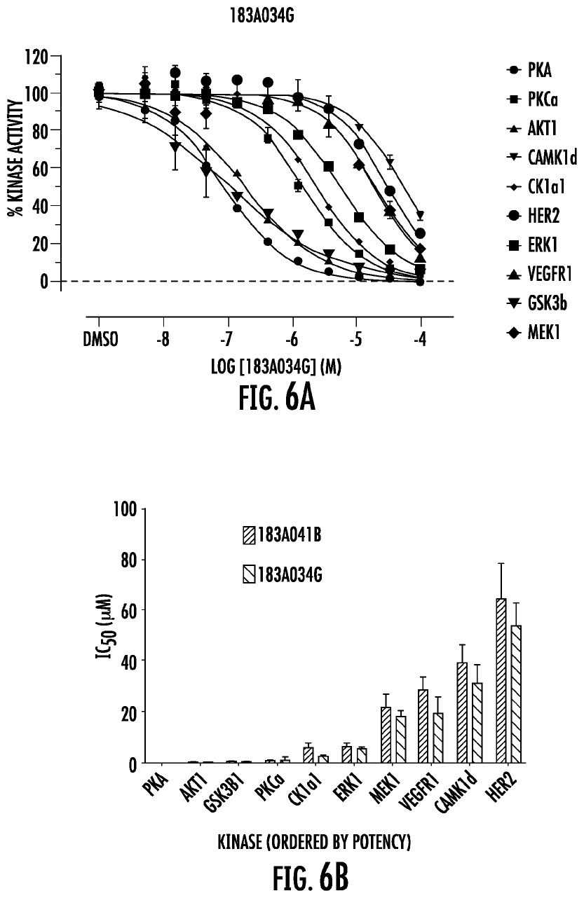

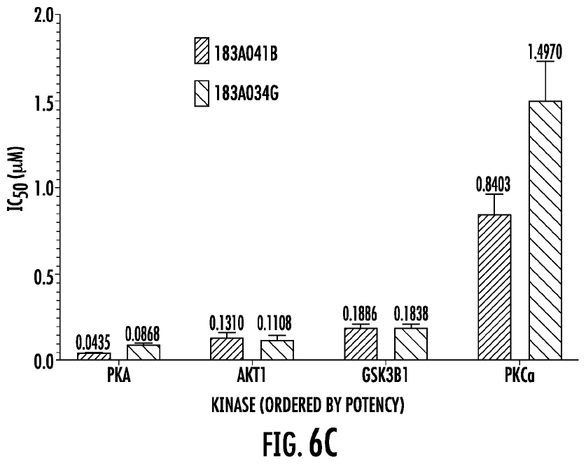

Dose-response studies against 10 kinases belonging to divergent phylogenetic groups were performed for compounds 1 and 1a to investigate their selectivity and potency against the human kinome. Both compounds showed comparable potency and selectivity against the 10 kinases with PKA, GSK3β, and AKT1 as the most potent targets (IC₅₀ values between 43 and 189 nM). Less potent but still prominent inhibition was observed against PKCa, CK1a1, and ERK1 (IC₅₀ values between 0.8 and 6.6 μM) while only moderate activities were displayed against VEGFR1, MEK1, HER2, and CAMK1d (IC₅₀ values between 18 and 65 μM). The semi-synthetic analogue 1a was further subjected to an expanded kinase profiling against a whole panel of 370 kinases. At 2 μM, 1a potently inhibited 101 kinases with >50% inhibition while it was totally inactive against 65 kinase (<5% inhibition). At 50 nM, 29 kinases emerged as more potent targets compared to PKA (78% inhibition) with most potent inhibition (>50% inhibition) observed for eight kinases, including PKG1a, STK39/STLK3, PKG1b, PKC-θ, CLK1, DYRK1/DYRK1A, DYRK2, and LATS1, mainly belonging to the AGC and CMGC groups of kinases (see FIGS. 5, 6A-6C, and 13-21).

Further dose-response studies of 1 was carried out against the 19 most sensitive kinases to 1a revealed similar selectivity with 9 more potent targets compared to PKA, including PKG1a, PKG1b, PKG2, PKCtheta, STK39, CLK1, LATS1, LATS2, and CLK2 (see Table 4). The curve fits are shown in FIGS. 76A-95B. Curve fits were performed where the enzyme activities at the highest concentration of compounds were less than 65%. The differential was calculated as follows: IC₅₀ (kinase)/IC₅₀ (PKG1a).

TABLE 4
IC₅₀ Values (nM)

| | Aplithianine A | Differential |
|---|---|---|
| Kinase | (Compound 1) | (as compared to PKG1a) |
| PKG1a | 11.4 | 1.00 |
| PKG1b | 15.7 | 1.37 |
| PKG2 | 35.1 | 3.08 |
| PKCtheta | 37.5 | 3.29 |
| STK39 | 41.3 | 3.63 |
| CLK1 | 63.4 | 5.57 |
| LATS1 | 66.4 | 5.83 |
| LATS2 | 75.6 | 6.64 |
| CLK2 | 77.5 | 6.81 |
| PKA | 88.1 | 7.74 |
| DYRK1A | 114.0 | 10.01 |
| DYRK1B | 135.2 | 11.87 |
| PKCnu | 135.8 | 11.92 |
| DYRK2 | 151.2 | 13.27 |
| CLK4 | 153.7 | 13.49 |
| PKCg | 159.3 | 13.99 |
| PKCeta | 183.2 | 16.08 |
| PKCd | 191 | 16.77 |
| DYRK3 | 217.9 | 19.13 |

As the data demonstrates, the compounds of aspects of the invention are active kinase inhibitors.

### Example 9

This example provides an exemplary synthesis for exemplary thiazine carboxamides disclosed herein.

Synthesis of Acid Scaffold-A. Acid Scaffold-A was synthesized from the commercially available H-1 and H-2. In particular, H-1 was reacted with H-2 in presence of Xantphos Pd G3 and Cs₂CO₃ to afford H-3, which was reacted with KOH to afford Acid Scaffold-A, as set forth in Scheme 2.

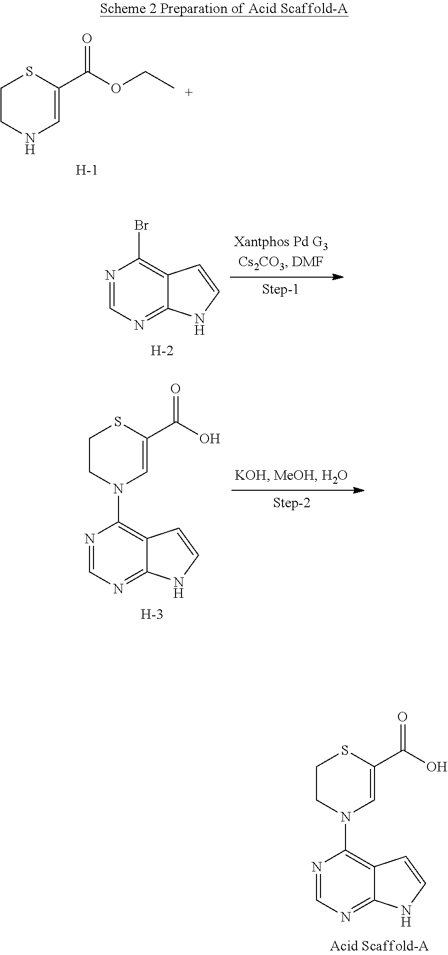

Preparation of Ethyl 4-(7H-pyrrolo[2,3-d]pyrimidin-4-yl)-3,4-dihydro-2H-1,4-thiazine-6-carboxylate (H-3): To the mixture of ethyl 3,4-dihydro-2H-1,4-thiazine-6-carboxylate (H-1,1 equiv., 100 mg) and 4-bromo-7H-pyrrolo[2,3-d]pyrimidine (H-2, 1.1 equiv., 125 mg) was added Xantphos Pd G3 (10 mol %, 55 mg), Cs₂CO₃ (3 equiv., 560 mg), and DMF (5 mL). The reaction vial was filled with N₂ and capped tightly. The reaction mixture was then stirred vigorously at 110° C. overnight before it was dried down under vacuum. The residue was re-dissolved in DMSO followed by preparative HPLC purification using a Gemini 5 μm NX-C18 column (110 Å, 250×21.2 mm) with a flow rate of 10 mL/min (eluted with 10% to 100% MeCN in 0.1% TFA) to yield H-3 (109 mg, 65% yield) as a pale white solid. ¹H NMR (600 MHz, DMSO-d₆): δ 12.21 (s, 1H), 8.84 (s, 1H), 8.44 (s, 1H), 7.49 (s, 1H), 6.67 (s, 1H), 4.35 (br s, 2H), 4.20 (br s, 2H), 3.13 (br s, 2H), 1.24 (br s, 3H); ¹³C NMR (150 MHz, DMSO-d₆): δ 164.7, 152.8, 152.6, 149.9, 133.3, 125.0, 104.5, 100.7, 100.1, 60.4, 45.3, 23.9, 14.3; HRESIMS m/z 291.0910, [M+H]⁺ (calcd for C₁₃H₁₅N₄O₂S, 291.0916).

Preparation of 4-(7H-pyrrolo[2,3-d]pyrimidin-4-yl)-3,4-dihydro-2H-1,4-thiazine-6-carboxylic acid (Acid Scaffold-A): H-3 (109 mg) was reacted with KOH (3 equiv., 62 mg) in MeOH (5 mL) and H₂O (5 mL) and stirred at 95° C. for 2 h. The reaction solution was acidified with 2M HCl (1.4 mL) and then dried down under vacuum. The crude product was then washed and desalted with H₂O (1 mL×3 times) to yield Acid Scaffold-A (80 mg, 81% yield) as a pale white solid. ¹H NMR (600 MHz, DMSO-d₆): δ 12.18 (s, 1H), 8.79 (s, 1H), 8.43 (s, 1H), 7.47 (s, 1H), 6.65 (s, 1H), 4.33 (br s, 2H), 3.12 (br s, 2H); ¹³C NMR (150 MHz, DMSO-d₆): δ 166.2, 152.7, 152.6, 149.9, 132.9, 124.8, 104.4, 101.7, 100.1, 44.9, 24.0; HRESIMS m/z 263.0608, [M+H]⁺ (calcd for C₁₁H₁₁N₄O₂S, 263.0603).

Synthesis of Acid Scaffold-B. Acid Scaffold-B was synthesized from the commercially available H-1 and H-4. In particular, H-1 was reacted with H-4 in presence of Xantphos Pd G3 and Cs₂CO₃ to afford H-5 which was reacted with KOH to afford Acid Scaffold-B, as set forth in Scheme 3.

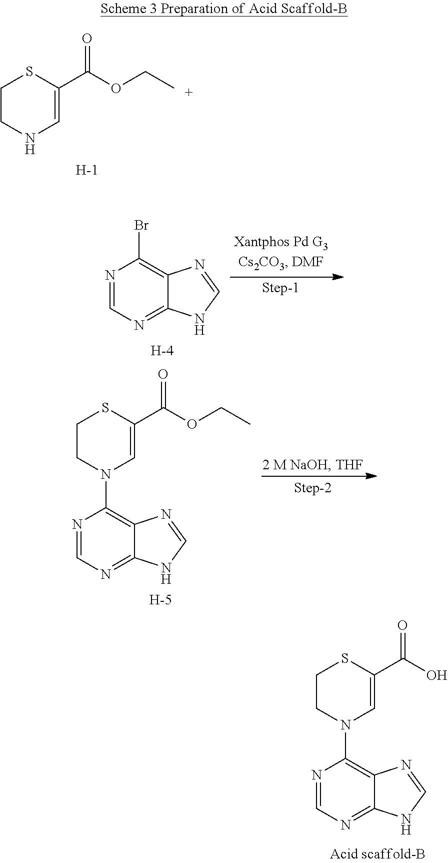

Preparation of Ethyl 4-(7H-purin-6-yl)-3,4-dihydro-2H-1,4-thiazine-6-carboxylate (H-5): To the mixture of ethyl 3,4-dihydro-2H-1,4-thiazine-6-carboxylate (H-1, 1 equiv., 100 mg) and 6-bromopurine (H-4, 1.5 equiv., 170 mg) was added Xantphos Pd G3 (10 mol %, 55 mg), Cs₂CO₃ (3 equiv., 560 mg), and DMF (5 mL). The reaction vial was filled with N₂ and capped tightly. The reaction mixture was then stirred vigorously at 110° C. overnight before it was dried down under vacuum. The residue was re-dissolved in DMSO followed by preparative HPLC purification using a Gemini 5 μm NX-C18 column (110 Å, 250×21.2 mm) with a flow rate of 10 mL/min (eluted with 10% to 100% MeCN in 0.1% TFA) to yield H-5 (61 mg, 36% yield) as a pale white solid. ¹H NMR (600 MHz, DMSO-d₆): δ 13.56 (s, 1H), 9.77 (s, 1H), 8.52 (s, 1H), 8.44 (s, 1H), 4.55 (br s, 2H), 4.20 (q, J=7.1 Hz, 2H), 3.16 (m, 2H), 1.24 (t, J=7.1 Hz, 3H); ¹³C NMR (150 MHz, DMSO-d₆): δ 164.7, 153.0, 151.3, 149.5, 141.7, 134.6, 120.4, 102.1, 60.5, 43.5, 23.8, 14.4; HRESIMS m/z 292.0865, [M+H]⁺ (calcd for C₁₂H₁₄N₅O₂S, 292.0863).

Preparation of 4-(7H-purin-6-yl)-3,4-dihydro-2H-1,4-thiazine-6-carboxylic acid (Acid Scaffold-B): H-5 (61 mg) was dissolved in 2 M NaOH (3.5 mL) and THF (3.5 mL) and stirred at room temperature overnight. The reaction solution was acidified with 2 M HCl (5 mL) and then dried down under vacuum. The crude product was then washed and desalted with H₂O (1 mL×3 times) to yield Acid Scaffold-B (49 mg, 90% yield) as a pale orange solid. ¹H NMR (600 MHz, DMSO-d₆): δ 13.53 (s, 1H), 12.56 (brs, 1H), 9.78 (s, 1H), 8.50 (s, 1H), 8.43 (s, 1H), 4.51 (br s, 2H), 3.13 (m, 2H); ¹³C NMR (150 MHz, DMSO-d₆): δ 166.3, 152.9, 151.3, 149.5, 141.6, 134.3, 120.2, 103.2, 43.1, 23.7; HRESIMS m/z 264.0554, [M+H]⁺ (calcd for C₁₀H₁₀N₅O₂S, 264.0550).

Synthesis of Acid Scaffold-C. Acid Scaffold-C was synthesized from the commercially available H-6 and H-4. In particular, H-6 was reacted with H-4 in presence of diisopropylethylamine (DIPEA) to afford H-7, which was reacted with KOH to afford Acid Scaffold-C, as set forth in Scheme 4.

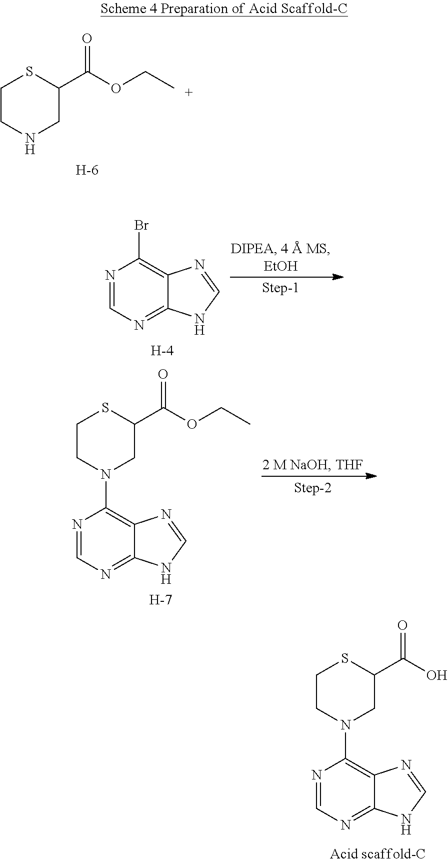

Preparation of Ethyl 4-(7H-purin-6-yl)thiomorpholine-2-carboxylate (H-7): To the mixture of ethyl morpholine-2-carboxylate (H-6, 1 equiv., 100 mg), 6-bromopurine (H-4, 1.1 equiv., 113 mg), 4 Å MS was added DIPEA (3 equiv., 375 μL) and EtOH (5 mL). The reaction mixture was then stirred vigorously at 95° C. overnight before it was dried down under vacuum. The residue was re-dissolved in DMSO followed by preparative HPLC purification using a Gemini 5 μm NX-C18 column (110 Å, 250×21.2 mm) with a flow rate of 10 m/min (eluted with 25% MeCN in 0.1% TFA) to yield H-7 (150 mg, 90% yield) as a white solid. ¹H NMR (600 MHz, methanol-d₄): δ 8.40 (s, 1H), 8.20 (s, 1H), 5.23 (br s, 1H), 5.02 (br s, 1H), 4.48 (d, J=13.6 Hz, 1H), 4.25 (br s, 1H), 4.03 (m, 2H), 3.71 (dd, J=3.3, 5.2 Hz, 1H), 3.18 (m, 1H), 2.76 (m, 1H), 1.09 (t, J=7.1 Hz, 3H); ¹³C NMR (150 MHz, methanol-d₄): δ 172.2, 154.1, 149.0, 148.6, 141.1, 119.5, 62.5, 50.2, 49.4, 40.7, 26.5, 14.2; HRESIMS m/z 294.1020, [M+H]⁺ (calcd for C₁₂H₁₆N₅O₂S, 294.1025).

Preparation of 4-(7H-purin-6-yl)thiomorpholine-2-carboxylic acid (Acid Scaffold-C): H-7 (150 mg) was dissolved in 2 M NaOH (5 mL) and THF (5 mL) and stirred at room temperature overnight. The reaction solution was acidified with 2 M HCl (6 mL) and then dried down under vacuum. The crude product was then washed and desalted with H₂O (1 mL×3 times) to yield Acid Scaffold-C (120 mg, 88% yield) as a white solid. ¹H NMR (600 MHz, methanol-d₄): δ 8.24 (s, 1H), 8.02 (s, 1H), 5.03 (d, J=13.3 Hz, 1H), 4.86 (br s, 1H), 4.50 (br s, 1H), 4.31 (m, 1H), 3.66 (dd, J=3.2, 7.5 Hz, 1H), 2.97 (m, 1H), 2.78 (m, 1H); ¹³C NMR (150 MHz, methanol-d₄): δ 173.8, 155.1, 153.0, 152.3, 1³9.4, 120.5, 50.1, 48.6, 42.1, 27.1; HRESIMS m/z 266.0714, [M+H]⁺ (calcd for C₁₀H₁₂N₅O₂S, 266.0712).

Synthesis of Thiazine Carboxamides. Each of the following compounds was synthesized by the general procedure for Amide Coupling set forth in Scheme 5.

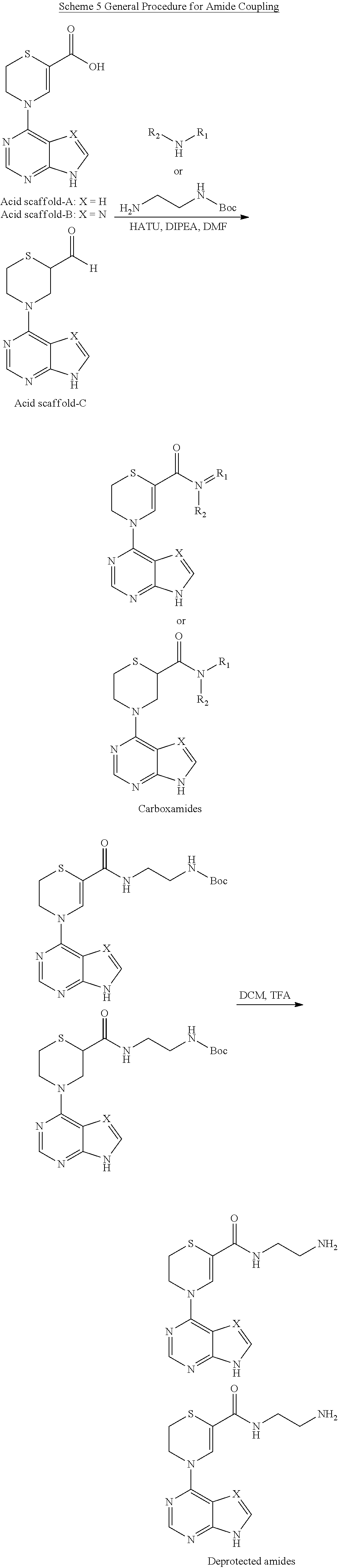

To the mixture of Acid Scaffold-A, B, or C (1 equiv.) and primary/secondary amine or N-Boc-ethylenediamine (1.5 equiv.) was added HATU (1.5 equiv.), DIPEA (10 equiv.), and DMF. The reaction mixture was then stirred vigorously at room temperature overnight before it was dried down under vacuum. The residue was re-dissolved in DMSO followed by preparative HPLC purification using a Gemini 5 μm NX-C18 column (110 Å, 250×21.2 mm) with a flow rate of 10 mL/min (eluted with 10% to 100% MeCN in 0.1% TFA) to afford the carboxamide or Boc-protected amide product. The purified Boc-protected amide product was deprotected in DCM: TFA (2:1) followed by HPLC purification using a Synergi 5 μm Hydro-RP column (110 Å, 250×21.2 mm) with a flow rate of 10 mL/min (eluted with 10-100% MeCN in 0.1% TFA) to yield the deprotected amide product.

### Preparation of N-(2-aminoethyl)-4-(7H-purin-6-yl)-3,4-dihydro-2H-1,4-thiazine-6-carboxamide (TFA salt) (183A049E)

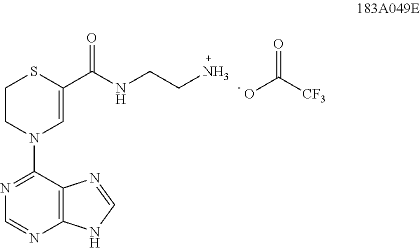 [SMILES: NCCNC(=O)C1=CN(c2ncnc3[nH]cnc23)CCS1 ; O=C(O)C(F)(F)F]

N-(2-aminoethyl)-4-(7H-purin-6-yl)-3,4-dihydro-2H-1,4-thiazine-6-carboxamide (TFA salt) (183A049E) (183A049E) was obtained (33% yield) as an off white solid. ¹H NMR (600 MHz, DMSO-d₆): δ 13.50 (s, 1H), 9.45 (br s, 1H), 8.49 (s, 1H), 8.40 (s, 1H), 7.88 (t, J=5.7 Hz, 1H), 7.79 (br s, 3H), 4.61 (br s, 2H), 3.40 (m, 2H), 3.17 (m, 2H), 2.92 (m, 2H); ¹³C NMR (150 MHz, DMSO-d₆): δ 165.3, 152.9, 151.4, 149.8, 141.3, 130.4, 120.2, 105.6, 44.1, 38.9, 37.4, 24.2; HRESIMS m/z 306.1133, [M+H]⁺ (calcd for C₁₂H₁₆N₇OS, 306.1137).

### Preparation of N-(2-aminoethyl)-4-(7H-pyrrolo[2,3-d]pyrimidin-4-yl)-3,4-dihydro-2H-1,4-thiazine-6-carboxamide (TFA salt) (183A056C)

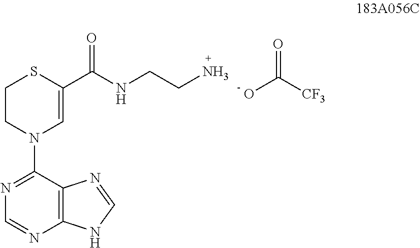 [SMILES: NCCNC(=O)C1=CN(c2ncnc3[nH]cnc23)CCS1 ; O=C(O)C(F)(F)F]

N-(2-aminoethyl)-4-(7H-pyrrolo[2,3-d]pyrimidin-4-yl)-3,4-dihydro-2H-1,4-thiazine-6-carboxamide (TFA salt) (183A056C) was obtained (70% yield) as a colorless solid. ¹H NMR (600 MHz, DMSO-d₆): δ 12.18 (s, 1H), 8.67 (s, 1H), 8.42 (s, 1H), 7.90 (t, J=5.7 Hz, 1H), 7.82 (br s, 3H), 7.46 (br s, 1H), 6.71 (br s, 1H), 4.36 (m, 2H), 3.41 (m, 2H), 3.13 (m, 2H), 2.93 (m, 2H); ¹³C NMR (150 MHz, DMSO-d₆): δ 165.3, 153.0, 152.5, 149.9, 129.7, 124.6, 104.6, 104.4, 100.5, 45.4, 38.9, 37.3, 24.4; HRESIMS m/z 305.1182, [M+H]⁺ (caled for C₁₃H₁₇N₆OS, 305.1179).

### Preparation of N-(2-aminoethyl)-4-(7H-purin-6-yl)thiomorpholine-2-carboxamide (TFA salt) (183A05C)

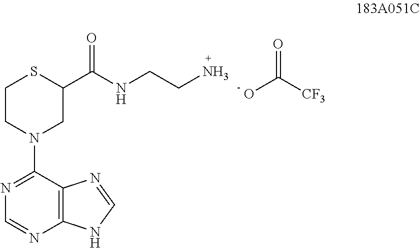 [SMILES: NCCNC(=O)C1CN(c2ncnc3[nH]cnc23)CCS1 ; O=C(O)C(F)(F)F]

N-(2-aminoethyl)-4-(7H-purin-6-yl)thiomorpholine-2-carboxamide (TFA salt) (183A051C) was obtained (87% yield) as an off white solid. ¹H NMR (600 MHz, Methanol-d₄): δ 8.25 (s, 1H), 8.06 (s, 1H), 4.91 (m, 1H), 4.80 (m, 1H), 4.60 (m, 1H), 4.38 (m, 1H), 3.65 (dd, J=3.1, 7.3 Hz, 1H), 3.49 (m, 1H), 3.35 (m, 1H), 3.02 (m, 2H), 3.00 (m, 1H), 2.81 (m, 1H); ¹³C NMR (150 MHz, Methanol-d₄): δ 173.7, 155.0, 152.6, 152.2, 139.7, 120.1, 50.3, 48.9, 43.0, 40.7, 38.4, 27.3; HRESIMS m/z 308.1289, [M+H]⁺ (caled for C₁₂H₁₈N₇OS, 308.1294).

### Preparation of N-methoxy-N-methyl-4-(7H-purin-6-yl)-3,4-dihydro-2H-1,4-thiazine-6-carboxamide (183A047C)

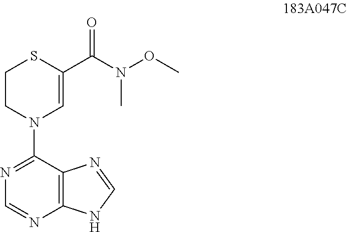 [SMILES: CON(C)C(=O)C1=CN(c2ncnc3[nH]cnc23)CCS1]

N-methoxy-N-methyl-4-(7H-purin-6-yl)-3,4-dihydro-2H-1,4-thiazine-6-carboxamide (183A047C) was obtained (93% yield) as a pale orange solid. ¹H NMR (600 MHz, DMSO-d₆): δ 13.50 (s, 1H), 9.68 (s, 1H), 8.49 (s, 1H), 8.40 (s, 1H), 4.52 (br s, 2H), 3.70 (s, 3H), 3.17 (s, 3H), 3.08 (m, 2H); ¹³C NMR (150 MHz, DMSO-d₆): δ 166.5, 152.8, 151.3, 149.8, 141.3, 133.2, 120.2, 104.4, 61.1, 44.8, 33.6, 24.5; HRESIMS m/z 307.0974, [M+H]⁺ (calcd for C₁₂H₁₅N₆O₂S, 307.0972).

### Preparation of N-((1-methyl-1H-imidazol-2-yl)methyl)-4-(7H-purin-6-yl)-3,4-dihydro-2H-1,4-thiazine-6-carboxamide (183A046B)

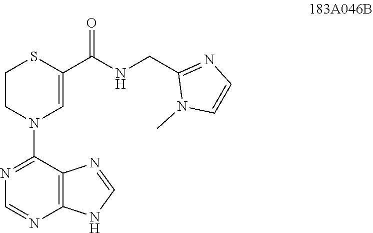 [SMILES: Cn1ccnc1CNC(=O)C1=CN(c2ncnc3[nH]cnc23)CCS1]

N-((1-methyl-1H-imidazol-2-yl)methyl)-4-(7H-purin-6-yl)-3,4-dihydro-2H-1,4-thiazine-6-carboxamide (183A046B) was obtained (32% yield) as an off white solid. ¹H NMR (600 MHz, DMSO-d₆): δ 13.54 (br s, 1H), 9.54 (s, 1H), 8.51 (t, J=5.1 Hz, 1H), 8.50 (s, 1H), 7.63 (d, J=1.9 Hz, 1H), 7.58 (d, J=1.9 Hz, 1H), 4.61 (d, J=5.1 Hz, 2H), 4.59 (m, 2H), 3.84 (s, 3H), 3.17 (m, 2H); ¹³C NMR (150 MHz, DMSO-d₆): δ 165.8, 153.3, 151.7, 150.2, 144.7, 141.8, 131.8, 124.0, 120.6, 118.7, 105.0, 44.4, 34.7, 34.5, 24.5; HRESIMS m/z 357.1240, [M+H]⁺ (calcd for C₁₅H₁₇N₈OS, 357.1246).

### Preparation of (4-(7H-purin-6-yl)-3,4-dihydro-2H-1,4-thiazin-6-yl)(morpholino)methanone (183A047F)

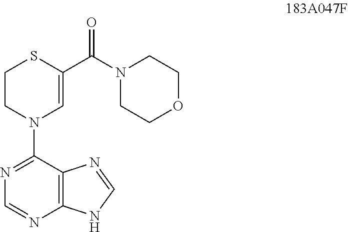 [SMILES: O=C(C1=CN(c2ncnc3[nH]cnc23)CCS1)N1CCOCC1]

(4-(7H-purin-6-yl)-3,4-dihydro-2H-1,4-thiazin-6-yl)(morpholino)methanone (183A047F) was obtained (60% yield) as an off white solid. ¹H NMR (600 MHz, DMSO-d₆): δ 9.04 (br s, 1H), 8.47 (s, 1H), 8.41 (s, 1H), 4.83 (m, 2H), 3.63 (m, 4H), 3.58 (m, 4H). 3.21 (m, 2H); ¹³C NMR (150 MHz, DMSO-d₆): δ 166.9, 152.1, 151.1, 149.8, 141.2, 128.3, 119.7, 106.2, 66.4 (2C), 45.6 (2C), 43.0, 24.5; HRESIMS m/z 333.1138, [M+H]⁺ (calcd for C₁₄H₁₇N₆O₂S, 333.1134).

### Preparation of N-(2-acetamidoethyl)-4-(7H-purin-6-yl)-3,4-dihydro-2H-1,4-thiazine-6-carboxamide (183A050B)

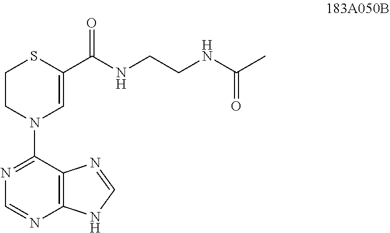 [SMILES: CC(=O)NCCNC(=O)C1=CN(c2ncnc3[nH]cnc23)CCS1]

N-(2-acetamidoethyl)-4-(7H-purin-6-yl)-3,4-dihydro-2H-1,4-thiazine-6-carboxamide (183A050B) was obtained (79% yield) as an off white solid. ¹H NMR (600 MHz, DMSO-d₆): δ 9.38 (br s, 1H), 8.48 (s, 1H), 8.40 (s, 1H), 7.97 (t, J=5.6 Hz, 1H), 7.75 (t, J=5.6 Hz, 1H), 4.58 (br s, 2H), 3.21 (m, 2H), 3.16 (m, 2H), 3.15 (m, 2H), 1.80 (s, 3H); ¹³C NMR (150 MHz, DMSO-d₆): δ 169.6, 164.6, 152.8, 151.3, 149.9, 141.2, 129.8, 120.1, 106.4, 43.9, 39.3, 38.4, 24.2, 22.7; HRESIMS m/z 348.1246, [M+H]⁺ (calcd for C₁₄H₁₈N₇O₂S, 348.1243).

### Preparation of N-(2-methoxyethyl)-4-(7H-purin-6-yl)-3,4-dihydro-2H-1,4-thiazine-6-carboxamide (183A050C)

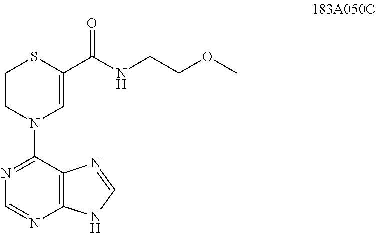 [SMILES: COCCNC(=O)C1=CN(c2ncnc3[nH]cnc23)CCS1]

N-(2-methoxyethyl)-4-(7H-purin-6-yl)-3,4-dihydro-2H-1,4-thiazine-6-carboxamide (183A050C) was obtained (99% yield) as an off white solid. ¹H NMR (600 MHz, DMSO-d₆): δ 9.39 (br s, 1H), 8.48 (s, 1H), 8.40 (s, 1H), 7.61 (t, J=5.6 Hz, 1H), 4.58 (br s, 2H), 3.39 (m, 2H), 3.33 (m, 2H), 3.25 (s, 3H), 3.16 (m, 2H); ¹³C NMR (150 MHz, DMSO-d₆): δ 164.5, 152.8, 151.3, 149.9, 141.2, 129.9, 120.1, 106.2, 70.5, 58.0, 43.9, 38.9, 24.2; HRESIMS m/z 321.1139, [M+H]⁺ (calcd for C₁₃H₁₇N₆O₂S, 321.1134).

### Preparation of N-(2-hydroxyethyl)-4-(7H-purin-6-yl)-3,4-dihydro-2H-1,4-thiazine-6-carboxamide (183A050D)

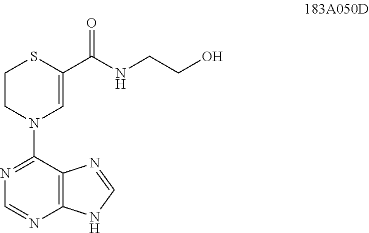 [SMILES: O=C(NCCO)C1=CN(c2ncnc3[nH]cnc23)CCS1]

N-(2-hydroxyethyl)-4-(7H-purin-6-yl)-3,4-dihydro-2H-1,4-thiazine-6-carboxamide (183A050D) was obtained (84% yield) as an off white solid. ¹H NMR (600 MHz, DMSO-d₆): δ 9.39 (br s, 1H), 8.48 (s, 1H), 8.39 (s, 1H), 7.54 (t, J=5.6 Hz, 1H), 4.58 (br s, 2H), 3.44 (t, 0.1=6.4 Hz, 2H), 3.23 (m, 2H), 3.16 (m, 2H); ¹³C NMR (150 MHz, DMSO-d₆): δ 164.5, 152.8, 151.3, 149.9, 141.2, 129.8, 120.1, 106.2, 59.8, 43.9, 42.1, 24.2; HRESIMS m/z 307.0982, [M+H]⁺ (calcd for C₁₂H₁₅N₆O₂S, 307.0977).

### Preparation of N-(2-(methylamino)ethyl)-4-(7H-purin-6-yl)-3,4-dihydro-2H-1,4-thiazine-6-carboxamide (TFA salt) (183A050E)

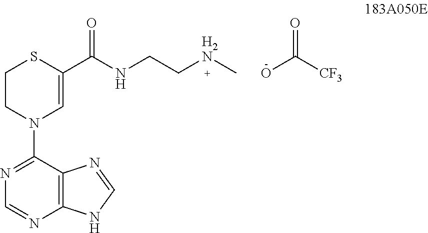

N-(2-(methylamino)ethyl)-4-(7H-purin-6-yl)-3,4-dihydro-2H-1,4-thiazine-6-carboxamide (TFA salt) (183A050E) was obtained (45% yield) as an off white solid. ¹H NMR (600 MHz, DMSO-d₆): δ 9.47 (br s, 1H), 8.50 (s, 1H), 8.41 (s, 1H), 8.30 (br s, 2H), 7.91 (t, J=5.6 Hz, 1H), 4.61 (br s, 2H), 3.44 (m, 2H), 3.18 (m, 2H), 3.02 (m, 2H), 2.58 (t, J=5.4 Hz, 3H); ¹³C NMR (150 MHz, DMSO-d₆): δ 165.4, 152.8, 151.3, 149.8, 141.4, 130.5, 120.1, 105.6, 48.5, 44.1, 36.1, 32.9, 24.2, HRESIMS m/z 320.1290, [M+H]⁺ (calced for C₁₃H₁₈N₇OS, 320.1294).

Preparation of N-methyl-4-(7H-purin-6-yl)-3,4-dihydro-2H-1,4-thiazine-6-carboxamide (183A046C): N-methyl-4-(7H-purin-6-yl)-3,4-dihydro-2H-1,4-thiazine-6-carboxamide (183A046C) was prepared as set forth in Scheme 6.

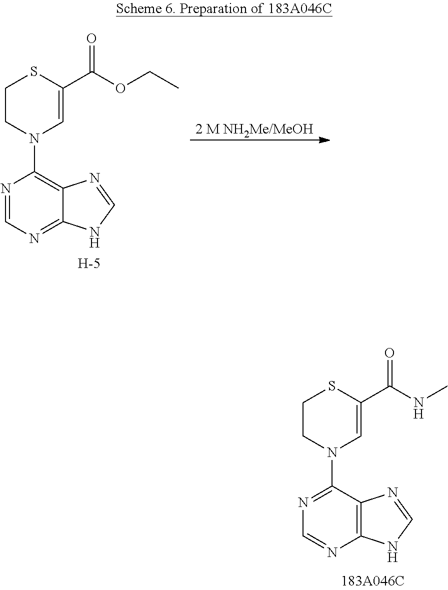

H-5 (6 mg) was stirred in 2 M NH₂Me/MeOH solution (5 mL) in the presence of 4 Å MS at room temperature overnight before it was dried down under vacuum. The residue was re-dissolved in DMSO followed by semi-preparative HPLC purification using a Gemini 5 μm NX-C18 column (110 Å, 250×10 mm) with a flow rate of 4 mL/min (eluted with 20% MeCN in 0.1% TFA) to afford 183A046C (4.5 mg). N-methyl-4-(7H-purin-6-yl)-3,4-dihydro-2H-1,4-thiazine-6-carboxamide (183A046C) was obtained (71% yield) as an off white solid. ¹H NMR (600 MHz, DMSO-d₆): δ 13.48 (br s, 1H), 9.36 (s, 1H), 8.47 (s, 1H), 8.39 (s, 1H), 7.64 (q, J=4.6 Hz, 1H), 7.58 (d, J=1.9 Hz, 1H), 4.57 (m, 2H), 4.59 (m, 2H), 3.15 (m, 2H), 2.67 (d, J=4.6 Hz, 3H); ¹³C NMR (150 MHz, DMSO-d₆): δ 164.9, 152.8, 151.3, 149.9, 141.1, 129.4, 120.0, 106.6, 43.7, 26.5, 24.2; HRESIMS m/z 277.0870, [M+H]⁺ (calcd for C₁₁H₁₃N₆S, 277.0872).

Preparation of N-(2-(picolinamido)ethyl)-4-(7H-purin-6-yl)-3,4-dihydro-2H-1,4-thiazine-6-carboxamide (183A049C): N-(2-(picolinamido)ethyl)-4-(7H-purin-6-yl)-3,4-dihydro-2H-1,4-thiazine-6-carboxamide (183A049C) was prepared as set forth in Scheme 7.

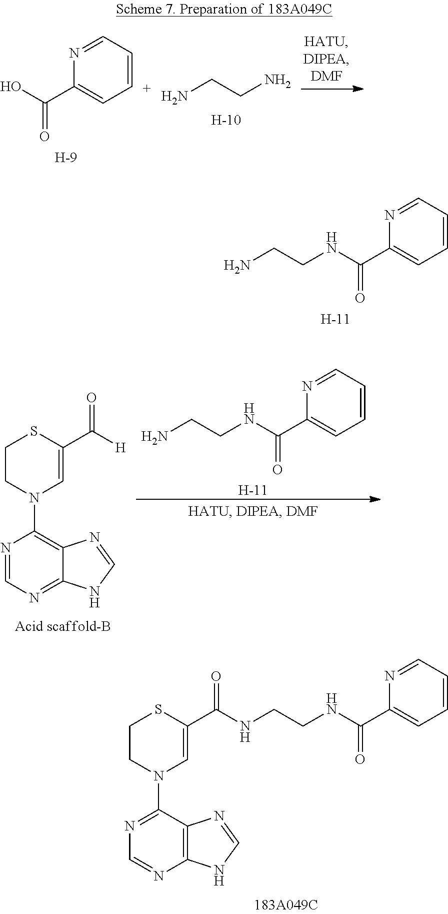

To the mixture of pyridine-2-carboxylic acid (H-9, 1 equiv., 20 mg) and ethylenediamine (H-10, 10 equiv., 108 μL) was added HATU (1.5 equiv., 93 mg), DIPEA (10 equiv., 283 μL), and DMF (2 mL). The reaction mixture was then stirred at room temperature overnight before it was dried down under vacuum. The residue was re-dissolved in DMSO followed by semi-preparative HPLC purification using a Synergy 5 μm Polar-RP column (110 Å, 250×10 mm) with a flow rate of 4 mL/min (eluted with 5% MeCN in 0.1% TFA) to yield H-11 (13.6 mg, 50% yield). To the mixture of Acid Scaffold-B (1 equiv., 4.7 mg) and H-11 (1.5 equiv., 6.0 mg) was added HATU (1.5 equiv., 10 mg), DIPEA (10 equiv., 30 μL), and DMF (0.5 mL). The reaction mixture was then stirred vigorously at room temperature overnight before it was dried down under vacuum. The residue was re-dissolved in DMSO followed by semi-preparative HPLC purification using a Gemini 5 μm NX-C18 column (110 Å, 250×10 mm) with a flow rate of 4 mL/min (eluted with 23% MeCN in 0.1% TFA) to afford 183A049C (3.7 mg). N-(2-(picolinamido)ethyl)-4-(7H-purin-6-yl)-3,4-dihydro-2H-1,4-thiazine-6-carboxamide (183A049C) was obtained (50% yield) as an off white solid. ¹H NMR (600 MHz, DMSO-d₆): δ 9.37 (br s, 1H), 8.97 (t, J=5.8 Hz, 1H), 8.65 (d, J=4.8 Hz, 1H), 8.48 (s, 1H), 839 (s, 1H), 8.05 (d, J=7.7 Hz, 1H), 8.01 (t, 0.1=7.7 Hz, 1H), 7.88 (t, J=5.4 Hz, 1H), 7.61 (dd, J=4.8, 7.7 Hz, 1H), 4.59 (br s, 2H), 3.45 (m, 2H), 3.38 (m, 2H), 3.16 (m, 2H); ¹³C NMR (150 MHz, DMSO-d₆): δ 164.8, 164.2, 152.7, 151.2, 149.8 (2C), 148.4, 141.2, 138.1, 129.7, 126.7, 122.1, 120.0, 106.6, 43.9, 39.4, 39.0, 24.2; HRESIMS m/z 411.1356, [M+H]⁺ (calcd for C₁₈H₁₉N₈O₂S, 411.1351).

Preparation of N-(2-benzamidoethyl)-4-(7H-purin-6-yl)-3,4-dihydro-2H-1,4-thiazine-6-carboxamide (183A049F): N-(2-benzamidoethyl)-4-(7H-purin-6-yl)-3,4-dihydro-2H-1,4-thiazine-6-carboxamide (183A049F) was prepared as set forth in Scheme 8.

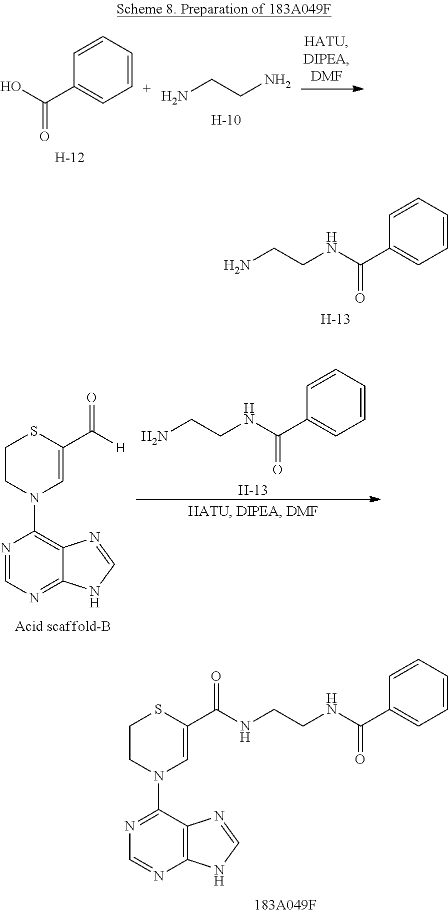

To the mixture of benzoic acid (H-12, 1 equiv., 20 mg) and ethylenediamine (H-10, 3 equiv., 32 L) was added HATU (1.5 equiv., 93 mg), DIPEA (10 equiv., 283 μL), and DMF (2 mL). The reaction mixture was then stirred at room temperature overnight before it was dried down under vacuum. The residue was re-dissolved in DMSO followed by semi-preparative HPLC purification using a Synergy 5 μm Polar-RP column (110 Å, 250×10 mm) with a flow rate of 4 mL/min (eluted with 10% MeCN in 0.1% TFA) to yield H-13 (8.7 mg, 33% yield). To the mixture of Acid Scaffold-B (1 equiv., 3.7 mg) and H-13 (8.7 mg) was added HATU (1.5 equiv., 8 mg), DIPEA (10 equiv., 24 μL), and DMF (0.4 mL). The reaction mixture was then stirred vigorously at room temperature overnight before it was dried down under vacuum. The residue was re-dissolved in DMSO followed by semi-preparative HPLC purification using a Gemini 5 μm NX-C18 column (110 Å, 250×10 mm) with a flow rate of 4 m/min (eluted with 30% MeCN in 0.1% TFA) to afford 183A049F (2.5 mg). N-(2-benzamidoethyl)-4-(7H-purin-6-yl)-3,4-dihydro-2H-1,4-thiazine-6-carboxamide (183A049F) was obtained (43% yield) as an off white solid. ¹H NMR (600 MHz, DMSO-d₆): δ 9.38 (br s, 1H), 8.57 (t, J=5.4 Hz, 1H), 8.48 (s, 1H), 8.39 (s, 1H), 7.87 (t, J=5.4 Hz, 1H), 7.85 (d, J=7.5 Hz, 2H), 7.46 (t, J=7.5 Hz, 2H), 7.52 (t, J=7.5 Hz, 1H), 4.58 (br s, 2H), 3.38 (m, 4H), 3.16 (m, 2H); ¹³C NMR (150 MHz, DMSO-d₆): δ 166.6, 164.8, 152.7, 151.2, 149.9, 141.3, 134.5, 131.2, 129.7, 128.4 (2C), 127.3 (2C), 120.0, 106.6, 43.9, 39.3 (2C), 24.2; HRESIMS m/z 410.1395, [M+H]⁺ (calcd for C₁₉H₂₀N₇O₂S, 410.1399).

### Example 10

This example provides the IC₅₀ value for inhibitory activity against an enzymatically active chimeric protein, JPKAcα, which is found in almost all FLHCC patients with greater than 10-fold overexpression of the fusion kinase in tumor cells relative to wild-type PKA (wt-PKA) expression in adjacent normal liver tissue.

Compounds were tested to determine an IC₅₀ value for inhibitory activity against RIα2:JPKAcα₂ chimeric kinase holoenzyme. A 3× dose-response curve was set up over a final compound concentration range of 0-10 μM containing 1 μM cAMP, 50 μM ATP, and 0.4% DMSO in 100 mM Tris-HCl pH 7.5 (all final concentrations). A no cAMP/ATP control was also included for background normalization. Using 12-channel multichannel pipettes, quadruplicate reactions were initiated by the addition of 20 μL of compound/cAMP/ATP solution to reaction wells containing 40 μL of 1.5× concentration PKA holoenzyme (chimeric) and biotinylated substrate protein (0.5 nM Chimeric Kinase Holoenzyme or 0.66 nM wt-kinase holoenzyme, 50 M biotinylated substrate, in kinase buffer. After initiation by addition, the reaction was allowed to proceed for 45 min prior to the addition of 15 μL of 0.5 M EDTA to quench the reactions. Quenched reactions were then transferred to prepared assay binding plates and the ELISA was developed as described above for the primary screening assay. For each reaction well, observed RFU were converted to normalized % activity measurements as described above using the No cAMP/ATP wells as the low control and the vehicle control (0 μM) as the high control. The % JPKAcα Activity curves for compound 183A056C is set forth in FIG. 106.

The % normalized activity measures were then fit to the following equation using a nonlinear regression least-squares fit with a variable slope (GraphPad Prism Software, San Diego, CA) an IC₅₀ value was calculated using the following formula.

$$\% normalized activity = \frac{100}{( 1 + {10}^{( ( {logIC}_{50} - log [ I ] ) \times Hill slope )} )}$$

The mean (from three dose response curves) JPKAcα IC₅₀ values are set forth in Table 5.

TABLE 5
JPKAcα IC₅₀ values

| Compound | JPKAcα IC₅₀ (nm)* | Compound | JPKAcα IC₅₀ (nm)* |
|---|---|---|---|
| 183A034G | B | 183A050E | C |
| 183A049E | B | 183A051C | D |
| 183A049F | D | 183A050D | D |
| 183A050B | D | 183A049C | D |
| 183A050C | D | 183A056C | A |

*A <0.1 μM; B is 0.1 μM-1 μM; C is 1.01 μM-10 μM; D is >10 μM

### Example 11

This example provides kinome profiling and the results of dose response testing of compounds of an aspect of the invention.

Pan human kinome profiling (370 kinases) and subsequent 10-point dose response testing against a panel of 30 selected kinases were conducted by Reaction Biology Corp. (Malvern, PA, USA) using the radiometric HOTSPOTT™ kinase assay (Anastassiadis, et al., Nat. Biotechnol., 29(11): 1039-U117 (2011)). All kinase reactions were carried out at 10 μM ATP. Both Compound 1 and Compound 3 were tested at two concentrations (2 μM and 50 nM) in the pan human kinome profiling and using a 3-fold serial dilution starting at 20 μM for the 10-point dose response testing of Compound 1. Complete method descriptions and kinome composition are available from Reaction Biology Corporation. Graphical representation of the inhibitory activities of Compound 1 and Compound 3 across the tested kinome was accomplished through the use of the CORAL software package (Metz, et al., Cell Syst., 7(3): 347-350 el (2018)) (see FIGS. 5A-5C and 15-18).

The kinase profiling revealed potent inhibition of select serine/threonine kinases in the CLK, DYRK, and PKG families with IC50 values ranging from ˜11-90 nM for Compound 1. The kinase profiling also revealed the kinase selectivity profiles of Compound 1 and Compound 3 with the PKG, CLK, and DYRK families as the most sensitive target classes (FIGS. 111 and 112). The DYRK/CLK kinases belong to the CMGC group of serine/threonine kinases. These kinases are involved in a variety of pathological processes, such as neurodegenerative diseases (e.g., Down syndrome, Alzheimer's disease), diabetes, solid cancers (e.g., glioblastoma, breast, and pancreatic cancers), leukemias, and infections caused by virus and parasites. The cGMP-dependent protein kinases (PKGs) are important regulators of the cellular nitric oxide (NO)-signaling pathway. Dysregulation of PKG signaling is associated with most forms of cardiac disease including heart failure. In addition, PKG from Plasmodium falciparum: has also been identified and validated as a target for anti-malaria chemotherapy.

It was unexpected that the modification of the imidazole moiety by monobromination improved the potency of Compound 3 against the DYRK kinases by at least 2-fold when tested at 50 nM though the overall selectivity profiles amongst the whole panel of 370 kinases were very similar between Compound 1 and Compound 3 (FIG. 111, Tables 6-8 below). Table 6 shows the kinome profiling of Compound 1 against a panel of 370 Human Protein Kinases. The data for the top 50 hits when Compound 1 was tested at 50 nM were shown in Table 6. The averaged inhibition rates were obtained from a single experiment performed in duplicate. Table 7 shows the kinome profiling of Compound 3 against a Panel of 370 Human Protein Kinases. The data for the top 50 hits when Compound 3 was tested at 50 nM were shown in Table 7. The averaged inhibition rates were obtained from a single experiment performed in duplicate. Table 8 shows the concentrations (nM) of Aplithianine Analogs that Cause a 50% Decrease in the Catalytic Activity (IC₅₀) of Twenty Selected Kinases. The averaged IC₅₀ values were obtained from a single experiment performed in duplicate or from two independent studies.

TABLE 6

| | 1 (2 μM) | 1 (50 nM) | IC₅₀ (nM) |
|---|---|---|---|
| Kinases | % inhibition | % inhibition | control compound^{a} |
| PAG1α | 98.8 | 81.2 | 0.95 |
| PKG1β | 97.7 | 65.1 | 3.4 |
| PKCθ | 97.9 | 62.6 | 0.25 |
| CLK1 | 97.3 | 61.4 | 5.4 |
| CLK2 | 97.8 | 57.7 | 2.8 |
| PKG2 | 99.4 | 55.2 | 2.7 |
| STLK3 | 87.3 | 50.5 | 13 |
| PKD2 | 93.1 | 38.2 | 1.6 |
| LATS1 | 96.1 | 35.0 | 18 |
| LATS2 | 92.9 | 34.2 | 8.3 |
| IRAK2 | 85.0 | 30.7 | 2.1 |
| PKCγ | 92.8 | 30.5 | 0.16 |
| PKAα | 91.9 | 28.6 | 1.3 |
| CLK4 | 84.7 | 27.1 | 147 |
| ROCK1 | 88.6 | 27.0 | 0.71 |
| PKCε | 92.7 | 25.8 | 0.13 |
| HASPIN | 88.4 | 25.6 | 15 |
| AKT1 | 83.7 | 25.1 | 2.6 |
| PKC [symbol] /PKD3 | 88.9 | 25.0 | 1.1 |
| TK33 | 80.5 | 23.6 | 27 |
| DYRK1A | 95.4 | 23.1 | 1.9 |
| DYRK1B | 95.3 | 22.7 | 0.95 |
| FLT3 | 88.1 | 22.2 | 0.84 |
| MPSK1 | 49.5 | 22.0 | 181 |
| RAF1 | 47.0 | 21.6 | 4.4^{b} |
| BMPR2 | 74.0 | 21.3 | 255 |
| PKCμ/PKD1 | 89.2 | 21.3 | 1.8 |
| DYRK2 | 91.2 | 20.6 | 154 |
| NLK | 43.0 | 19.6 | 96 |
| ROCK2 | 93.3 | 19.6 | 0.69 |
| PKAβ | 94.6 | 18.9 | 1.4 |
| PKN1 | 87.7 | 18.8 | 0.67 |
| PKN3 | 80.4 | 18.6 | 3.8 |
| smMLCK | 91.1 | 18.2 | 12 |
| DYRK3 | 90.9 | 15.9 | 23 |
| PKCη | 83.6 | 15.8 | 0.34 |
| WNK1 | 10.7 | 14.1 | 13350 |
| P70S6K | 87.3 | 14.0 | 0.34 |
| BMX | 40.0 | 13.9 | 4.5 |
| YSK1 | 55.6 | 13.5 | 2.4 |
| MSK2 | 75.5 | 13.3 | 2.3 |
| MST3 | −15.6 | 13.2 | 4.6 |
| RSK1 | 82.0 | 13.0 | 0.09 |
| BRAF | 35.1 | 12.9 | 8.8^{b} |
| EPHB3 | 20.2 | 12.8 | 929 |
| DCAMKL1 | 14.2 | 12.8 | 85 |
| PKCδ | 81.1 | 12.6 | 0.14 |
| CDK2 | 49.1 | 12.6 | 1.5 |
| CDK9 | 61.3 | 12.4 | 13 |
| RSK2 | 66.0 | 12.3 | 0.19 |

^{a}Staurosporine as control;
^{b}GW5074 as control [symbol]
indicates data missing or illegible when filed

TABLE 7

| | 3 (2 μM) | 3 (50 nM) | IC₅₀ (nM) |
|---|---|---|---|
| Kinases | % inhibition | % inhibition | control compound [symbol] |
| PKG1α | 97.4 | 72.1 | 1.3 |
| PKG1β | 97.9 | 68.8 | 4.7 |
| STLK3 | 86.6 | 65.5 | 19 |
| PKCθ | 97.9 | 64.1 | 0.32 |
| CLK1 | 96.1 | 61.7 | 13 |
| DYRK1A | 99.2 | 60.7 | 2. [symbol] |
| DYRK2 | 98.3 | 55.9 | 109 |
| LATS1 | 97.3 | 51.5 | 18 |
| DYRK3 | 98.0 | 49.8 | 47 |
| DYRK1B | 100.1 | 49.1 | 1.2 |
| CLK2 | 97.2 | 46.1 | 5.7 |
| PKG2 | 91.7 | 43.1 | 3.7 |
| MPSK1 | 67.8 | 38.5 | 290 |
| STK33 | 78.4 | 35.8 | 45 |
| smMCK | 93.4 | 33.5 | 23 |
| NEK1 | 96.5 | 33.1 | 17 |
| HASPIN | [symbol] 8.8 | 32.9 | 14 |
| PKC [symbol] /PKD3 | 94.4 | 30.3 | 0.84 |
| PKCη | 91.8 | 29.7 | 0.35 |
| MAP3K19 | 92.8 | 29.5 | 26 |
| PKN3 | [symbol] 8.3 | 29.2 | 5.4 |
| LATS2 | 83.8 | 29.1 | 9.8 |
| PKCδ | 93.4 | 29.0 | 0.12 |
| AKT1 | 84.3 | 26.5 | 3.5 |
| PKCγ | 91.7 | 25.7 | 0.19 |
| CLK4 | 96.8 | 24.0 | 75 |
| CDK6 | 40.4 | 22.0 | 11 |
| FYN | 44.0 | 21.6 | 2.5 |
| CDK7 | 44.3 | 21.6 | 28 |
| PKAα | 90.2 | 21.5 | 2.2 |
| TNIK | 68.7 | 21.5 | 0.41 |
| PKCμ/PKD1 | 91.4 | 21.4 | 2.1 |
| SGK2 | 62.2 | 20.8 | 22 |
| CDK2 | 50.8 | 20.5 | 2.6 |
| MAPKAPK5 | 15.2 | 20.3 | 572 |
| CK2 [symbol] 2 | 46.5 | 19.7 | 371 |
| MYO3A | 29.9 | 19.3 | 15 |
| PKD2 | 95.6 | 19.1 | 1.2 |
| NEK5 | 90.4 | 18.7 | 50 |
| PKAβ | 91.3 | 18.7 | 3.8 |
| ROCK2 | 77.6 | 18.6 | 0.59 |
| PRKX | 92.8 | 17.6 | 1.1 |
| ERK7 | 60.2 | 17.1 | [symbol] .7 |
| MAP2K1 | 16.9 | 17.1 | 89 |
| FLT3 | 60.5 | 16.8 | 0.75 |
| RIPK2 | 34.0 | 16.6 | 199 |
| LIMK1 | 62.8 | 15.2 | 0.95 |
| DYRK4 | 72.2 | 14.9 | 3127 [symbol] |
| GSK3α | 64.1 | 14.8 | 9.5 |
| NEK3 | 79.9 | 14.7 | 185 [symbol] |

^{a}Staurosporine as control; [symbol]
GW5074 as control; [symbol]
JNK-IN-7 as control [symbol]
indicates data missing or illegible when filed

TABLE 8

| Kinase | 1 | Control [symbol] |
|---|---|---|
| PKG1α | 11 | 1.2 |
| PKG1β | 15 | 3.3 |
| PKG2 | 34 | 3.3 |
| STLK3 | 35 | 19 |
| PKCθ | 36 | 0.29 |
| PKCδ | 184 | 0.15 |
| PKCη | 202 | 0.39 |
| PKCγ | 234 | 0.17 |
| PKCv/PKD3 | 130 | 0.9 |
| LATS1 | 64 | 16 |
| LATS2 | 70 | 8.1 |
| CLK1 | 64 | 12 |
| CLK2 | 71 | 6.1 |
| CLK3 | 14100 | 1963 |
| CLK4 | 153 | 7 [symbol] |
| DYRK1A | 119 | 2.7 |
| DYRK1B | 134 | 1.2 |
| DYRK2 | 142 | 106 |
| DYRK3 | 202 | 38 |
| DYRK4 | 15200 | 3656^{b} |
| AKT1 | 179 | 2.7 |
| CAMK1δ | 52305 | 0.22 |
| CK1α1 | 1834 | 15510 |
| ERBB2/HER2 | 26450 | 47 |
| ERK1 | 5715 | 4.2 [symbol] |
| FLT1/VEGFR1 | 18810 | 11 |
| GSK3β | 99 | 4.6 |
| MEK1 | 23595 | 26 |
| PKAα | 84 | 1.3 |
| PKCα | 1324 | 0.18 |

^{a} Staurosporine as control;
^{b}GW5074 as control;
^{c} SCH772 as control. [symbol]
indicates data missing or illegible when filed

FIGS. 107A and 107B are graphs showing % kinase activity at 50 nM (FIG. 107A) and 2 μM (FIG. 107B). Differences in activity among the compounds is more pronounced at 2 μM concentration. FIG. 108 shows the top 10 kinases with the most activity (left to right for each listed kinase are bars for compounds 183A034G/Aplithianine A (1), 183A041B/Aplithianine A₁ (1a), and 183A049E). FIGS. 109 and 110 also show the percentage of kinase activity for 183A034G/Aplithianine A (1). 183A041B/Aplithianine A₁ (1a), and 183A049E for several kinases.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments and aspects of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound of formula (I) 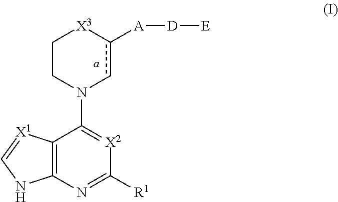
wherein [symbol]
is a single or double bond,
X¹ and X² are each independently CH, CR⁶, or N;
X³ is S, S═O, or S(═O)₂;
R¹ is H or —NR²R³;
R² is H or C₁-C₃ alkyl;
R³ is an aryl;
R⁶ is C₁-C₃ alkyl;
A is optional and, when present, is —C(O)—, —C(O)O—, —C(O)NH—, —C(O)N(C₁-C₃-akyl)-; —C(O)NH—(C₁-C₆ alkyl)-NHC(O)—, —NH;
D is optional and, when present, is a C₁-C₆ alkyl, C₃-C₈ cycloalkyl, C₃-C₈ heterocycloalkyl, —(C₁-C₆ alkyl)-O—, —(C₁-C₆ alkyl)-NH—, —(C₁-C₃ alkyl)-O—(C₁-C₃ alkyl)-, or —(C₁-C₃ alkyl)-NH—(C₁-C₃ alkyl)-, wherein the alkyl group or cycloalkyl group of any of the foregoing is optionally substituted with one or more substituents selected from hydroxy, C₁-C₆ alkyl, amino, C₁-C₆ alkylamino, or di-C₁-C₆ alkyl-amino; —NH-aryl, and a combination thereof; and
E is:
aryl or heteroaryl, optionally substituted with one or more substituents selected from C₁-C₆ alkyl or alkoxy, —(C₁-C₆ alkyl)-OH, —(C₁-C₆ alkyl)-COOH, —(C₁-C₆ alkyl)-NH₂, halo, nitro, hydroxy, amino, C₁-C₆ alkylamino, di-C₁-C₆ alkyl-amino; —NH-aryl, C₁-C₆ haloalkyl, C₃-C₈ cycloalkyl or heterocycloalkyl, fused C₃-C₈ cycloalkyl or heterocycloalkyl, aryl or heteroaryl, fused aryl or heteroaryl, —CN, —(C₁-C₃ alkyl)-CN, carbonyl, and a combination thereof;
amino, C₁-C₆ alkylamino, or di-C₁-C₆ alkyl-amino; or —NH-aryl;
C₁-C₆ alkyl or alkoxy, optionally substituted with one or more substituents selected from hydroxy, C₁-C₆ alkyl, amino, C₁-C₆ alkylamino, or di-C₁-C₆ alkyl-amino; —NH-aryl, and a combination thereof;
C₃-C₈ cycloalkyl or heterocycloalkyl, optionally substituted with one or more substituents selected from hydroxy, C₁-C₆ alkyl, amino, C₁-C₆ alkylamino, di-C₁-C₆ alkyl-amino; —NH-aryl, C₃-C₈ cycloalkyl or heterocycloalkyl, fused C₃-C₈ cycloalkyl or heterocycloalkyl, aryl or heteroaryl, fused aryl or heteroaryl, and a combination thereof;
—C(O)NH₂;
—C(O)OH;
—C(O)H;
—N—(C₁-C₆ alkyl)-acrylamide;
halogen; or
hydrogen;
or a pharmaceutically acceptable salt thereof.

2. The compound of claim 1, wherein [symbol]
is a single or double bond,
X¹ and X² are each independently CH or N;
X³ is S, S═O, or S(═O)₂;
R¹ is H or —NR²R³;
R² is H or C₁-C₃ alkyl;
R³ is an aryl;
A is optional and, when present, is —C(O)—, —C(O)O—, —C(O)NH—, —C(O)N(C₁-C₃-akyl)-; —C(O)NH—(C₁-C₆ alkyl)-NHC(O)—, —NH;
D is optional and, when present, is a C₁-C₆ alkyl, —(C₁-C₆ alkyl)-O—, —(C₁-C₆ alkyl)-NH—, —(C₁-C₃ alkyl)-O—(C₁-C₃ alkyl)-, or —(C₁-C₃ alkyl)-NH—(C₁-C₃ alkyl)-, wherein the alkyl group of any of the foregoing is optionally substituted with hydroxy; and
E is:
aryl or heteroaryl, optionally substituted with C₁-C₆ alkyl or alkoxy, halo, nitro, hydroxy, C₁-C₆ haloalkyl, —CN, —(C₁-C₃ alkyl)-CN, or carbonyl;
amino, C₁-C₆ alkylamino, or di-C₁-C₆ alkyl-amino; or —NH-aryl;
C₁-C₆ alkyl or alkoxy, optionally substituted with hydroxy;
C₃-C₈ cycloalkyl or heterocycloalkyl, optionally substituted with hydroxy;
—C(O)NH₂;
—C(O)OH;
—C(O)H;
—N—(C₁-C₆ alkyl)-acrylamide;
halogen; or
hydrogen;
or a pharmaceutically acceptable salt thereof.

3. The compound of claim 1, wherein [symbol] is a double bond.

4. The compound of claim 1, wherein the compound of formula (I) is of formula (Ia): 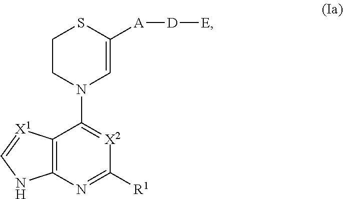
or a pharmaceutically acceptable salt thereof.

5. The compound of claim 1, wherein the compound of formula (I) is of formula (Ib): 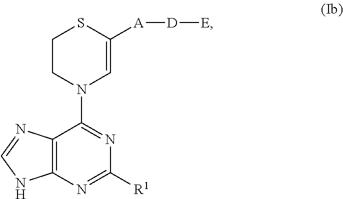
or a pharmaceutically acceptable salt thereof.

6. The compound of claim 1, wherein R¹ is H.

7. The compound of claim 1, wherein:
(i) A and D are absent, and E is halogen; —C(O)OH; —C(O)H; aryl or heteroaryl, optionally substituted with C₁-C₃ alkyl, or halo; or C₃-C₈ cycloalkyl or heterocycloalkyl, optionally substituted with hydroxy;
(ii) A is absent; D is a C₁-C₆ alkyl, optionally a C₁-C₃ alkyl; and E is —C(O)NH₂ or a C₃-C₈ cycloalkyl or heterocycloalkyl, optionally substituted with hydroxy;
(iii) A is —NH—, —C(O)NH— or —C(O)N(C₁-C₃-akyl)-; D is absent or is a C₁-C₆ alkyl, optionally a C₁-C₃ alkyl; and E is amino, C₁-C₆ alkylamino, or di-C₁-C₆ alkyl-amino; or —NH-aryl; C₁-C₆ alkyl or alkoxy or C₁-C₃ alkyl or alkoxy, optionally substituted with hydroxy;
or aryl or heteroaryl, optionally substituted with C₁-C₃ alkyl or alkoxy, halo, nitro, hydroxy, C₁-C₃ haloalkyl, —CN, —(C₁-C₃ alkyl)-CN, or carbonyl;
(iv) A is absent, D is —(C₁-C₆ alkyl)-O—, —(C₁-C₆ alkyl)-NH—, —(C₁-C₃ alkyl)-O—(C₁-C₃ alkyl)-, or —(C₁-C₃ alkyl)-NH—(C₁-C₃ alkyl)-, wherein the alkyl groups of any of the foregoing is optionally substituted with hydroxy and wherein the alkyl groups of any of the foregoing are optionally branched; and E is C₁-C₆ alkyl or C₃-C₈ cycloalkyl or heterocycloalkyl, optionally substituted with hydroxy;
(v) A is —C(O)NH—(C₁-C₆ alkyl)-NHC(O)—, optionally C(O)NH—(C₁-C₃ alkyl)-NHC(O)—; D is absent; and E is C₁-C₆ alkyl or C₁-C₃ alkyl; or aryl or heteroaryl, optionally substituted with C₁-C₃ alkyl or alkoxy, halo, nitro, hydroxy, C₁-C₆ haloalkyl, —CN, —(C₁-C₃ alkyl)-CN, or carbonyl;
(vi) A is —C(O)O—; D is absent; and E is C₁-C₆ alkyl; or
(vii) A is —C(O)—; D is absent; and E is heterocyloalkyl.

8. The compound of claim 1, wherein the compound of formula (I) is of formula (Ic): 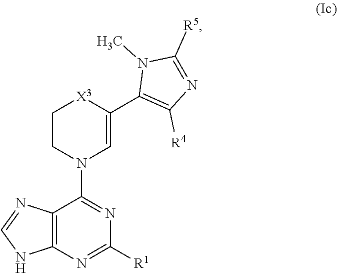
wherein R⁴ and R⁵ are the same or different and each is H or halo, or a pharmaceutically acceptable salt thereof.

9.-10. (canceled)

11. The compound of claim 1, wherein the compound is not aplithianine A:

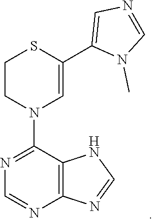 [SMILES: Cn1cncc1C1=CN(c2ncnc3nc[nH]c23)CCS1]

12. The compound of claim 1, wherein the compound of formula (I) is selected from 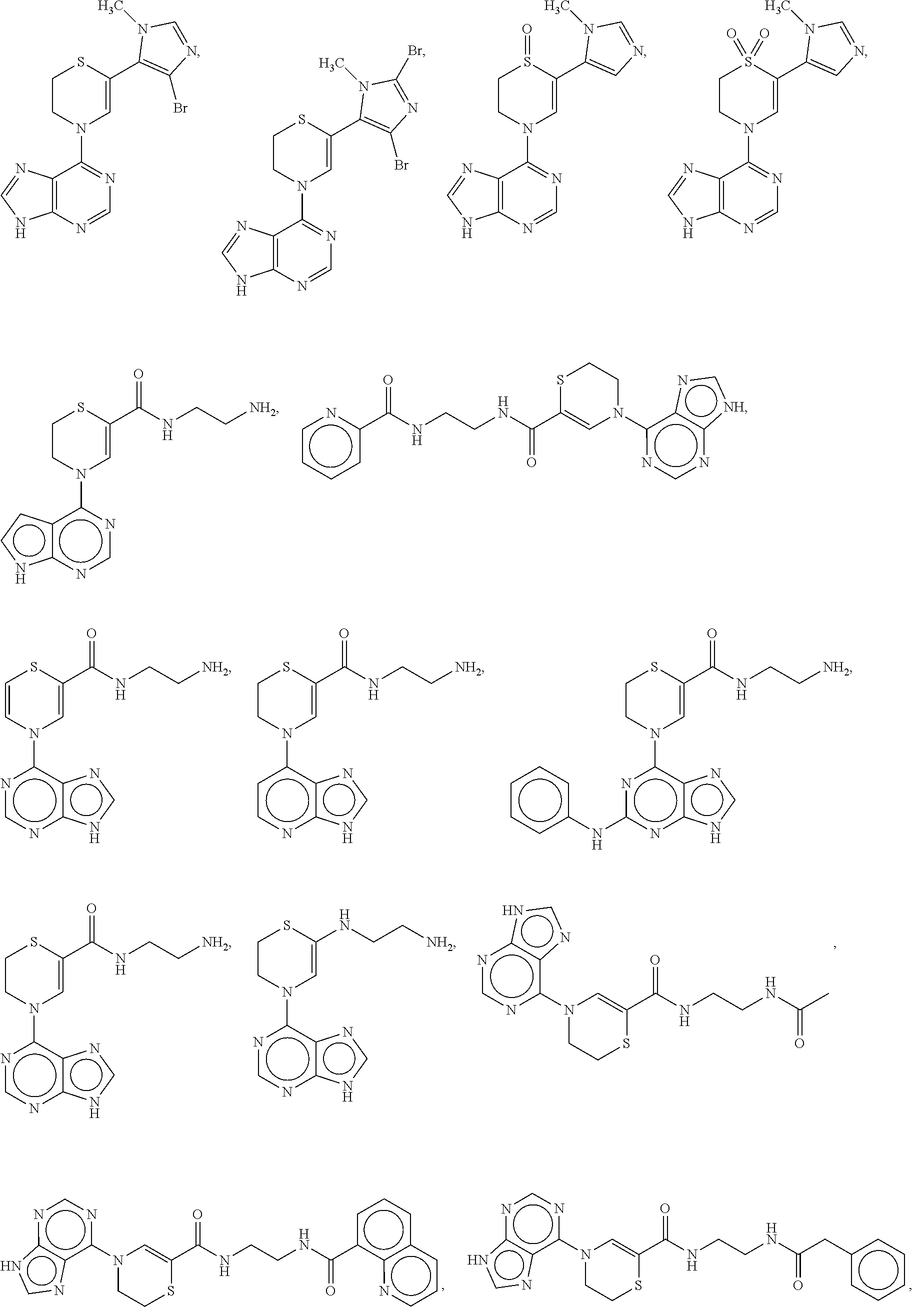 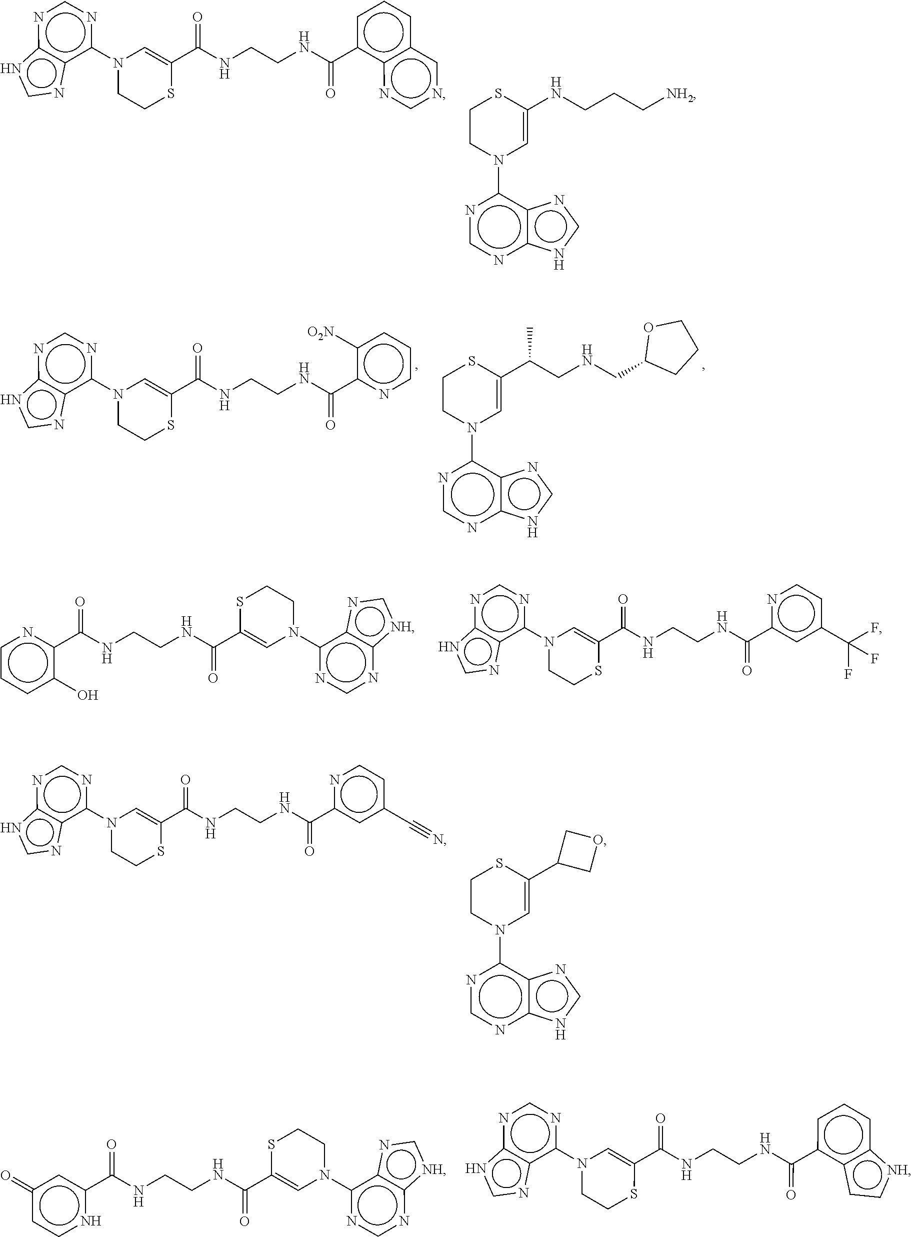 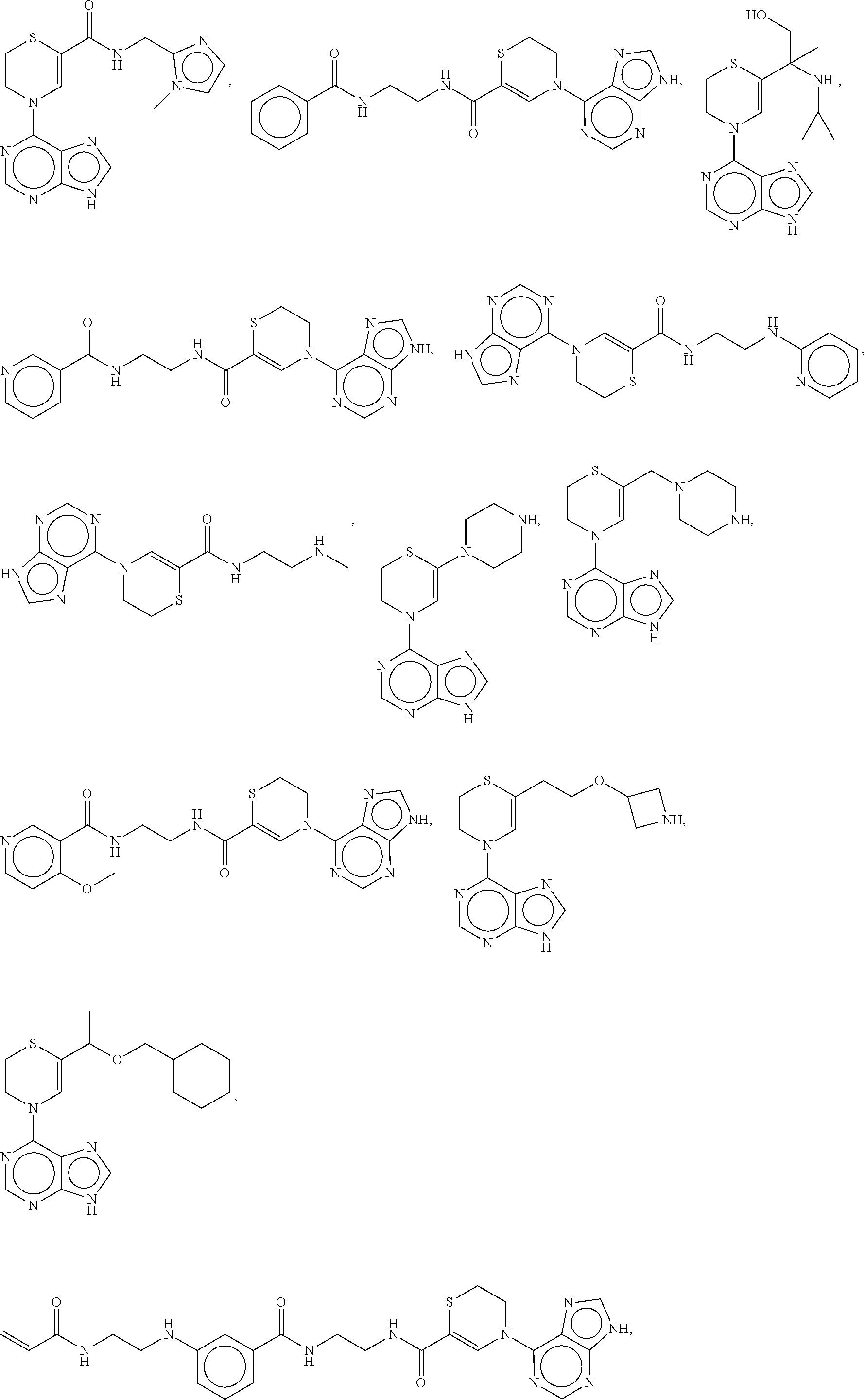 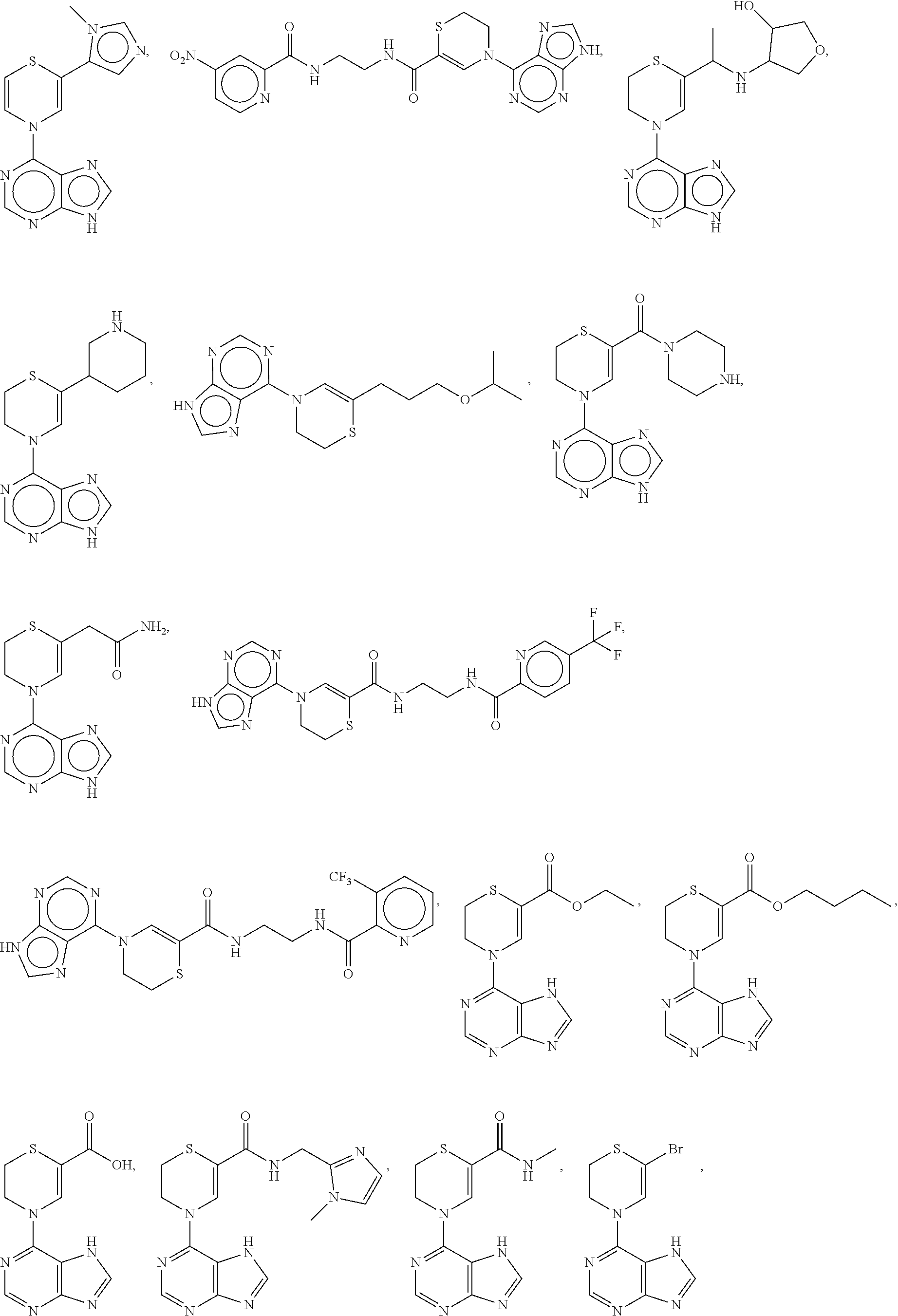 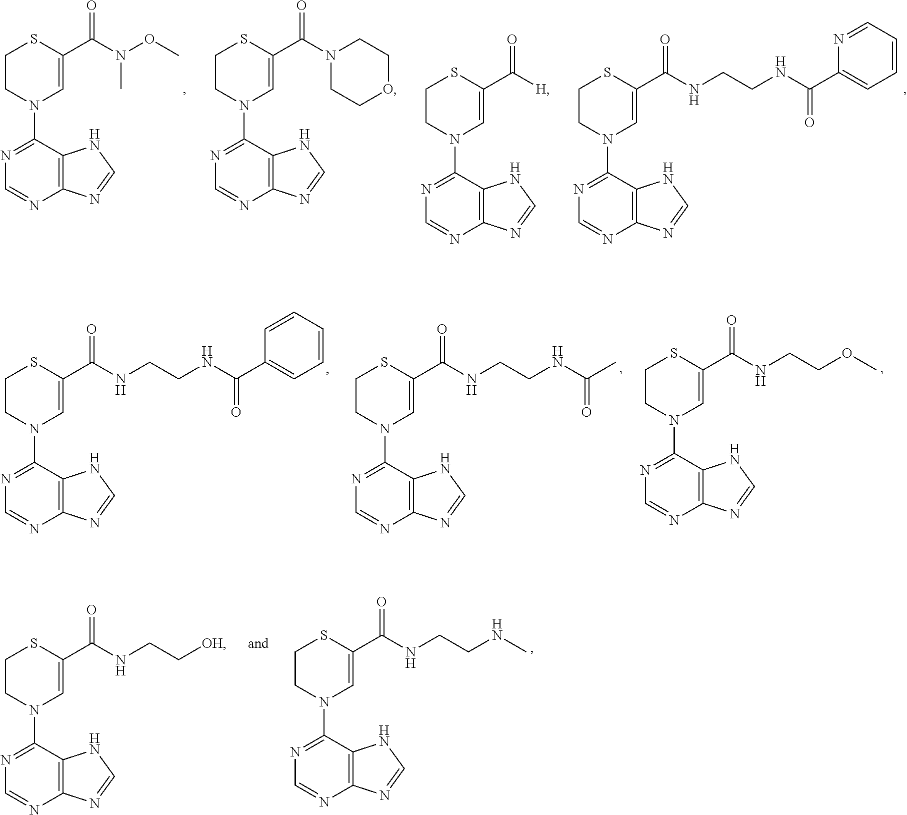
or a pharmaceutically acceptable salt thereof.

13. The compound of claim 12, wherein the compound of formula (I) is selected from 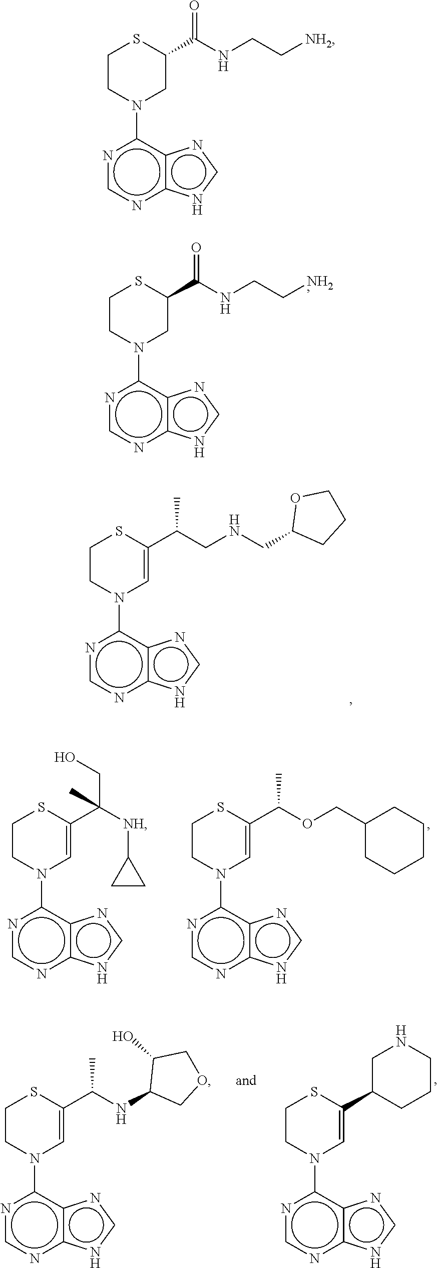
or a pharmaceutically acceptable salt thereof.

14. A compound 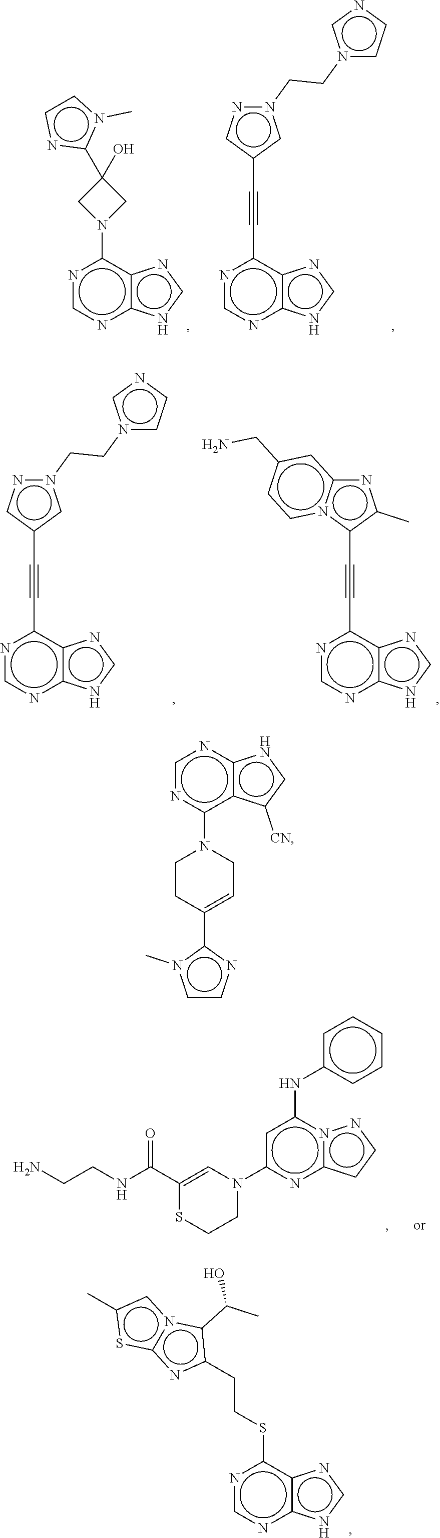
or a pharmaceutically acceptable salt thereof.

15. A pharmaceutical composition comprising a compound of claim 1 and a pharmaceutical carrier.

16. A pharmaceutical composition comprising 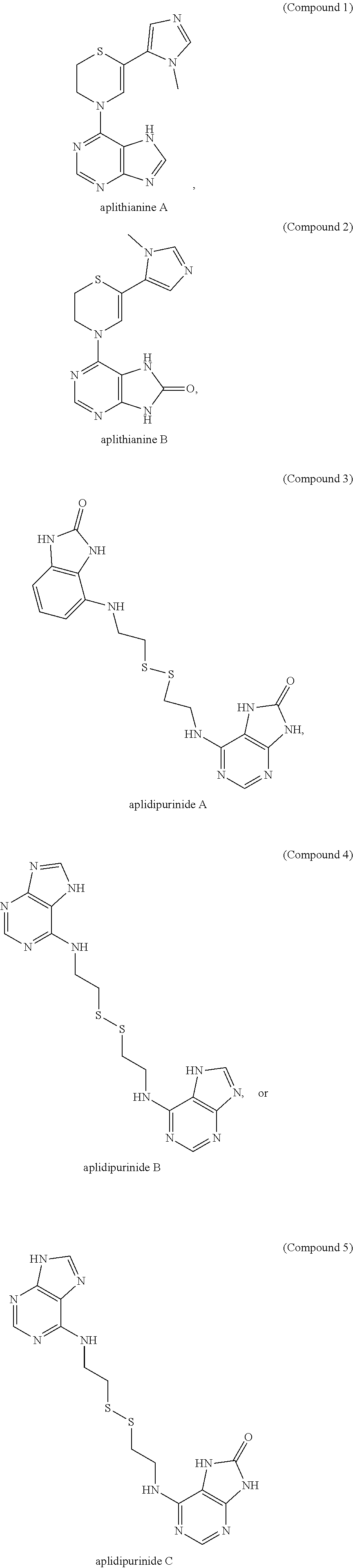 [SMILES: Cn1cncc1C1=CN(c2ncnc3nc[nH]c23)CCS1 ; Cn1cncc1C1=CN(c2ncnc3[nH]c(=O)[nH]c23)CCS1 ; O=c1[nH]c2cccc(NCCSSCCNc3ncnc4[nH]c(=O)[nH]c34)c2[nH]1 ; c1nc(NCCSSCCNc2ncnc3nc[nH]c23)c2[nH]cnc2n1 ; O=c1[nH]c2ncnc(NCCSSCCNc3ncnc4[nH]cnc34)c2[nH]1]
with at least 80% purity, and a pharmaceutical carrier.

17. A method of inhibiting kinase activity in a subject, the method comprising administering to the subject a compound of claim 1.

18.-22. (canceled)

23. A method of suppressing the immune system in a subject, the method comprising administering to the subject a compound of claim 1.

24. A method of preventing organ rejection in a subject, the method comprising administering to the subject a compound of claim 1.

25. A method of treating cancer in a subject, the method comprising administering to the subject a compound of claim 1.

26.-29. (canceled)

30. A method of treating diabetic neuropathic pain in a subject, the method comprising administering to the subject a compound of claim 1.

31. A method of treating malaria in a subject, the method comprising administering to the subject a compound of claim 1.

32. A method of treating an infection associated with a protozoa in a subject, the method comprising administering to the subject a compound claim 1.

33. (canceled)

34. A method of making aplithianine A 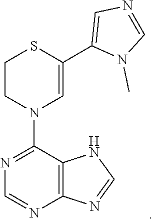 [SMILES: Cn1cncc1C1=CN(c2ncnc3nc[nH]c23)CCS1]
comprising coupling a purine-thiazine conjugate having the following structure: 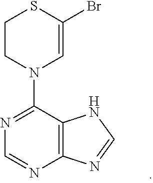 [SMILES: BrC1=CN(c2ncnc3nc[nH]c23)CCS1]
with an imidazole having the following structure: 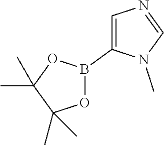
to provide apithianine A.

35. (canceled)

36. A method of preparing a compound of claim 8: 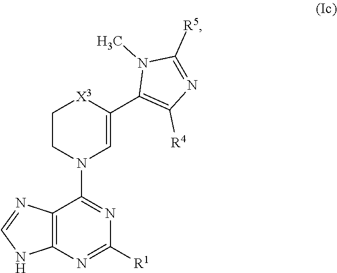
wherein at least one of R³ or R⁴ is halogen;
which method comprises halogenating a compound of formula:

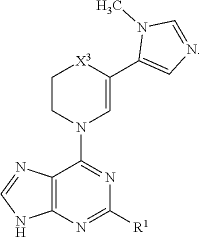

37.-38. (canceled)
